(19) United States
(12) Patent Application Publication
(10) Pub. No.: US 2026/0076973 A1
(43) Pub. Date: Mar. 19, 2026

(54) Compounds and methods for treating diseases caused by SARS-CoV-2 and other coronaviruses

(71) Applicant: Viradem, Inc., Redondo Beach, CA (US)

(72) Inventors: Anatolii R Demchenko, Chernigiv (UA); Eugene Stavtsev, Redondo Beach, CA (US); Sergii Demchenko, Kyiv (UA); David Ezra Gordon, Atlanta, GA (US); Valery Rostislavovich Polyakov, Oakley, CA (US)

(73) Assignee: Viradem, Inc., Redondo Beach, CA (US)

(21) Appl. No.: 19/110,080

(22) PCT Filed: Jan. 12, 2024
(86) PCT No.: PCT/US2024/011398

§ 371 Date: Mar. 9, 2025

Related U.S. Application Data
(60) Provisional application No. 63/480,013, filed on Jan. 15, 2023.

(51) Int. Cl. A61K 31/5377; A61P 31/14

(52) U.S. Cl. CPC A61K 31/5377; A61P 31/14

(57) ABSTRACT

Provided are compounds and methods of treating diseases caused by a Coronaviridae virus such as SARS-CoV-2, SARS-CoV, and MERS-CoV coronaviruses by administering nitrogen-containing heterocyclic compounds. SARS (Severe Acute Respiratory Syndrome), MERS (Middle East Respiratory Syndrome), and, particularly, COVID-19 are highly contagious diseases with high mortality rate responsible for tens of millions of infections and almost two million deaths around the globe.

### CROSS REFERENCE TO RELATED APPLICATION

This application claims the benefit of U.S. Provisional Application No. 63/480,013 filed Jan. 15, 2023, the contents of which are hereby incorporated by reference in its entirety.

### PRIOR ART

Sørensen M D; Sørensen B; Gonzalez-Dosal R; Melchjorsen C J; Weibel J; Wang J; Jun C W; Huanming Y; Kristensen P (May 2006). “Severe acute respiratory syndrome (SARS): development of diagnostics and antivirals”//Annals of the New York Academy of Sciences. 1067 (1): 500-5. 2006-05-0.
Distribution of confirmed cases of MERS-CoV by place of infection and month of onset, from March 2012 to 2 Dec. 2019 European Centre for Disease Prevention and Control (6 Dec. 2019).
Montalbetti, Christian A. G. N.; Coulter, Thomas S.; Uddin, Muhammed K.; Reignier, Serge G.; Magaraci, Filippo; Granaes, Charlotta; Krog-Jensen, Christian; Felding, Jakob; Tetrahedron Letters; vol. 47; nb. 33; (2006); p. 5973-5975.
Treatment of prostate cancer, melanoma or hepatic cancer US 2007 244 110A1.
Sunduru, Naresh; Gupta, Leena; Chauhan, Prem M. S.; Chaturvedi, Vinita; Dwivedi, Richa; Sinha, Sudhir; European Journal of Medicinal Chemistry; vol. 45; nb. 8; (2010); p. 3335-3345.
Patent No 67510 Ukraine, International Filing Date: 21 Jul. 2011; Publication Date: 27 Feb. 2012.
Patent No 86038 Ukraine, International Filing Date: 12 Jun. 2013; Publication Date: 10.12.20131.
Patent No 95732 Ukraine, International Filing Date: 15 May 2014; Publication Date: 12 Jan. 2015.
Patent No 100048 Ukraine, International Filing Date: 10 Dec. 2014; Publication Date: 10 Jul. 2015.
Patent No 116386 Ukraine, International Filing Date: 9 Feb. 2016; Publication Date: 12 Mar. 2018.
Patent No 118704 Ukraine, International Filing Date: 12 Jan. 2017; Publication Date: 25 Feb. 2019.
Patent No 118887 Ukraine, International Filing Date: 20 Dec. 2016; Publication Date: 25 Mar. 2019.
Patent No 120051 Ukraine, International Filing Date: 24 Nov. 2016; Publication Date: 25 Sep. 2019.
Patent No 137987 Ukraine, International Filing Date: 15 May 2019; Publication Date: 11 Nov. 2019.
Patent No 122175 Ukraine, International Filing Date: 15 Nov. 2018; Publication Date: 25 Sep. 2020.
Patent No 134396 Ukraine, International Filing Date: 27 Dec. 2018; Publication Date: 10 May 2019.
Patent No 122187 Ukraine, International Filing Date: 15 Jan. 2019; Publication Date: 25 Sep. 2020.
A. M. Demchenko, O. V. Moskalenko, V. V. Sukhoveev, A. I. Barchyna. Synthesis and antiviral activity of 2-(4,6-dimorpholine-4-yl-1,3,5-triazine-2-yl)-N-arylhydrazinecarbothioamides for Flu A N1H1 virus//Pharmacology and drug toxicology.-2019.-No 1, v. 13.-P. 35-41.
Michael O'Donnell, Jean-Damien Charrier. A complementary route to diaminopyrimidines through regioselective SNAr amination reactions/Tetrahedron.-2015.-v. 71.-1515-1522.

### BACKGROUND OF THE INVENTION

SARS-CoV, MERS-CoV, and SARS-CoV-2 are a family of coronaviruses.

SARS (Severe Acute Respiratory Syndrome)—a respiratory viral disease, the first case of which was reported in November 2002 in southern China. The disease is characterized by viral pneumonia, rapidly progressing to respiratory failure. Cases were detected in 29 countries, with a total of 8098 cases, of which 774 were fatal. The largest number of cases was recorded in China, Hong Kong, Taiwan, Singapore and Canada. During the 2003 SARS outbreak, about 10% of patients with a confirmed diagnosis of SARS-CoV died. Mortality was much higher in patients over 65 years of age, with a case fatality rate approaching 50% for this subgroup of patients.

MERS (Middle East Respiratory Syndrome)—an inflammatory respiratory disease caused by a virus of the genus Betacoronavirus, subfamily Coronaviridae. The first cases of the new virus were reported in Saudi Arabia in early fall 2012. Of the more than 50 reported MERS cases by June 2013, about half were fatal. As of the beginning of 2020, up to 2506 cases of the disease were registered in the world, up to 912 people died. Fatality rate of about 36%. MERS-CoV virus cases continue to be reported each year.

SARS-CoV-2 (Severe acute respiratory syndrome-related coronavirus 2)—single-stranded RNA virus belonging to the genus Betacoronavirus. First identified in December 2019, it causes a dangerous infectious disease—COVID-19. The coronavirus SARS-CoV-2 is due to the ongoing COVID-19 pandemic. The infection can occur in the form of a mild acute respiratory viral infection, or in a severe form. Complications of severe cases can include pneumonia or respiratory failure with risk of death. The transmission of infection occurs by droplets, by touching the infected parts of the body to the mucous membranes, also through tactile touch to food. As of Jan. 9, 2024, 701.2 million cases of infection have been confirmed, 6.965 million deaths.

### SUMMARY OF THE INVENTION

Simple and often known chemical compounds are shown to be effective inhibitors of coronaviruses. A lot of such compounds are not cytotoxic for host cells and can provide an inexpensive way of treating infections caused by several viruses belonging to the Coronaviridae family.

### DETAILED DESCRIPTION OF THE INVENTION

The present disclosure provides a method of treating a disease caused by SARS-CoV-2 and other coronaviruses, comprising administering to a subject in need thereof a therapeutically effective amount of a compound of Formula I:

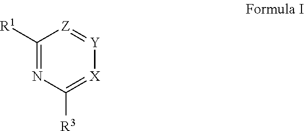

or a pharmaceutically acceptable salt thereof;
wherein:
   each X, Y, and Z is independently selected from N or C—R²;
   each R¹, R², and R³ is independently from the group consisting of hydrogen, halogen, nitro, cyano, hydroxyl, amino, alkyl, cycloalkyl, optionally substituted cycloalkyl, (cycloalkyl)alkyl, bicycloalkyl, spiroalkyl, optionally substituted alkyl, haloalkyl, hydroxyalkyl, alkoxy, haloalkoxy, alkylthio, alkoxyalkyl, (cyano)alkyl, alkenyl, optionally substituted alkenyl, alkynyl, cycloalkylenyl, optionally substituted alkynyl, sulfonamide, alkylcarbonyl, arylcarbonyl, alkylsulfonyl, arylsulfonyl, mercaptoalkyl, carboxy, carboxamido, ureido, (heterocyclo)alkyl, (heteroaryl)alkyl, (amino)(heteroaryl)alkyl, (amino)(aryl)alkyl, heteroalkyl, heterocyclo, optionally substituted heterocyclo, hetero-substituted cycloalkyl, hetero-substituted bicycloalkyl, heteroaryl, optionally substituted heteroaryl, hetero-substituted spiroalkyl, aryl, optionally substituted aryl, N-substituted optionally substituted aryl, N-substituted heteroaryl, N-substituted optionally substituted heteroaryl, aryloxy, heteroaryloxy, aralkyloxy, alkylamino, dialkylamino, hydroxyalkylamino, cycloalkylamino, aralkylamino, (cycloalkyl)alkylamino, aralkyl, (aryl)alkyl, (heterocyclo)alkylamino, (amino)alkyl, heteroalkylenyl, heterocyclenyl, N-aminoacid,

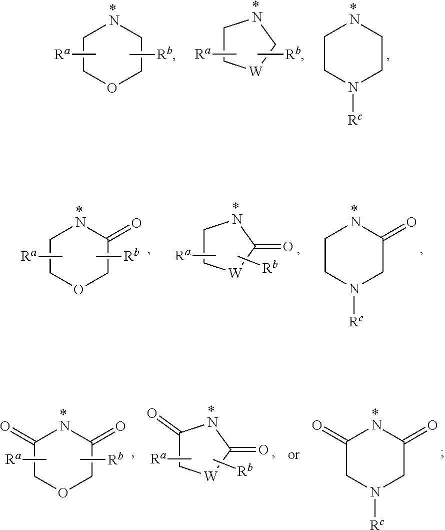

R¹ and R² could be the same, but, in this case, always different from R³,
   wherein:
      each R^{a}, R^{b}, and R^{c} independently selected from the group consisting of hydrogen, C₁-C₁₆ alkyl, or an optionally substituted alkyl, aryl, optionally substituted aryl, and heteroalkyl.

The term “halogen” as used herein by itself or as part of another group refers to —Cl, —F, —Br, or —I.

The term “nitro” as used herein by itself or as part of another group refers to —NO2.

The term “cyano” as used herein by itself or as part of another group refers to —CN.

The term “hydroxyl” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently halogen, nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R)C(═O)R, —N(R)S(═O)₂R, —C(═O)R, —S(═O)R, or —S(═O)₂R.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂-groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, spiro-fused rings, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R)C(═O)R, —N(R)S(═O)₂R, —C(═O)R, —S(═O)R, —S(═O)₂R, or —OR. Where R is C₁-C₁₆ alkyl, or an optionally substituted alkyl, aryl or an optionally substituted aryl, heteroalkyl.

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., —S(═O)—, or sulfone, i.e., —S(═O)₂—.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as oxazine-1,4; 3,4-dihydro-2H-benzo[1,4]oxazine, 4H-benzo[1,4]oxazine-3-one, 3,4-dihydro-2H-benzo[1,4]thiazine, 4H-benzo[1,4]thiazine-3-one, 1,2,3,4-tetrahydroquinoxaline, 3,4-dihydro-1H-quinoxalin-2-one, 10H-phenoxazine, 2,3-dihydrobenzooxalole, 3H-benzooxazol-2-one, indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is an 8- to 12-membered cyclic group containing two rings and one or two nitrogen atoms. The heterocyclo can be linked to the rest of the molecule through any available carbon or nitrogen atom.

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, oxy optionally substituted alkyl, (heterocyclo)alkyl, (heteroaryl)alkyl, —N(R)C(═O)R, —N(R)S(═O)₂R, —C(═O)R, —S(═O)R, —S(═O)₂R, or —OR. Where R is C₁-C₁₆ alkyl, or an optionally substituted alkyl, aryl or an optionally substituted aryl, heteroalkyl.

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl, 2,3-di-methylphenyl, 3,4-di-methylphenyl, 2,5-di-methylphenyl, 2,6-di-methylphenyl, 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, b-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R)C(═O)R, —N(R)S(═O)₂R, —C(═O)R, —S(═O)R, —S(═O)₂R, or —OR. Where R is C₁-C₁₆ alkyl, or an optionally substituted alkyl, aryl or an optionally substituted aryl, heteroalkyl.

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups.

The term “sulfonamido” as used herein by itself or as part of another group refers to a radical of the formula —SO₂NRR₁, wherein R and R₁ are each independently hydrogen, alkyl, optionally substituted cycloalkyl, optionally substituted heterocyclo, optionally substituted aryl, or optionally substituted heteroaryl; or R and R₁ taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “carboxamido” as used herein itself or as part of another group refers to a radical of the formula —C(═O)NRR₁, wherein R and R₁ are each independently hydrogen, alkyl, optionally substituted cycloalkyl, optionally substituted heterocyclo, optionally substituted aryl, optionally substituted heteroaryl, aralkyl, (heteroaryl)alkyl, or (heterocyclo)alkyl; or R and R₁ taken together with the nitrogen to which they are attached form a 3- to 8-membered optionally substituted heterocyclo group. Non-limiting exemplary carboxamido groups include —C(═O)NH₂, —C(═O)N(H)CH₃, and —C(═O)N(H)Ph

The term “ureido” as used herein by itself or as part of another group refers to a radical of the formula —NR—C(═O)—NRR₁, wherein R is hydrogen or alkyl; and R and R₁ are each independently hydrogen, alkyl, optionally substituted cycloalkyl, optionally substituted heterocyclo, optionally substituted aryl, or optionally substituted heteroaryl, or R and R₁ taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl.

The term “(amino)(heteroaryl)alkyl” as used herein by itself or as part of another group refers to an alkyl group substituted with one optionally substituted heteroaryl group and one amino group. In one embodiment, the heteroaryl is an optionally substituted 5- to 9-membered heteroaryl group. In another embodiment, the heteroaryl is an optionally substituted 5- or 6-membered heteroaryl group. In one embodiment, the alkyl is a C₁-C₆ alkyl. In another embodiment, the alkyl is a C₁-C₄ alkyl. In another embodiment, the alkyl is a C₁ or C₂ alkyl.

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “(amino)(aryl)alkyl” as used herein by itself or as part of another group refers to an alkyl group substituted with one amino group and one optionally substituted aryl group. In one embodiment, the amino group is —NH₂, alkylamino, or dialkylamino. In one embodiment, the aryl group is an optionally substituted phenyl. In one embodiment, the alkyl is a C₁-C₆ alkyl. In another embodiment, the alkyl is a C₁-C₄ alkyl.

The term “amino” as used by itself or as part of another group refers to a radical of the formula —NRR₁, wherein R and R₁ are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R is C₁-C₆alkyl and R₁ is hydrogen. In one embodiment, R is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R and R₁ are each independently C₁-C₆ alkyl. In one embodiment, R and R₁ are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R is (hydroxyl)alkyl and R₁ is hydrogen or C₁-C₄ alkyl.

In another embodiment, the amino is a “cycloalkylamino,” i.e., an amino group wherein R is optionally substituted cycloalkyl and R₁ is hydrogen or C₁-C₄ alkyl.

In another embodiment, the amino is an “aralkylamino,” i.e., an amino group wherein R is aralkyl and R₁ is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(cycloalkyl)alkylamino,” i.e., an amino group wherein R is (cycloalkyl)alkyl and R₁ is hydrogen or C₁-C₄ alkyl.

In another embodiment, the amino is a “(heterocyclo)alkylamino,” i.e., an amino group wherein R is (heterocyclo)alkyl and R₁ is hydrogen or C₁-C₄ alkyl.

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

In the present disclosure, the term “alkylenyl” as used herein by itself or part of another group refers to a divalent form of an alkyl group, wherein the alkyl group is either unsubstituted or substituted with one or two groups independently selected from the group consisting of optionally substituted phenyl and optionally substituted 5- or 6-membered heteroaryl. In one embodiment, the alkylenyl is a divalent form of a C₁-C₁₂ alkyl. In one embodiment, the alkylenyl is a divalent form of a C₁-C₁₀ alkyl. In one embodiment, the alkylenyl is a divalent form of a C₁-C₈ alkyl. In one embodiment, the alkylenyl is a divalent form of an unsubstituted C₁-C₆ alkyl. In another embodiment, the alkylenyl is a divalent form of an unsubstituted C₁-C₄ alkyl. In another embodiment, the alkylenyl is a divalent form of a C₁-C₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In one embodiment, the heteroalkylenyl is a divalent form of a 3- to 20-membered heteroalkyl. In another embodiment, the heteroalkylenyl is a divalent form of a 3- to 10-membered heteroalkyl. In another embodiment, the heteroalkylenyl is a divalent form of a 3- to 8-membered heteroalkyl. In another embodiment, the heteroalkylenyl is a divalent form of a 3- to 6-membered heteroalkyl. In another embodiment, the heteroalkylenyl is a divalent form of a 3- to 4-membered heteroalkyl. In another embodiment, the heteroalkylenyl is a radical of the formula —(CH₂CH₂O)u1- wherein u1 is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted 4- to 8-membered heterocyclo group. In one embodiment, the heterocyclenyl is a divalent form of an optionally substituted azetidine. In one embodiment, the heterocyclenyl is a divalent form of an optionally substituted piperidinyl.

The term “cycloalkylenyl” as used herein by itself or part of another group refers to a divalent form of an optionally substituted C₄-C₆ cycloalkyl group. In one embodiment, the cycloalkylenyl is a 4-membered cycloalkylenyl. In another embodiment, the cycloalkylenyl is a 5-membered cycloalkylenyl. In another embodiment, the cycloalkylenyl is a 6-membered cycloalkylenyl.

The term “N-aminoacid” as used by itself or as part of another group refers to any —R or —S amino acid independently selected from:

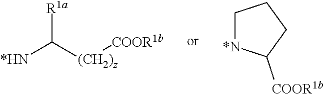

wherein,
R^{1a} is independently selected from H, CH₃, CH(CH₃)₂), CH₂CH(CH₃)₂, CH(CH₃)CH₂CH₃, CH₂OH, CH₂SH, CH₂CH₂SCH₃, CH₂CH₂SeCH₃, CH(OH)CH₃, CH₂CONH₂, CH₂CH₂CONH₂, CH₂COOH, CH₂CH₂COOH, CH₂CH₂CH₂CH₂NH₂, CH₂CH₂CH₂NHC(NH)NH₂, CH₂Ph, CH₂C₆H₄OH, CH₂(β-indolyl), CH₂(imidazolyl-4(5)), alkyl, optionally substituted alkyl, haloalkyl, cycloalkyl, optionally substituted cycloalkyl, (heterocyclo)alkyl;
z is selected from 0, 1, 2, 3, 4, 5, or 6;
R^{1b} is independently selected from H or alkyl.

The term “N substituted” as used herein by itself or as part of another group refers to a substituted nitrogen attached to the central ring in Formula I.

The term “oxy optionally substituted alkyl” as used herein by itself or as part of another group refers to a substituted oxygen covalently bound to any atom of the claimed molecule and to the above-mentioned optionally substituted alkyl.

The present disclosure also encompasses any of the compounds of the disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

Biological tests have been carried out in Southern Research by: Raj Kalkeri, Ph. D, MBA & J. Robert Bostwick, Ph. D Director, HTS Center Southern Research 2000 Ninth Avenue South Birmingham, AL 35205 205-581-2686

Compounds from supplied as dry powder were solubilized in DMSO at 10 mM or further diluted to 333 μM and stored at 4° C. until the day of the assays. A set of positive control antiviral compounds were included in each of the assays performed.

Screening Strategy: A cell-based assay measuring the cytopathic effect (CPE) of the virus infecting Vero E6 host cells was used in screening. The CPE reduction assay is a popular and widely used assay format to screen for antiviral agents because of its ease of use in high throughput screening (HTS). In this assay, host cells infected with virus die as a consequence of the virus infection. The CPE reduction assay indirectly monitors the effect of antiviral agents acting through various molecular mechanisms by measuring the viability of host cells three days after inoculation with virus. Anti-viral compounds are identified as those that protect the host cells from the cytopathic effect of the virus, thereby increasing viability.

Compound preparation: Compound solutions (80 μL of either 10 mM or 333 μM stock solutions in 100% DMSO) were transferred into wells in columns 3-22 of an empty ECHO plate (stock plate). Compounds were diluted 3-fold by transferring 17 μL of each sample from the stock plate into another ECHO plate with 34 μL DMSO in each well and mixing. This process was repeated to create 8 more plates of serially diluted sample, each plate containing a 3-fold diluted sample of the previous plate. A 30 nL aliquot for each diluted sample was dispensed into corresponding wells of assay plates using an ECH0550 acoustic liquid handling system. An equal amount of DMSO was added to control wells in columns 1, 2, 23, and 24. The final assay concentration range was 30-0.002 μM prepared from the 10 mM stocks in the initial screen and 1000-0.05 nM prepared from the 333 μM stocks in the second screen.

Method for measuring antiviral effect of compounds: Vero E6 cells were grown in MEM supplemented with 10% HI FBS and harvested in MEM/1% PS supplemented 2% HI FBS on the day of assay. Assay ready plates pre-drugged with test compounds were prepared in the BSL-2 lab by adding 5 μL assay media to each well. The plates and cells were then passed into the BSL-3 facility. Cells were batch inoculated with SARS CoV-2 (M.O.I.˜0.002) which results in 5% cell viability 72 hours post infection. A 25 μL aliquot of virus inoculated cells (4,000 Vero E6 cells/well) was added to each well in columns 3-24 of the assay plates. The wells in columns 23-24 contained only virus infected cells for the 0% CPE reduction controls. Prior to virus inoculation, a 25 μL aliquot of cells was added to columns 1-2 of each plate for the cell only 100% CPE reduction controls. After incubating plates at 37° C./5% CO2 and 90% humidity for 72 hours, 30 μL of Cell Titer-Glo (Promega) was added to each well. Luminescence was read using a BMG CLARIOstar plate reader following incubation at room temperature for 10 minutes to measure cell viability. Plates were sealed with a clear cover and surface decontaminated prior to luminescence reading.

Method for measuring cytotoxic effect of compounds: Compound cytotoxicity was assessed in a BSL-2 counter screen as follows: VeroE6 cells in media were added in 25 μl aliquots (4,000 cells/well) to each well of assay ready plates prepared with test compounds as above. Cells only (100% viability) and cells treated with hyamine at 100 μM final concentration (0% viability) serve as the high and low signal controls, respectively, for cytotoxic effect in the assay. DMSO was maintained at a constant concentration for all wells as dictated by the dilution factor of stock test compound concentrations. After incubating plates at 37° C./5% CO2 and 90% humidity for 72 hours, 30 μl Cell Titer-Glo (Promega) was added to each well. Luminescence was read using a BMG PHERAstar plate reader following incubation at room temperature for 10 minutes to measure cell viability.

Data analysis: For all assays the raw data from plate readers were imported into ActivityBase where values were associated with compound IDs and test concentrations. For the antiviral CPE reduction assay, raw signal values are converted to % CPE reduction by the following formula:

% CPE reduction=100×(test cmpd value−mean value infected cell controls)/(mean value uninfected cell controls−mean value infected cell controls).

For the cell viability assay measuring compound cytotoxicity, % cell viability is calculated as follows:

% viability=100*(test cmpd value−mean low signal control)/(mean high signal control−mean low signal control).

EC₅₀ and CC₅₀ values were calculated from a four-parameter logistic fit of data using the Xlfit module of ActivityBase. Raw data for both antiviral activity and toxicity with a graphical representation of the data are provided in separate excel files submitted to the client. Summary data for the reference compounds and test articles is provided in Table 1.

TABLE 1
Antiviral activity of Reference Compounds in the SARS-CoV-2 (WA1)
CPE assay, Round 2. (Concentration range of 1000-0.05 nM)

| Compound | | | | Antiviral | Cytotoxicity | Selectivity Index |
|---|---|---|---|---|---|---|
| ID | Batch | Supplier | Supplier ID | IC₅₀ (μM) | CC₅₀ (μM) | (CC₅₀/IC₅₀) |
| AB01968659 | 01 | Sigma | Calpain Inhibitor IV | 0.24 | >7.17 | >29.9 |
| AB00053257 | 08 | SRI Repository | Hydroxychloroquine | 2.1 | >30.00 | >14.3 |
| AB00053436 | 10 | SRI Repository | Chloroquine | 4.03 | >30.00 | >7.4 |
| AB01952209 | 04 | Selleck | Remdesivir | 10.8 | >30.00 | >2.8 |

TABLE 2
Antiviral activity of VIRADEM Compounds in the SARS-CoV-2(WA1)
CPE assay, Round 2. (Concentration range of 1000-0.05 nM)

| | IC50 | Compound | IC50 | Compound | IC50 | Compound | IC50 |
|---|---|---|---|---|---|---|---|
| Compound ID | (uM) | ID | (uM) | ID | (uM) | ID | (uM) |
| VIRADEM- | 0.02 | VIRADEM- | 0.29 | VIRADEM- | >1.00 | VIRADEM- | >1.00 |
| 11847 | | 12262 | | 6410 | | 11940 | |
| VIRADEM- | 0.02 | VIRADEM- | 0.30 | VIRADEM- | >1.00 | VIRADEM- | >1.00 |
| 15270 | | 11982 | | 6411 | | 12036 | |
| VIRADEM- | 0.02 | VIRADEM- | 0.31 | VIRADEM- | >1.00 | VIRADEM- | >1.00 |
| 12329 | | 10886 | | 6626 | | 12037 | |
| VIRADEM- | 0.03 | VIRADEM- | 0.32 | VIRADEM- | >1.00 | VIRADEM- | >1.00 |
| 11775 | | 12188 | | 7003 | | 12180 | |
| VIRADEM- | 0.04 | VIRADEM- | 0.32 | VIRADEM- | >1.00 | VIRADEM- | >1.00 |
| 11676 | | 11566 | | 7016 | | 12185 | |
| VIRADEM- | 0.04 | VIRADEM- | 0.32 | VIRADEM- | >1.00 | VIRADEM- | >1.00 |
| 11771 | | 11778 | | 7157 | | 12194 | |
| VIRADEM- | 0.04 | VIRADEM- | 0.32 | VIRADEM- | >1.00 | VIRADEM- | >1.00 |
| 11923 | | 11789 | | 8067 | | 12198 | |
| VIRADEM- | 0.04 | VIRADEM- | 0.32 | VIRADEM- | >1.00 | VIRADEM- | >1.00 |
| 15263 | | 15267 | | 8416 | | 12199 | |
| VIRADEM- | 0.04 | VIRADEM- | 0.33 | VIRADEM- | >1.00 | VIRADEM- | >1.00 |
| 12334 | | 11766 | | 8757 | | 12218 | |
| VIRADEM- | 0.04 | VIRADEM- | 0.33 | VIRADEM- | >1.00 | VIRADEM- | >1.00 |
| 11884 | | 11770 | | 8798 | | 12220 | |
| VIRADEM- | 0.04 | VIRADEM- | 0.33 | VIRADEM- | >1.00 | VIRADEM- | >1.00 |
| 11913 | | 11926 | | 8832 | | 12237 | |
| VIRADEM- | 0.04 | VIRADEM- | 0.34 | VIRADEM- | >1.00 | VIRADEM- | >1.00 |
| 12296 | | 11935 | | 8843 | | 12238 | |
| VIRADEM- | 0.04 | VIRADEM- | 0.34 | VIRADEM- | >1.00 | VIRADEM- | >1.00 |
| 10897 | | 12304 | | 8895 | | 12240 | |
| VIRADEM- | 0.05 | VIRADEM- | 0.34 | VIRADEM- | >1.00 | VIRADEM- | >1.00 |
| 11682 | | 11683 | | 8920 | | 12241 | |
| VIRADEM- | 0.06 | VIRADEM- | 0.35 | VIRADEM- | >1.00 | VIRADEM- | >1.00 |
| 11777 | | 11759 | | 9006 | | 12246 | |
| VIRADEM- | 0.08 | VIRADEM- | 0.35 | VIRADEM- | >1.00 | VIRADEM- | >1.00 |
| 15279 | | 15231 | | 9042 | | 12257 | |
| VIRADEM- | 0.08 | VIRADEM- | 0.36 | VIRADEM- | >1.00 | VIRADEM- | >1.00 |
| 10853 | | 11787 | | 9082 | | 12271 | |
| VIRADEM- | 0.08 | VIRADEM- | 0.37 | VIRADEM- | >1.00 | VIRADEM- | >1.00 |
| 15277 | | 11782 | | 9163 | | 12275 | |
| VIRADEM- | 0.08 | VIRADEM- | 0.37 | VIRADEM- | >1.00 | VIRADEM- | >1.00 |
| 10884 | | 12390 | | 9585 | | 12277 | |
| VIRADEM- | 0.08 | VIRADEM- | 0.37 | VIRADEM- | >1.00 | VIRADEM- | >1.00 |
| 11671 | | 11569 | | 9591 | | 12278 | |
| VIRADEM- | 0.08 | VIRADEM- | 0.37 | VIRADEM- | >1.00 | VIRADEM- | >1.00 |
| 11765 | | 15367 | | 9667 | | 12288 | |
| VIRADEM- | 0.10 | VIRADEM- | 0.38 | VIRADEM- | >1.00 | VIRADEM- | >1.00 |
| 11665 | | 11786 | | 9794 | | 12300 | |
| VIRADEM- | 0.11 | VIRADEM- | 0.38 | VIRADEM- | >1.00 | VIRADEM- | >1.00 |
| 12252 | | 12270 | | 10257 | | 12303 | |
| VIRADEM- | 0.11 | VIRADEM- | 0.38 | VIRADEM- | >1.00 | VIRADEM- | >1.00 |
| 15271 | | 10852 | | 10846 | | 12307 | |
| VIRADEM- | 0.11 | VIRADEM- | 0.38 | VIRADEM- | >1.00 | VIRADEM- | >1.00 |
| 11667 | | 11666 | | 10848 | | 12312 | |
| VIRADEM- | 0.11 | VIRADEM- | 0.42 | VIRADEM- | >1.00 | VIRADEM- | >1.00 |
| 12211 | | 11793 | | 10849 | | 12314 | |
| VIRADEM- | 0.11 | VIRADEM- | 0.44 | VIRADEM- | >1.00 | VIRADEM- | >1.00 |
| 11781 | | 15262 | | 10867 | | 12324 | |
| VIRADEM- | 0.11 | VIRADEM- | 0.45 | VIRADEM- | >1.00 | VIRADEM- | >1.00 |
| 12254 | | 12305 | | 10874 | | 12359 | |
| VIRADEM- | 0.11 | VIRADEM- | 0.46 | VIRADEM- | >1.00 | VIRADEM- | >1.00 |
| 15264 | | 15230 | | 10879 | | 12409 | |
| VIRADEM- | 0.11 | VIRADEM- | 0.47 | VIRADEM- | >1.00 | VIRADEM- | >1.00 |
| 12407 | | 13159 | | 10881 | | 12434 | |
| VIRADEM- | 0.12 | VIRADEM- | 0.50 | VIRADEM- | >1.00 | VIRADEM- | >1.00 |
| 15356 | | 15240 | | 10882 | | 12435 | |
| VIRADEM- | 0.12 | VIRADEM- | 0.50 | VIRADEM- | >1.00 | VIRADEM- | >1.00 |
| 11141 | | 11663 | | 10885 | | 12458 | |
| VIRADEM- | 0.12 | VIRADEM- | 0.51 | VIRADEM- | >1.00 | VIRADEM- | >1.00 |
| 15278 | | 12253 | | 10887 | | 12511 | |
| VIRADEM- | 0.12 | VIRADEM- | 0.51 | VIRADEM- | >1.00 | VIRADEM- | >1.00 |
| 11797 | | 12333 | | 10890 | | 12665 | |
| VIRADEM- | 0.12 | VIRADEM- | 0.56 | VIRADEM- | >1.00 | VIRADEM- | >1.00 |
| 11490 | | 12251 | | 10891 | | 12687 | |
| VIRADEM- | 0.12 | VIRADEM- | 0.58 | VIRADEM- | >1.00 | VIRADEM- | >1.00 |
| 11882 | | 12322 | | 10892 | | 12713 | |
| VIRADEM- | 0.12 | VIRADEM- | 0.61 | VIRADEM- | >1.00 | VIRADEM- | >1.00 |
| 11773 | | 15247 | | 10893 | | 13002 | |
| VIRADEM- | 0.12 | VIRADEM- | 0.61 | VIRADEM- | >1.00 | VIRADEM- | >1.00 |
| 11491 | | 10258 | | 10896 | | 13044 | |
| VIRADEM- | 0.12 | VIRADEM- | 0.64 | VIRADEM- | >1.00 | VIRADEM- | >1.00 |
| 11849 | | 11888 | | 10898 | | 13072 | |
| VIRADEM- | 0.12 | VIRADEM- | 0.67 | VIRADEM- | >1.00 | VIRADEM- | >1.00 |
| 15225 | | 12323 | | 10901 | | 13075 | |
| VIRADEM- | 0.12 | VIRADEM- | 0.72 | VIRADEM- | >1.00 | VIRADEM- | >1.00 |
| 11681 | | 12235 | | 10902 | | 13077 | |
| VIRADEM- | 0.12 | VIRADEM- | 0.73 | VIRADEM- | >1.00 | VIRADEM- | >1.00 |
| 11660 | | 12327 | | 10903 | | 13078 | |
| VIRADEM- | 0.12 | VIRADEM- | 0.79 | VIRADEM- | >1.00 | VIRADEM- | >1.00 |
| 11942 | | 12331 | | 11041 | | 13115 | |
| VIRADEM- | 0.12 | VIRADEM- | 0.84 | VIRADEM- | >1.00 | VIRADEM- | >1.00 |
| 12197 | | 10894 | | 11045 | | 13160 | |
| VIRADEM- | 0.13 | VIRADEM- | 0.84 | VIRADEM- | >1.00 | VIRADEM- | >1.00 |
| 11887 | | 11936 | | 11046 | | 13290 | |
| VIRADEM- | 0.13 | VIRADEM- | 0.85 | VIRADEM- | >1.00 | VIRADEM- | >1.00 |
| 11914 | | 15229 | | 11139 | | 13348 | |
| VIRADEM- | 0.13 | VIRADEM- | 0.95 | VIRADEM- | >1.00 | VIRADEM- | >1.00 |
| 11784 | | 12433 | | 11146 | | 13440 | |
| VIRADEM- | 0.13 | VIRADEM- | 0.99 | VIRADEM- | >1.00 | VIRADEM- | >1.00 |
| 11686 | | 10883 | | 11482 | | 14298 | |
| VIRADEM- | 0.13 | VIRADEM- | 0.99 | VIRADEM- | >1.00 | VIRADEM- | >1.00 |
| 12272 | | 11761 | | 11499 | | 14302 | |
| VIRADEM- | 0.13 | VIRADEM- | >1.00 | VIRADEM- | >1.00 | VIRADEM- | >1.00 |
| 11776 | | 59 | | 11503 | | 14303 | |
| VIRADEM- | 0.14 | VIRADEM- | >1.00 | VIRADEM- | >1.00 | VIRADEM- | >1.00 |
| 15266 | | 551 | | 11510 | | 14305 | |
| VIRADEM- | 0.15 | VIRADEM- | >1.00 | VIRADEM- | >1.00 | VIRADEM- | >1.00 |
| 15255 | | 619 | | 11511 | | 14308 | |
| VIRADEM- | 0.15 | VIRADEM- | >1.00 | VIRADEM- | >1.00 | VIRADEM- | >1.00 |
| 11788 | | 792 | | 11515 | | 14309 | |
| VIRADEM- | 0.15 | VIRADEM- | >1.00 | VIRADEM- | >1.00 | VIRADEM- | >1.00 |
| 11987 | | 1400 | | 11522 | | 15106 | |
| VIRADEM- | 0.15 | VIRADEM- | >1.00 | VIRADEM- | >1.00 | VIRADEM- | >1.00 |
| 12292 | | 2327 | | 11528 | | 15164 | |
| VIRADEM- | 0.16 | VIRADEM- | >1.00 | VIRADEM- | >1.00 | VIRADEM- | >1.00 |
| 11673 | | 2427 | | 11567 | | 15199 | |
| VIRADEM- | 0.17 | VIRADEM- | >1.00 | VIRADEM- | >1.00 | VIRADEM- | >1.00 |
| 10854 | | 2711 | | 11571 | | 15201 | |
| VIRADEM- | 0.17 | VIRADEM- | >1.00 | VIRADEM- | >1.00 | VIRADEM- | >1.00 |
| 11795 | | 2720 | | 11575 | | 15209 | |
| VIRADEM- | 0.17 | VIRADEM- | >1.00 | VIRADEM- | >1.00 | VIRADEM- | >1.00 |
| 11785 | | 2877 | | 11579 | | 15220 | |
| VIRADEM- | 0.17 | VIRADEM- | >1.00 | VIRADEM- | >1.00 | VIRADEM- | >1.00 |
| 12408 | | 2878 | | 11584 | | 15234 | |
| VIRADEM- | 0.18 | VIRADEM- | >1.00 | VIRADEM- | >1.00 | VIRADEM- | >1.00 |
| 15168 | | 2881 | | 11588 | | 15243 | |
| VIRADEM- | 0.18 | VIRADEM- | >1.00 | VIRADEM- | >1.00 | VIRADEM- | >1.00 |
| 11783 | | 2887 | | 11590 | | 15248 | |
| VIRADEM- | 0.19 | VIRADEM- | >1.00 | VIRADEM- | >1.00 | VIRADEM- | >1.00 |
| 11889 | | 2888 | | 11592 | | 15272 | |
| VIRADEM- | 0.19 | VIRADEM- | >1.00 | VIRADEM- | >1.00 | VIRADEM- | >1.00 |
| 11798 | | 2892 | | 11593 | | 15273 | |
| VIRADEM- | 0.19 | VIRADEM- | >1.00 | VIRADEM- | >1.00 | VIRADEM- | >1.00 |
| 12265 | | 2992 | | 11596 | | 15288 | |
| VIRADEM- | 0.20 | VIRADEM- | >1.00 | VIRADEM- | >1.00 | VIRADEM- | >1.00 |
| 11674 | | 3087 | | 11597 | | 15295 | |
| VIRADEM- | 0.20 | VIRADEM- | >1.00 | VIRADEM- | >1.00 | VIRADEM- | >1.00 |
| 11878 | | 3188 | | 11664 | | 15355 | |
| VIRADEM- | 0.20 | VIRADEM- | >1.00 | VIRADEM- | >1.00 | VIRADEM- | >1.00 |
| 12227 | | 3621 | | 11680 | | 15361 | |
| VIRADEM- | 0.21 | VIRADEM- | >1.00 | VIRADEM- | >1.00 | VIRADEM- | >1.00 |
| 15258 | | 3876 | | 11687 | | 16516 | |
| VIRADEM- | 0.22 | VIRADEM- | >1.00 | VIRADEM- | >1.00 | VIRADEM- | >1.00 |
| 12248 | | 3878 | | 11688 | | 16523 | |
| VIRADEM- | 0.22 | VIRADEM- | >1.00 | VIRADEM- | >1.00 | VIRADEM- | >1.00 |
| 11941 | | 3905 | | 11758 | | 19239 | |
| VIRADEM- | 0.23 | VIRADEM- | >1.00 | VIRADEM- | >1.00 | VIRADEM- | >1.00 |
| 11685 | | 3923 | | 11762 | | 19499 | |
| VIRADEM- | 0.23 | VIRADEM- | >1.00 | VIRADEM- | >1.00 | VIRADEM- | >1.00 |
| 12280 | | 4221 | | 11768 | | 20859 | |
| VIRADEM- | 0.23 | VIRADEM- | >1.00 | VIRADEM- | >1.00 | VIRADEM- | >1.00 |
| 11921 | | 4815 | | 11799 | | 20864 | |
| VIRADEM- | 0.24 | VIRADEM- | >1.00 | VIRADEM- | >1.00 | VIRADEM- | >1.00 |
| 12249 | | 4833 | | 11808 | | 20867 | |
| VIRADEM- | 0.25 | VIRADEM- | >1.00 | VIRADEM- | >1.00 | VIRADEM- | >1.00 |
| 11684 | | 4835 | | 11809 | | 20871 | |
| VIRADEM- | 0.26 | VIRADEM- | >1.00 | VIRADEM- | >1.00 | VIRADEM- | >1.00 |
| 11881 | | 4892 | | 11846 | | 22057 | |
| VIRADEM- | 0.27 | VIRADEM- | >1.00 | VIRADEM- | >1.00 | VIRADEM- | >1.00 |
| 15166 | | 5650 | | 11883 | | 22536 | |
| VIRADEM- | 0.27 | VIRADEM- | >1.00 | VIRADEM- | >1.00 | VIRADEM- | >1.00 |
| 11794 | | 5655 | | 11924 | | 6414 | |
| VIRADEM- | 0.29 | VIRADEM- | >1.00 | VIRADEM- | >1.00 | VIRADEM- | >1.00 |
| 11679 | | 5687 | | 6284 | | 6285 | |

The overall assay performances were acceptable. In CPE assay, cell viability in the presence and absence of virus infection exhibited the expected assay window. Positive controls for SARS-CoV-2 (Calpain Inhibitor IV, Remdesivir, Chloroquine) showed dose dependent antiviral effect and worked as expected in the CPE assay.

In the 2nd round of screening 22 compounds showed IC₅₀ of <0.1 μM, 90 compounds showed IC₅₀ of 0.1-0.5 μM and 17 compounds showed IC₅₀ of 0.51-0.99 μM. Thus, 129 compounds showed IC₅₀ values<1 μM in the second round and 21 compounds exhibited IC₅₀ values between 1 μM and 30 μM in the first round.

Overall, these results show impressive antiviral potency of the disclosed compounds in the in vitro antiviral assays. Some of the disclosed compounds showed significantly better antiviral efficacy compared to Remdesivir (SARS-CoV-2 polymerase inhibitor), tested in the assay as a positive control (IC₅₀ of <0.1 μM vs 7-10 μM respectively). It might be useful to focus on the data from 21 compounds exhibited IC₅₀ values between 1 μM and 30 μM in the first round and 129 compounds which showed IC₅₀ values<1 μM in the second round for further analysis.

### Method for SARS CoV-2 Nanoluciferase Reporter Virus Assay, Southern Research.

Method for measuring antiviral effect of compounds: A549 cells expressing ACE-2 (obtained from Ralph Barric at UNC) are grown in DMEM high glucose supplemented with 20% HI FBS, 1% NEAA, 100 μg/ml Blasticidin and split 1:6 every three days (remove Blasticidin from the media one passage before using the cells in the assay). On the day of assay, the cells are harvested in DMEM supplemented with 2% HI FBS, 1% HEPES, 1% Pen/Strep. Assay ready plates pre-drugged with test compounds are prepared in the BSL-2 lab by adding 5 μL assay media to each well. The plates and cells are then passed into the BSL-3 facility. A working stock of SARS CoV-2 nanoluciferase reporter virus (NLRV) passaged five times in A549 cells expressing ACE2 is diluted 6000-fold in media containing 160,000 cells per mL (MOI˜0.002) and stirred at 200 RMP for approximately 10 minutes. A 25 μL aliquot of virus inoculated cells (4000 cells) is added to each well in columns 3-24 of the assay plates. The wells in columns 23-24 do not contain test compounds, only virus infected cells for the 0% inhibition controls. Prior to virus inoculation, a 25 μL aliquot of cells is added to columns 1-2 (no test compounds) of each plate for the cell only 100% inhibition controls. After incubating plates at 37° C./5% CO2 and 90% humidity for 72 hours, 30 μL of NanoGlo (Promega) is added to each well. Luminescence is read using a BMG CLARIOstar plate reader (bottom read) following incubation at room temperature for 10 minutes to measure luciferase activity as an index of virus titer. Plates are sealed with a clear cover and surface decontaminated prior to luminescence reading.

Method for measuring cytotoxic effect of compounds: Compound cytotoxicity is assessed in a BSL-2 counter screen as follows: Host cells in media are added in 25 μl aliquots (4000 cells/well) to each well of assay ready plates prepared with test compounds as above. Cells only (100% viability) and cells treated with hyamine at 100 μM final concentration (0% viability) serve as the high and low signal controls, respectively, for cytotoxic effect in the assay. DMSO is maintained at a constant concentration for all wells as dictated by the dilution factor of stock test compound concentrations. After incubating plates at 37° C./5% CO2 and 90% humidity for 72 hours, 30 μl Cell Titer-Glo (Promega) is added to each well. Luminescence is read using a BMG PHERAstar plate reader following incubation at room temperature for 10 minutes to measure cell viability.

Data analysis: For all assays the raw data from plate readers are imported into ActivityBase where values are associated with compound IDs and test concentrations.

For the antiviral NLRV assay, raw signal values are converted to % inhibition by the following formula: % inhibition=100×(test cmpd value−mean value infected cell controls)/(mean value uninfected cell controls−mean value infected cell controls).

For the cell viability assay measuring compound cytotoxicity, % cell viability is calculated as follows: % viability=100*(test cmpd value−mean low signal control)/(mean high signal control−mean low signal control).

EC50 and CC50 values are calculated from a four-parameter logistic fit of data using the Xlfit module of ActivityBase with top and bottom constrained to 100 and 0%, respectively.

TABLE 3
Antiviral activity for SARS-CoV-2(delta variant)
for human lung cells A549.
luciferase reporter SARS-CoV-2 WA1 virus used
to infect A549-ACE2 cells (Southern Research)

| Compound ID | IC50(uM) | IC90(uM) |
|---|---|---|
| VIRADEM-11918 | 0.019 | 0.029 |
| VIRADEM-11847 | 0.032 | 0.041 |
| VIRADEM-15225 | 0.094 | 0.167 |
| VIRADEM-11141 | 0.097 | 0.118 |
| VIRADEM-12226 | 0.103 | 0.432 |
| VIRADEM-11686 | 0.116 | 0.287 |
| VIRADEM-11671 | 0.130 | 0.235 |
| VIRADEM-12296 | 0.136 | 0.324 |
| VIRADEM-10853 | 0.150 | 0.287 |
| VIRADEM-11884 | 0.168 | 0.293 |

TABLE 4
Antiviral activity for SARS-CoV-2(delta variant) for VeroE6 cells.
CPE Assay Methodology remained the same as for WA1 assay
(see [94-102]). Southern Research.
VeroE6 Delta Variant Data

| Compound Id | IC50 (uM) | IC90 (uM) |
|---|---|---|
| VIRADEM-11847 | 0.036 | 0.046 |
| VIRADEM-11918 | 0.041 | 0.060 |
| VIRADEM-15286 | 0.100 | 0.161 |
| VIRADEM-15277 | 0.112 | 0.129 |
| VIRADEM-12296 | 0.123 | 0.147 |
| VIRADEM-11141 | 0.125 | 0.150 |
| VIRADEM-10853 | 0.144 | 0.173 |
| VIRADEM-15225 | 0.152 | 0.250 |
| VIRADEM-12226 | 0.169 | 0.209 |
| VIRADEM-11686 | 0.174 | 0.231 |
| VIRADEM-11884 | 0.175 | 0.272 |
| VIRADEM-11671 | 0.189 | 0.214 |
| VIRADEM-11923 | 0.191 | 0.240 |
| VIRADEM-11775 | 0.193 | 0.364 |
| VIRADEM-15278 | 0.194 | 0.388 |
| VIRADEM-11682 | 0.226 | 0.286 |
| VIRADEM-11921 | 0.242 | 0.298 |
| VIRADEM-11913 | 0.245 | 0.331 |
| VIRADEM-12211 | 0.258 | 0.321 |
| VIRADEM-15287 | 0.259 | 0.503 |
| VIRADEM-11777 | 0.268 | 0.326 |
| VIRADEM-11998 | 0.278 | 0.414 |
| VIRADEM-11490 | 0.299 | 0.405 |
| VIRADEM-11773 | 0.301 | 0.352 |
| VIRADEM-11887 | 0.314 | 0.401 |
| VIRADEM-11672 | 0.316 | 0.413 |
| VIRADEM-12324 | 0.321 | >1.00 |
| VIRADEM-11920 | 0.329 | 0.374 |
| VIRADEM-11685 | 0.338 | 0.356 |
| VIRADEM-11665 | 0.341 | 0.392 |
| VIRADEM-11675 | 0.344 | 0.426 |
| VIRADEM-15263 | 0.344 | 0.431 |
| VIRADEM-11926 | 0.352 | 0.432 |
| VIRADEM-10897 | 0.353 | 0.377 |
| VIRADEM-12273 | 0.353 | 0.411 |
| VIRADEM-11684 | 0.357 | 0.411 |
| VIRADEM-12252 | 0.357 | 0.425 |
| VIRADEM-11676 | 0.359 | 0.440 |
| VIRADEM-11681 | 0.367 | 0.435 |
| VIRADEM-12329 | 0.368 | 0.465 |
| VIRADEM-12265 | 0.370 | 0.469 |
| VIRADEM-11786 | 0.371 | 0.396 |
| VIRADEM-12406 | 0.373 | 0.449 |
| VIRADEM-11914 | 0.382 | 0.437 |
| VIRADEM-11941 | 0.383 | 0.457 |
| VIRADEM-11772 | 0.388 | 0.458 |
| VIRADEM-10884 | 0.389 | 0.477 |
| VIRADEM-11771 | 0.401 | 0.502 |
| VIRADEM-11787 | 0.404 | 0.446 |
| VIRADEM-11889 | 0.423 | 0.514 |
| VIRADEM-11680 | 0.428 | 0.521 |
| VIRADEM-12334 | 0.432 | 0.508 |
| VIRADEM-12272 | 0.463 | 0.799 |
| VIRADEM-11667 | 0.464 | 0.518 |
| VIRADEM-11882 | 0.470 | 0.635 |
| VIRADEM-11881 | 0.513 | 1.199 |
| VIRADEM-11784 | 0.541 | 0.779 |
| VIRADEM-11794 | 0.545 | 0.855 |
| VIRADEM-12408 | 0.547 | 0.672 |
| VIRADEM-12254 | 0.548 | 0.668 |
| VIRADEM-12280 | 0.554 | 0.679 |
| VIRADEM-11785 | 0.559 | 0.690 |
| VIRADEM-11916 | 0.559 | 0.707 |
| VIRADEM-12125 | 0.584 | 1.371 |
| VIRADEM-11394 | 0.592 | >3.00 |
| VIRADEM-11783 | 0.592 | 0.770 |
| VIRADEM-11766 | 0.603 | 0.752 |
| VIRADEM-12227 | 0.606 | 0.700 |
| VIRADEM-11776 | 0.608 | 0.693 |
| VIRADEM-10898 | 0.724 | >3.00 |
| VIRADEM-11797 | 0.733 | 0.839 |
| VIRADEM-10854 | 0.771 | 1.942 |
| VIRADEM-11982 | 0.813 | 1.404 |
| VIRADEM-11780 | 0.913 | 1.057 |
| VIRADEM-11670 | 0.934 | >1.00 |
| VIRADEM-12251 | 0.951 | 1.166 |
| VIRADEM-11934 | 1.018 | 1.236 |
| VIRADEM-11935 | 1.021 | 1.178 |
| VIRADEM-15229 | 1.035 | 1.192 |
| VIRADEM-12326 | 1.125 | 1.333 |
| VIRADEM-11004 | 1.126 | 1.341 |
| VIRADEM-11677 | 1.173 | 1.228 |
| VIRADEM-13159 | 1.196 | 1.464 |
| VIRADEM-11781 | 1.283 | 1.371 |
| VIRADEM-12407 | 1.339 | 1.513 |
| VIRADEM-11852 | 1.371 | 1.726 |
| VIRADEM-10900 | 1.659 | 1.642 |
| VIRADEM-12390 | 1.821 | 2.109 |
| VIRADEM-11660 | 1.861 | >3.00 |
| VIRADEM-11936 | 2.019 | 2.252 |
| VIRADEM-11673 | 2.630 | 1.316 |
| VIRADEM-11683 | 2.839 | 2.525 |

Antiviral activity in 229E and OC43 viruses in Huh7.5 cells. Assays were carried out at Emory University by Lefteris Michailidis, PhD. Assistant Professor, Laboratory of Biochemical Pharmacology

Protection from OC43 or 229E coronavirus infection was quantified using a cytopathic effect assay in Huh7.5 cells. Cells were pre-treated with a titration of each compound for 1 hour, after which viral inoculum was added. Infected cultures were fixed with formaldehyde either 2 days after infection (229E) or 3 days after infection (OC43). The secondary antibody Alexa Fluorophore 594 goat anti-mouse IgG (H+L) (Invitrogen) was used to label the primary anti-OC43 antibody and anti-229E antibody. Cells were counterstained with DAPI to label nuclei. Images were captured using the Cytation 7 Multimode Reader (BioTek) and the level of infection was determined using the Gen5 image analysis software (BioTek). Remdesivir was run with all experiments as a positive control for infection inhibition.

Separately, cytotoxicity for each compound (CC50 values) were assessed by titration of compounds on the cells for the same period, and viability analyzed using PrestoBlue viability assay.

For OC43 experiments, drugs were titrated in two-fold titrations over 9 concentrations (range: 10000 nM-39.1 nM). For 229E experiments, drugs were titrated in three-fold titrations over 9 concentrations (range: 10000 nM-1.5 nM). All experiments were performed in biological triplicate.

To calculate IC50 and CC50 values, cell counts were normalized to untreated controls, concentrations (measured in nM) were log transformed, and IC50 and CC50 values were calculated using the nonlinear regression function “Dose-Response—Inhibition” in Graphpad Prism.

TABLE 5
Antiviral activity in 229E virus in Huh7.5 cells.
229E = an alphacoronavirus, drugs tested with Huh7.5
cells (Emory university)

| Compound ID | 229E IC50 |
|---|---|
| VIRADEM-11847 | 138 nM |
| VIRADEM-11141 | 260 nM |
| VIRADEM-11686 | 486 nM |
| VIRADEM-11671 | 506 nM |
| VIRADEM-12226 | 300 nM |
| Remdesivir | 13 nM |

TABLE 5
Antiviral activity in OCE43 virus in Huh7.5 cells.
OC43 = another betacoronavirus, drugs tested with Huh7.5
cells (Emory University)

| Compound ID | OC43 IC50 | |
|---|---|---|
| VIRADEM-11847 | 992.1 | nM |
| VIRADEM-11141 | 1.65 | uM |
| VIRADEM-11686 | 3 | uM |
| VIRADEM-11671 | 1.8 | uM |
| VIRADEM-12226 | 1.98 | uM |
| Remdesivir | 9.7 | nM |

As the compounds of the invention, mention can be preferably made of the following examples:

### EXAMPLES

The present invention will be more fully described by way of the following examples which in no way serve to limit the scope of the invention, but rather are presented for illustrative purposes.

### Examples of Synthesis of Compounds

Some of the heterocyclic compounds for use in the methods of the present invention were prepared according to the procedures described in prior applications or alternatively according to synthetic techniques well known in the art.

2,4-Dichloro-6-morpholin-4-yl-[1,3,5]triazine was obtained by the method [Tetrahedron Letters; vol. 47; nb. 33; (2006); p. 5973-5975]:

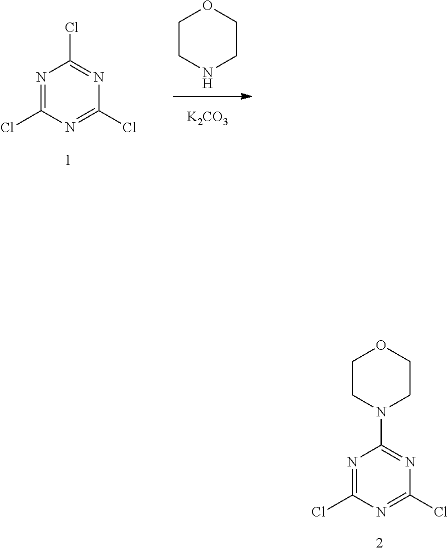

To a solution of cyanuric acid (92.2 g, 0.50 mol) in dry acetone (500 ml) was added 69.5 g (0.5 mol) anhydrous potassium carbonate K₂CO₃ and dropwise of morpholine (43.6 ml, 0.5 mol) at 0° C. with vigorous stirring. The resulting mixture was stirring at 2 hours and left for 12 hours at room temperature. Then acetone was evaporated in vacuum. 200 ml of water was added to the dry residue. White precipitates were collected and washed with water to give 105.8 g of 2,4-dichloro-6-morpholino-1,3,5-triazine as white crystal in 90% yield. M.p.=137° C. C₇H₈Cl₂N₄O. MS m/z: 236.0 [(M+H)+]

2-Chloro-4,6-dimorpholin-4-yl-[1,3,5]triazine (Compound 1) was obtained by the method [Patent US 2007 244 110A1]:

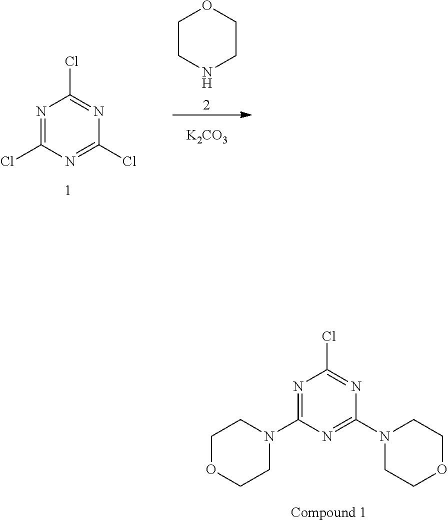

To a solution of cyanuric acid (92.2 g, 0.50 mol) in dry acetone (800 ml) was added 137.0 g (1.0 mol) anhydrous potassium carbonate K₂CO₃ and dropwise of morpholine (87.2 ml, 1.0 mol) at 20° C. with vigorous stirring. The resulting mixture was stirring at 2 hours and left for 12 hours at room temperature. Then acetone was evaporated in vacuum. 400 ml of water was added to the dry residue. White precipitates were collected and washed with water to give 242.8 g of 2-chloro-4,6-dimorpholin-4-yl-1,3,5-triazine as white crystal in 85% yield. C₁₁H₁₆ClN₅O₂. MS m/z: 286.2 [(M+H)+].

Synthesis of (4-chloro-6-morpholin-4-yl-[1,3,5]triazin-2-yl)-phenylamine (Compound 2) was obtained by the method [European Journal of Medicinal Chemistry; vol. 45; nb. 8; (2010); p. 3335-3345]:

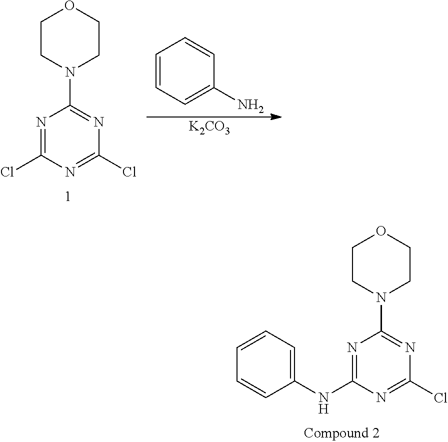

A mixture of 2,4-dichloro-6-morpholino-1,3,5-triazine (23.5 g, 0.1 mol), aniline (9.5 ml, 0.1 mol) and anhydrous potassium carbonate (13.9 g, 0.1 mol) in DMF (100 ml) was stirred at room temperature for 4 hours. The resulting mixture was poured into water. White precipitates were collected and washed with water. The solid was dried under reduced pressure to give 4-chrolo-6-morpholin-4-yl-[1,3,5]-triazin-2-yl)-phenylamine (24.2 g,) as white crystal in 83% yield. C₁₃H₁₄ClN₅O. MS m/z: 292.7 [(M+H)+]. ¹H NMR (400 MHz, DMSO-d₆) δ: 3.65 (m, 4H, CH₂NCH₂), 3.74 (m, 4H, CH₂OCH₂), 7.05 (t, 1H, 2C₆H₅), 7.23 (t, 2H, C₆H₅), 7.64 (d, 2H, C₆H₅), 10.1 (s, 1H, NH).

Synthesis of N-(4-methoxyphenyl)-6-morpholin-4-yl-N¹-phenyl-[1,3,5]triazine-2,4-diamine hydrochloride) was obtained by the method [Patent No 67510 Ukraine](Compound 3) (VIRADEM-011788):

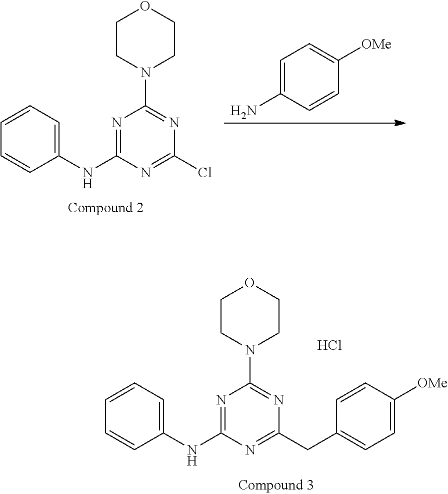 [SMILES: Clc1nc(Nc2ccccc2)nc(N2CCOCC2)n1 ; COc1ccc(N)cc1 ; COc1ccc(Cc2nc(Nc3ccccc3)nc(N3CCOCC3)n2)cc1 ; Cl]

Yield 68%. M.p.=181-183° C. C₂₀H₂₃N₆O₂. MS m/z: 379.2 [(M+H)+]. ¹H NMR (400 MHz, DMSO-d₆) δ: 3.67 (m, 4H, CH₂NCH₂), 3.71 (m, 4H, CH₂OCH₂), 3.74 (s, 3H, OCH₃), 6.89 and 7.57 (d-d, 4H, C₆H₄), 7.02 (t, 1H, 2C₆H₅), 7.30 (t, 2H, C₆H₅), 7.70 (d, 2H, 2C₆H₅), 9.51 (s, 1H, NH), 9.60 (s, 1H, NH).

Synthesis of N-benzo[1,3]dioxol-5-yl-N¹-(3-fluorophenyl)-6-morpholin-4-yl-[1,3,5]triazine-2,4-diamine hydrochloride) was obtained by the method [Patent No 86038 Ukraine](Compound 4) (VIRADEM-011854):

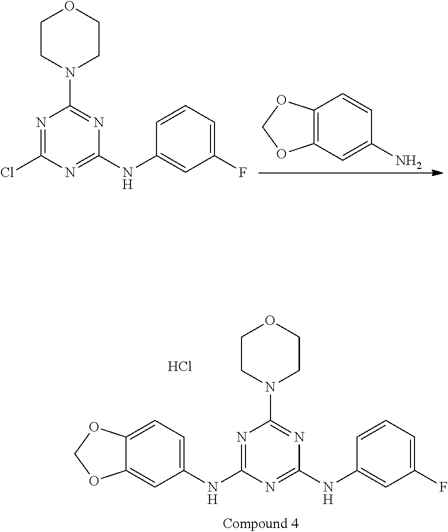

Yield 63%. M.p.=170-172° C. C₂₀H₂₀ClFN₆O₃. MS m/z: 411.2 [(M+H)+]. ¹H NMR (400 MHz, DMSO-d₆) 3.65 (m, 4H, CH₂NCH₂), 3.74 (m, 4H, CH₂OCH₂), δ: 6.01 (s, 2H, CH₂OCH₂), 6.81-7.72 (m, 7H, C₆H₃+C₆H₄), 9.70 (s, 1H, 1NH), 9.95 (s, 1H, 1NH).

Synthesis of N-benzo[1,3]dioxol-5-yl-N¹-(3-fluorophenyl)-6-morpholin-4-yl-[1,3,5]triazine-2,4-diamine hydrochloride) was obtained by the method [Patent No 86038 Ukraine](Compound 4) (VIRADEM-011854):

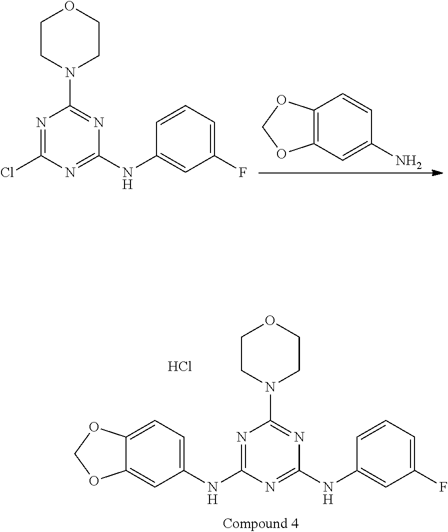

Yield 63%. M.p.=170-172° C. C₂₀H₂₀ClFN₆O₃. MS m/z: 411.2 [(M+H)+]. ¹H NMR (400 MHz, DMSO-d₆) 3.65 (m, 4H, CH₂NCH₂), 3.74 (m, 4H, CH₂OCH₂), δ: 6.01 (s, 2H, CH₂OCH₂), 6.81-7.72 (m, 7H, C₆H₃+C₆H₄), 9.70 (s, 1H, 1NH), 9.95 (s, 1H, 1NH)

Synthesis of N-(3,4-dimethylphenyl)-6-morpholin-4-yl-N¹-p-tolyl-[1,3,5]triazine-2,4-diamine was obtained by the method [Patent No 95732 Ukraine](Compound 5) (VIRADEM-011141):

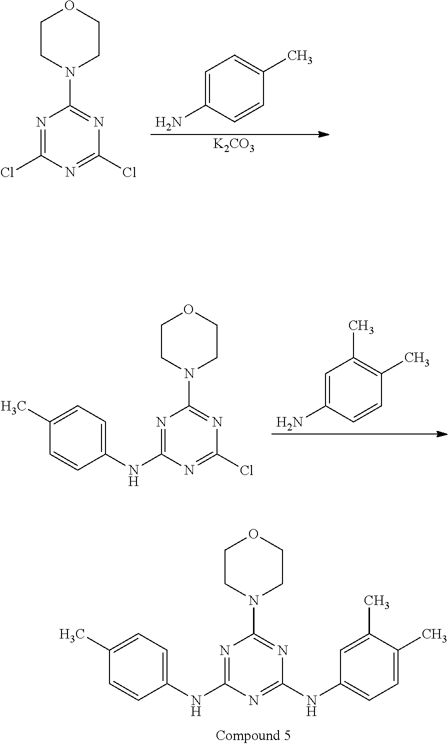

Yield 69%. M.p.=219-222° C. C₂₂H₂₇ClN₆O₂. MS m/z: 391.3 [(M+H)+]. ¹H NMR (400 MHz, DMSO-d₆) δ: 2.16 (s, 3H, CH₃), 2.27 (s, 3H, CH₃), 3.67 (m, 4H, CH₂NCH₂), 3.78 (m, 4H, CH₂OCH₂), 7.16 and 7.52 (d-d, 4H, C₆H₄), 7.10-7.43 (m, 3H, C₆H₃), 10.1 (s, 2H, 2NH).

Synthesis of N,N¹-bis-benzo[1,3]dioxol-5-yl-6-morpholin-4-yl-[1,3,5]triazine-2,4-diamine was obtained by the method [Patent No 100048 Ukraine](Compound 6) (VIRADEM-011566):

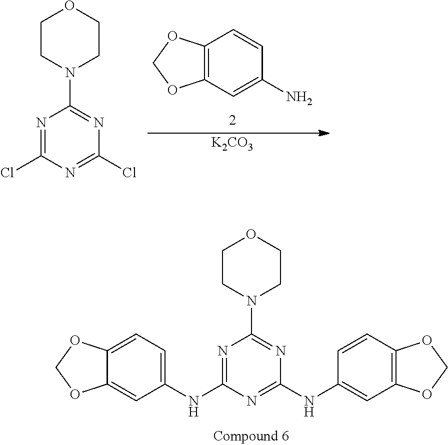

Yield 63%. M.p.=170-172° C. C₂₁H₂₀NO₅. MS m/z: 437.2 [(M+H)+]. ¹H NMR (400 MHz, DMSO-d₆) δ: 3.65 (m, 4H, CH₂NCH₂), 3.71 (m, 4H, CH₂OCH₂), 5.97 (s, 4H, 2-OCH₂O—), 6.80-7.48 (m, 6H, 2C₆H₃), 9.06 (s, 2H, 2NH).

Synthesis of N-(3-Chloro-4-methylphenyl)-N¹-(3-fluorophenyl)-6-morpholin-4-yl-[1,3,5]triazine-2,4-diamine was obtained by the method [Patent No 116386 Ukraine](Compound 7) (VIRADEM-012292):

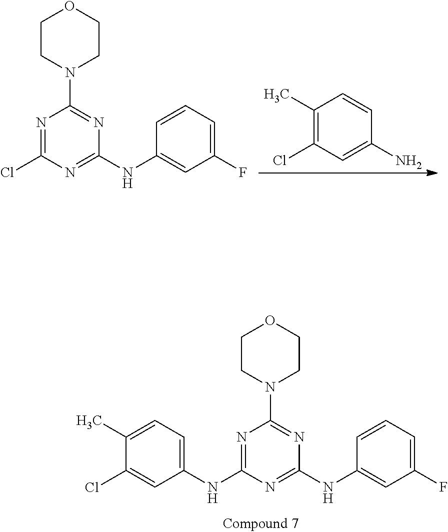

Yield 68%. M.p.=198-200° C. C₂₀H₂₀ClFN₆O. MS m/z: 415.5 [(M+H)+]. ¹H NMR (400 MHz, DMSO-d₆) δ: 2.27 (s, 3H, CH₃), 3.66 (m, 4H, CH₂NCH₂), 3.75 (m, 4H, CH₂OCH₂), 6.74-7.86 (m, 7H, C₆H₄+C₆H₃), 9.40 (s, 1H, NH), 9.47 (s, 1H, NH).

Synthesis of (3,4-dimethylphenyl)-(4,6-dimorpholin-4-yl-[1,3,5]triazin-2-yl)-amine was obtained by the method [Patent No 118704 Ukraine](Compound 8) (VIRADEM-010884):

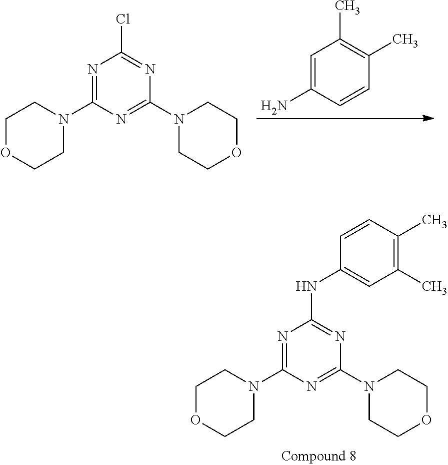

Yield 75%. M.p.=195-195° C. C₁₉H₂₇ClN₆O₂. MS m/z: 371.2 [(M+H)+]. ¹H NMR (400 MHz, DMSO-d₆) δ: 2.17 (s, 3H, CH₃), 2.19 (s, 3H, CH₃), 3.63 (m, 8H, 2-CH₂NCH₂—), 3.74 (m, 8H, 2-CH₂OCH₂—), 7.06 (d, 1H, C₆H₃), 7.37 (s, 1H, C₆H₃), 7.38 (d, 1H, C₆H₃), 9.95 (s, 1H, NH).

Synthesis of 1-{3-[4-(4-chlorophenylamino)-6-morpholin-4-yl-[1,3,5]triazin-2-ylamino]-phenyl}-ethanone was obtained by the method [Patent No 118887 Ukraine](Compound 9) (VIRADEM-011882):

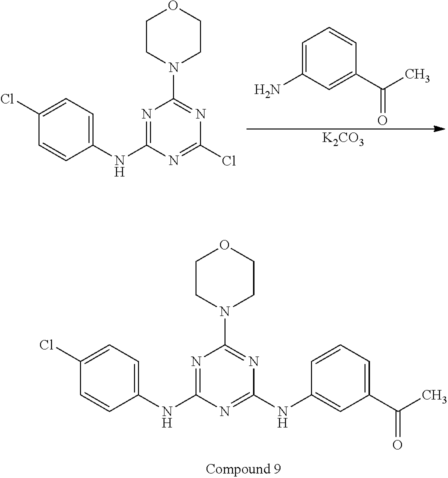

Yield 76%. M.p.=228-230° C. C₂₁H₂₁ClN₆O₂. MS m/z: 425.6 [(M+H)+]. ¹H NMR (400 MHz, DMSO-d₆) δ: 2.58 (s, 3H, COCH₃), 3.64 (m, 4H, CH₂NCH₂), 3.77 (m, 4H, CH₂OCH₂), 7.42-8.36 (m, 3H, C₆H₃), 7.32 and 7.77 (d-d, 4H, C₆H₄), 9.38 (s, 1H, 1NH), 9.46 (s, 1H, 1NH).

Synthesis of N-(3,4-Dimethylphenyl)-6-morpholin-4-yl-N¹-(3-trifluoromethylphenyl)-[1,3,5]triazine-2,4-diamine was obtained by the method [Patent No 120051 Ukraine](Compound 10) (VIRADEM-011914):

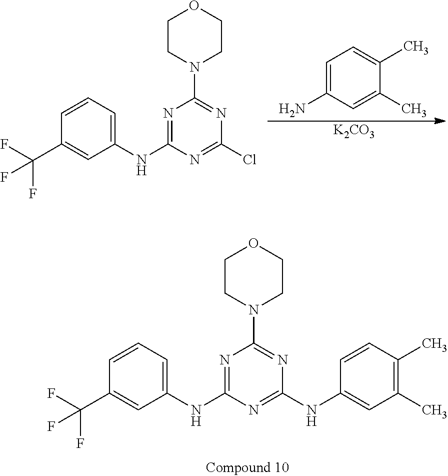

Yield 73%. M.p.=175-176° C. C₂₂H₂₃F₃N₆O. MS m/z: 445.2 [(M+H)+]. ¹H NMR (400 MHz, DMSO-d₆) δ: 2.15 (s, 3H, CH₃), 2.26 (s, 3H, CH₃), 3.68 (m, 4H, CH₂NCH₂), 3.77 (m, 4H, CH₂OCH₂), 7.11-8.19 (m, 7H, C₆H₃+C₆H₄), 9.12 (s, 1H, 1NH), 9.45 (s, 1H, 1NH).

Synthesis of N-(3,4-Dimethoxyphenyl)-N¹-(4-fluorophenyl)-6-morpholin-4-yl-[1,3,5]triazine-2,4-diamine was obtained by the method [Patent No 137987 Ukraine](Compound 11) (VIRADEM-011881):

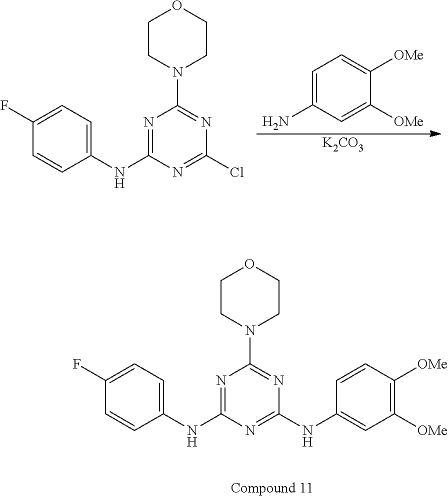

Yield 87%. M.p.=153-154° C. C₂₁H₂₃FN₆O₃. MS m/z: 427.2 [(M+H)+]. ¹H NMR (400 MHz, DMSO-d₆) δ: 3.66 (m, 4H, CH₂NCH₂), 3.70 (m, 4H, CH₂OCH₂), 3.72 (s, 3H, OCH₃), 3.75 (s, 3H, OCH₃), 6.86-7.75 (m, 7H, C₆H₃+C₆H₄), 8.98 (s, 1H, 1NH), 9.11 (s, 1H, 1NH).

Synthesis of N,N¹-bis-(2-chlorophenyl)-6-morpholin-4-yl-[1,3,5]triazine-2,4-diamine was obtained by the method [Patent No 122175 Ukraine](Compound 12) (VIRADEM-014303):

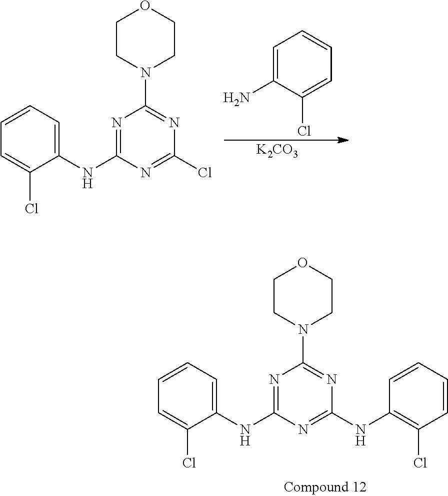

Yield 57%. M.p.=162-163° C. C₂₃H₂₉ClN₆O. MS m/z: 405.4 [(M+H)+]. ¹H NMR (400 MHz, DMSO-d₆) δ: 3.65 (m, 4H, CH₂NCH₂), 3.76 (m, 4H, CH₂OCH₂), 7.12-7.78 (m, 8H, 2C₆H₄), 9.41 (s, 2H, 2NH).

Synthesis of N-(3,5-Dichlorophenyl)-N¹-(4-ethylphenyl)-6-morpholin-4-yl-[1,3,5]triazine-2,4-diamine was obtained by the method [Patent No 134396 Ukraine](Compound 13) (VIRADEM-012327):

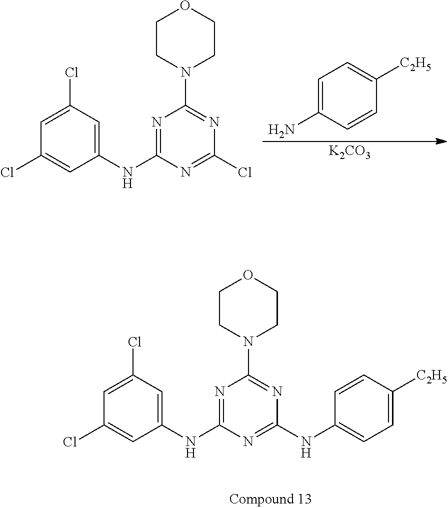

Yield 81%. M.p.=140-141° C. C₂₁H₂₂Cl₂N₆O. MS m/z: 446.0 [(M+H)+]. ¹H NMR (400 MHz, DMSO-d₆) δ: 1.18 (t, 3H, CH₂CH₃), 2.56 (q, 2H, CH₂CH₃), 3.66 (m, 4H, CH₂NCH₂), 3.75 (m, 4H, CH₂OCH₂), 7.10 (s, 1H, C₆H₃), 7.14 and 7.56 (d-d, 4H, C₆H₄), 6.75-7.78 (m, 4H, C₆H₄), 7.85 (s, 2H, C₆H₃), 9.30 (s, 1H, 1NH), 9.56 (s, 1H, 1NH).

Synthesis of N-(4-methoxyphenyl)-6-morpholin-4-yl-N¹-m-tolyl-[1,3,5]triazine-2,4-diamine hydrochloride was obtained by the method [Patent No 122187 Ukraine](Compound 14) (VIRADEM-011685):

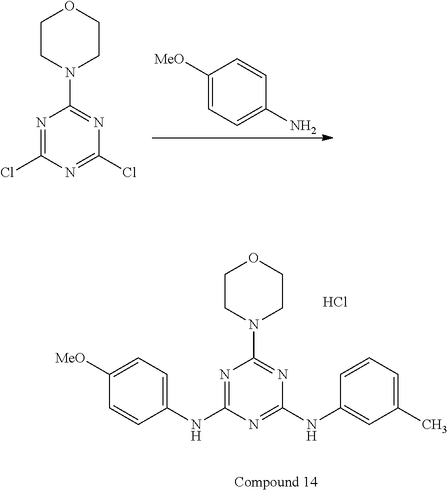 [SMILES: Clc1nc(Cl)nc(N2CCOCC2)n1 ; COc1ccc(N)cc1 ; COc1ccc(Nc2nc(Nc3cccc(C)c3)nc(N3CCOCC3)n2)cc1 ; Cl]

Yield 73%. M.p.=201-202° C. C₂₁H₂₅ClN₆O₂. MS m/z: 393.0 [(M+H)+]. ¹H NMR (500 MHz, DMSO-d₆) δ: 2.29 (s, 3H, CH₃), 3.69 (m, 4H, CH₂NCH₂), 3.79 (s, 3H, OCH₃), 3.80 (m, 4H, CH₂OCH₂), 6.89-7.48 (m, 4H, C₆H₄), 6.93 and 7.53 (d-d, 4H, C₆H₄), 10.3 (wide s, 1H, NH), 10.6 (wide s, 1H, NH). ¹³C-NMR (150 MHz, DMSO-d₆) δ: 21.6, 44.7, 56.7, 66.0, 114.4, 118.7, 122.0, 123.7, 124.1, 125.2, 130.6, 138.5, 156.6

Synthesis of 6-morpholin-4-yl-N,N¹-diphenyl-[1,3,5]triazine-2,4-diamine (Compound 15) (VIRADEM-010895):

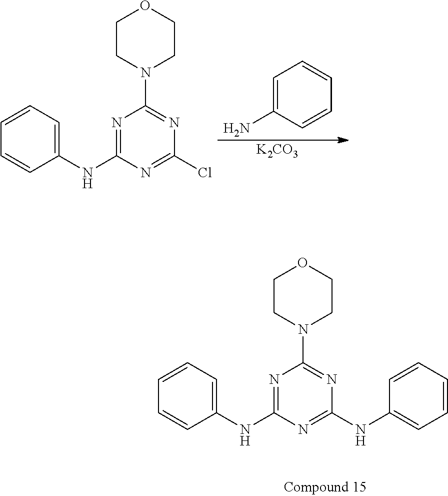

A mixture of (4-chloro-6-morpholin-4-yl-[1,3,5]triazin-2-yl)-phenylamine (1.17 g, 0.004 mol), 0.38 ml, 0.004 mol) and anhydrous potassium carbonate (0.56 g, 0.004 mol) in acetonitrile (40 ml) was stirred at room temperature for 30 minutes. Then refluxed for 2 hours. After cooling, the resulting mixture was poured into water (100 ml). White precipitates were collected and washed with water. The solid was dried under reduced pressure. Yield 0.96 g (69%). C₁₉H₂₀N₆O. MS m/z: 349.3 [(M+H)+]. ¹H NMR (400 MHz, DMSO-d₆) δ: 3.64 (m, 4H, CH₂NCH₂), 3.75 (m, 4H, CH₂OCH₂), 7.08 (t, 2H, 2C₆H₅), 7.34 (t, 4H, 2C₆H₅), 7.66 (d, 4H, 2C₆H₅), 10.1 (s, 1H, NH).

Other compounds were prepared analogously to the compounds 3-15 from the appropriate starting materials.

N-(3-Chlorophenyl)-6-morpholin-4-yl-N¹-phenyl-[1,3,5]triazine-2,4-diamine hydrochloride (Compound 16) (VIRADEM-011795): Yield 74%. M.p.=186-187° C. C₁₉H₁₉Cl₂N₆O. MS m/z: 383.6 [(M+H)+]. ¹H NMR (400 MHz, DMSO-d₆) δ: 3.65 (m, 4H, CH₂NCH₂), 3.74 (m, 4H, CH₂OCH₂), 7.05-7.93 (m, 9H, C₆H₄+C₆H₅), 10.0 (s, 1H, NH), 10.2 (s, 1H, NH).

N-(4-Chlorophenyl)-6-morpholin-4-yl-N¹-phenyl-[1,3,5]triazine-2,4-diamine hydrochloride (Compound 17) (VIRADEM-011796): Yield 77%. M.p.=226-227° C. C₁₉H₂₀Cl₂N₆O. MS m/z: 383.6 [(M+H)+]. ¹H NMR (400 MHz, DMSO-d₆) δ: 3.58 (m, 4H, CH₂NCH₂), 3.66 (m, 4H, CH₂OCH₂), 7.01-7.77 (m, 9H, C₆H₄+C₆H₅), 9.45 (s, 1H, NH), 9.57 (s, 1H, NH).

N-(3,5-Dimethylphenyl)-6-morpholin-4-yl-N¹-phenyl-[1,3,5]triazine-2,4-diamine hydrochloride (Compound 18) (VIRADEM-011797): Yield 71%. M.p.=202-3° C. C₂₁H₂₅ClN₆O. MS m/z: 377.5 [(M+H)+]. ¹H NMR (400 MHz, DMSO-d₆) δ: 2.27 (s, 6H, 2CH₃), 3.66 (m, 4H, CH₂NCH₂), 3.76 (m, 4H, CH₂OCH₂), 6.72-7.67 (m, 8H, C₆H₃+C₆H₅), 9.98 (s, 1H, NH), 10.1 (s, 1H, NH)

6-Morpholine-4-yl-N-phenyl-N¹-m-tolyl-[1,3,5]triazine-2,4-diamine hydrochloride (Compound 19) (VIRADEM-011798): Yield 74%. M.p.=204-5° C. C₂₀H₂₃ClN₆O. MS m/z: 363.4 [(M+H)+]. ¹H NMR (400 MHz, DMSO-d₆) δ: 2.30 (s, 3H, CH₃), 3.69 (m, 4H, CH₂NCH₂), 3.78 (m, 4H, CH₂OCH₂), 6.88-7.68 (m, 9H, C₆H₄+C₆H₅), 9.87 (s, 2H, 2NH).

N-(3,4-Dimethylphenyl)-6-morpholin-4-yl-N¹-phenyl-[1,3,5]triazine-2,4-diamine hydrochloride (Compound 20) (VIRADEM-011887): Yield 70%. M.p.=202-3° C. C₂₁H₂₅CIN₆O. MS m/z: 377.5 [(M+H)+]. ¹H NMR (400 MHz, CDCl3) δ: 2.25 (s, 6H, 2CH3), 3.76 (m, 4H, CH2NCH2), 3.86 (m, 4H, CH2OCH2), 7.05-7.56 (m, 8H, C₆H₃+C₆H₅), 9.57 (s, 1H, NH), 9.76 (s, 1H, NH).

N-(4-Fluorophenyl)-6-morpholin-4-yl-N¹-phenyl-[1,3,5]triazine-2,4-diamine hydrochloride (Compound 21) (VIRADEM-011888): Yield 78%. M.p.=207-8° C. C₂₉H₂₀ClFN₆O. MS m/z: 367.4 [(M+H)+]. ¹H NMR (400 MHz, DMSO-d₆) δ: 3.67 (m, 4H, CH₂NCH₂), 3.78 (m, 4H, CH₂OCH₂), 7.04-7.69 (m, 9H, C₆H₄+C₆H₅), 9.91 (s, 2H, 2NH).

6-Morpholine-4-yl-N-phenyl-N¹-p-tolyl-[1,3,5]triazine-2,4-diamine hydrochloride (Compound 22) (VIRADEM-011889): Yield 75%. M.p.=200-1° C. C₂₀H₂₃ClN₆O. MS m/z: 363.4 [(M+H)+]. ¹H NMR (400 MHz, DMSO-d₆) δ: 2.27 (s, 3H, CH₃), 3.66 (m, 4H, CH₂NCH₂), 3.75 (m, 4H, CH₂OCH₂), 7.05 (t, 1H, 2C₆H₅), 7.32 (t, 2H, C₆H₅), 7.13 and 7.54 (d-d, 4H, C₆H₄), 7.68 (d, 2H, 2C₆H₅), 9.85 (s, 2H, 2NH).

N-(3-Fluorophenyl)-6-morpholin-4-yl-N¹-phenyl-[1,3,5]triazine-2,4-diamine hydrochloride (Compound 23) (VIRADEM-011982): Yield 73%. M.p.=M.p.=199-200° C. ° C. C₁₉H₂₀ClFN₆O. MS m/z: 367.4 [(M+H)+]. ¹H NMR (400 MHz, DMSO-d₆) δ: 3.68 (m, 4H, CH₂NCH₂), 3.78 (m, 4H, CH₂OCH₂), 6.84-7.69 (m, 9H, C₆H₄+C₆H₅), 9.82 (s, 1H, NH), 9.93 (s, 1H, NH).

N-(3-Chloro-4-methylphenyl)-6-morpholin-4-yl-N¹-phenyl-[1,3,5]triazine-2,4-diamine hydrochloride (Compound 24) (VIRADEM-011987): Yield 77%. M.p.=208-9° C. C₂₀H₂₂Cl₂N₆O. MS m/z: 397.9 [(M+H)+]. ¹H NMR (400 MHz, DMSO-d₆) δ: 2.27 (s, 3H, CH₃), 3.65 (m, 4H, CH₂NCH₂), 3.76 (m, 4H, CH₂OCH₂), 7.03-7.88 (m, 8H, C₆H₃+C₆H₅), 9.63 (s, 1H, NH), 9.71 (s, 1H, NH).

N-(4-Ethoxyphenyl)-6-morpholin-4-yl-N¹-phenyl-[1,3,5]triazine-2,4-diamine hydrochloride (Compound 25) (VIRADEM-011998): Yield 65%. M.p.=227-228° C. C₂₁H₂₅ClN₆O₂. MS m/z: 393.5 [(M+H)+]. ¹H NMR (400 MHz, DMSO-d₆) δ: 1.32 (t, 3H, CH₃), 3.65 (m, 4H, CH₂NCH₂), 3.74 (m, 4H, CH₂OCH₂), 4.15 (q, 2H, CH₂), 6.89 and 7.51 (d-d, 4H, C₆H₄), 7.01 (t, 1H, 2C₆H₅), 7.32 (t, 2H, C₆H₅), 7.64 (d, 2H, 2C₆H₅), 9.83 (s, 1H, NH), 9.92 (s, 1H, NH).

N-(3,4-Dichlorophenyl)-6-morpholin-4-yl-N¹-phenyl-[1,3,5]triazine-2,4-diamine (Compound 26) (VIRADEM-012033): Yield 78%. M.p.=168-169° C. C₁₉H₁₈Cl₂N₆O. MS m/z: 418.2 [(M+H)+]. ¹H NMR (400 MHz, DMSO-d₆) δ: 3.66 (m, 4H, CH₂NCH₂), 3.75 (m, 4H, CH₂OCH₂), 6.97-8.15 (m, 8H, C₆H₃+C₆H₅), 9.31 (s, 1H, NH), 9.50 (s, 1H, NH).

1-[3-(4-Morpholin-4-yl-6-phenylamino-[1,3,5]triazin-2-ylamino)-phenyl]-ethanone hydrochloride (Compound 27) (VIRADEM-012125): Yield 80%. M.p.=212-213° C. C₂₁H₂₃ClN₆O₂. MS m/z: 391.4 [(M+H)+]. ¹H NMR (400 MHz, DMSO-d₆) δ: 2.56 (s, 3H, COCH₃), 3.69 (m, 4H, CH₂NCH₂), 3.80 (m, 4H, CH₂OCH₂), 7.03-8.33 (m, 9H, C₆H₅+C₆H₄), 9.86 (s, 1H, NH), 10.0 (s, 1H, NH).

6-Morpholine-4-yl-N-phenyl-N¹-(3-trifluoromethylphenyl)-[1,3,5]triazine-2,4-diamine hydrochloride (Compound 28) (VIRADEM-012235): Yield 77%. M.p.=151-2° C. C₂₀H₂₀ClF₃N₆O. MS m/z: 417.2 [(M+H)+]. ¹H NMR (400 MHz, DMSO-d₆) δ: 3.67 (m, 4H, CH₂NCH₂), 3.76 (m, 4H, CH₂OCH₂), 6.97-8.21 (m, 9H, C₆H₄+C₆H₅), 9.27 (s, 1H, NH), 9.55 (s, 1H, NH).

N-(3-Methoxyphenyl)-6-morpholin-4-yl-N¹-phenyl-[1,3,5]triazine-2,4-diamine hydrochloride (Compound 29) (VIRADEM-012252): Yield 62%. M.p.=203-4° C. C₂₀H₂₃ClN₆O₂. MS m/z: 379.3 [(M+H)+]. ¹H NMR (400 MHz, DMSO-d₆) δ: 3.69 (m, 4H, CH₂NCH₂), 3.73 (s, 3H, OCH₃), 3.79 (m, 4H, CH₂OCH₂), 6.64-7.69 (m, 9H, C₆H₅+C₆H₄), 10.0 (s, 2H, 2NH).

N,N¹-Dimethyl-6-morpholine-4-yl-N,N¹-diphenyl-[1,3,5]triazine-2,4-diamine (Compound 30) (VIRADEM-014284): Yield 67%. C₂₁H₂₄N₆O. MS m/z: 377.5 [(M+H)+].

N-Methyl-6-morpholine-4-yl-N,N¹-diphenyl-[1,3,5]triazine-2,4-diamine (Compound 31) (VIRADEM-015161): Yield 70%. M.p.=172-173° C. C₂₀H₂₂N₆O. MS m/z: 363.2 [(M+H)+].

6-Morpholine-4-yl-N-phenyl-N¹-(4-trifluoromethylphenyl)-[1,3,5]triazine-2,4-diamine (Compound 32) (VIRADEM-015166): Yield 77%. M.p.=211-212° C. C₂₀H₁₉F₃N₆O. MS m/z: 417.2 [(M+H)+].

N-(3,4-Dimethylphenyl)-N¹-methyl-6-morpholin-4-yl-N¹-phenyl-[1,3,5]triazine-2,4-diamine (Compound 33) (VIRADEM-015168): Yield 71%. M.p.=165-6° C. C₂₂H₂₆N₆O. MS m/z: 391.3 [(M+H)+]. ¹H NMR (400 MHz, DMSO-d₆) δ: 2.17 (s, 3H, CH₃), 2.28 (s, 3H, CH₃), 3.18 (s, 3H, CH₃), 3.67 (m, 4H, CH₂NCH₂), 3.78 (m, 4H, CH₂OCH₂), 7.09-7.58 (m, 8H, C₆H₅+C₆H₃), 10.0 (s, 2H, 2NH).

6-Morpholine-4-yl-N-phenyl-N¹-pyridin-3-yl-[1,3,5]triazine-2,4-diamine (Compound 34) (VIRADEM-015169): Yield 62%. C₁₈H₁₉N₇O. MS m/z: 350.2 [(M+H)+].

N-Methyl-6-morpholine-4-yl-N-phenyl-N¹-p-tolyl-[1,3,5]triazine-2,4-diamine (Compound 35) (15175): Yield 68%. M.p.=187-188° C. C₂₁H₂₄N₆O. MS m/z: 377.3 [(M+H)+].

N-(2,5-Dichlorophenyl)-6-morpholine-4-yl-N-phenyl-[1,3,5]triazine-2,4-diamine (Compound 36) (VIRADEM-015177): Yield 72%. M.p.=176-177° C. C₁₉H₁₈Cl₂N₆O. MS m/z: 417.2 [(M+H)+].

[4-(3,5-Dimethylpiperidin-1-yl)-6-morpholin-4-yl-[1,3,5]triazin-2-yl]-phenylamine (Compound 37) (15216): Yield 61%. C₂₀H₂₈N₆O. M.p.=182-183° C. MS m/z: 417.2 [(M+H)+]. ¹H NMR (400 MHz, DMSO-d₆) δ: 0.88 (d, 6H, 2CH₃), 1.49-2.25 (m, 6H), 3.59 (m, 4H, CH₂NCH₂), 3.68 (m, 4H, CH₂OCH₂), 4.64 (m, 2H), 6.92 (t, 1H, 2C₆H₅), 7.24 (t, 2H, C₆H₅), 7.69 (d, 2H, 2C₆H₅), 9.05 (s, 1H, NH).

6-Morpholin-4-yl-N-phenethyl-N¹-phenyl-[1,3,5]triazine-2,4-diamine (Compound 38) (VIRADEM-015217): Yield 73%. M.p.=122-123° C. C₂₁H₂₄N₆O. MS m/z: 377.3 [(M+H)+].

[4-Morpholin-4-yl-6-(4-phenylpiperazin-1-yl)-[1,3,5]triazin-2-yl]-phenylamine (Compound 39) (VIRADEM-015241): Yield 70%. M.p.=181-182° C. C₂₃H₂₇N₇O. MS m/z: 418.2 [(M+H)+].

N-(4-Methoxyphenyl)-N¹-methyl-6-morpholin-4-yl-N¹-phenyl-[1,3,5]triazine-2,4-diamine (Compound 40) (VIRADEM-015257): Yield 69%. M.p.=166-167° C. C₂₁H₂₄NO₂. MS m/z: 393.3 [(M+H)+].

(4-Azepan-1-yl-6-morpholin-4-yl-[1,3,5]triazin-2-yl-phenylamine (Compound 41) (VIRADEM-015297): Yield 65%. M.p.=226-227° C. C₁₉H₂₆N₆O. MS m/z: 355.4 [(M+H)+].

1-(4-Morpholin-4-yl-6-phenylamino-[1,3,5]triazin-2-yl)-piperidine-4-carboxylic acid amide (Compound 42) (VIRADEM-015351): Yield 69%. M.p.=212-213° C. C₁₉H₂₅N₇O. MS m/z: 384.2 [(M+H)+].

N-(2,3-Dihydrobenzo[1,4]dioxin-6-yl-6-morpholin-4-yl-N¹-phenyl-[1,3,5]triazine-2,4-diamine (Compound 43) (VIRADEM-015354): Yield 72%. M.p.=171-2° C. C₂₁H₂₂NO₃. MS m/z: 407.1 [(M+H)+].

[4-(2,6-Dimethylmorpholin-4-yl)-6-morpholin-4-yl-[1,3,5]triazin-2-yl]-phenylamine (Compound 44) (VIRADEM-015360): Yield 67%. M.p.=183-184° C. C₁₉H₂₆N₆O₂. MS m/z: 371.3 [(M+H)+].

N-(3,4-Dimethoxyphenyl)-6-morpholin-4-yl-N¹-phenyl-[1,3,5]triazine-2,4-diamine (Compound 45) (VIRADEM-015365): Yield 75%. M.p.=164-165° C. C₂₁H₂₄NO₃. MS m/z: 409.2 [(M+H)+].

N-(3-Chloro-4-methoxyphenyl)-6-morpholin-4-yl-N¹-phenyl-[1,3,5]triazine-2,4-diamine (Compound 46) (VIRADEM-015366): Yield 75%. M.p.=181-182° C. C₂₁H₂₄NO₃. MS m/z: 413.1 [(M+H)+].

N-(4-Butylphenyl)-6-morpholin-4-yl-N¹-phenyl-[1,3,5]triazine-2,4-diamine (Compound 47) (VIRADEM-015367): Yield 66%. M.p.=144-145° C. C₂₃H₂₈N₆O. MS m/z: 405.4 [(M+H)+].

N-(4-Ethoxyphenyl)-N¹,N¹,N²,N²-tetramethyl-[1,3,5]triazine-2,4,6-triamine (Compound 48) (VIRADEM-009799): Yield 56%. M.p.=215-216° C. C₁₅H₂₂N₆O. MS m/z: 303.1 [(M+H)+]. ¹H NMR (400 MHz, DMSO-d₆) δ: 1.34 (t, 3H, CH₃), 3.15 (m, 12H, 2CH₃NCH₃), 4.04 (q, 2H, CH₂), 6.93 and 7.52 (d-d, 4H, C₆H₄), 10.7 (s, 1H, NH).

6-Morpholine-4-yl-N,N¹-di-p-tolyl-[1,3,5]triazine-2,4-diamine hydrochloride (Compound 49) (VIRADEM-010029): Yield 76%. M.p.=258-259° C. C₂₁H₂₅ClN₆O. MS m/z: 377.2 [(M+H)+]. ¹H NMR (400 MHz, DMSO-d₆) δ: 2.28 (s, 6H, 2CH₃), 3.66 (m, 4H, CH₂NCH₂), 3.75 (m, 4H, CH₂OCH₂), 7.15 and 7.52 (d-d, 8H, 2C₆H₄), 10.1 (s, 2H, 2NH).

N,N¹-Bis-(3-methoxyphenyl)-6-morpholine-4-yl-[1,3,5]triazine-2,4-diamine hydrochloride (Compound 50) (VIRADEM-012263): Yield 61%. C₂₁H₂₅ClN₆O₃. MS m/z: 409.2 [(M+H)+]. ¹H NMR (400 MHz, DMSO-d₆) δ: 3.66 (m, 4H, CH₂NCH₂), 3.74 (m, 4H, CH₂OCH₂), 3.79 (s, 3H, OCH₃), 6.63-7.34 (m, 8H, 2C₆H₄), 10.0 (s, 2H, 2NH).

N,N¹-Bis-(3-fluorophenyl)-6-morpholine-4-yl-[1,3,5]triazine-2,4-diamine hydrochloride (Compound 51) (VIRADEM-010852): Yield 66%. M.p.=215-6° C. C₁₉H₁₉ClF₂N₆O. MS m/z: 385.2 [(M+H)+]. ¹H NMR (400 MHz, DMSO-d₆) δ: 3.64 (m, 4H, CH₂NCH₂), 3.76 (m, 4H, CH₂OCH₂), 6.82-7.78 (m, 8H, 2C₆H₄), 9.79 (s, 2H, 2NH).

N,N¹-Bis-(3,4-dimethylphenyl)-6-morpholine-4-yl-[1,3,5]triazine-2,4-diamine hydrochloride (Compound 52) (VIRADEM-010853): Yield 70%. M.p.=220-1° C. C₂₃H₂₉ClN₆O. MS m/z: 405.3 [(M+H)+]. ¹H NMR (400 MHz, DMSO-d₆) δ: 2.21 (s, 12H, 4CH₃), 3.68 (m, 4H, CH₂NCH₂), 3.79 (m, 4H, CH₂OCH₂), 7.08-7.39 (m, 6H, 2C₆H₃), 9.94 (s, 2H, 2NH).

N,N¹-Bis-(3,5-dimethylphenyl)-6-morpholine-4-yl-[1,3,5]triazine-2,4-diamine hydrochloride (Compound 53) (VIRADEM-010854): Yield 77%. M.p.=223-224° C. C₂₃H₂₉ClN₆O. MS m/z: 405.3 [(M+H)+]. ¹H NMR (400 MHz, DMSO-d₆) δ: 2.24 (s, 12H, 4CH₃), 3.70 (m, 4H, CH₂NCH₂), 3.79 (m, 4H, CH₂OCH₂), 6.74 (s, 2H, 2C₆H₃), 7.36 (s, 4H, 2C₆H₃), 9.98 (s, 2H, 2NH).

N,N¹-Bis-(2-fluorophenyl)-6-morpholine-4-yl-[1,3,5]triazine-2,4-diamine hydrochloride (Compound 54) (VIRADEM-010896): Yield 64%. M.p.=196-197° C. C₁₉H₁₉ClF₂N₆O. MS m/z: 385.2 [(M+H)+]. ¹H NMR (400 MHz, DMSO-d₆) δ: 3.61 (m, 4H, CH₂NCH₂), 3.73 (m, 4H, CH₂OCH₂), 7.14-7.73 (m, 8H, 2C₆H₄), 9.00 (s, 2H, 2NH).

6-Morpholine-4-yl-N,N¹-di-m-tolyl-[1,3,5]triazine-2,4-diamine hydrochloride (Compound 55) (VIRADEM-010897): Yield 73%. M.p.=195-196° C. C₂₁H₂₅ClN₆O. MS m/z: 377.2 [(M+H)+]. ¹H NMR (400 MHz, DMSO-d₆) δ: 2.26 (s, 6H, 2CH₃), 3.68 (m, 4H, CH₂NCH₂), 3.77 (m, 4H, CH₂OCH₂), 6.89-7.58 (m, 8H, 2C₆H₄), 10.0 (s, 2H, 2NH).

N,N¹-Bis-(4-ethylphenyl)-6-morpholine-4-yl-[1,3,5]triazine-2,4-diamine hydrochloride (Compound 56) (VIRADEM-010898): Yield 74%. M.p.=210-211° C. C₂₃H₂₉ClN₆O. MS m/z: 405.3 [(M+H)+]. ¹H NMR (400 MHz, DMSO-d₆) δ: 1.27 (t, 6H, 2CH₂CH₃), 2.59 (q, 4H, 2CH₂CH₃) 3.69 (m, 4H, CH₂NCH₂), 3.78 (m, 4H, CH₂OCH₂), 7.18 and 7.56 (d-d, 8H, 2C₆H₄), 10.1 (s, 2H, 2NH).

N,N¹-Bis-(4-fluorophenyl)-6-morpholine-4-yl-[1,3,5]triazine-2,4-diamine hydrochloride (Compound 57) (VIRADEM-010899): Yield 69%. M.p.=236-237° C. C₁₉H₁₉ClF₂N₆O. MS m/z: 385.2 [(M+H)+]. ¹H NMR (400 MHz, DMSO-d₆) δ: 3.66 (m, 4H, CH₂NCH₂), 3.74 (m, 4H, CH₂OCH₂), 7.15-7.67 (m, 8H, 2C₆H₄), 9.96 (s, 2H, 2NH).

N,N¹-Bis-(4-methoxyphenyl)-6-morpholine-4-yl-[1,3,5]triazine-2,4-diamine hydrochloride (Compound 58) (VIRADEM-010900): Yield 79%. M.p.=197-198° C. C₂₁H₂₅ClN₆O₃. MS m/z: 409.2 [(M+H)+]. ¹H NMR (400 MHz, DMSO-d₆) δ: 3.67 (m, 4H, CH₂NCH₂), 3.75 (m, 4H, CH₂OCH₂), 3.77 (s, 6H, 2OCH₃), 6.94 and 7.52 (d-d, 8H, 2C₆H₄), 10.2 (s, 2H, 2NH).

N,N¹-Bis-(4-chlorophenyl)-6-morpholine-4-yl-[1,3,5]triazine-2,4-diamine hydrochloride (Compound 59) (VIRADEM-010901): Yield 83%. M.p.>260° C. C₁₉H₁₉Cl₃N₆O. MS m/z: 418.1 [(M+H)+]. ¹H NMR (400 MHz, DMSO-d₆) δ: 3.65 (m, 4H, CH₂NCH₂), 3.74 (m, 4H, CH₂OCH₂), 7.16 and 7.75 (d-d, 8H, 2C₆H₄), 9.69 (s, 2H, 2NH).

N,N¹-Bis-(3,4-dimethoxyphenyl)-6-morpholine-4-yl-[1,3,5]triazine-2,4-diamine (Compound 60) (VIRADEM-011004): Yield 69%. M.p.=188-189° C. C₂₃H₂₈NO₅. MS m/z: 469.3 [(M+H)+]. ¹H NMR (400 MHz, DMSO-d₆) δ: 3.65 (m, 4H, CH₂NCH₂), 3.74 (m, 4H, CH₂OCH₂), 3.77 (s, 12H, 4OCH₃), 6.84-7.42 (m, 6H, 2C₆H₃), 8.89 (s, 2H, 2NH).

N-(4-chlorophenyl)-N¹-(4-methoxyphenyl)-6-morpholin-4-yl-[1,3,5]triazine-2,4-diamine (Compound 61) (VIRADEM-011040): Yield 73%. M.p.=233-234° C. C₂₀H₂₁N₆O₂. MS m/z: 413.7 [(M+H)+]. ¹H NMR (400 MHz, DMSO-d₆) δ: 3.66 (m, 4H, CH₂NCH₂), 3.75 (m, 4H, CH₂OCH₂), 3.77 (s, 3H, OCH₃), 6.85-7.74 (m, 8H, 2C₆H₄), 8.90 (s, 1H, NH), 9.73 (s, 1H, NH).

N-(3,4-Dichlorophenyl)-N¹,N²-diethyl-[1,3,5]triazine-2,4,6-triamine (Compound 62) (VIRADEM-011046): Yield 63%. M.p.=187-188° C. C₁₃H₁₇Cl₃N₆. MS m/z: 328.0 [(M+H)+]. ¹H NMR (400 MHz, DMSO-d₆) δ: 1.21 (t, 6H, 2CH₃), 3.40 (m, 4H, 2CH₂), 7.38-8.12 (m, 3H, C₆H₃), 8.30-10.3 (m, 3H, 3NH).

N,N¹-Bis-(3,5-dichlorophenyl)-6-morpholine-4-yl-[1,3,5]triazine-2,4-diamine (Compound 63) (VIRADEM-011148): Yield 80%. M.p.=237-238° C. C₁₉H₁₆Cl₄N₆O. MS m/z: 487.1 [(M+H)+]. ¹H NMR (400 MHz, DMSO-d₆) δ: 3.66 (m, 4H, CH₂NCH₂), 3.75 (m, 4H, CH₂OCH₂), 7.09 (s, 2H, 2C₆H₃), 7.84 (s, 4H, 2C₆H₃), 9.50 (s, 2H, 2NH).

N,N¹-Bis-(4-carboethoxyphenyl)-6-morpholine-4-yl-[1,3,5]triazine-2,4-diamine (Compound 64) (VIRADEM-011149): Yield 71%. M.p.=217-218° C. C₂₅H₂₈N₆O₅. MS m/z: 493.4 [(M+H)+]. ¹H NMR (400 MHz, DMSO-d₆) δ: 1.32 (t, 4H, COOCH₂CH₃), 3.69 (m, 4H, CH₂NCH₂), 3.79 (m, 4H, CH₂OCH₂), 4.29 (q, 4H, COOCH₂CH₃), 7.09 (s, 2H, 2C₆H₃), 7.88 and 7.92 (d-d, 8H, 2C₆H₄), 9.66 (s, 2H, 2NH).

6-Morpholine-4-yl-N,N′-bis-(3-trifluorophenyl)-[1,3,5]triazine-2,4-diamine (Compound 65) (VIRADEM-011150): Yield 74%. M.p.=155-156° C. C₂₁H₁₈F₆N₆O. MS m/z: 485.2 [(M+H)+]. ¹H NMR (400 MHz, DMSO-d₆) δ: 3.66 (m, 4H, CH₂NCH₂), 3.75 (m, 4H, CH₂OCH₂), 7.25-8.19 (m, 8H, 2C₆H₄), 9.45 (s, 2H, 2NH).

N,N,N¹,N¹-Tetramethyl-6-morpholin-4-yl-[1,3,5]triazine-2,4-diamine hydrochloride (Compound 66) (VIRADEM-011151): Yield 58%. M.p.=147-148° C. C₂₃H₂₈N₆O₅. MS m/z: 469.3 [(M+H)+]. ¹H NMR (400 MHz, DMSO-d₆) δ: 3.03 (s, 12H, 2CH₃NCH₃), 3.58 (m, 4H, CH₂NCH₂), 3.65 (m, 4H, CH₂OCH₂).

N,N,N¹,N¹-Tetramethyl-N²-p-tolyl-[1,3,5]triazine-2,4,6-triamine hydrochloride (Compound 67) (VIRADEM-011243): Yield 56%. M.p.=241-242° C. C₁₄H₂₁ClN₆. MS m/z: 303.1 [(M+H)+]. ¹H NMR (400 MHz, DMSO-d₆) δ: 2.30 (s, 3H, CH₃), 3.16 (m, 12H, 2CH₃NCH₃), 7.18 and 7.52 (d-d, 4H, C₆H₄), 10.7 (s, 1H, NH).

N-(4-Chlorophenyl)-6-morpholine-4-yl-N¹-p-tolyl[1,3,5]triazine-2,4-diamine hydrochloride (Compound 68) (VIRADEM-011245): Yield 79%. M.p.>260° C. C₂₀H₂₂N₆O. MS m/z: 397.7 [(M+H)+]0.1H NMR (400 MHz, DMSO-d₆) δ: 2.28 (s, 3H, CH₃), 3.68 (m, 4H, CH₂NCH₂), 3.77 (m, 4H, CH₂OCH₂), 7.18 and 7.53 (d-d, 4H, C₆H₄), 7.37 and 7.72 (d-d, 4H, C₆H₄), 9.91 (s, 1H, NH), 10.1 (s, 1H, 1NH).

N,N¹-Bis-(4-ethoxyphenyl)-6-morpholine-4-yl-[1,3,5]triazine-2,4-diamine (Compound 69) (VIRADEM-011394): Yield 71%. M.p.=210-211° C. C₂₃H₂₈N₆O₃. MS m/z: 437.3 [(M+H)+]. ¹H NMR (400 MHz, DMSO-d₆) δ: 1.29 (t, 6H, 2 OCH₂CH₃), 3.61 (m, 4H, CH₂NCH₂), 3.70 (m, 4H, CH₂OCH₂), 4.00 (q, 4H, 2OCH₂CH₃), 6.83 and 7.58 (d-d, 8H, 2C₆H₄), 8.95 (s, 2H, 2NH).

N-(3-Chloro-4-fluorophenyl)-6-morpholine-4-yl-N¹-p-tolyl[1,3,5]triazine-2,4-diamine hydrochloride (Compound 70) (VIRADEM-011395): Yield 77%. M.p.=244-245° C. C₂₀H₂₂ClFN₆O. MS m/z: 415.6 [(M+H)+]. ¹H NMR (400 MHz, DMSO-d₆) δ: 2.25 (s, 3H, CH₃), 3.67 (m, 4H, CH₂NCH₂), 3.76 (m, 4H, CH₂OCH₂), 7.08 and 7.55 (d-d, 4H, C₆H₄), 7.27-8.09 (m, 3H, C₆H₃), 9.11 (s, 1H, NH), 9.31 (s, 1H, 1NH).

N-(4-Ethoxyphenyl)-N¹-(4-fluorophenyl)-6-morpholine-4-yl-[1,3,5]triazine-2,4-diamine hydrochloride (Compound 71) (VIRADEM-011397): Yield 71%. M.p.=227-228° C. C₂₁H₂₄ClFN₆O₂. MS m/z: 411.2 [(M+H)+]. ¹H NMR (400 MHz, DMSO-d₆) δ: 1.31 (s, 3H, OCH₂CH₃), 3.66 (m, 4H, CH₂NCH₂), 3.75 (m, 4H, CH₂OCH₂), 3.98 (q, 2H, OCH₂CH₃), 6.92 and 7.51 (d-d, 4H, C₆H₄), 7.16-7.64 (m, 4H, C₆H₄), 9.96 (s, 1H, NH), 10.0 (s, 1H, 1NH).

N-(4-Ethoxyphenyl)-6-morpholine-4-yl-N¹-p-tolyl-[1,3,5]triazine-2,4-diamine hydrochloride (Compound 72) (VIRADEM-011398): Yield 77%. M.p.=226-227° C. C₂₂H₂₇ClN₆O₂. MS m/z: 411.2 [(M+H)+]. ¹H NMR (400 MHz, DMSO-d₆) δ: 1.32 (s, 3H, OCH₂CH₃), 2.30 (s, 3H, CH₃), 3.68 (m, 4H, CH₂NCH₂), 3.77 (m, 4H, CH₂OCH₂), 4.05 (q, 2H, OCH₂CH₃), 6.92 and 7.51 (d-d, 4H, C₆H₄), 7.16 and 7.51 (d-d, 4H, C₆H₄), 10.1 (s, 2H, 2NH).

N-(4-Ethylphenyl)-6-morpholine-4-yl-N¹-p-tolyl-[1,3,5]triazine-2,4-diamine hydrochloride (Compound 73) (VIRADEM-011399): Yield 72%. M.p.=191-192° C. C₂₂H₂₇ClN₆O. MS m/z: 391.2 [(M+H)+]. ¹H NMR (400 MHz, DMSO-d₆) δ: 1.19 (s, 3H, CH₂CH₃), 2.26 (s, 3H, CH₃), 3.65 (m, 4H, CH₂NCH₂), 3.74 (m, 4H, CH₂OCH₂), 3.76 (q, 2H, CH₂CH₃), 7.11 and 7.51 (d-d, 4H, C₆H₄), 7.14 and 7.55 (d-d, 4H, C₆H₄), 9.83 (s, 1H, 1NH), 9.95 (s, 1H, 1NH).

N-(4-Chlorophenyl)-N¹,N²-diethyl-1,3,5]triazine-[2,4,6-triamine hydrochloride (Compound 74) (VIRADEM-011428): Yield 60%. M.p.=140-141° C. C₁₃H₁₈Cl₂N₆. MS m/z: 293.5 [(M+H)+]. ¹H NMR (400 MHz, DMSO-d₆) δ: 1.22 (t, 6H, 2CH₃), 3.41 (m, 4H, 2CH₂), 7.36 and 7.70 (d-d, 4H, C₆H₄), 8.28-10.2 (m, 3H, 3NH).

N,N¹-Bis-(3-chloro-4-methylphenyl)-6-morpholine-4-yl-[1,3,5]triazine-2,4-diamine (Compound 75) (VIRADEM-011486): Yield 76%. M.p.=201-202° C. C₂₁H₂₂Cl₂N₆O. MS m/z: 446.1 [(M+H)+]. ¹H NMR (400 MHz, DMSO-d₆) δ: 2.27 (s, 3H, CH₃), 3.66 (m, 4H, CH₂NCH₂), 3.77 (m, 4H, CH₂OCH₂), 7.05-8.05 (m, 3H, C₆H₃), 10.1 (s, 2H, 2NH).

N-(3,4-Dichlorophenyl)-6-morpholine-4-yl-N¹-p-tolyl-[1,3,5]triazine-2,4-diamine (Compound 76) (VIRADEM-011487): Yield 79%. M.p.=232-233° C. C₂₀H₂₀Cl₂N₆O. MS m/z: 432.1 [(M+H)+]. ¹H NMR (400 MHz, DMSO-d₆) δ: 2.27 (s, 3H, CH₃), 3.66 (m, 4H, CH₂NCH₂), 3.75 (m, 4H, CH₂OCH₂), 7.12 and 7.52 (d-d, 4H, C₆H₄), 7.37-8.10 (m, 3H, C₆H₃), 9.85 (s, 1H, 1NH), 10.0 (s, 1H, 1NH).

N-(3-Chloro-4-methylphenyl)-6-morpholine-4-yl-N¹-p-tolyl-[1,3,5]triazine-2,4-diamine hydrochloride (Compound 77) (VIRADEM-011488): Yield 75%. M.p.=234-235° C. C₂₁H₂₄Cl₂N₆O. MS m/z: 411.7 [(M+H)+]. ¹H NMR (400 MHz, DMSO-d₆) δ: 2.17 (s, 3H, CH₃), 2.28 (s, 3H, CH₃), 3.65 (m, 4H, CH₂NCH₂), 3.74 (m, 4H, CH₂OCH₂), 7.11 and 7.53 (d-d, 4H, C₆H₄), 7.32-8.12 (m, 3H, C₆H₃), 9.89 (s, 1H, 1NH), 10.1 (s, 1H, 1NH).

N-(4-Chlorophenyl)-N¹-(4-fluorophenyl)-6-morpholine-4-yl-[1,3,5]triazine-2,4-diamine hydrochloride (Compound 78) (VIRADEM-011489): Yield 81%. M.p.=258-259° C. C₁₉H₁₉Cl₂FN₆O. MS m/z: 401.6 [(M+H)+]. ¹H NMR (400 MHz, CDCl₃) δ: 3.76 (m, 4H, CH₂NCH₂), 3.83 (m, 4H, CH₂OCH₂), 7.06-7.51 (m, 8H, C₆H₄+C₆H₄), 10.4 (s, 1H, 1NH), 10.5 (s, 1H, 1NH), 12.6 (s, 1H, HCl).

N-(3,4-Dimethylphenyl)-N¹-(4-fluorophenyl)-6-morpholine-4-yl-[1,3,5]triazine-2,4-diamine hydrochloride (Compound 79) (VIRADEM-011490): Yield 75%. M.p.=203-205° C. C₂₁H₂₄ClFN₆O₂. MS m/z: 395.2 [(M+H)+]. ¹H NMR (400 MHz, DMSO-d₆) δ: 2.19 (s, 3H, CH₃), 2.21 (s, 3H, CH₃), 3.69 (m, 4H, CH₂NCH₂), 3.78 (m, 4H, CH₂OCH₂), 7.09-7.67 (m, 7H, C₆H₃+C₆H₄), 10.1 (s, 2H, 2NH).

N-(4-Fluorophenyl)-6-morpholine-4-yl-N¹-p-tolyl-[1,3,5]triazine-2,4-diamine hydrochloride (Compound 80) (VIRADEM-011491): Yield 71%. M.p.=225-226° C. C₂₀H₂₂ClFN₆O. MS m/z: 381.2 [(M+H)+]. ¹H NMR (400 MHz, DMSO-d₆) δ: 2.28 (s, 3H, CH₃), 3.68 (m, 4H, CH₂NCH₂), 3.77 (m, 4H, CH₂OCH₂), 7.14-7.66 (m, 8H, C₆H₄+C₆H₄), 10.2 (s, 2H, 2NH).

N,N¹-Diethyl-N²-(4-phenoxyphenyl)-[1,3,5]triazine-2,4,6-triamine (Compound 81) (VIRADEM-011567): Yield 64%. M.p.=142-143° C. C₁₉H₂₂N₆O. MS m/z: 351.5 [(M+H)+]. ¹H NMR (400 MHz, DMSO-d₆) δ: 1.21 (t, 6H, 2CH₃), 3.40 (m, 4H, 2CH₂), 7.05-7.67 (m, 9H, C₆H₅+C₆H₄), 8.30-10.1 (m, 3H, 3NH).

N-(4-Fluorophenyl)-N¹-(4-methoxyphenyl)-6-morpholin-4-yl-[1,3,5]triazine-2,4-diamine (Compound 82) (VIRADEM-011568): Yield 75%. M.p.=210-211° C. C₂₀H₂₂ClFN₆O₂. MS m/z: 397.2 [(M+H)+]. ¹H NMR (400 MHz, DMSO-d₆) δ: 3.68 (m, 4H, CH₂NCH₂), 3.74 (m, 4H, CH₂OCH₂), 3.76 (s, 3H, OCH₃), 6.93 and 7.52 (d-d, 4H, C₆H₄), 7.16-7.67 (m, 4H, C₆H₄), 10.2 (s, 2H, 2NH).

N-(3,5-Dimethylphenyl)-N¹-(4-Fluorophenyl)-6-morpholin-4-yl-[1,3,5]triazine-2,4-diamine hydrochloride (Compound 83) (VIRADEM-011569): Yield 77%. M.p.=191-193° C. C₂₁H₂₄ClFN₆O. MS m/z: 395.2 [(M+H)+]. ¹H NMR (400 MHz, DMSO-d₆) δ: 2.22 (s, 6H, 2CH₃), 3.69 (m, 4H, CH₂NCH₂), 3.78 (m, 4H, CH₂OCH₂), 6.73 (s, 1H, C₆H₃), 7.25 (s, 2H, C₆H₃), 7.16-7.66 (m, 4H, C₆H₄), 10.2 (s, 2H, 2NH).

N-(4-Methoxyphenyl)-6-morpholine-4-yl-N¹-p-tolyl-[1,3,5]triazine-2,4-diamine hydrochloride (Compound 84) (VIRADEM-011570): Yield 73%. M.p.=225-226° C. C₂₁H₂₅ClN₆O₂. MS m/z: 392.2 [(M+H)+]. ¹H NMR (400 MHz, DMSO-d₆) δ: 2.28 (s, 3H, CH₃), 3.68 (m, 4H, CH₂NCH₂), 3.77 (m, 4H, CH₂OCH₂), 3.79 (s, 3H, OCH₃), 6.91 and 7.51 (d-d, 4H, C₆H₄), 7.16 and 7.52 (d-d, 4H, C₆H₄), 10.0 (s, 2H, 2NH).

N-(3,4-Dichlorophenyl)-N¹-(4-methoxyphenyl)-6-morpholin-4-yl-[1,3,5]triazine-2,4-diamine (Compound 85) (VIRADEM-011658): Yield 81%. M.p.=220-221° C. C₂₀H₂₀Cl₂N₆O₂. MS m/z: 448.1 [(M+H)+]. ¹H NMR (400 MHz, DMSO-d₆) δ: 3.66 (m, 4H, CH₂NCH₂), 3.75 (m, 4H, CH₂OCH₂), 3.77 (s, 3H,)CH₃), 6.92 and 7.52 (d-d, 4H, C₆H₄), 7.38-8.11 (m, 3H, C₆H₃), 9.88 (s, 1H, 1NH), 10.1 (s, 1H, 1NH).

4-[4-(4-Fluorophenylamino)-6-morpholin-4-yl-[1,3,5]triazin-2-ylamino]benzoic acid ethyl ester (Compound 86) (VIRADEM-011660): Yield 70%. M.p.=177-178° C. C₂₂H₂₃FN₆O₃. MS m/z: 439.2 [(M+H)+]. ¹H NMR (400 MHz, DMSO-d₆) δ: 1.32 (t, 3H, COOCH₂CH₃), 3.67 (m, 4H, CH₂NCH₂), 3.76 (m, 4H, CH₂OCH₂), 4.29 (s, 2H, COOCH₂CH₃), 7.11 and 7.75 (d-d, 4H, C₆H₄), 7.87-7.91 (d-d, 4H, C₆H₄), 9.28 (s, 1H, 1NH), 9.57 (s, 1H, 1NH).

N-(3,4-Dichlorophenyl)-N¹-(3-fluorophenyl)-6-morpholin-4-yl-[1,3,5]triazine-2,4-diamine (Compound 87) (VIRADEM-011662): Yield 78%. M.p.=250-251° C. C₁₉H₁₇Cl₂FN₆O. MS m/z: 436.0 [(M+H)+].

N-(3,4-Dichlorophenyl)-N¹-(4-fluorophenyl)-6-morpholin-4-yl-[1,3,5]triazine-2,4-diamine (Compound 88) (VIRADEM-011663): Yield 80%. M.p.=185-186° C. C₁₉H₁₇Cl₂FN₆O. MS m/z: 436.0 [(M+H)+].

N-(4-Butylphenyl)-N¹-(4-methoxyphenyl)-6-morpholin-4-yl-[1,3,5]triazine-2,4-diamine (Compound 89) (VIRADEM-011664): Yield 68%. M.p.=173-175° C. C₂₄H₃₀N₆O₂. MS m/z: 435.3 [(M+H)+].

N-(3-Chloro-4-methylphenyl)-N¹-(4-chlorophenyl)-6-morpholin-4-yl-[1,3,5]triazine-2,4-diamine (Compound 90) (VIRADEM-011665): Yield 77%. M.p.=237-238° C. C₂₀H₂₀Cl₂N₆O. MS m/z: 432.1 [(M+H)+].

N-(3-Chloro-4-fluorophenyl)-N¹-(4-methoxyphenyl)-6-morpholin-4-yl-[1,3,5]triazine-2,4-diamine (Compound 91) (VIRADEM-011666): Yield 77%. M.p.=209-210° C. C₂₀H₂₀ClFN₆O₂. MS m/z: 431.6 [(M+H)+].

N-(3-Chloro-4-methylphenyl)-N¹-(4-methoxyphenyl)-6-morpholin-4-yl-[1,3,5]triazine-2,4-diamine (Compound 92) (VIRADEM-011667): Yield 71%. M.p.=207-208° C. C₂₁H₂₃ClN₆O₂. MS m/z: 432.1 [(M+H)+].

N-(3-Chloro-4-fluorophenyl)-N¹-(4-fluorophenyl)-6-morpholin-4-yl-[1,3,5]triazine-2,4-diamine (Compound 93) (VIRADEM-011669): Yield 75%. M.p.=225-226° C. C₁₉H₁₇ClF₂N₆O. MS m/z: 419.6 [(M+H)+].

N-(4-Chlorophenyl)-N¹-(4-ethylphenyl)-6-morpholin-4-yl-[1,3,5]triazine-2,4-diamine hydrochloride (Compound 94) (VIRADEM-011670): Yield 73%. M.p.=237-238° C. C₂₁H₂₄Cl₂N₆O. MS m/z: 448.1 [(M+H)+].

N-(4-Chlorophenyl)-N¹-(3,4-dimethylphenyl)-6-morpholin-4-yl-[1,3,5]triazine-2,4-diamine hydrochloride (Compound 95) (VIRADEM-011671): Yield 78%. M.p.=241-242° C. C₂₁H₂₄Cl₂N₆O. MS m/z: 448.1 [(M+H)+].

N-(4-Chlorophenyl)-N¹-(3,5-dimethylphenyl)-6-morpholin-4-yl-[1,3,5]triazine-2,4-diamine hydrochloride (Compound 96) (11672): Yield 80%. M.p.=234-235° C. C₂₁H₂₄Cl₂N₆O. MS m/z: 448.1 [(M+H)+].

N-(3-Chloro-4-methylphenyl)-6-morpholin-4-yl-N¹-m-tolyl-[1,3,5]triazine-2,4-diamine hydrochloride (Compound 97) (VIRADEM-011673): Yield 81%. M.p.=206-207° C. C₂₁H₂₄Cl₂N₆O. MS m/z: 411.0 [(M+H)+]. ¹H NMR (500 MHz, DMSO-d₆) δ: 2.28 (s, 6H, 2CH₃), 3.68 (m, 4H, CH₂NCH₂), 3.77 (m, 4H, CH₂OCH₂), 6.90-7.76 (m, 7H, C₆H₃+C₆H₄), 10.2 (s, 2H, 2NH). ³C-NMR (150 MHz, DMSO-d₆) δ: 19.4, 21.6, 44.5, 66.1, 118.5, 119.9, 121.3, 121.8, 124.7, 129.0, 126.4, 131.5, 133.4, 134.8, 137.9, 138.4.

N-Benzo[1,3]dioxol-5-yl-N¹-(4-fluorophenyl)-6-morpholin-4-yl-[1,3,5]triazine-2,4-diamine hydrochloride (Compound 98) (VIRADEM-011674): Yield 70%. M.p.=207-208° C. C₂₀H₂₀ClFN₆O₃. MS m/z: 411.2 [(M+H)+].

N-(4-Ethylphenyl)-N¹-(4-methoxyphenyl)-6-morpholin-4-yl-[1,3,5]triazine-2,4-diamine hydrochloride (Compound 99) (VIRADEM-011675): Yield 65%. M.p.=205-206° C. C₂₂H₂₇ClN₆O₂. MS m/z: 407.2 [(M+H)+].

N-(3,4-Dimethylphenyl)-N¹-(4-methoxyphenyl)-6-morpholin-4-yl-[1,3,5]triazine-2,4-diamine hydrochloride (Compound 100) (VIRADEM-011676): Yield 73%. M.p.=212-213° C. C₂₂H₂₇ClN₆O₂. MS m/z: 407.2 [(M+H)+].

N-(3,5-Dimethylphenyl)-N¹-(4-methoxyphenyl)-6-morpholin-4-yl-[1,3,5]triazine-2,4-diamine hydrochloride (Compound 101) (VIRADEM-011677): Yield 72%. M.p.=211-212° C. C₂₂H₂₇ClN₆O₂. MS m/z: 407.2 [(M+H)+].

N-(3-Chlorophenyl)-N¹-(3-fluorophenyl)-6-morpholin-4-yl-[1,3,5]triazine-2,4-diamine hydrochloride (Compound 102) (VIRADEM-011678): Yield 70%. M.p.=195-196° C. C₁₉H₁₉Cl₂FN₆O. MS m/z: 401.6 [(M+H)+].

N-(4-Chlorophenyl)-N¹-(3-fluorophenyl)-6-morpholin-4-yl-[1,3,5]triazine-2,4-diamine hydrochloride (Compound 102) (VIRADEM-011679): Yield 70%. M.p.=213-214° C. C₁₉H₁₉Cl₂FN₆O. MS m/z: 401.6 [(M+H)+].

N-(4-Chlorophenyl)-6-morpholin-4-yl-N¹-m-tolyl-[1,3,5]triazine-2,4-diamine hydrochloride (Compound 103) (VIRADEM-011680): Yield 77%. M.p.=213-215° C. C₂₀H₂₂Cl₂N₆O. MS m/z: 397.6 [(M+H)+]. ¹H NMR (400 MHz, DMSO-d₆) δ: 2.27 (s, 3H, CH₃), 3.65 (m, 4H, CH₂NCH₂), 3.76 (m, 4H, CH₂OCH₂), 6.88-7.49 (m, 4H, C₆H₄), 7.37 and 7.71 (d-d, 4H, C₆H₄), 9.83 (s, 1H, NH), 9.96 (s, 1H, NH).

N-(3-Fluorophenyl)-N¹-(4-methoxyphenyl)-6-morpholin-4-yl-[1,3,5]triazine-2,4-diamine hydrochloride (Compound 104) (VIRADEM-011681): Yield 75%. M.p.=195-196° C. C₂₀H₂₂ClFN₆O₂. MS m/z: 433.6 [(M+H)+]. ¹H NMR (400 MHz, DMSO-d₆) δ: 3.69 (m, 4H, CH₂NCH₂), 3.77 (m, 4H, CH₂OCH₂), 3.76 (s, 3H, OCH₃), 6.80-7.73 (m, 4H, C₆H₄), 6.91 and 7.54 (d-d, 4H, C₆H₄), 9.58 (s, 1H, NH), 9.81 (s, 1H, NH).

N-(3,4-Dimethylphenyl)-N¹-(3-fluorophenyl)-6-morpholin-4-yl-[1,3,5]triazine-2,4-diamine hydrochloride (Compound 105) (VIRADEM-011682): Yield 71%. M.p.=202-203° C. C₂₁H₂₄ClFN₆O. MS m/z: 395.2 [(M+H)+].

N-(3,5-Dimethylphenyl)-N¹-(3-fluorophenyl)-6-morpholin-4-yl-[1,3,5]triazine-2,4-diamine hydrochloride (Compound 106) (VIRADEM-011683): Yield 73%. M.p.=204-205° C. C₂₁H₂₄ClFN₆O. MS m/z: 395.2 [(M+H)+].

N-(4-Ethylphenyl)-N¹-(4-fluorophenyl)-6-morpholin-4-yl-[1,3,5]triazine-2,4-diamine hydrochloride (Compound 107) (VIRADEM-011684): Yield 69%. M.p.=193-194° C. C₂₁H₂₄ClFN₆O. MS m/z: 395.2 [(M+H)+].

N-(3,4-Dimethylphenyl)-6-morpholin-4-yl-N¹-m-tolyl-[1,3,5]triazine-2,4-diamine hydrochloride (Compound 108) (VIRADEM-011686): Yield 77%. M.p.=210-211° C. C₂₂H₂₇ClN₆O. MS m/z: 391.2 [(M+H)+]. ¹H NMR (500 MHz, DMSO-d₆) δ: 2.18 (s, 6H, 2CH₃), 2.28 (s, 3H, CH₃), 3.69 (m, 4H, CH₂NCH₂), 3.78 (m, 4H, CH₂OCH₂), 6.92-7.45 (m, 7H, C₆H₃+C₆H₄), 10.2 (wide s, 2H, 2NH). ³C-NMR (150 MHz, DMSO-d₆) δ: 19.2, 20.0, 21.5, 44.7, 66.0, 118.7, 119.3, 122.0, 122.9, 125.2, 129.1, 130.1, 137.0, 138.5, 148.3, 162.5, 167.8. Anal. calcd for C₂₂H₂₇ClN₆O, % N, 19.6. Found, % N, 19.9. MS m/z: 391.2. [(M+H)+].

N-(3-Fluorophenyl)-N¹-(2-fluorophenyl)-6-morpholin-4-yl-[1,3,5]triazine-2,4-diamine hydrochloride (Compound 109) (VIRADEM-011687): Yield 67%. M.p.=165-166° C. C₁₉H₁₉ClF₂N₆O. MS m/z: 385.1 [(M+H)+].

N-(3-Fluorophenyl)-N¹-(4-fluorophenyl)-6-morpholin-4-yl-[1,3,5]triazine-2,4-diamine hydrochloride (Compound 110) (VIRADEM-011688): Yield 71%. M.p.=193-194° C. C₁₉H₁₉ClF₂N₆O. MS m/z: 385.1 [(M+H)+].

N-(3,5-Dichlorophenyl)-N¹-(4-methoxyphenyl)-6-morpholin-4-yl-[1,3,5]triazine-2,4-diamine (Compound 111) (VIRADEM-011757): Yield 78%. M.p.=163-164° C. C₂₀H₂₀Cl₂N₆O₂. MS m/z: 448.0 [(M+H)+].

N-(3,4-Dichlorophenyl)-N¹-(4-ethyphenyl)-6-morpholin-4-yl-[1,3,5]triazine-2,4-diamine (Compound 112) (VIRADEM-011758): Yield 73%. M.p.=206-207° C. C₂₁H₂₂Cl₂N₆O. MS m/z: 446.0 [(M+H)+].

N-(3,4-Dichlorophenyl)-N¹-(3,5-dimethyphenyl)-6-morpholin-4-yl-[1,3,5]triazine-2,4-diamine (Compound 113) (VIRADEM-011759): Yield 77%. M.p.=171-172° C. C₂₁H₂₂Cl₂N₆O. MS m/z: 446.1 [(M+H)+].

N-(3-Chlorophenyl)-N¹-(3,4-dimethoxyphenyl)-6-morpholin-4-yl-[1,3,5]triazine-2,4-diamine (Compound 114) (VIRADEM-011760): Yield 70%. M.p.=127-128° C. C₂₁H₂₃ClN₆O₃. MS m/z: 443.7 [(M+H)+].

N-(4-Butylphenyl)-N¹-(3-chlorophenyl)-6-morpholin-4-yl-[1,3,5]triazine-2,4-diamine (Compound 115) (VIRADEM-011761): Yield 67%. M.p.=161-162° C. C₂₃H₂₇ClN₆O. MS m/z: 439.7 [(M+H)+].

N-(4-Butylphenyl)-N¹-(4-chlorophenyl)-6-morpholin-4-yl-[1,3,5]triazine-2,4-diamine (Compound 116) (VIRADEM-011762): Yield 73%. M.p.=190-191° C. C₂₃H₂₇ClN₆O. MS m/z: 439.7 [(M+H)+].

N-(3,4-Dimethoxyphenyl)-N¹-(4-methoxyphenyl)-6-morpholin-4-yl-[1,3,5]triazine-2,4-diamine (Compound 117) (VIRADEM-011763): Yield 66%. M.p.=184-185° C. C₂₂H₂₆N₆O₄. MS m/z: 439.2 [(M+H)+].

N-(3,4-Dimethoxyphenyl)-N¹-(3,5-dimethylphenyl)-6-morpholin-4-yl-[1,3,5]triazine-2,4-diamine (Compound 118) (VIRADEM-011941): Yield 69%. M.p.=210-211° C. C₂₃H₂₈N₆O₃. MS m/z: 437.0 [(M+H)+]. ¹H NMR (500 MHz, DMSO-d₆) δ: 2.20 (s, 6H, 2CH₃), 3.64 (m, 4H, CH₂NCH₂), 3.71 (s, 6H, 2OCH₃), 3.73 (m, 4H, CH₂OCH₂), 6.58-7.35 (m, 6H, C₆H₃+C₆H₃), 8.97 (s, 2H, 2NH).

N-(3,4-Dimethoxyphenyl)-N¹-(2,3-dimethylphenyl)-6-morpholin-4-yl-[1,3,5]triazine-2,4-diamine (Compound 119) (VIRADEM-011764): Yield 56%. M.p.=150-151° C. C₂₃H₂₈N₆O₃. MS m/z: 437.0 [(M+H)+].

N-(3,5-Dichlorophenyl)-6-morpholin-4-yl-N¹-m-tolyl-[1,3,5]triazine-2,4-diamine (Compound 120) (VIRADEM-011765): Yield 82%. M.p.=217-218° C. C₂₀H₂₂Cl₂N₆O. MS m/z: 432.2 [(M+H)+]. ¹³C-NMR (125 MHz, DMSO-d₆) δ: 21.8, 44.0, 66.4, 118.0, 121.0, 121.4, 123.5, 128.9, 134.2, 138.1, 137.0, 140.1, 143.3, 164.4, 164.5, 165.0. Anal. calcd for C₂₂H₂₇ClN₆O, % N, 19.6. Found, % N, 19.9.

N-Benzo[1,3]dioxol-5-yl-N¹-(4-methoxyphenyl)-6-morpholin-4-yl-[1,3,5]triazine-2,4-diamine (Compound 121) (VIRADEM-011766): Yield 69%. M.p.=216-217° C. C₂₁H₂₂NO₄. MS m/z: 423.2 [(M+H)+].

N-(3-Chloro-4-fluorophenyl)-N¹-(3-fluorophenyl)₆-morpholin-4-yl-[1,3,5]triazine-2,4-diamine (Compound 122) (VIRADEM-011767): Yield 75%. M.p.=165-167° C. C₁₉H₁₇ClF₂N₆O. MS m/z: 419.5 [(M+H)+].

N-(3,4-Dimethylhenyl)-N¹,N²-diethyl-[1,3,5]triazine-2,4,6-triamine hydrochloride (Compound 123) (VIRADEM-011768): Yield 57%. M.p.=117-118° C. C₁₅H₂₃ClN₆. MS m/z: 287.0 [(M+H)+].

N-(3,5-Dimethylhenyl)-N¹,N²-diethyl-[1,3,5]triazine-2,4,6-triamine hydrochloride (Compound 124) (VIRADEM-011769): Yield 53%. M.p.=98-99° C. C₁₅H₂₃ClN₆. MS m/z: 287.0 [(M+H)+].

N-(3-Chlorophenyl)-N¹-(4-ethylphenyl)-6-morpholin-4-yl-[1,3,5]triazine-2,4-diamine hydrochloride (Compound 125) (VIRADEM-011770): Yield 73%. M.p.=193-194° C. C₂₁H₂₄Cl₂N₆O. MS m/z: 411.6 [(M+H)+]. ¹H NMR (400 MHz, DMSO-d₆) δ: 1.18 (t, 3H, CH₂CH₃), 2.57 (t, 3H, CH₂CH₃), 3.68 (m, 4H, CH₂NCH₂), 3.76 (m, 4H, CH₂OCH₂), 7.04-7.91 (m, 8H, C₆H₄+C₆H₄), 9.58 (s, 1H, 1NH), 9.74 (s, 1H, 1NH).

N-(3-Chlorophenyl)-N¹-(3,4-dimethylphenyl)-6-morpholin-4-yl-[1,3,5]triazine-2,4-diamine hydrochloride (Compound 126) (VIRADEM-011771): Yield 73%. M.p.=208-209° C. C₂₁H₂₄Cl₂N₆O. MS m/z: 411.6 [(M+H)+]. ¹H NMR (400 MHz, DMSO-d₆) δ: 2.19 (s, 3H, CH₃), 2.21 (s, 3H, CH₃), 3.69 (m, 4H, CH₂NCH₂), 3.78 (m, 4H, CH₂OCH₂), 7.07-7.84 (m, 7H, C₆H₃+C₆H₄), 9.74 (s, 1H, 1NH), 9.94 (s, 1H, 1NH).

N-(3-Chlorophenyl)-N¹-(3,5-dimethylphenyl)-6-morpholin-4-yl-[1,3,5]triazine-2,4-diamine hydrochloride (Compound 127) (VIRADEM-011772): Yield 75%. M.p.=223-224° C. C₂₁H₂₄Cl₂N₆O. MS m/z: 411.6 [(M+H)+]. ¹H NMR (400 MHz, DMSO-d₆) δ: 2.28 (s, 6H, 2CH₃), 3.72 (m, 4H, CH₂NCH₂), 3.82 (m, 4H, CH₂OCH₂), 6.73-7.77 (m, 7H, C₆H₃+C₆H₄), 9.93 (s, 1H, 1NH), 10.1 (s, 1H, 1NH).

N-(4-Methoxyphenyl)-N¹-(2-methoxyphenyl)-6-morpholin-4-yl-[1,3,5]triazine-2,4-diamine hydrochloride (Compound 128) (VIRADEM-011773): Yield 65%. M.p.=131-132° C. C₂₁H₂₅ClN₆O₃. MS m/z: 409.2 [(M+H)+]. ¹H NMR (400 MHz, DMSO-d₆) δ: 3.68 (m, 4H, CH₂NCH₂), 3.75 (s, 3H, OCH₃), 3.78 (m, 4H, CH₂OCH₂), 3.86 (s, 3H, OCH₃), 6.93-7.92 (m, 8H, C₆H₄+C₆H₄), 9.40 (s, 1H, 1NH), 10.3 (s, 1H, 1NH).

1-{3-[4-(3-Fluorophenylamino)-6-morpholin-4-yl-[1,3,5]triazin-2-ylamino]-phenyl}-ethanone hydrochloride (Compound 129) (VIRADEM-011774): Yield 71%. M.p.=197-198° C. C₂₁H₂₂ClFN₆O₂. MS m/z: 409.2 [(M+H)+].

N-Benzo[1,3]dioxol-5-yl-6-morpholin-4-yl-N¹-m-tolyl-[1,3,5]triazine-2,4-diamine hydrochloride (Compound 130) (VIRADEM-011775): Yield 74%. M.p.=231-232° C. C₂₁H₂₃ClN₆O₃. MS m/z: 407.2 [(M+H)+]. ¹H NMR (500 MHz, DMSO-d₆) δ: 2.28 (s, 3H, CH₃), 3.68 (m, 4H, CH₂NCH₂), 3.76 (m, 4H, CH₂OCH₂), 6.01 (s, 2H, OCH₂O), 6.88-7.44 (m, 7H, C₆H₃+C₆H₄), 10.2 (s, 2H, 2NH).

N-(3,5-Dimethylphenyl)-N¹-(4-ethylphenyl)-6-morpholin-4-yl-[1,3,5]triazine-2,4-diamine hydrochloride (Compound 131) (VIRADEM-011776): Yield 69%. M.p.=182-183° C. C₂₃H₂₉ClN₆O. MS m/z: 405.2 [(M+H)+]. ¹H NMR (400 MHz, DMSO-d₆) δ: 1.18 (t, 3H, CH₂CH₃), 2.25 (q, 2H, CH₂CH₃), 2.58 (s, 6H, 2CH₃), 3.69 (m, 4H, CH₂NCH₂), 3.78 (m, 4H, CH₂OCH₂), 6.73 (s, 1H, C₆H₃), 7.27 (s, 2H, C₆H₃), 7.18 and 7.54 (d-d, 4H, C₆H₄), 9.96 (s, 2H, 2NH).

N-(3,4-Dimethylphenyl)-N¹-(3,5-dimethylphenyl)-6-morpholin-4-yl-[1,3,5]triazine-2,4-diamine hydrochloride (Compound 132) (VIRADEM-011777): Yield 71%. M.p.=196-197° C. C₂₃H₂₉ClN₆O. MS m/z: 405.2 [(M+H)+].

1-[3-(4-Morpholin-4-yl-6-m-tolylamino-[1,3,5]triazin-2-ylamino)-phenyl]-ethanone hydrochloride (Compound 133) (VIRADEM-011778): Yield 75%. M.p.=226-227° C. C₂₂H₂₅ClN₆O₂. MS m/z: 405.2 [(M+H)+]. ¹H NMR (500 MHz, DMSO-d₆) δ: 2.27 (s, 3H, CH₃), 2.54 (s, 3H, COCH₃), 3.69 (m, 4H, CH₂NCH₂), 3.80 (m, 4H, CH₂OCH₂), 6.90-8.28 (m, 8H, C₆H₄+C₆H₄), 10.3 (s, 1H, 1NH), 10.4 (s, 1H, 1NH).

N-(3-Chlorophenyl)-N¹-(4-fluorophenyl)-6-morpholin-4-yl-[1,3,5]triazine-2,4-diamine hydrochloride (Compound 133) (VIRADEM-011779): Yield 72%. M.p.=211-212° C. C₁₉H₁₉Cl₂FN₆O. MS m/z: 401.6 [(M+H)+].

N-(3-Chlorophenyl)-6-morpholin-4-yl-N¹-p-tolyl-[1,3,5]triazine-2,4-diamine hydrochloride (Compound 134) (VIRADEM-011780): Yield 77%. M.p.=218-219° C. C₂₀H₂₂Cl₂N₆O. MS m/z: 397.2 [(M+H)+].

N-(3-Chlorophenyl)-6-morpholin-4-yl-N¹-m-tolyl-[1,3,5]triazine-2,4-diamine hydrochloride (Compound 135) (VIRADEM-011781): Yield 71%. M.p.=236-236° C. C₂₀H₂₂Cl₂N₆O. MS m/z: 397.2 [(M+H)+]. ¹H NMR (500 MHz, DMSO-d₆) δ: 2.29 (s, 3H, CH₃), 3.68 (m, 4H, CH₂NCH₂), 3.77 (m, 4H, CH₂OCH₂), 6.89-7.82 (m, 8H, C₆H₄+C₆H₄), 10.1 (s, 1H, 1NH), 10.2 (s, 1H, 1NH).

N-(4-Ethylphenyl)-N¹-(3-fluorophenyl)-6-morpholin-4-yl-[1,3,5]triazine-2,4-diamine hydrochloride (Compound 136) (VIRADEM-011782): Yield 75%. M.p.=181-182° C. C₂₁H₂₄ClFN₆O. MS m/z: 395.2 [(M+H)+].

N-(4-Ethylphenyl)-6-morpholin-4-yl-N¹-m-tolyl-[1,3,5]triazine-2,4-diamine hydrochloride (Compound 137) (VIRADEM-011783): Yield 69%. M.p.=218-219° C. C₂₂H₂₇ClN₆O. MS m/z: 391.0 [(M+H)+]. ¹H NMR (500 MHz, DMSO-d₆) δ: 1.17 (t, 3H, CH₂CH₃), 2.29 (s, 3H, CH₃), 2.58 (q, 2H, CH₂CH₃), 3.69 (m, 4H, CH₂NCH₂), 3.78 (m, 4H, CH₂OCH₂), 6.92-7.50 (m, 8H, C₆H₄+C₆H₄), 10.3 (s, 2H, 2NH).

N-(3,5-Dimethylphenyl)-6-morpholin-4-yl-N¹-m-tolyl-[1,3,5]triazine-2,4-diamine hydrochloride (Compound 138) (VIRADEM-011784): Yield 73%. M.p.=214-215° C. C₂₂H₂₇ClN₆O. MS m/z: 391.2 [(M+H)+]. ¹H NMR (500 MHz, DMSO-d₆) δ: 2.24 (s, 6H, 2CH₃), 2.28 (s, 3H, CH₃), 3.69 (m, 4H, CH₂NCH₂), 3.78 (m, 4H, CH₂OCH₂), 6.76-7.47 (m, 7H, C₆H₃+C₆H₄), 10.4 (s, 2H, 2NH).

N-(3-Fluorophenyl)-6-morpholin-4-yl-N¹-p-tolyl-[1,3,5]triazine-2,4-diamine hydrochloride (Compound 139) (VIRADEM-011784): Yield 70%. M.p.=191-192° C. C₂₀H₂₂ClFN₆O. MS m/z: 381.2 [(M+H)+].

N-(3-Fluorophenyl)-6-morpholin-4-yl-N¹-m-tolyl-[1,3,5]triazine-2,4-diamine hydrochloride (Compound 140) (VIRADEM-011786): Yield 67%. M.p.=218-219° C. C₂₀H₂₂ClFN₆O. MS m/z: 381.2 [(M+H)+]. ¹H NMR (500 MHz, DMSO-d₆) δ: 2.29 (s, 3H, CH₃), 3.68 (m, 4H, CH₂NCH₂), 3.77 (m, 4H, CH₂OCH₂), 6.84-7.70 (m, 8H, C₆H₄+C₆H₄), 9.99 (s, 1H, 1NH), 10.1 (s, 1H, 1NH).

N-(4-Fluorophenyl)-6-morpholin-4-yl-N¹-m-tolyl-[1,3,5]triazine-2,4-diamine hydrochloride (Compound 141) (VIRADEM-011787): Yield 75%. M.p.=223-224° C. C₂₀H₂₂ClFN₆O. MS m/z: 381.2 [(M+H)+]. ¹H NMR (500 MHz, DMSO-d₆) δ: 2.28 (s, 3H, CH₃), 3.68 (m, 4H, CH₂NCH₂), 3.77 (m, 4H, CH₂OCH₂), 6.91-7.63 (m, 8H, C₆H₄+C₆H₄), 10.3 (s, 2H, 2NH).

6-Morpholin-4-yl-N-m-tolyl-N¹-p-tolyl-[1,3,5]triazine-2,4-diamine hydrochloride (Compound 142) (VIRADEM-011789): Yield 70%. M.p.=216-217° C. C₂₁H₂₅ClN₆O. MS m/z: 377.2 [(M+H)+]. ¹H NMR (500 MHz, DMSO-d₆) δ: 2.27 (s, 3H, CH₃), 2.29 (s, 3H, CH₃), 3.69 (m, 4H, CH₂NCH₂), 3.78 (m, 4H, CH₂OCH₂), 6.92-7.49 (m, 8H, C₆H₄+C₆H₄), 10.4 (s, 2H, 2NH).

N-(3,5-Dichlorophenyl)-N¹-(3,5-dimethylphenyl)-6-morpholin-4-yl-[1,3,5]triazine-2,4-diamine (Compound 143) (VIRADEM-011792): Yield 78%. M.p.=182-183° C. C₂₁H₂₂Cl₂N₆O. MS m/z: 446.1 [(M+H)+].

N-(3,4-Dimethoxyphenyl)-N¹-(3-fluorophenyl)-6-morpholin-4-yl-[1,3,5]triazine-2,4-diamine (Compound 144) (VIRADEM-011793): Yield 78%. M.p.=174-175° C. C₂₁H₂₃FN₆O₃. MS m/z: 427.2 [(M+H)+].

N-(4-Ethoxyphenyl)-6-morpholin-4-yl-N¹-m-tolyl-[1,3,5]triazine-2,4-diamine hydrochloride (Compound 145) (VIRADEM-011794): Yield 75%. M.p.=218-219° C. C₂₂H₂₇ClN₆O₂. MS m/z: 407.0 [(M+H)+]. ¹H NMR (500 MHz, DMSO-d₆) δ: 1.31 (s, 3H, OCH₂CH₃), 2.28 (s, 3H, CH₃), 3.69 (m, 4H, CH₂NCH₂), 3.78 (m, 4H, CH₂OCH₂), 4.00 (s, 2H, OCH₂CH₃), 6.91-7.48 (m, 8H, C₆H₄+C₆H₄), 10.3 (s, 1H, 1NH), 10.6 (s, 1H, 1NH).

4-[4-(4-Ethylphenylamino)-6-morpholin-4-yl-[1,3,5]-2-ylamino]-benzoic acid ethyl ester (Compound 146) (VIRADEM-011845): Yield 69%. M.p.=191-192° C. C₂₄H₂₈N₆O₃. MS m/z: 449.2 [(M+H)+].

N-(4-Methoxyphenyl)-6-morpholin-4-yl-N¹-(3-trifluoromethylphenyl)-[1,3,5]triazine-2,4-diamine (Compound 147) (VIRADEM-011846): Yield 78%. M.p.=180-181° C. C₂₁H₂₁F₃NO₂. MS m/z: 447.2 [(M+H)+]. ¹H NMR (400 MHz, DMSO-d₆) δ: 3.66 (m, 4H, CH₂NCH₂), 3.74 (s, 3H, OCH₃), 3.75 (m, 4H, CH₂OCH₂), 6.87 and 7.58 (d-d, 4H, C₆H₄), 7.26-8.20 (m, 4H, C₆H₄), 9.05 (s, 1H, 1NH), 9.44 (s, 1H, 1NH).

N-{4-[4-(4-Chlorophenylamino)-6-morpholin-4-yl-[1,3,5]triazin-2-ylamino]-phenyl}-acetamide (Compound 148) (VIRADEM-011847): Yield 89%. M.p.>260° C. C₂₁H₂₂ClN₇O₂. MS m/z: 440.6 [(M+H)+]. ¹H NMR (400 MHz, DMSO-d₆) δ: 2.03 (s, 3H, NHCOCH₃), 3.66 (m, 4H, CH₂NCH₂), 3.74 (m, 4H, CH₂OCH₂), 7.30 and 7.61 (d-d, 4H, C₆H₄), 7.48 and 7.78 (d-d, 4H, C₆H₄), 9.16 (s, 1H, 1NH), 9.31 (s, 1H, 1NH), 9.83 (s, 1H, 1NH).

N-(3-Chloro-4-fluorophenyl)-N¹-(3-chlorophenyl)-6-morpholin-4-yl-[1,3,5]triazine-2,4-diamine (Compound 149) (VIRADEM-011848): Yield 80%. M.p.=155-156° C. C₁₉H₁₇Cl₂FN₆O. MS m/z: 436.0 [(M+H)+].

N-(3-Chloro-4-methylphenyl)-N¹-(3-chlorophenyl)-6-morpholin-4-yl-[1,3,5]triazine-2,4-diamine (Compound 150) (VIRADEM-011849): Yield 76%. M.p.=163-164° C. C₂₀H₂₀Cl₂N₆O. MS m/z: 432.0 [(M+H)+].

N-(3-Chloro-4-fluorophenyl)-N¹-(4-ethylphenyl)-6-morpholin-4-yl-[1,3,5]triazine-2,4-diamine (Compound 151) (VIRADEM-011850): Yield 73%. M.p.=217-218° C. C₂₁H₂₂ClFN₆O. MS m/z: 429.7 [(M+H)+].

N-(3-Chloro-4-fluorophenyl)-N¹-(3,5-dimethylphenyl)-6-morpholin-4-yl-[1,3,5]triazine-2,4-diamine (Compound 152) (VIRADEM-011851): Yield 76%. M.p.=153-154° C. C₂₁H₂₂ClFN₆O. MS m/z: 429.7 [(M+H)+]. ¹H NMR (400 MHz, DMSO-d₆) δ: 2.21 (s, 3H, 2CH₃), 3.66 (m, 4H, CH₂NCH₂), 3.74 (m, 4H, CH₂OCH₂), 6.63-8.01 (m, 6H, C₆H₃+C₆H₃), 9.15 (s, 1H, 1NH), 9.38 (s, 1H, 1NH).

N-(3-Chlorophenyl)-N¹-(4-ethoxyphenyl)-6-morpholin-4-yl-[1,3,5]triazine-2,4-diamine (Compound 153) (VIRADEM-011852): Yield 77%. M.p.=183-184° C. C₂₁H₂₃ClN₆O₂. MS m/z: 427.7 [(M+H)+].

N-(3-Chlorophenyl)-N¹-(3-chlorophenyl)-6-morpholin-4-yl-[1,3,5]triazine-2,4-diamine (Compound 154) (VIRADEM-011853): Yield 83%. M.p.=234-235° C. C₁₉H₁₈Cl₂N₆O. MS m/z: 418.0 [(M+H)+].

N-(3,4-Dichlorophenyl)-N¹-(3,4-dimethylphenyl)-6-morpholin-4-yl-[1,3,5]triazine-2,4-diamine (Compound 155) (VIRADEM-011878): Yield 80%. M.p.=243-244° C. C₂₁H₂₂Cl₂N₆O. MS m/z: 446.1 [(M+H)+].

N-(3,5-Dichlorophenyl)-6-morpholin-4-yl-N¹-p-tolyl-[1,3,5]triazine-2,4-diamine (Compound 156) (VIRADEM-011879): Yield 77%. M.p.=191-192° C. C₂₀H₂₀Cl₂N₆O. MS m/z: 432.0 [(M+H)+].

N-Benzo[1,3]dioxol-5-yl-N¹-(4-chlorophenyl)-6-morpholin-4-yl-[1,3,5]triazine-2,4-diamine (Compound 157) (VIRADEM-011880): Yield 71%. M.p.=222-224° C. C₂₀H₁₉ClN₆O₃. MS m/z: 427.6 [(M+H)+].)+]. ¹H NMR (400 MHz, DMSO-d₆) δ: 3.60 (m, 4H, CH₂NCH₂), 3.73 (m, 4H, CH₂OCH₂), 5.95 (s, 2H, OCH₂O), 6.80-7.43 (m, 3H, C₆H₃), 7.26 and 7.74 (d-d, 4H, C₆H₄), 9.22 (s, 1H, 1NH), 9.33 (s, 1H, 1NH).

1-{3-[4-(4-Fluorophenylamino)-6-morpholin-4-yl-[1,3,5]triazin-2-ylamino]-phenyl}-ethanone hydrochloride (Compound 158) (VIRADEM-011883): Yield 73%. M.p.=204-205° C. C₂₁H₂₂ClFN₆O₂. MS m/z: 409.2 [(M+H)+].

N-(3,4-Dimethylphenyl)-N¹-(4-ethylphenyl)-6-morpholin-4-yl-[1,3,5]triazine-2,4-diamine hydrochloride (Compound 159) (VIRADEM-011884): Yield 73%. M.p.=218-219° C. C₂₃H₂₉ClN₆O. MS m/z: 405.3 [(M+H)+].

1-[3-(4-Morpholin-4-yl-6-p-tolylamino-[1,3,5]triazin-2-ylamino)-phenyl]-ethanone hydrochloride (Compound 160) (VIRADEM-011885): Yield 75%. M.p.=234-235° C. C₂₂H₂₅ClN₆O₂. MS m/z: 405.2 [(M+H)+].

N-(4-Chlorophenyl)-N¹-(2-fluorophenyl)-6-morpholin-4-yl-[1,3,5]triazine-2,4-diamine hydrochloride (Compound 161) (VIRADEM-011886): Yield 70%. M.p.=211-212° C. C₁₉H₁₉Cl₂FN₆O. MS m/z: 401.6 [(M+H)+].

4-[4-(3,4-Dimethylphenylamino)-6-morpholin-4-yl-[1,3,5]-2-ylamino]-benzoic acid ethyl ester (Compound 162) (VIRADEM-011913): Yield 65%. M.p.=168-169° C. C₂₄H₂₈N₆O₃. MS m/z: 449.3 [(M+H)+].

N-(4-Dimethoxyphenyl)-N¹-(3,4-dimethylphenyl)-6-morpholin-4-yl-[1,3,5]triazine-2,4-diamine (Compound 163) (VIRADEM-011915): Yield 65%. M.p.=142-143° C. C₂₃H₂₈N₆O₃. MS m/z: 437.2 [(M+H)+].

4-(4-Morpholin-4-yl-6-p-tolylamino[1,3,5]-2-ylamino)-benzoic acid ethyl ester (Compound 164) (VIRADEM-011916): Yield 73%. M.p.=216-217° C. C₂₃H₂₆N₆O₃. MS m/z: 435.3 [(M+H)+].

N-(4-Fluorophenyl)-6-morpholin-4-yl-N¹-(3-trifluoromethylphenyl)-[1,3,5]triazine-2,4-diamine (Compound 165) (VIRADEM-011917): Yield 78%. M.p.=161-162° C. C₂₀H₁₈F₄N₆O. MS m/z: 435.0 [(M+H)+].

N-{4-[4-(3,4-Dimethylphenylamino)-6-morpholin-4-yl-[1,3,5]triazin-2-ylamino]phenyl}-acetamide (Compound 166) (VIRADEM-011918): Yield 81%. M.p.=248-249° C. C₂₃H₂₇N₇O₄. MS m/z: 434.2 [(M+H)+].

6-Morpholin-4-yl-N-p-tolyl-N¹-(3-trifluoromethylphenyl)-[1,3,5]triazine-2,4-diamine (Compound 167) (VIRADEM-011919): Yield 78%. M.p.=201-201° C. C₂₁H₂₁F₃N₆O. MS m/z: 431.2 [(M+H)+].

N-(3-Chloro-4-fluorophenyl)-N¹-(3,4-dimethylphenyl)-6-morpholin-4-yl-[1,3,5]triazine-2,4-diamine (Compound 168) (VIRADEM-011920): Yield 69%. M.p.=198-199° C. C₂₁H₂₂ClFN₆O. MS m/z: 429.6 [(M+H)+].

N-(3-Chloro-4-methylphenyl)-N¹-(3,4-dimethylphenyl)-6-morpholin-4-yl-[1,3,5]triazine-2,4-diamine (Compound 169) (VIRADEM-011921): Yield 74%. M.p.=206-207° C. C₂₂H₂₅ClN₆O. MS m/z: 425.6 [(M+H)+].

N-(3,4-Dimethoxyphenyl)-6-morpholin-4-yl-N¹-p-tolyl-[1,3,5]triazine-2,4-diamine (Compound 170) (VIRADEM-011922): Yield 69%. M.p.=163-164° C. C₂H₂₂NO₃. MS m/z: 423.2 [(M+H)+].

N-Benzo[1,3]dioxol-5-yl-N¹-(3,4-dimethylphenyl)-6-morpholin-4-yl-[1,3,5]triazine-2,4-diamine (Compound 171) (VIRADEM-011923): Yield 77%. M.p.=203-204° C. C₂₂H₂₄NO₃. MS m/z: 421.2 [(M+H)+].

N-(4-Butylphenyl)-6-morpholin-4-yl-N¹-p-tolyl-[1,3,5]triazine-2,4-diamine (Compound 172) (VIRADEM-011924): Yield 70%. M.p.=174-175° C. C₂₄H₃₀N₆O. MS m/z: 419.3 [(M+H)+].

1-{3-[4-(3,4-Dimethylphenylamino)-6-morpholin-4-yl-[1,3,5]triazin-2-ylamino]-phenyl}-ethanone (Compound 173) (VIRADEM-011925): Yield 75%. M.p.=164-165° C. C₂₃H₂₆NO₂. MS m/z: 419.2 [(M+H)+].

N-(3-Chloro-4-methylphenyl)-N¹-(4-fluorophenyl)-6-morpholin-4-yl-[1,3,5]triazine-2,4-diamine (Compound 174) (VIRADEM-011926): Yield 77%. M.p.=196-197° C. C₂₀H₂₀ClFN₆O. MS m/z: 415.6 [(M+H)+].

4-(4-Morpholin-4-yl-6-m-tolylamino-[1,3,5]-2-ylamino]-benzoic acid ethyl ester (Compound 175) (VIRADEM-011933): Yield 66%. M.p.=172-173° C. C₂₃H₂NO₃. MS m/z: 435.3 [(M+H)+].

4-[4-(3,5-Dimethylphenylamino)-6-morpholin-4-yl-[1,3,5]-2-ylamino]-benzoic acid ethyl ester (Compound 176) (VIRADEM-011934): Yield 68%. M.p.=223-224° C. C₂₄H₂₈N₆O₃. MS m/z: 449.3 [(M+H)+].

N-(4-Butylphenyl)-N¹-(3,4-dimethylphenyl)-6-morpholin-4-yl-[1,3,5]triazine-2,4-diamine (Compound 177) (VIRADEM-011935): Yield 72%. M.p.=144-145° C. C₂₅H₃₂N₆O. MS m/z: 433.2 [(M+H)+].

N-(4-Butylphenyl)-6-morpholin-4-yl-N¹-m-tolyl-[1,3,5]triazine-2,4-diamine (Compound 178) (VIRADEM-011936): Yield 75%. M.p.=152-153° C. C₂₄H₃₀N₆O. MS m/z: 419.3 [(M+H)+].

N-(3,5-Dimethylphenyl)-6-morpholin-4-yl-N¹-(3-trifluoromethylphenyl)-[1,3,5]triazine-2,4-diamine (Compound 179) (VIRADEM-011938): Yield 78%. M.p.=133-134° C. C₂₂H₂₃F₃N₆O. MS m/z: 445.2 [(M+H)+].

N-(4-Butylphenyl)-N¹-(4-fluorophenyl)-6-morpholin-4-yl-[1,3,5]triazine-2,4-diamine (Compound 180) (VIRADEM-011940): Yield 70%. M.p.=151-152° C. C₂₃H₂₇FN₆O. MS m/z: 423.2 [(M+H)+].

N-(3,4-Dimethylphenyl)-N¹-(2-fluorophenyl)-6-morpholin-4-yl-[1,3,5]triazine-2,4-diamine hydrochloride (Compound 181) (VIRADEM-011942): Yield 77%. M.p.=172-173° C. C₂₃H₂₄ClFN₆O. MS m/z: 431.6 [(M+H)+].

1-{3-[4-(4-Methoxyphenylamino)-6-morpholin-4-yl-[1,3,5]triazin-2-ylamino]-phenyl}-ethanone (Compound 181) (VIRADEM-011983): Yield 79%. M.p.=193-194° C. C₂₂H₂₄NO₃. MS m/z: 421.2 [(M+H)+].

6-Morpholin-4-yl-N-m-tolyl-N¹-(3-trifluoromethylphenyl)-[1,3,5]triazine-2,4-diamine (Compound 182) (VIRADEM-011984): Yield 71%. M.p.=124-125° C. C₂₁H₂₁F₃N₆O. MS m/z: 431.2 [(M+H)+].

N-(3-Chloro-4-fluorophenyl)-N¹-(3-chloro-4-methylphenyl)-6-morpholin-4-yl-[1,3,5]triazine-2,4-diamine (Compound 183) (VIRADEM-011985): Yield 69%. M.p.=183-184° C. C₂₀H₁₉Cl₂FN₆O. MS m/z: 450.0 [(M+H)+].

1-{3-[4-(3-Chloro-4-fluorophenylamino)-6-morpholin-4-yl-[1,3,5]triazin-2-ylamino]-phenyl}-ethanone (Compound 184) (VIRADEM-011986): Yield 72%. M.p.=201-202° C. C₂₁H₂₀ClFN₆O₂. MS m/z: 443.5 [(M+H)+].

1-{3-[4-(4-Ethoxyphenylamino)-6-morpholin-4-yl-[1,3,5]triazin-2-ylamino]-phenyl}-ethanone (Compound 185) (VIRADEM-011995): Yield 78%. M.p.=226-227° C. C₂₃H₂₆NO₃. MS m/z: 435.2 [(M+H)+].

N-(3-Chloro-4-methylphenyl)-N¹-(4-ethylphenyl)-6-morpholin-4-yl-[1,3,5]triazine-2,4-diamine (Compound 186) (VIRADEM-011996): Yield 71%. M.p.=198-199° C. C₂₂H₂₅ClN₆O. MS m/z: 425.6 [(M+H)+].

1-{3-[4-(3-Chloro-4-methylphenylamino)-6-morpholin-4-yl-[1,3,5]triazin-2-ylamino]-phenyl}-ethanone (Compound 187) (VIRADEM-012010): Yield 75%. M.p.=154-155° C. C₂₂H₂₃ClN₆O₂. MS m/z: 439.6 [(M+H)+].

1-{3-[4-(4-Butylphenylamino)-6-morpholin-4-yl-[1,3,5]triazin-2-ylamino]-phenyl}-ethanone (Compound 188) (VIRADEM-012037): Yield 69%. M.p.=142-143° C. C₂₅H₃₀N₆O₂. MS m/z: 447.3 [(M+H)+].

1-[3-(4-Morpholin-4-yl-6-m-tolylamino-[1,3,5]triazin-2-ylamino)-phenyl]-ethanone (Compound 189) (VIRADEM-012042): Yield 72%. M.p.=156-157° C. C₂₂H₂₄NO₂. MS m/z: 405.3 [(M+H)+].

1-{3-[4-(2-Fluorophenylamino)-6-morpholin-4-yl-[1,3,5]triazin-2-ylamino]-phenyl}-ethanone hydrochloride (Compound 190) (VIRADEM-012126): Yield 65%. M.p.=182-183° C. C₂₁H₂₂ClFN₆O₂. MS m/z: 409.2 [(M+H)+].

1-{3-[4-(4-Ethylphenylamino)-6-morpholin-4-yl-[1,3,5]triazin-2-ylamino]-phenyl}-ethanone (Compound 191) (VIRADEM-012187): Yield 73%. M.p.=192-193° C. C₂₃H₂₆N₆O₂. MS m/z: 419.2 [(M+H)+].

1-{3-[4-(3-Chlorophenylamino)-6-morpholin-4-yl-[1,3,5]triazin-2-ylamino]-phenyl}-ethanone (Compound 192) (VIRADEM-012188): Yield 78%. M.p.=162-163° C. C₂₁H₂₁ClN₆O₂. MS m/z: 425.6 [(M+H)+].

N-(4-Methoxyphenyl)-N¹-(3-methoxyphenyl)-6-morpholin-4-yl-[1,3,5]triazine-2,4-diamine hydrochloride (Compound 193) (VIRADEM-012197): Yield 69%. M.p.=185-186° C. C₂₁H₂₅ClN₆O₃. MS m/z: 409.2 [(M+H)+].

N-(3-Methoxyphenyl)-6-morpholin-4-yl-N¹-p-tolyl-[1,3,5]triazine-2,4-diamine hydrochloride (Compound 194) (VIRADEM-012211): Yield 71%. M.p.=211-212° C. C₂₁H₂₅ClN₆O₂. MS m/z: 393.2 [(M+H)+].

N-(3-Fluorophenyl)-N¹-(3-methoxyphenyl)-6-morpholin-4-yl-[1,3,5]triazine-2,4-diamine hydrochloride (Compound 195) (VIRADEM-012221): Yield 71%. M.p.=206-207° C. C₂₀H₂₂ClFN₆O₂. MS m/z: 397.2 [(M+H)+].

N-(3,4-Dimethylphenyl)-N¹-(3-methoxyphenyl)-6-morpholin-4-yl-[1,3,5]triazine-2,4-diamine hydrochloride (Compound 196) (VIRADEM-012226): Yield 68%. M.p.=187-188° C. C₂₀H₂₇ClN₆O₂. MS m/z: 407.3 [(M+H)+].

N-(3-Methoxyphenyl)-6-morpholin-4-yl-N¹-m-tolyl-[1,3,5]triazine-2,4-diamine hydrochloride (Compound 197) (VIRADEM-012227): Yield 70%. M.p.=233-234° C. C₂₁H₂₅ClN₆O₂. MS m/z: 393.2 [(M+H)+]. ¹H NMR (500 MHz, DMSO-d₆) δ: 2.29 (s, 3H, CH₃), 3.69 (m, 4H, CH₂NCH₂), 3.72 (s, 3H, OCH₃), 3.78 (m, 4H, CH₂OCH₂), 6.68-7.45 (m, 8H, C₆H₄+C₆H₄), 10.3 (s, 2H, 2NH).

N-(3-Chloro-4-fluorophenyl)-N¹-(3-methoxyphenyl)-6-morpholin-4-yl-[1,3,5]triazine-2,4-diamine (Compound 198) (VIRADEM-012245): Yield 75%. M.p.=153-154° C. C₂₀H₂₀ClFN₆O₂. MS m/z: 431.6 [(M+H)+].

N-(3,5-Dimethylphenyl)-N¹-(4-isopropylphenyl)-6-morpholin-4-yl-[1,3,5]triazine-2,4-diamine (Compound 199) (VIRADEM-012246): Yield 77%. M.p.=115-116° C. C₂₄H₃₀N₆O. MS m/z: 419.3 [(M+H)+].

N-(3-Chloro-4-fluorophenyl)-N¹-(4-isopropylphenyl)-6-morpholin-4-yl-[1,3,5]triazine-2,4-diamine (Compound 200) (VIRADEM-012247): Yield 71%. M.p.=156-157° C. C₂₂H₂₄ClFN₆O. MS m/z: 443.6 [(M+H)+].

N-(3-Methoxyphenyl)-6-morpholin-4-yl-N¹-(3-trifluoromethylphenyl)-[1,3,5]triazine-2,4-diamine (Compound 201) (VIRADEM-012248): Yield 76%. M.p.=132-133° C. C₂₁H₂₁F₃NO₂. MS m/z: 447.1 [(M+H)+].

N-(3-Chloro-4-methylphenyl)-N¹-(4-isopropylphenyl)-6-morpholin-4-yl-[1,3,5]triazine-2,4-diamine (Compound 202) (VIRADEM-012249): Yield 72%. M.p.=194-195° C. C₂₃H₂₇ClN₆O. MS m/z: 439.6 [(M+H)+].

N-(3,4-Dichlorophenyl)-N¹-(3-methoxyphenyl)-6-morpholin-4-yl-[1,3,5]triazine-2,4-diamine (Compound 203) (VIRADEM-012249): Yield 79%. M.p.=162-163° C. C₂₀H₂₀Cl₂N₆O₂. MS m/z: 448.0 [(M+H)+].

N-(4-Isoproylphenyl)-6-morpholin-4-yl-N¹-m-tolyl-[1,3,5]triazine-2,4-diamine hydrochloride (Compound 204) (VIRADEM-012251): Yield 73%. M.p.=218-219° C. C₂₃H₂₉ClN₆O. MS m/z: 405.2 [(M+H)+]. ¹H NMR (500 MHz, DMSO-d₆) δ: 1.18 (d, 6H, CH(CH₃)₂), 2.29 (s, 3H, CH₃), 2.86 (m, 1H, CH(CH₃)₂), 3.69 (m, 4H, CH₂NCH₂), 3.78 (m, 4H, CH₂OCH₂), 6.92-7.54 (m, 8H, C₆H₄+C₆H₄), 10.4 (s, 2H, 2NH).

N-(4-Isoproylphenyl)-6-morpholin-4-yl-N¹-p-tolyl-[1,3,5]triazine-2,4-diamine hydrochloride (Compound 205) (VIRADEM-012253): Yield 70%. M.p.=205-206° C. C₂₃H₂₉ClN₆O. MS m/z: 405.2 [(M+H)+]. ¹H NMR (400 MHz, DMSO-d₆) δ: 1.17 (d, 6H, CH(CH₃)₂), 2.28 (s, 3H, CH₃), 2.87 (m, 1H, CH(CH₃)₂), 3.68 (m, 4H, CH₂NCH₂), 3.77 (m, 4H, CH₂OCH₂), 6.89-7.56 (m, 8H, C₆H₄+C₆H₄), 10.3 (s, 2H, 2NH).

N-(3-Chloro-4-methylphenyl)-N¹-(3-methoxyphenyl)-6-morpholin-4-yl-[1,3,5]triazine-2,4-diamine (Compound 206) (VIRADEM-012254): Yield 77%. M.p.=180-181° C. C₂₁H₂₃ClN₆O₂. MS m/z: 427.6 [(M+H)+].

N-(3-Fluorophenyl)-N¹-(4-isopropylphenyl)-6-morpholin-4-yl-[1,3,5]triazine-2,4-diamine hydrochloride (Compound 207) (VIRADEM-012255): Yield 70%. M.p.=177-178° C. C₂₂H₂₆ClFN₆O. MS m/z: 409.2 [(M+H)+].

N-(4-Fluorophenyl)-N¹-(4-isopropylphenyl)-6-morpholin-4-yl-[1,3,5]triazine-2,4-diamine hydrochloride (Compound 208) (VIRADEM-012256): Yield 75%. M.p.=210-211° C. C₂₂H₂₆ClFN₆O. MS m/z: 409.2 [(M+H)+].

N-(4-Fluorophenyl)-N¹-(3-methoxyphenyl)-6-morpholin-4-yl-[1,3,5]triazine-2,4-diamine hydrochloride (Compound 209) (VIRADEM-012262): Yield 73%. M.p.=204-206° C. C₂₀H₂₂ClFN₆O₂. MS m/z: 397.2 [(M+H)+]. ¹H NMR (400 MHz, DMSO-d₆) δ: 3.67 (m, 4H, CH₂NCH₂), 3.71 (m, 4H, CH₂OCH₂), 7.16-7.69 (m, 8H, C₆H₄+C₆H₄), 9.80 (s, 2H, 2NH).

N-(4-Ethylphenyl)-N¹-(3-methoxyphenyl)-6-morpholin-4-yl-[1,3,5]triazine-2,4-diamine hydrochloride (Compound 210) (VIRADEM-012265): Yield 70%. M.p.=203-204° C. C₂₂H₂₇ClN₆O₂. MS m/z: 407.2 [(M+H)+].

N-(3,5-Dimethylphenyl)-N¹-(2-methoxyphenyl)-6-morpholin-4-yl-[1,3,5]triazine-2,4-diamine hydrochloride (Compound 211) (VIRADEM-012270): Yield 70%. M.p.=146-147° C. C₂₂H₂₇ClN₆O₂. MS m/z: 407.2 [(M+H)+].

N-(4-Ethylphenyl)-6-morpholin-4-yl-N¹-(3-trifluoromethylphenyl)-[1,3,5]triazine-2,4-diamine (Compound 212) (VIRADEM-012271): Yield 65%. M.p.=177-178° C. C₂₂H₂₃F₃N₆O. MS m/z: 445.2 [(M+H)+].

N-(4-Ethoxyphenyl)-N¹-(3-methoxyphenyl)-6-morpholin-4-yl-[1,3,5]triazine-2,4-diamine (Compound 213) (VIRADEM-012272): Yield 76%. M.p.=197-198° C. C₂₂H₂₆N₆O₃. MS m/z: 423.2 [(M+H)+].

N-(3-Ethoxyphenyl)-N¹-(4-ethylphenyl)-6-morpholin-4-yl-[1,3,5]triazine-2,4-diamine (Compound 214) (VIRADEM-012273): Yield 71%. M.p.=211-212° C. C₂₃H₂₈N₆O₂. MS m/z: 421.3 [(M+H)+].

N-(3,5-Dimethylphenyl)-N¹-(3-methoxyphenyl)-6-morpholin-4-yl-[1,3,5]triazine-2,4-diamine hydrochloride (Compound 215) (VIRADEM-012280): Yield 77%. M.p.=202-203° C. C₂₂H₂₇ClN₆O₂. MS m/z: 407.2 [(M+H)+].

N,N¹-Bis-(4-isopropylphenyl)-6-morpholin-4-yl-[1,3,5]triazine-2,4-diamine (Compound 216) (VIRADEM-012288): Yield 69%. M.p.=183-185° C. C₂₅H₃₂N₆O. MS m/z: 433.3 [(M+H)+].

N-(4-Chlorophenyl)-N¹-(4-ethoxyphenyl)-6-morpholin-4-yl-[1,3,5]triazine-2,4-diamine hydrochloride (Compound 217) (VIRADEM-012294): Yield 70%. M.p.=172-173° C. C₂₁H₂₄Cl₂N₆O₂. MS m/z: 427.6 [(M+H)+]. ¹H NMR (400 MHz, DMSO-d₆) δ: 1.32 (t, 3H, OCH₂CH₃), 3.66 (m, 4H, CH₂NCH₂), 3.75 (m, 4H, CH₂OCH₂), 4.00 (q, 2H, OCH₂CH₃), 6.26-7.75 (m, 8H, C₆H₄+C₆H₄), 9.71 (s, 1H, 1NH), 9.95 (s, 1H, 1NH).

N-(3,4-Dimethoxyphenyl)-N¹-(4-ethylphenyl)-6-morpholin-4-yl-[1,3,5]triazine-2,4-diamine (Compound 218) (VIRADEM-012296): Yield 79%. M.p.=131-132° C. C₂₃H₂₈N₆O₃. MS m/z: 437.3 [(M+H)+]. ¹H NMR (400 MHz, DMSO-d₆) δ: 1.61 (t, 3H, CH₂CH₃), 2.54 (q, 2H, CH₂CH₃), 3.64 (m, 4H, CH₂NCH₂), 3.72 (s, 6H, 2OCH₃), 3.74 (m, 4H, CH₂OCH₂), 6.84-7.43 (m, 3H, C₆H₃), 7.09-7.63 (d-d, 4H, C₆H₄), 8.99 (s, 1H, 1NH), 9.04 (s, 1H, 1NH).

N-(4-Butylphenyl)-N¹-(4-ethylphenyl)-6-morpholin-4-yl-[1,3,5]triazine-2,4-diamine (Compound 219) (VIRADEM-012300): Yield 73%. M.p.=167-168° C. C₂₅H₃₂N₆O. MS m/z: 433.3 [(M+H)+]. 0.90 (t, 3H, CH₃), 1.17 (t, 3H, CH₂CH₃), 1.31 (m, 2H, CH₂), 1.54 (m, 2H, CH₂), 2.52 (m, 2H, CH₂), 2.55 (t, 2H, CH₂CH₃), 3.65 (m, 4H, CH₂NCH₂), 3.73 (m, 4H, CH₂OCH₂), 7.07 and 7.61 (d-d, 4H, C₆H₄), 7.10 and 7.62 (d-d, 4H, C₆H₄), 9.01 (s, 2H, 2NH).

N-(3,5-Dichlorophenyl)-N¹-(3-fluorophenyl)-6-morpholin-4-yl-[1,3,5]triazine-2,4-diamine (Compound 220) (VIRADEM-012301): Yield 80%. M.p.=191-192° C. C₁₉H₁₇Cl₂FN₆O. MS m/z: 436.0 [(M+H)+]. ¹H NMR (400 MHz, DMSO-d₆) δ: 3.66 (m, 4H, CH₂NCH₂), 3.75 (m, 4H, CH₂OCH₂), 6.79-7.84 (m, 7H, C₆H₃+C₆H₄), 9.55 (s, 1H, 1NH), 9.62 (s, 1H, 1NH).

1-{3-[4-(3-Acetylphenylamino)-6-morpholin-4-yl-[1,3,5]triazin-2-ylamino]-phenyl}-ethanone (Compound 221) (VIRADEM-012302): Yield 78%. M.p.=202-203° C. C₂₃H₂₄N₆O₃. MS m/z: 433.2 [(M+H)+].

N-(4-Butylphenyl)-N¹-(3-fluorophenyl)-6-morpholin-4-yl-[1,3,5]triazine-2,4-diamine (Compound 222) (VIRADEM-012301): Yield 75%. M.p.=152-153° C. C₂₃H₂₇FN₆O. MS m/z: 423.3 [(M+H)+]. ¹H NMR (400 MHz, DMSO-d₆) δ: 0.90 (t, 3H, CH₃), 1.29 (m, 2H, CH₂), 1.54 (m, 2H, CH₂), 2.54 (m, 2H, CH₂), 3.65 (m, 4H, CH₂NCH₂), 3.74 (m, 4H, CH₂OCH₂), 6.74-7.82 (m, 8H, C₆H₄+C₆H₄), 9.20 (s, 1H, 1NH), 9.38 (s, 1H, 1NH).

N-(4-Ethoxyphenyl)-N¹-(4-isopropylphenyl)-6-morpholin-4-yl-[1,3,5]triazine-2,4-diamine (Compound 223) (VIRADEM-012301): Yield 73%. M.p.=185-186° C. C₂₄H₃₀N₆O₂. MS m/z: 435.3 [(M+H)+]. ¹H NMR (400 MHz, DMSO-d₆) δ: 1.18 (d, 6H, CH(CH₃)₂), 1.32 (t, 3H, OCH₂CH₃), 2.83 (m, 1H, CH(CH₃)₂), 3.64 (m, 4H, CH₂NCH₂), 3.72 (m, 4H, CH₂OCH₂), 4.00 (q, 2H, OCH₂CH₃), 6.83 and 7.58 (d-d, 4H, C₆H₄), 7.12 and 7.61 (d-d, 4H, C₆H₄), 8.96 (s, 1H, 1NH), 9.03 (s, 1H, 1NH).

1-{3-[4-(4-Isopropylphenylamino)-6-morpholin-4-yl-[1,3,5]triazin-2-ylamino]-phenyl}-ethanone (Compound 224) (VIRADEM-012305): Yield 82%. M.p.=171-172° C. C₂₄H₂₈N₆O₂. MS m/z: 433.3 [(M+H)+]. ¹H NMR (400 MHz, DMSO-d₆) δ: 1.19 (d, 6H, CH(CH₃)₂), 2.83 (m, 1H, CH(CH₃)₂), 2.53 (s, 3H, COCH₃), 3.64 (m, 4H, CH₂NCH₂), 3.73 (m, 7H, CH₂OCH₂), 6.86 and 7.60 (d-d, 4H, C₆H₄), 7.29-8.33 (d-d, 4H, C₆H₄), 9.11 (s, 1H, 1NH), 9.25 (s, 1H, 1NH).

N-(4-Isopropylphenyl)-N¹-(4-methoxyphenyl)-6-morpholin-4-yl-[1,3,5]triazine-2,4-diamine (Compound 225) (VIRADEM-012322): Yield 75%. M.p.=205-206° C. C₂₃H₂₈N₆O₂. MS m/z: 421.3 [(M+H)+]. ¹H NMR (400 MHz, DMSO-d₆) δ: 1.18 (d, 6H, CH(CH₃)₂), 2.83 (m, 1H, CH(CH₃)₂), 3.64 (m, 4H, CH₂NCH₂), 3.72 (m, 7H, OCH₃+CH₂OCH₂), 6.85 and 7.61 (d-d, 4H, C₆H₄), 7.13 and 7.60 (d-d, 4H, C₆H₄), 9.00 (s, 1H, 1NH), 9.05 (s, 1H, 1NH).

N-(3-Chlorophenyl)-N¹-(4-isopropylphenyl)-6-morpholin-4-yl-[1,3,5]triazine-2,4-diamine (Compound 226) (VIRADEM-012323): Yield 79%. M.p.=144-146° C. C₂₂H₂₅ClN₆O. MS m/z: 425.6 [(M+H)+]. ¹H NMR (400 MHz, DMSO-d₆) δ: 1.19 (d, 6H, CH(CH₃)₂), 2.84 (m, 1H, CH(CH₃)₂), 3.66 (m, 4H, CH₂NCH₂), 3.75 (m, 4H, CH₂OCH₂), 6.97-7.96 (m, 4H, C₆H₄), 7.16 and 7.60 (d-d, 4H, C₆H₄), 9.21 (s, 1H, 1NH), 9.37 (s, 1H, 1NH).

N-(4-Chlorophenyl)-N¹-(4-isopropylphenyl)-6-morpholin-4-yl-[1,3,5]triazine-2,4-diamine (Compound 227) (VIRADEM-012324): Yield 85%. M.p.=239-240° C. C₂₂H₂₅ClN₆O. MS m/z: 425.6 [(M+H)+]. ¹H NMR (400 MHz, DMSO-d₆) δ: 1.19 (d, 6H, CH(CH₃)₂), 2.85 (m, 1H, CH(CH₃)₂), 3.66 (m, 4H, CH₂NCH₂), 3.74 (m, 4H, CH₂OCH₂), 7.15 and 7.61 (d-d, 4H, C₆H₄), 7.29 and 7.78 (d-d, 4H, C₆H₄), 9.11 (s, 1H, 1NH), 9.26 (s, 1H, 1NH).

N-(4-Ethoxyphenyl)-N¹-(3-fluorophenyl)-6-morpholin-4-yl-[1,3,5]triazine-2,4-diamine hydrochloride (Compound 228) (VIRADEM-012326): Yield 70%. M.p.=225-226° C. C₂₁H₂₄ClFN₆O₂. MS m/z: 447.6 [(M+H)+]. ¹H NMR (400 MHz, DMSO-d₆) δ: 1.32 (t, 3H, OCH₂CH₃), 3.64 (m, 4H, CH₂NCH₂), 3.73 (m, 4H, CH₂OCH₂), 3.98 (q, 2H, OCH₂CH₃), 6.87 and 7.55 (d-d, 4H, C₆H₄), 6.75-7.78 (m, 4H, C₆H₄), 9.42 (s, 1H, 1NH), 9.66 (s, 1H, 1NH).

N-(3,4-Dimethoxyphenyl)-N¹-(4-methoxyphenyl)-6-morpholin-4-yl-[1,3,5]triazine-2,4-diamine (Compound 229) (VIRADEM-012328): Yield 69%. M.p.=147-148° C. C₂₂H₂₆N₆O₄. MS m/z: 439.2 [(M+H)+]. ¹H NMR (400 MHz, DMSO-d₆) δ: 3.64 (m, 4H, CH₂NCH₂), 3.71 (s, 9H, 3OCH₃), 3.75 (m, 4H, CH₂OCH₂), 6.50-7.48 (m, 7H, C₆H₃+C₆H₄), 9.05 (s, 1H, 1NH), 9.21 (s, 1H, 1NH).

4-[4-(3-Methoxyphenylamino)-6-morpholin-4-yl-[1,3,5]triazin-2-ylamino]-benzoic acid methyl ester (Compound 230) (VIRADEM-012329): Yield 79%. M.p.=177-178° C. C₂₂H₂₄N₆O₄. MS m/z: 437.2 [(M+H)+]. 3.67 (m, 4H, CH₂NCH₂), 3.74 (s, 3H, OCH₃), 3.77 (m, 4H, CH₂OCH₂), 3.82 (s, 3H, COOCH₃), 6.56-7.43 (m, 4H, C₆H₄), 7.87 and 7.93 (d-d, 4H, C₆H₄), 9.29 (s, 1H, 1NH), 9.62 (s, 1H, 1NH).

1-{3-[4-(3-Methoxyphenylamino)-6-morpholin-4-yl-[1,3,5]triazin-2-ylamino]-phenyl}-ethanone (Compound 231) (VIRADEM-012330): Yield 77%. M.p.=97-98° C. C₂₂H₂₄N₆O₃. MS m/z: 421.2 [(M+H)+]. ¹H NMR (400 MHz, DMSO-d₆) δ: 2.54 (s, 3H, COCH₃), 3.67 (m, 4H, CH₂NCH₂), 3.72 (s, 3H, OCH₃), 3.78 (m, 4H, CH₂OCH₂), 6.53-8.36 (m, 8H, C₆H₄+C₆H₄), 9.19 (s, 1H, 1NH), 9.40 (s, 1H, 1NH).

N-(3-Fluorophenyl)-6-morpholin-4-yl-N¹-(3-trifluoromethylphenyl)-[1,3,5]triazine-2,4-diamine (Compound 232) (VIRADEM-012331): Yield 75%. M.p.=129-130° C. C₂₀H₁₈F₄N₆O. MS m/z: 435.0 [(M+H)+]. ¹H NMR (400 MHz, DMSO-d₆) δ: 3.67 (m, 4H, CH₂NCH₂), 3.76 (m, 4H, CH₂OCH₂), 6.77-8.19 (m, 8H, C₆H₄+C₆H₄), 9.49 (s, 1H, 1NH), 9.63 (s, 1H, 1NH).

N-(3,5-Dichlorophenyl)-N¹-(3-methoxyphenyl)-6-morpholin-4-yl-[1,3,5]triazine-2,4-diamine (Compound 233) (VIRADEM-012332): Yield 76%. M.p.=104-105° C. C₂₀H₂₀Cl₂N₆O₂. MS m/z: 448.0 [(M+H)+]. ¹H NMR (400 MHz, DMSO-d₆) δ: 3.66 (m, 4H, CH₂NCH₂), 3.72 (s, 3H, OCH₃), 3.75 (m, 4H, CH₂OCH₂), 6.52-7.88 (m, 7H, C₆H₃+C₆H₄), 9.36 (s, 1H, 1NH), 9.61 (s, 1H, 1NH).

N-(4-Butylphenyl)-N¹-(3-methoxyphenyl)-6-morpholin-4-yl-[1,3,5]triazine-2,4-diamine (Compound 234) (VIRADEM-012333): Yield 69%. M.p.=136-137° C. C₂₄H₃₀N₆O₂. MS m/z: 435.3 [(M+H)+]. ¹H NMR (400 MHz, DMSO-d₆) δ: 0.90 (t, 3H, CH₃), 1.31 (m, 2H, CH₂), 1.31 (m, 2H, CH₂), 1.53 (m, 2H, CH₂), 2.53 (m, 2H, CH₂), 3.65 (m, 4H, CH₂NCH₂), 3.71 (s, 3H, OCH₃), 3.74 (m, 4H, CH₂OCH₂), 7.08 and 7.61 (d-d, 4H, C₆H₄), 6.53-7.44 (m, 4H, C₆H₄), 9.10 (s, 1H, 1NH), 9.14 (s, 1H, 1NH).

N-(3,4-Dimethylphenyl)-N¹-(2-methoxyphenyl)-6-morpholin-4-yl-[1,3,5]triazine-2,4-diamine hydrochloride (Compound 235) (VIRADEM-012334): Yield 77%. M.p.=153-154° C. C₂₂H₂₇ClN₆O₂. MS m/z: 407.2 [(M+H)+]. ¹H NMR (400 MHz, DMSO-d₆) δ: 2.19 (s, 6H, 2CH₃), 3.66 (m, 4H, CH₂NCH₂), 3.75 (m, 4H, CH₂OCH₂), 3.86 (s, 3H, OCH₃), 6.96-7.38 (m, 7H, C₆H₃+C₆H₄), 8.96 (s, 1H, 1NH), 9.91 (s, 1H, 1NH).

N-(2-Methoxyphenyl)-6-morpholin-4-yl-N¹-m-tolyl-[1,3,5]triazine-2,4-diamine hydrochloride (Compound 236) (VIRADEM-012335): Yield 67%. M.p.=200-201° C. C₂₁H₂₅ClN₆O₂. MS m/z: 393.2 [(M+H)+]. ¹H NMR (500 MHz, DMSO-d₆) δ: 2.28 (s, 6H, 2CH₃), 3.69 (m, 4H, CH₂NCH₂), 3.78 (m, 4H, CH₂OCH₂), 3.85 (s, 3H, OCH₃), 6.92-7.87 (m, 8H, C₆H₄+C₆H₄), 9.39 (s, 1H, 1NH), 10.3 (s, 1H, 1NH).

N-(4-Fluorophenyl)-N¹-(2-methoxyphenyl)-6-morpholin-4-yl-[1,3,5]triazine-2,4-diamine hydrochloride (Compound 237) (VIRADEM-012356): Yield 71%. M.p.=184-185° C. C₂₀H₂₂ClFN₆O₂. MS m/z: 397.1 [(M+H)+]. NMR (400 MHz, DMSO-d₆) δ: 3.67 (m, 4H, CH₂NCH₂), 3.76 (m, 4H, CH₂OCH₂), 3.84 (s, 3H, OCH₃), 6.89-7.88 (m, H, C₆H₃+C₆H₄), 9.15 (s, 1H, 1NH), 9.56 (s, 1H, 1NH).

N-(3,4-Dichlorophenyl)-N¹-(2-methoxyphenyl)-6-morpholin-4-yl-[1,3,5]triazine-2,4-diamine (Compound 238) (VIRADEM-012387): Yield 67%. M.p.=170-171° C. C₂₀H₂₀Cl₂N₆O₂. MS m/z: 448.0 [(M+H)+]. NMR (400 MHz, DMSO-d₆) δ: 3.66 (m, 4H, CH₂NCH₂), 3.75 (m, 4H, CH₂OCH₂), 3.83 (s, 3H, OCH₃), 6.91-7.89 (m, H, C₆H₃+C₆H₄), 9.20 (s, 1H, 1NH), 10.2 (s, 1H, 1NH).

N-(4-Ethylphenyl)-N¹-(2-methoxyphenyl)-6-morpholin-4-yl-[1,3,5]triazine-2,4-diamine hydrochloride (Compound 239) (VIRADEM-012388): Yield 65%. M.p.=194-195° C. C₂₂H₂₇ClN₆O₂. MS m/z: 407.2 [(M+H)+]. ¹H NMR (400 MHz, DMSO-d₆) δ: 1.19 (t, 3H, CH₂CH₃), 2.55 (q, 2H, CH₂CH₃), 3.65 (m, 4H, CH₂NCH₂), 3.74 (m, 4H, CH₂OCH₂), 3.81 (s, 3H, OCH₃), 6.85-7.79 (m, 8H, C₆H₄+C₆H₄), 9.39 (s, 1H, 1NH), 10.1 (s, 1H, 1NH).

N-(3-Chloro-4-fluorophenyl)-N¹-(2-methoxyphenyl)-6-morpholin-4-yl-[1,3,5]triazine-2,4-diamine (Compound 240) (VIRADEM-012389): Yield 69%. M.p.=168-169° C. C₂₀H₂₀ClFN₆O₂. MS m/z: 431.5 [(M+H)+]. ¹H NMR (400 MHz, DMSO-d₆) δ: 3.66 (m, 4H, CH₂NCH₂), 3.75 (m, 4H, CH₂OCH₂), 3.82 (s, 3H, OCH₃), 6.85-7.94 (m, 7H, C₆H₃+C₆H₄), 9.25 (s, 1H, 1NH), 10.2 (s, 1H, 1NH).

N-(3-Fluorophenyl)-N¹-(2-methoxyphenyl)-6-morpholin-4-yl-[1,3,5]triazine-2,4-diamine hydrochloride (Compound 241) (VIRADEM-012390): Yield 67%. M.p.=135-136° C. C₂₀H₂₂ClFN₆O₂. MS m/z: 397.2 [(M+H)+]. ¹H NMR (400 MHz, DMSO-d₆) δ: 3.68 (m, 4H, CH₂NCH₂), 3.77 (m, 4H, CH₂OCH₂), 3.83 (s, 3H, OCH₃), 6.88-7.89 (m, 8H, C₆H₄+C₆H₄), 9.21 (s, 1H, 1NH), 10.3 (s, 1H, 1NH).

N-(2-Methoxyphenyl)-N¹-(3-methoxyphenyl)-6-morpholin-4-yl-[1,3,5]triazine-2,4-diamine hydrochloride (Compound 242) (VIRADEM-012391): Yield 62%. M.p.=157-158° C. C₂₁H₂₅ClN₆O₃. MS m/z: 409.2 [(M+H)+]. ¹H NMR (400 MHz, DMSO-d₆) δ: 3.66 (m, 4H, CH₂NCH₂), 3.75 (m, 4H, CH₂OCH₂), 3.83 (s, 6H, 2OCH₃), 6.68-7.91 (m, 8H, C₆H₄+C₆H₄), 9.43 (s, 1H, 1NH), 10.3 (s, 1H, 1NH).

N-(4-Ethoxyphenyl)-N¹-(2-methoxyphenyl)-6-morpholin-4-yl-[1,3,5]triazine-2,4-diamine (Compound 243) (VIRADEM-012406): Yield 66%. M.p.=166-167° C. C₂₂H₂₆N₆O₃. MS m/z: 423.3 [(M+H)+]. ¹H NMR (400 MHz, DMSO-d₆) δ: 1.32 (t, 3H, OCH₂CH₃), 3.66 (m, 4H, CH₂NCH₂), 3.75 (m, 4H, CH₂OCH₂), 3.83 (s, 3H, OCH₃), 4.00 (q, 2H, OCH₂CH₃), 6.68-7.91 (m, 8H, C₆H₄+C₆H₄), 9.43 (s, 1H, 1NH), 10.3 (s, 1H, 1NH).

N-(3-Chloro-4-methylphenyl)-N¹-(2-methoxyphenyl)-6-morpholin-4-yl-[1,3,5]triazine-2,4-diamine (Compound 244) (VIRADEM-012407): Yield 74%. M.p.=168-169° C. C₂₁H₂₃ClN₆O₂. MS m/z: 427.6 [(M+H)+]. ¹H NMR (400 MHz, DMSO-d₆) δ: 2.52 (s, 3H, CH₃), 3.65 (m, 4H, CH₂NCH₂), 3.73 (m, 4H, CH₂OCH₂), 3.84 (s, 3H, OCH₃), 6.93-8.05 (m, 8H, C₆H₄+C₆H₄), 9.37 (s, 2H, 2NH).

N-(2-Methoxyphenyl)-6-morpholin-4-yl-N¹-p-tolyl-[1,3,5]triazine-2,4-diamine hydrochloride (Compound 245) (VIRADEM-012408): Yield 71%. M.p.=168-169° C. C₂₁H₂₅ClN₆O₂. MS m/z: 293.2 [(M+H)+]. ¹H NMR (400 MHz, DMSO-d₆) δ: 2.29 (s, 3H, CH₃), 3.69 (m, 4H, CH₂NCH₂), 3.78 (m, 4H, CH₂OCH₂), 3.86 (s, 6H, 2OCH₃), 6.96-7.93 (m, 8H, C₆H₄+C₆H₄), 9.21 (s, 1H, 1NH), 10.2 (s, 1H, 1NH).

N-(3-Chloro-4-methylphenyl)-N¹-cyclohexyl-6-morpholin-4-yl-[1,3,5]triazine-2,4-diamine hydrochloride (Compound 246) (VIRADEM-012433): Yield 67%. M.p.=185-186° C. C₂₀H₂₈Cl₂N₆O. MS m/z: 403.2 [(M+H)+].

N-Cyclohexyl-N¹-(3,5-dimethylphenyl)-6-morpholin-4-yl-[1,3,5]triazine-2,4-diamine hydrochloride (Compound 247) (VIRADEM-013159): Yield 70%. M.p.=227-228° C. C₂₁H₃₁ClN₆O. MS m/z: 383.1 [(M+H)+]. ¹H NMR (400 MHz, CDCl₃) δ: 1.34-2.05 (m, 10H, C₆H₁₁), 2.29 (s, 6H, 2CH₃), 3.73 (m, 4H, CH₂NCH₂), 3.85 (m, 4H, CH₂OCH₂), 6.75 (s, 1H, C₆H₃), 7.15 (s, 2H, C₆H₃), 8.08 (s, 1H, 1NH), 10.1 (s, 1H, 1NH), 12.7 (s, 1H, 1HCl).

N-Cyclohexyl-N¹-(3,5-dimethylphenyl)-6-morpholin-4-yl-[1,3,5]triazine-2,4-diamine hydrochloride (Compound 248) (VIRADEM-013160): Yield 74%. M.p.=215-216° C. C₂₁H₃₁ClN₆O₂. MS m/z: 399.2 [(M+H)+]. ¹H NMR (400 MHz, DMSO-d₆) δ: 1.32 (t, 3H, OCH₂CH₃), 1.18-1.87 (m, 10H, C₆H₁₁), 3.66 (m, 4H, CH₂NCH₂), 3.75 (m, 4H, CH₂OCH₂), 4.00 (q, 2H, OCH₂CH₃), 6.89 and 7.48 (d-d, 4H, C₆H₄), 7.15 (s, 2H, C₆H₃), 8.64 (s, 1H, 1NH), 10.6 (s, 1H, 1NH).

6-Morpholin-4-yl-N,N¹-di-o-tolyl-[1,3,5]triazine-2,4-diamine (Compound 249) (VIRADEM-014275): Yield 61%. C₂₁H₂₄N₆O. MS m/z: 377.1 [(M+H)+]. ¹H NMR (400 MHz, DMSO-d₆) δ: 2.16 (s, 6H, 2CH₃), 3.66 (m, 4H, CH₂NCH₂), 3.75 (m, 4H, CH₂OCH₂), 6.79-7.65 (m, 8H, C₆H₄+C₆H₄), 9.28 (s, 1H, 1NH), 9.56 (s, 1H, 1NH).

N,N¹-Bis-(2-ethylphenyl)-6-morpholin-4-yl-[1,3,5]triazine-2,4-diamine (Compound 250) (VIRADEM-014282): Yield 63%. M.p.=148-149° C. C₂₃H₂₉ClN₆O. MS m/z: 405.4 [(M+H)+]. ¹H NMR (400 MHz, DMSO-d₆) δ: 1.19 (t, 3H, CH₂CH₃), 2.54 (q, 2H, CH₂CH₃), 3.66 (m, 4H, CH₂NCH₂), 3.75 (m, 4H, CH₂OCH₂), 6.92-7.68 (m, 8H, C₆H₄+C₆H₄), 9.33 (s, 1H, 1NH), 9.76 (s, 1H, 1NH).

N,N¹-Bis-(2,4-dimethoxyphenyl)-6-morpholin-4-yl-[1,3,5]triazine-2,4-diamine (Compound 251) (VIRADEM-014283): Yield 65%. M.p.=160-161° C. C₂₃H₂₈N₆O₅. MS m/z: 469.2 [(M+H)+]. ¹H NMR (400 MHz, DMSO-d₆) δ: 3.66 (m, 4H, CH₂NCH₂), 3.73 (s, 6H, 2OCH₃), 3.75 (m, 4H, CH₂OCH₂), 3.85 (s, 6H, 2OCH₃), 6.87-7.73 (m, 8H, 2C₆H₄), 8.99 (s, 1H, 1NH), 9.13 (s, 1H, 1NH).

6-Morpholine-4-yl-N,N′-dipyridin-3-yl-[1,3,5]triazine-2,4-diamine (Compound 252) (VIRADEM-014288): Yield 73%. M.p.=>260° C. C₁₇H₁₈N₈O. MS m/z: 351.0 [(M+H)+].

N,N¹-Dibenzyl-6-morpholin-4-yl-[1,3,5]triazine-2,4-diamine (Compound 253) (VIRADEM-014290): Yield 59%. M.p.=163-164° C. C₂₁H₂₄N₆O. MS m/z: 377.2 [(M+H)+].

N,N¹-Bis-(2-methoxyphenyl)-6-morpholin-4-yl-[1,3,5]triazine-2,4-diamine hydrochloride (Compound 254) (VIRADEM-014297): Yield 63%. M.p.=213-214° C. C₂₁H₂₅ClN₆O₃. MS m/z: 409.2 [(M+H)+]. ¹H NMR (400 MHz, DMSO-d₆) δ: 3.65 (m, 4H, CH₂NCH₂), 3.76 (m, 4H, CH₂OCH₂), 3.84 (s, 6H, 2OCH₃), 6.82-7.71 (m, 8H, 2C₆H₄), 9.23 (s, 2H, 2NH).

N,N¹-Bis-(2,4-dimethylphenyl)-6-morpholin-4-yl-[1,3,5]triazine-2,4-diamine hydrochloride (Compound 255) (VIRADEM-014298): Yield 59%. M.p.=224-225° C. C₂₃H₂₉ClN₆O. MS m/z: 405.4 [(M+H)+]. ¹H NMR (400 MHz, DMSO-d₆) δ: 2.04 (s, 6H, 2CH₃), 2.39 (s, 6H, 2CH₃), 3.65 (m, 4H, CH₂NCH₂), 3.76 (m, 4H, CH₂OCH₂), 7.08-7.85 (m, 6H, 2C₆H₃), 9.71 (s, 2H, 2NH).

N,N¹-Bis-(5-chloro-2-methoxyphenyl)-6-morpholin-4-yl-[1,3,5]triazine-2,4-diamine (Compound 256) (VIRADEM-014304): Yield 61%. M.p.=210-211° C. C₂₁H₂₂Cl₂N₆O₃. MS m/z: 478.1 [(M+H)+]. ¹H NMR (400 MHz, DMSO-d₆) δ: 3.66 (m, 4H, CH₂NCH₂), 3.77 (m, 4H, CH₂OCH₂), 3.83 (s, 6H, 2OCH₃), 6.79-7.77 (m, 6H, 2C₆H₃), 9.13 (s, 2H, 2NH).

N,N¹-Bis-(2,5-dimethylphenyl)-6-morpholin-4-yl-[1,3,5]triazine-2,4-diamine hydrochloride (Compound 257) (VIRADEM-014305): Yield 65%. M.p.=213-214° C. C₂₃H₂₉ClN₆O. MS m/z: 405.4 [(M+H)+]. ¹H NMR (400 MHz, DMSO-d₆) δ: 2.07 (s, 6H, 2CH₃), 2.19 (s, 6H, 2CH₃), 3.66 (m, 4H, CH₂NCH₂), 3.75 (m, 4H, CH₂OCH₂), 7.12-7.83 (m, 6H, 2C₆H₃), 9.77 (s, 2H, 2NH).

N,N¹-Bis-(2,3-dimethylphenyl)-6-morpholin-4-yl-[1,3,5]triazine-2,4-diamine hydrochloride (Compound 258) (VIRADEM-014306): Yield 61%. M.p.=226-227° C. C₂₃H₂₉ClN₆O. MS m/z: 405.4 [(M+H)+]. ¹H NMR (400 MHz, DMSO-d₆) δ: 2.08 (s, 6H, 2CH₃), 2.23 (s, 6H, 2CH₃), 3.65 (m, 4H, CH₂NCH₂), 3.74 (m, 4H, CH₂OCH₂), 7.18-7.78 (m, 6H, 2C₆H₃), 9.74 (s, 2H, 2NH).

N,N¹-Bis-(2,5-dichlorophenyl)-6-morpholin-4-yl-[1,3,5]triazine-2,4-diamine (Compound 259) (VIRADEM-014308): Yield 66%. M.p.=181-182° C. C₁₉H₁₆Cl₄N₆O. MS m/z: 487.1 [(M+H)+].

N,N¹-Diisopropyl-N²-p-tolyl-[1,3,5]triazine-2,4,6-triamine (Compound 260) (VIRADEM-014377): Yield 53%. M.p.=115-116° C. C₁₆H₂₄N₆. MS m/z: 301.2 [(M+H)+].

N,N′-Bis-(2,5-dimethoxyphenyl)-6-morpholin-4-yl-[1,3,5]triazine-2,4-diamine (Compound 261) (VIRADEM-014935): Yield 54%. M.p.=103-104° C. C₂₃H₂₈N₆O₅. MS m/z: 467.3 [(M+H)+].

N-(2,5-Dichlorophenyl)-6-morpholin-4-yl-N¹-p-tolyl-[1,3,5]triazine-2,4-diamine (Compound 262) (VIRADEM-015159): Yield 63%. M.p.=208-209° C. C₂₀H₂₀Cl₂N₆O. MS m/z: 432.1 [(M+H)+].

1-[4-(4-Morpholin-4-yl-6-p-tolylamino-[1,3,5]triazin-2-ylamino)-phenyl]-ethanone (Compound 263) (VIRADEM-015160): Yield 78%. M.p.=232-233° C. C₂₂H₂₄N₆O₂. MS m/z: 405.3 [(M+H)+].

1-{4-[3,4-Dimethylphenylamino)-6-morpholin-4-yl-[1,3,5]triazin-2-ylamino]-phenyl}-ethanone (Compound 264) (VIRADEM-015162): Yield 75%. M.p.=220-221° C. C₂₃H₂₆N₆O₂. MS m/z: 419.3 [(M+H)+].

N-(3,4-Dimethylphenyl)-6-morpholin-4-yl-N¹-pyridin-3-yl-[1,3,5]triazine-2,4-diamine (Compound 265) (VIRADEM-015167): Yield 59%. M.p.>260° C. C₂₀H₂₃N₇O. MS m/z: 378.2 [(M+H)+]. ¹H NMR (400 MHz, DMSO-d₆) δ: 2.18 (s, 6H, 2CH₃), 3.65 (m, 4H, CH₂NCH₂), 3.74 (m, 4H, CH₂OCH₂), 7.05-7.58 (m, 6H, C₆H₃+C₆H₃), 9.34 (s, 2H, 2NH).

N-(5-Chloro-2-methoxyphenyl)-6-morpholin-4-yl-N¹-p-tolyl-[1,3,5]triazine-2,4-diamine (Compound 266) (VIRADEM-015170): Yield 71%. M.p.=191-192° C. C₂₁H₂₃ClN₆O₂. MS m/z: 427.7 [(M+H)+]. ¹H NMR (400 MHz, DMSO-d₆) δ: 2.25 (s, 3H, CH₃), 3.64 (m, 4H, CH₂NCH₂), 3.72 (m, 4H, CH₂OCH₂), 3.86 (s, 3H, OCH₃), 7.04-7.26 (m, 6H, 2C₆H₃), 7.08 and 7.52 (d-d, 4H, C₆H₄), 9.28 (s, 2H, 2NH).

N-(5-Chloro-2-methoxyphenyl)-N¹-(3,4-dimethylphenyl)-6-morpholin-4-yl-[1,3,5]triazine-2,4-diamine (Compound 267) (VIRADEM-015171): Yield 68%. M.p.=157-158° C. C₂₂H₂₅ClN₆O₂. MS m/z: 441.6 [(M+H)+]. ¹H NMR (400 MHz, DMSO-d₆) δ:2.19 (s, 6H, 2CH₃), 3.66 (m, 4H, CH₂NCH₂), 3.75 (m, 4H, CH₂OCH₂), 3.86 (s, 3H, OCH₃), 6.97-7.37 (m, 6H, 2C₆H₃), 7.08 and 7.52 (d-d, 4H, C₆H₄), 8.98 (s, 1H, NH), 9.79 (s, 1H, NH).

6-Morpholin-4-yl-N-pyridin-3-yl-N¹-p-tolyl-[1,3,5]triazine-2,4-diamine (Compound 268) (VIRADEM-015172): Yield 58%. M.p.=>260° C. C₁₉H₂₁N₇O. MS m/z: 364.2 [(M+H)+].

(4-Methoxyphenyl)-[4-morpholin-4-yl-6-(4-phenylpiperazin-1-yl)-[1,3,5]triazin-2-yl]-amine (Compound 269) (VIRADEM-015197): Yield 77%. M.p.=211-212° C. C₂₄H₂₉N₇O₂. MS m/z: 448.4 [(M+H)+].

N-(1,1-Dioxotetrahydro-1lambda*6*-thiophen-3-yl)-N¹-(4-methoxyphenyl)-6-morpholin-4-yl-[1,3,5]triazine-2,4-diamine (Compound 270) (VIRADEM-015205): Yield 70%. M.p.=246-247° C. C₁₈H₂₄N₆O₄S. MS m/z: 421.3 [(M+H)+].

[4-(4-Methylpiperidin-1-yl)-6-morpholin-4-yl-[1,3,5]triazin-2-yl]-p-tolylamine (Compound 271) (VIRADEM-015206): Yield 75%. M.p.=157-158° C. C₂₀H₂₈N₆O. MS m/z: 369.2 [(M+H)+].

(3,4-Dimethylphenyl)-[4-(3,5-dimethylpiperidin-1-yl)-6-morpholin-4-yl-[1,3,5]triazin-2-yl]-amine (Compound 272) (VIRADEM-015207): Yield 75%. M.p.=204-205° C. C₂₂H₃₂N₆O. MS m/z: 397.3 [(M+H)+].

N-Benzo[1,3]dioxol-5-yl-N¹-(3,4-dimethylphenyl)-6-morpholin-4-yl-[1,3,5]triazine-2,4-diamine (Compound 273) (VIRADEM-015208): Yield 79%. M.p.=144-145° C. C₂₃H₂₆N₆O₃. MS m/z: 435.3 [(M+H)+].

(2,5-Dimethylphenyl)-6-morpholin-4-yl-N¹-phenyl-[1,3,5]triazine-2,4-diamine (Compound 274) (VIRADEM-015210): Yield 71%. M.p.=148-149° C. C₂₁H₂₄N₆O. MS m/z: 377.2 [(M+H)+].

N-(1,1-Dioxotetrahydro-1lambda*6*-thiophen-3-yl)-6-morpholin-4-yl-N¹-phenyl-[1,3,5]triazine-2,4-diamine (Compound 275) (VIRADEM-015211): Yield 72%. M.p.=224-225° C. C₁₇H₂₂N₆O₄S. MS m/z: 391.2 [(M+H)+].

N-(3-Ethylphenyl)-N¹-(4-methoxyphenyl)-6-morpholin-4-yl-[1,3,5]triazine-2,4-diamine (Compound 276) (VIRADEM-015212): Yield 77%. M.p.=133-134° C. C₂₂H₂₆N₆O₂. MS m/z: 407.2 [(M+H)+]. ¹H NMR (400 MHz, DMSO-d₆) δ: 1.19 (t, 3H, CH₂CH₃), 2.55 (q, 2H, CH₂CH₃), 3.66 (m, 4H, CH₂NCH₂), 3.71 (s, 3H, OCH₃), 3.75 (m, 4H, CH₂OCH₂), 6.84 and 7.57 (d-d, 4H, C₆H₄), 6.95-7.68 (m, 4H, C₆H₄), 9.28 (s, 1H, 1NH), 9.46 (s, 1H, 1NH).

[4-(3,5-Dimethylpiperidin-1-yl)-6-morpholin-4-yl-[1,3,5]triazin-2-yl]-(4-methoxyphenyl)-amine (Compound 277) (VIRADEM-015213): Yield 73%. M.p.=179-180° C. C₂₁H₃₀N₆O₂. MS m/z: 399.3 [(M+H)+]. ¹H NMR (400 MHz, DMSO-d₆) δ: 0.87 (d+m, 8H, CH₂₊₂CH₃), 1.49 (m, 2H, CH+CH), 2.22 (m, 2H, CH+CH), 3.62 (m, 4H, CH₂NCH₂), 3.70 (m, 4H, CH₂OCH₂), 3.72 (s, 3H, OCH₃), 4.63 (m, 2H, CH+CH), 6.84 and 7.56 (d-d, 4H, C₆H₄), 8.88 (s, 1H, 1NH).

(4-Methoxyphenyl)-[4-(4-methylpiperidin-1-yl)-6-morpholin-4-yl-[1,3,5]triazin-2-yl]amine (Compound 278) (VIRADEM-015220): Yield 79%. M.p.=173-174° C. C₂₀H₂₈N₆O₂. MS m/z: 385.2 [(M+H)+].

N-(3,4-Dimethylphenyl)-N¹-(2-ethoxyphenyl)-6-morpholin-4-yl-[1,3,5]triazine-2,4-diamine (Compound 279) (VIRADEM-015221): Yield 77%. M.p.=143-144° C. C₂₃H₂₈N₆O₂. MS m/z: 421.3 [(M+H)+].

[4-Morpholin-4-yl-6-(4-phenylpiperazin-1-4-yl)-[1,3,5]triazin-2-yl]p-tolylamine (Compound 280) (VIRADEM-015222): Yield 79%. M.p.=221-222° C. C₂₄H₂₉N₇O. MS m/z: 432.3 [(M+H)+].

(3,4-Dimethylphenyl)-[4-morpholin-4-yl-6-(4-phenylpiperazin-1-yl)-[1,3,5]triazin-2-yl]-amine (Compound 281) (VIRADEM-015223): Yield 75%. M.p.=152-153° C. C₂₅H₃₁N₇O. MS m/z: 446.3 [(M+H)+].

N-(3-Imidazol-1-yl-propyl)-6-morpholin-4-yl-N¹-p-tolyl-[1,3,5]triazine-2,4-diamine (Compound 282) (VIRADEM-015228): Yield 67%. M.p.>260° C. C₂₀H₂₆N₈O. MS m/z: 395.2 [(M+H)+].

(4-Azepan-1-yl-6-morpholin-4-yl-[1,3,5]triazin-2-yl)-p-tolyl-amine (Compound 283) (VIRADEM-015229): Yield 77%. M.p.=147-148° C. C₂₀H₂₈N₆O. MS m/z: 369.2 [(M+H)+].

(3,4-Dimethylphenyl)-[4-(4-methylpiperidin-1-yl)-6-morpholin-4-yl-[1,3,5]triazin-2-yl]-amine (Compound 284) (VIRADEM-015231): Yield 71%. M.p.=139-140° C. C₂₁H₃₀N₆O. MS m/z: 383.3 [(M+H)+].

N-(2-Methoxy-5-methylphenyl)-6-morpholin-4-yl-N¹-phenyl-[1,3,5]triazine-2,4-diamine (Compound 285) (VIRADEM-015232): Yield 73%. M.p.=157-158° C. C₂₁H₂₄N₆O₂. MS m/z: 393.2 [(M+H)+].

(3,4-Dimethylphenyl)-6-morpholin-4-yl-N¹-phenetyl-[1,3,5]triazine-2,4-diamine (Compound 286) (VIRADEM-015233): Yield 65%. M.p.=176-177° C. C₂₃H₂₈N₆O. MS m/z: 405.3 [(M+H)+].

[4-(3,5-Dimethylpiperidin-1-yl)-6-morpholin-4-yl-[1,3,5]triazin-2-yl]-p-tolyl-amine (Compound 287) (VIRADEM-015238): Yield 76%. M.p.=188-189° C. C₂₁H₃₀N₆O. MS m/z: 383.3 [(M+H)+]. ¹H NMR (400 MHz, DMSO-d₆) δ: 0.88 (d+m, 8H, CH₂₊₂CH₃), 1.49 (m, 2H, CH+CH), 2.23 (m, 5H, CH+CH+CH₃), 3.62 (m, 4H, CH₂NCH₂), 3.67 (m, 4H, CH₂OCH₂), 4.55 (m, 2H, CH+CH), 7.04 and 7.55 (d-d, 4H, C₆H₄), 8.95 (s, 1H, 1NH).

N-(4-Methoxyphenyl)-6-morpholin-4-yl-N¹-phenethyl-[1,3,5]triazine-2,4-diamine (Compound 288) (VIRADEM-015240): Yield 71%. M.p.=156-157° C. C₂₂H₂₆N₆O₂. MS m/z: 407.2 [(M+H)+].

4-(4-Morpholin-4-yl-6-p-tolylamino-[1,3,5]triazin-2-ylamino)-benzenesulfonamide (Compound 289) (VIRADEM-015244): Yield 81%. M.p.>260° C. C₂₀H₂₃N₇O₃S. MS m/z: 442.3 [(M+H)+].

N-(5-Chloro-2-methylphenyl)-N¹-(4-methoxyphenyl)-6-morpholin-4-yl-[1,3,5]triazine-2,4-diamine (Compound 290) (VIRADEM-015245): Yield 68%. M.p.=171-172° C. C₂₁H₂₃ClN₆O₂. MS m/z: 427.7 [(M+H)+].

N-(3,4-Dimethylphenyl)-6-morpholin-4-yl-N¹-(2,2,6,6-tetramethylpiperidin-4-yl)-[1,3,5]triazine-2,4-diamine (Compound 291) (VIRADEM-015247): Yield 72%. M.p.=170-171° C. C₂₄H₃₇N₇O. MS m/z: 440.3 [(M+H)+]. ¹H NMR (400 MHz, DMSO-d₆) δ: 1.02-1.74 (m, 16H, 2CH₂₊₄CH₃), 2.13 (s, 3H, CH₃), 2.17 (s, 3H, CH₃), 3.61 (m, 4H, CH₂NCH₂), 3.67 (m, 4H, CH₂OCH₂), 4.32 (m, 1H, CH), 6.75 (d, 1H, NH), 6.89-7.48 (m, 3H, C₆H₃), 8.65 (s, 1H, 1NH), 8.79 (s, 1H, 1NH).

3-[4-(4-Methoxyphenylamino)-6-morpholin-4-yl-[1,3,5]triazin-2-ylamino]-propan-1-ol (Compound 292) (VIRADEM-015254): Yield 57%. M.p.=125-126° C. C₁₇H₂₄N₆O₃. MS m/z: 361.2 [(M+H)+].

N-(2,3-Dimethylphenyl)-6-morpholin-4-yl-N¹-p-tolyl-[1,3,5]triazine-2,4-diamine (Compound 293) (VIRADEM-015258): Yield 69%. M.p.=145-146° C. C₂₂H₂₆N₆O. MS m/z: 391.3 [(M+H)+].

N-Cyclopentyl-N¹-(3,4-dimethylphenyl)-6-morpholin-4-yl-[1,3,5]triazine-2,4-diamine (Compound 294) (VIRADEM-015255): Yield 60%. M.p.=219-220° C. C₂₀H₂₈N₆O. MS m/z: 369.3 [(M+H)+].

N-(4-Methoxyphenyl)-6-morpholin-4-yl-N¹-(4-trifluoromethylphenyl)-[1,3,5]triazine-2,4-diamine (Compound 295) (VIRADEM-015259): Yield 72%. M.p.=251-252° C. C₂₁H₂₁F₃NO₂. MS m/z: 447.2 [(M+H)+].

N-(5-Chloro-2-methoxyphenyl)-N¹-(4-methoxyphenyl)-6-morpholin-4-yl-[1,3,5]triazine-2,4-diamine (Compound 296) (VIRADEM-015260): Yield 75%. M.p.=162-163° C. C₂₁H₂₃ClN₆O₃. MS m/z: 443.7 [(M+H)+].

N-(5-Chloro-4-methoxyphenyl)-N¹-(3,4-dimethoxyphenyl)-6-morpholin-4-yl-[1,3,5]triazine-2,4-diamine (Compound 297) (VIRADEM-015261): Yield 77%. M.p.=182-183° C. C₂₂H₂₅ClN₆O₂. MS m/z: 441.7 [(M+H)+].

N-(4-Methoxyphenyl)-6-morpholin-4-yl-N¹-naphthalen-[1,3,5]triazine-2,4-diamine (Compound 298) (VIRADEM-015262): Yield 79%. M.p.=206-207° C. C₂₄H₂₄N₆O₂. MS m/z: 429.2 [(M+H)+]. ¹H NMR (400 MHz, DMSO-d₆) δ: 3.62 (m, 4H, CH₂NCH₂), 3.67 (m, 7H, OCH₃+CH₂OCH₂), 6.70-8.04 (m, 11H, C₆H₄+C₁₀H₇), 8.91 (s, 1H, 1NH), 9.13 (s, 1H, 1NH).

N-(2,3-Dimethylphenyl)-6-morpholin-4-yl-N¹-quinolin-6-yl-[1,3,5]triazine-2,4-diamine (Compound 299) (VIRADEM-015263): Yield 77%. M.p.>260° C. C₂₄H₂₅N₇O. MS m/z: 428.3 [(M+H)+].

N-(3-Chloro-2-methylphenyl)-N¹-(3,4-dimethylphenyl)-6-morpholin-4-yl-[1,3,5]triazine-2,4-diamine (Compound 300) (VIRADEM-015264): Yield 71%. M.p.=204-205° C. C₂₂H₂₅ClN₆O. MS m/z: 425.7 [(M+H)+]. ¹H NMR (400 MHz, DMSO-d₆) δ: 2.08 (s, 3H, CH₃), 2.11 (s, 3H, CH₃), 2.23 (s, 3H, CH₃), 3.62 (m, 4H, CH₂NCH₂), 3.68 (m, 4H, CH₂OCH₂), 6.91-7.42 (m, 6H, C₆H₃+C₆H₃), 8.76 (s, 1H, 1NH), 8.94 (s, 1H, 1NH).

N-(3,4-Dimethylphenyl)-N¹-(3-ethylphenyl)-6-morpholin-4-yl-[1,3,5]triazine-2,4-diamine (Compound 301) (VIRADEM-015266): Yield 75%. M.p.=127-128° C. C₂₃H₂₈N₆O. MS m/z: 405.3 [(M+H)+].

N-(2-Ethylphenyl)-N¹-(4-methoxyphenyl)-6-morpholin-4-yl-[1,3,5]triazine-2,4-diamine (Compound 302) (VIRADEM-015267): Yield 70%. M.p.=126-127° C. C₂₂H₂₆N₆O₂. MS m/z: 407.2 [(M+H)+].

6-Morpholin-4-yl-N-quinolin-6-yl-N¹-p-tolyl-[1,3,5]triazine-2,4-diamine (Compound 303) (VIRADEM-015268): Yield 74%. M.p.=260-261° C. C₂₃H₂₃N₇O. MS m/z: 414.2 [(M+H)+].

N-(2,5-Dimethylphenyl)-N¹-(4-methoxyphenyl)-6-morpholin-4-yl-[1,3,5]triazine-2,4-diamine (Compound 304) (VIRADEM-015269): Yield 73%. M.p.=172-173° C. C₂₂H₂₆N₆O₂. MS m/z: 407.2 [(M+H)+].

N-(3,4-Dimethylphenyl)-6-morpholin-4-yl-N¹-naphthalen-1-yl-[1,3,5]triazine-2,4-diamine (Compound 305) (VIRADEM-015270): Yield 72%. C₂₂H₂₆N₆O₂. MS m/z: 427.3 [(M+H)+]. ¹H NMR (400 MHz, DMSO-d₆) δ: 2.20 (s, 6H, 2CH₃), 3.68 (m, 4H, CH₂NCH₂), 3.78 (m, 4H, CH₂OCH₂), 7.05-8.73 (m, 10H, C₆H₃+C₁₀H₇), 9.13 (s, 1H, 1NH), 9.54 (s, 1H, 1NH).

N-(5-Chloro-2-methylphenyl)-N¹-(3,4-dimethylphenyl)-6-morpholin-4-yl-[1,3,5]triazine-2,4-diamine (Compound 306) (VIRADEM-015271): Yield 77%. M.p.=178-179° C. C₂₂H₂₅ClN₆O. MS m/z: 425.7 [(M+H)+]. ¹H NMR (400 MHz, DMSO-d₆) δ: 2.10 (s, 3H, CH₃), 2.12 (s, 3H, CH₃), 2.21 (s, 3H, CH₃), 3.63 (m, 4H, CH₂NCH₂), 3.70 (m, 4H, CH₂OCH₂), 6.94-7.56 (m, 6H, C₆H₃+C₆H₃), 8.55 (s, 1H, 1NH), 8.99 (s, 1H, 1NH).

[4-(3,4-Dihydro-2H-quinolin-1-yl)-6-morpholin-4-yl-[1,3,5]triazin-2-yl]-(4-methoxyphenyl)-amine (Compound 307) (VIRADEM-015272): Yield 70%. M.p.=195-196° C. C₂₃H₂₆N₆O₂. MS m/z: 419.3 [(M+H)+].

6-Morpholin-4-yl-N-(2,2,6,6-tetramethylpiperidin-4-yl)-N¹-p-tolyl-[1,3,5]triazine-2,4-diamine (Compound 308) (VIRADEM-015273): Yield 67%. M.p.=170-171° C. C₂₃H₃₅N₇O. MS m/z: 426.3 [(M+H)+].)+]. ¹H NMR (400 MHz, DMSO-d₆) δ: 1.03-1.71 (m, 16H, 2CH₂₊₄CH₃), 2.22 (s, 3H, CH₃), 3.60 (m, 4H, CH₂NCH₂), 3.67 (m, 4H, CH₂OCH₂), 4.30 (m, 1H, CH), 6.78 (d, 1H, NH), 7.00 and 7.63 (d-d, 4H, C₆H₄), 8.74 (s, 1H, 1NH), 8.90 (s, 1H, 1NH).

1-{4-[4-(4-Methoxyphenylamino)-6-morpholin-4-yl-[1,3,5]triazin-2-ylamino]-phenyl}-ethanone (Compound 309) (VIRADEM-015274): Yield 82%. M.p.=216-217° C. C₂₂H₂₄N₆O₃. MS m/z: 421.2 [(M+H)+].

N-(2,4-Dimethoxyphenyl)-6-morpholin-4-yl-N¹-p-tolyl-[1,3,5]triazine-2,4-diamine (Compound 310) (VIRADEM-015275): Yield 68%. M.p.=173-174° C. C₂₂H₂₆N₆O₃. MS m/z: 423.3 [(M+H)+]. ¹H NMR (400 MHz, DMSO-d₆) δ: 2.23 (s, 3H, CH₃), 3.64 (m, 4H, CH₂NCH₂), 3.69 (m, 4H, CH₂OCH₂), 3.77 (s, 3H, OCH₃), 3.81 (s, 3H, OCH₃), 7.03 and 7.54 (d-d, 4H, C₆H₄), 6.50-7.74 (m, 3H, C₆H₃), 7.80 (s, 1H, 1NH), 9.07 (s, 1H, 1NH).

N-(5-Chloro-2-methylphenyl)-6-morpholin-4-yl-N¹-p-tolyl-[1,3,5]triazine-2,4-diamine (Compound 311) (VIRADEM-015276): Yield 71%. M.p.=178-179° C. C₂₁H₂₃ClN₆O. MS m/z: 411.7 [(M+H)+].

N-(4-Methoxyphenyl)-6-morpholin-4-yl-N¹-quinolin-6-yl-[1,3,5]triazine-2,4-diamine (Compound 312) (VIRADEM-015277): Yield 79%. M.p.>260° C. C₂₃H₂₃N₇O. MS m/z: 430.2 [(M+H)+]. ¹H NMR (400 MHz, DMSO-d₆) δ: 3.63 (m, 4H, CH₂NCH₂), 3.74 (m, 4H, CH₂OCH₂), 3.77 (s, 3H, OCH₃), 6.90 and 7.59 (d-d, 4H, C₆H₄), 7.44-8.73 (m, 6H, quinolin), 9.13 (s, 1H, 1NH), 9.52 (s, 1H, 1NH).

[4-(3,4-Dihydro-2-H-quinolin-1-yl)-6-morpholin-4-yl-[1,3,5]triazin-2-yl]-(3,4-dimethylphenyl)-amine (Compound 313) (VIRADEM-015278): Yield 75%. M.p.=181-182° C. C₂₄H₂₈N₆O. MS m/z: 417.3 [(M+H)+].

6-Morpholin-4-yl-N-naphtalen-1-yl-N¹-p-tolyl-[1,3,5]triazine-2,4-diamine (Compound 314) (VIRADEM-015279): Yield 82%. M.p.=219-220° C. C₂₄H₂₄N₆O. MS m/z: 413.3 [(M+H)+].

N-(3-Chloro-2-methylphenyl)-N¹-(4-methoxyphenyl)-6-morpholin-4-yl-[1,3,5]triazine-2,4-diamine (Compound 315) (VIRADEM-015280): Yield 73%. M.p.=172-173° C. C₂₁H₂₃ClN₆O₂. MS m/z: 427.7 [(M+H)+].

N-(2-Ethoxyphenyl)-6-morpholin-4-yl-N¹-p-tolyl-[1,3,5]triazine-2,4-diamine (Compound 316) (VIRADEM-015281): Yield 68%. M.p.=186-187° C. C₂₂H₂₆N₆O₂. MS m/z: 407.2 [(M+H)+].

N-(3-Chloro-4-methoxyphenyl)-6-morpholin-4-yl-N¹-p-tolyl-[1,3,5]triazine-2,4-diamine (Compound 317) (VIRADEM-015282): Yield 74%. M.p.=207-208° C. C₂₁H₂₃ClN₆O₂. MS m/z: 427.7 [(M+H)+]. ¹H NMR (400 MHz, DMSO-d₆) δ: 2.25 (s, 3H, CH₃), 3.65 (m, 4H, CH₂NCH₂), 3.73 (m, 4H, CH₂OCH₂), 3.82 (s, 3H, CH₃), 7.05-7.87 (m, 7H, C₆H₄+C₆H₃), 9.11 (s, 1H, 1NH), 9.15 (s, 1H, 1NH).

N-(2,4-Dimethylphenyl)-N¹-(4-methoxyphenyl)-6-morpholin-4-yl-[1,3,5]triazine-2,4-diamine (Compound 318) (VIRADEM-015285): Yield 70%. M.p.=216-217° C. C₂₂H₂₆N₆O₂. MS m/z: 407.2 [(M+H)+].

N-(3,4-Dimethylphenyl)-N¹-(2-ethoxyphenyl)-6-morpholin-4-yl-[1,3,5]triazine-2,4-diamine (Compound 319) (VIRADEM-015286): Yield 65%. M.p.=151-152° C. C₂₃H₂₈N₆O. MS m/z: 405.3 [(M+H)+].

N-(2-Ethylphenyl)-6-morpholin-4-yl-N¹-p-tolyl-[1,3,5]triazine-2,4-diamine (Compound 320) (VIRADEM-015287): Yield 74%. M.p.=136-137° C. C₂₂H₂₆N₆O. MS m/z: 391.2 [(M+H)+].

N-Furan-2-ylmethyl-N¹-(4-methoxyphenyl)-6-morpholin-4-yl-[1,3,5]triazine-2,4-diamine (Compound 321) (VIRADEM-015288): Yield 77%. M.p.=191-192° C. C₁₉H₂₂N₆O₃. MS m/z: 383.2 [(M+H)+]. ¹H NMR (400 MHz, DMSO-d₆) δ: 3.62 (m, 4H, CH₂NCH₂), 3.67 (m, 4H, CH₂OCH₂), 3.71 (s, 3H, OCH₃), 4.45 (m, 2H, CH₂), 6.22-7.28 (m, 3H, C₄H₃₀), 6.82 and 7.59 (d-d, 4H, C₆H₄), 8.77 (s, 1H, 1NH), 8.89 (s, 1H, 1NH).

N-Cyclopentyl-N¹-(4-methoxyphenyl)-6-morpholin-4-yl-[1,3,5]triazine-2,4-diamine (Compound 322) (VIRADEM-015289): Yield 63%. M.p.=158-159° C. C₁₉H₂₆N₆O₂. MS m/z: 371.3 [(M+H)+].

N-Furan-2-ylmethyl-N¹-6-morpholin-4-yl-N¹-p-tolyl-[1,3,5]triazine-2,4-diamine (Compound 323) (VIRADEM-015290): Yield 67%. M.p.=152-153° C. C₁₉H₂₂N₆O₂. MS m/z: 367.3 [(M+H)+]. ¹H NMR (400 MHz, DMSO-d₆) δ: 2.27 (s, 3H, CH₃), 3.63 (m, 4H, CH₂NCH₂), 3.68 (m, 4H, CH₂OCH₂), 4.44 (m, 2H, CH₂), 6.21-7.27 (m, 3H, C₄H₃₀), 7.08 and 7.64 (d-d, 4H, C₆H₄), 8.92 (s, 1H, 1NH), 9.18 (s, 1H, 1NH).

N-(2-Chloro-4-methoxyphenyl)-N¹-(4-methoxyphenyl)-6-morpholin-4-yl-[1,3,5]triazine-2,4-diamine (Compound 324) (VIRADEM-015350): Yield 71%. M.p.=164-165° C. C₂₁H₂₃ClN₆O₃. MS m/z: 443.7 [(M+H)+].

4-[4-(4-Methoxyphenylamino)-6-morpholin-4-yl-[1,3,5]triazin-2-ylamino]-benzamide (Compound 325) (VIRADEM-015356): Yield 79%. M.p.>260° C. C₂₁H₂₃N₇O₃. MS m/z: 422.2 [(M+H)+].

4-[4-(4-Methoxyphenylamino)-6-morpholin-4-yl-N¹-(2,2,6,6-tetramethylpiperidin-4-yl)-[1,3,5]triazin-2,4-diamine (Compound 326) (VIRADEM-015364): Yield 70%. M.p.=146-147° C. C₂₃H₃₅N₇O₂. MS m/z: 442.3 [(M+H)+].

N-(3-Chlorophenyl)-N′,N²-diisopropyl-[1,3,5]triazine-2,4,6-triamine (Compound 327) (VIRADEM-015446): Yield 51%. M.p.=123-124° C. C₁₅H₂₁ClN₆. MS m/z: 321.6 [(M+H)+].

N-(3,4-Dimethylphenyl)-6-morpholin-4-yl-N¹-(4-trifluoromethylphenyl)-[1,3,5]triazine-2,4-diamine (Compound 328) (VIRADEM-016437): Yield 79%. M.p.=236-237° C. C₂₂H₂₃F₃N₆O. MS m/z: 445.2 [(M+H)+].

N-Benzo[1,3]dioxol-5-ylmethyl-6-morpholin-4-yl-N¹-p-tolyl-[1,3,5]triazine-2,4-diamine (Compound 329) (VIRADEM-016438): Yield 73%. M.p.=194-195° C. C₂₂H₂₄N₆O₃. MS m/z: 421.2 [(M+H)+].

(4-Azepan-1-yl-6-morpholin-4-yl-[1,3,5]triazin-2-yl)-(4-methoxyphenyl)-amine (Compound 330) (VIRADEM-016510): Yield 75%. M.p.=136-137° C. C₂₀H₂₈N₆O₂. MS m/z: 385.2 [(M+H)+].

N-Cyclopentyl-6-morpholin-4-yl-N¹-p-tolyl-[1,3,5]triazine-2,4-diamine (Compound 331) (VIRADEM-016518): Yield 65%. M.p.=187-188° C. C₁₉H₂₆N₆O. MS m/z: 355.2 [(M+H)+].

N-(2,3-Dihydrobenzo[1,4]dioxin-6-yl)-6-morpholin-4-yl-N¹-p-tolyl-[1,3,5]triazine-2,4-diamine (Compound 332) (VIRADEM-016521): Yield 76%. M.p.=185-186° C. C₂₂H₂₄N₆O₃. MS m/z: 421.2 [(M+H)+].

N-(3-Fluorophenyl)-N¹,N²-diisopropyl-[1,3,5]triazine-2,4,6-triamine (Compound 333) (VIRADEM-016571): Yield 53%. M.p.=114-115° C. C₁₅H₂₁FN₆. MS m/z: 305.1 [(M+H)+].

N-(3-Chloro-2-methylphenyl)-N¹,N²-diisopropyl-[1,3,5]triazine-2,4,6-triamine (Compound 334) (VIRADEM-016572): Yield 50%. M.p.=118-119° C. C₁₆H₂₃ClN₆. MS m/z: 335.7 [(M+H)+].

N,N¹-Diisopropyl-N²-(4-phenoxyphenyl)-[1,3,5]triazine-2,4,6-triamine (Compound 335) (VIRADEM-016573): Yield 58%. M.p.=167-168° C. C₂₁H₂₆N₆. MS m/z: 379.2 [(M+H)+].

N-(3,4-Dimethylphenyl)-N¹,N²-dimethyl-6-morpholin-4-yl-[1,3,5]triazine-2,4-diamine (Compound 336) (VIRADEM-019803): Yield 67%. M.p.=143-144° C. C₁₇H₂₄N₆O. MS m/z: 329.2 [(M+H)+].

2-(4,6-dimorpholine-4-yl-1,3,5-triazine-2-yl)-N-phenylhydrazine-carbothioamide (Compound 337) (VIRADEM-010867) was obtained by method [16]: Yield (85%). Mp=188-190° C. C₁₈H₂₄N₈O₂S. ¹H NMR (400 MHz, DMSO-d₆) δ: 3.56 (m, 8H, 2CH₂NCH₂), 3.60 (m, 8H, 2CH₂OCH₂), 7.10-7.46 (M, 5H, Ph), 8.85 (c, 1H, NH), 9.47 (c, 1H, NH), 9.61 (c, 1H, NH).

(4,6-Dimorpholin-4-yl-[1,3,5]triazin-2-yl)-(4-ethoxyphenyl)-amine hydrochloride (Compound 338) (VIRADEM-009794): Yield 77%. M.p.=215-216° C. C₁₉H₂₇ClN₆O₃. MS m/z: 387.2 [(M+H)+]. 1H NMR (400 MHz, DMSO-d₆) δ: 1.31 (t, 3H, OCH₂CH₃), 3.66 (m, 8H, 2CH₂NCH₂), 3.75 (m, 8H, 2CH₂OCH₂), 4.00 (q, 2H, OCH₂CH₃), 6.89 and 7.48 (d-d, 4H, C₆H₄), 9.96 (s, 1H, 1NH).

(4-n-Butylphenyl)-(4,6-dimorpholin-4-yl-[1,3,5]triazin-2-yl)-amine hydrochloride (Compound 339) (VIRADEM-010257): Yield 75%. M.p.=190-191° C. C₂₁H₃₁ClN₆O₂. MS m/z: 399.3 [(M+H)+]. ¹H NMR (400 MHz, DMSO-d₆) δ: 0.90 (t, 3H, CH₃), 1.26 (m, 2H, CH₂), 1.54 (m, 2H, CH₂), 2.53 (m, 2H, CH₂), 3.65 (m, 8H, 2CH₂NCH₂), 3.74 (m, 8H, 2CH₂OCH₂), 7.14 and 7.52 (d-d, 4H, C₆H₄), 9.89 (s, 1H, 1NH).

(3,4-Dichlorophenyl)-(4,6-dimorpholin-4-yl-[1,3,5]triazin-2-yl)-amine hydrochloride (Compound 340) (VIRADEM-010258): Yield 79%. M.p.=196-197° C. C₁₇H₂₁Cl₃N₆O₂. MS m/z: 412.1 [(M+H)+]. ¹H NMR (400 MHz, DMSO-d₆) δ: 3.62 (m, 8H, 2-CH₂NCH₂—), 3.69 (m, 8H, 2-CH₂OCH₂—), 7.49-8.04 (m, 3H, C₆H₃), 9.74 (s, 1H, NH).

(4,6-Dimorpholin-4-yl-[1,3,5]triazin-2-yl)-phenylamine hydrochloride (Compound 341) (VIRADEM-010846): Yield 75%. M.p.=206-207° C. C₁₇H₂₃ClN₆O₂. MS m/z: 343.2 [(M+H)+]. ¹H NMR (400 MHz, DMSO-d₆) δ: 3.63 (m, 8H, 2-CH₂NCH₂—), 3.74 (m, 8H, 2-CH₂OCH₂—), 7.05-7.62 (m, 5H, C₆H₅), 9.95 (s, 1H, NH).

(4,6-Dimorpholin-4-yl-[1,3,5]triazin-2-yl)-m-tolyl-amine hydrochloride (Compound 342) (VIRADEM-010848): Yield 77%. M.p.=220-221° C. C₁₈H₂₅ClN₆O₂. MS m/z: 357.2 [(M+H)+]. ¹H NMR (400 MHz, DMSO-d₆) δ: 2.27 (s, 3H, CH₃), 3.64 (m, 8H, 2-CH₂NCH₂—), 3.75 (m, 8H, 2-CH₂OCH₂—), 6.86-7.45 (m, 4H, C₆H₄), 9.88 (s, 1H, NH).

(4,6-Dimorpholin-4-yl-[1,3,5]triazin-2-yl)-(4-fluorophenyl)-amine hydrochloride (Compound 343) (VIRADEM-010849): Yield 79%. M.p.=200-201° C. C₁₇H₂₂ClFN₆O₂. MS m/z: 361.1 [(M+H)+]. ¹H NMR (400 MHz, DMSO-d₆) δ: 3.61 (m, 8H, 2-CH₂NCH₂—), 3.74 (m, 8H, 2-CH₂OCH₂—), 7.15-7.64 (m, 4H, C₆H₄), 9.91 (s, 1H, NH).

(4,6-Dimorpholin-4-yl-[1,3,5]triazin-2-yl)-dimethylamine (Compound 344) (VIRADEM-010026): Yield 69%. M.p.=200-201° C. C₁₃H₂₂N₆O₂. MS m/z: 295.1 [(M+H)+]. ¹H NMR (400 MHz, DMSO-d₆) δ: 3.02 (s, 6H, CH₃NCH₃), 3.57 (m, 8H, 2-CH₂NCH₂—), 3.66 (m, 8H, 2-CH₂OCH₂—).

(4,6-Dimorpholin-4-yl-[1,3,5]triazin-2-yl)-hydrazine (Compound 345) (VIRADEM-010052): Yield 85%. C₁₁H₁₉N₇O₂. MS m/z: 382.1 [(M+H)+]. ¹H NMR (400 MHz, DMSO-d₆) δ: 3.57 (m, 8H, 2-CH₂NCH₂—), 3.66 (m, 8H, 2-CH₂OCH₂—), 4.16 (s, 2H, NH₂), 7.83 (s, 1H, NH).

(4,6-Dimorpholin-4-yl-[1,3,5]triazin-2-yl)-(2-fluorophenyl)-amine hydrochloride (Compound 346) (VIRADEM-010256): Yield 77%. C₁₇H₂₂ClFN₆O₂. MS m/z: 361.1 [(M+H)+]. ¹H NMR (400 MHz, DMSO-d₆) δ: 3.63 (m, 8H, 2-CH₂NCH₂—), 3.73 (m, 8H, 2-CH₂OCH₂—), 7.13-7.73 (m, 4H, C₆H₄), 10.1 (s, 1H, NH).

1-[3-(4,6-Dimorpholin-4-yl-[1,3,5]triazin-2-yl-[1,3,5]triazin-2-ylamino)-phenyl]-ethanone hydrochloride (Compound 347) (VIRADEM-010847): Yield 84%. M.p.=211-212° C. C₁₉H₂₅ClN₆O₃. MS m/z: 385.2 [(M+H)+]. ¹H NMR (400 MHz, DMSO-d₆) δ: 2.56 (s, 3H, COCH₃), 3.66 (m, 8H, 2-CH₂NCH₂—), 3.77 (m, 8H, 2-CH₂OCH₂—), 7.46 (t, 1H, C₆H₄), 7.61 (d, 1H, C₆H₄), 7.77 (d, 1H, C₆H₄), 8.44 (s, 1H, C₆H₄), 9.84 (s, 1H, NH).

(3,4-Dimethoxyphenyl)-(4,6-dimorpholin-4-yl-[1,3,5]triazin-2-yl)-amine hydrochloride (Compound 348) (VIRADEM-010850): Yield 81%. M.p.=216-217° C. C₁₉H₂₇ClN₆O₄. MS m/z: 403.2 [(M+H)+]. ¹H NMR (400 MHz, DMSO-d₆) δ: 3.66 (m, 8H, 2-CH₂NCH₂—), 3.75 (m, 14H, 2OCH₃+2-CH₂OCH₂—), 6.91 (d, 1H, C₆H₃), 7.05 (d, 1H, C₆H₃), 7.41 (s, 1H, C₆H₃), 9.64 (s, 1H, NH).

2,4,6-Trimorpholin-4-yl-[1,3,5]triazine (Compound 349) (VIRADEM-010855): Yield 81%. M.p.=>260° C. C₁₅H₂₄N₆O₃. MS m/z: 337.2 [(M+H)+]. ¹H NMR (400 MHz, DMSO-d₆) δ: 3.80 (m, 12H, 2-CH₂NCH₂—), 3.84 (m, 12H, 2-CH₂OCH₂—).

(4,6-Dimorpholin-4-yl-[1,3,5]triazin-2-yl)-(3-methoxyphenyl)-amine hydrochloride (Compound 350) (VIRADEM-010879): Yield 80%. M.p.=214-215° C. C₁₈H₂₅ClN₆O₃. MS m/z: 373.2 [(M+H)+]. 1H NMR (400 MHz, DMSO-d₆) δ: 3.65 (m, 8H, 2-CH₂NCH₂—), 3.74 (s, 3H, OCH₃), 3.75 (m, 8H, 2-CH₂OCH₂—), 6.61 (d, 1H, C₆H₄), 7.12 (d, 1H, C₆H₄), 7.22 (t, 1H, C₆H₄), 7.39 (s, 1H, C₆H₄), 9.95 (s, 1H, NH).

(4,6-Dimorpholin-4-yl-[1,3,5]triazin-2-yl)-(3-fluorophenyl)-amine hydrochloride (Compound 351) (VIRADEM-010880): Yield 81%. M.p.=221-222° C. C₁₇H₂₂ClFN₆O₂. MS m/z: 361.2 [(M+H)+]. 1H NMR (400 MHz, DMSO-d₆) δ: 3.64 (m, 8H, 2-CH₂NCH₂—), 3.73 (s, 3H, OCH₃), 3.75 (m, 8H, 2-CH₂OCH₂—), 6.84-7.60 (m, 4H, C₆H₄), 10.0 (s, 1H, NH).

(4,6-Dimorpholin-4-yl-[1,3,5]triazin-2-yl)-(2-methoxyphenyl)-amine hydrochloride (Compound 352) (VIRADEM-010881): Yield 73%. M.p.=195-196° C. C₁₈H₂₅ClN₆O₃. MS m/z: 373.2 [(M+H)+]. ¹H NMR (400 MHz, DMSO-d₆) δ: 3.67 (m, 8H, 2-CH₂NCH₂—), 3.76 (m, 8H, 2-CH₂OCH₂—), 3.82 (s, 3H, OCH₃), 6.96-8.08 (m, 4H, C₆H₄), 9.13 (s, 1H, NH).

(4,6-Dimorpholin-4-yl-[1,3,5]triazin-2-yl)-p-tolyl-amine hydrochloride (Compound 353) (VIRADEM-010882): Yield 85%. M.p.=212-213° C. C₁₈H₂₅ClN₆O₂. MS m/z: 357.2 [(M+H)+]. ¹H NMR (400 MHz, DMSO-d₆) δ: 2.26 (s, 3H, CH₃), 3.64 (m, 8H, 2-CH₂NCH₂—), 3.75 (m, 8H, 2-CH₂OCH₂—), 7.15 and 7.47 (d-d, 4H, C₆H₄), 10.3 (s, 1H, NH).

(4,6-Dimorpholin-4-yl-[1,3,5]triazin-2-yl)-(4-ethylphenyl)-amine hydrochloride (Compound 354) (VIRADEM-010883): Yield 82%. M.p.=223-224° C. C₁₉H₂₇ClN₆O₂. MS m/z: 371.2 [(M+H)+]. 1H NMR (400 MHz, DMSO-d₆) δ: 1.08 (t, 3H, CH₂CH₃), 2.27 (q, 2H, CH₂CH₃), 3.66 (m, 8H, 2-CH₂NCH₂—), 3.77 (m, 8H, 2-CH₂OCH₂—), 7.18 and 7.52 (d-d, 4H, C₆H₄), 10.1 (s, 1H, NH).

(4,6-Dimorpholin-4-yl-[1,3,5]triazin-2-yl)-(4-methoxyphenyl)-amine hydrochloride (Compound 356) (VIRADEM-010885): Yield 77%. M.p.=206-207° C. C₁₈H₂₅ClN₆O₃. MS m/z: 373.2 [(M+H)+]. ¹H NMR (400 MHz, DMSO-d₆) δ: 3.67 (m, 8H, 2-CH₂NCH₂—), 3.74 (s, 3H, OCH₃), 3.76 (m, 8H, 2-CH₂OCH₂—), 6.93 and 7.50 (d-d, 4H, C₆H₄), 10.1 (s, 1H, NH).

(3-Chloro-4-methylphenyl)-(4,6-dimorpholin-4-yl-[1,3,5]triazin-2-yl)-amine hydrochloride (Compound 357) (VIRADEM-010886): Yield 75%. M.p.=201-202° C. C₁₈H₂₄Cl₂N₆O₃. MS m/z: 391.6 [(M+H)+]. ¹H NMR (400 MHz, DMSO-d₆) δ: 2.23 (s, 3H, CH₃), 3.67 (m, 8H, 2-CH₂NCH₂—), 3.74 (m, 8H, 2-CH₂OCH₂—), 7.28 (d, 1H, C₆H₃), 7.48 (d, 1H, C₆H₃), 7.81 (s, 1H, C₆H₃), 9.89 (s, 1H, NH).

(3,5-Dimethylphenyl)-(4,6-dimorpholin-4-yl-[1,3,5]triazin-2-yl)-amine (Compound 358) (VIRADEM-010887): Yield 73%. M.p.=190-191° C. C₁₉H₂₇ClN₆O₂. MS m/z: 371.2 [(M+H)+]. ¹H NMR (400 MHz, DMSO-d₆) δ: 2.22 (s, 6H, 2CH₃), 3.67 (m, 8H, 2-CH₂NCH₂—), 3.75 (m, 8H, 2-CH₂OCH₂—), 6.67 (s, 1H, C₆H₃), 7.28 (s, 2H, C₆H₃), 9.61 (s, 1H, NH).

(3,5-Dichlorophenyl)-(4,6-dimorpholin-4-yl-[1,3,5]triazin-2-yl)-amine (Compound 359) (VIRADEM-010888): Yield 85%. M.p.=235-236° C. C₁₇H₁₉Cl₃N₆O₂. MS m/z: 412.2 [(M+H)+]. ¹H NMR (400 MHz, DMSO-d₆) δ: 3.63 (m, 8H, 2-CH₂NCH₂—), 3.72 (m, 8H, 2-CH₂OCH₂—), 7.22 (s, 1H, C₆H₃), 7.81 (s, 2H, C₆H₃), 9.62 (s, 1H, NH).

4-(4,6-Dimorpholin-4-yl-[1,3,5]triazin-2-ylamino-benzoic acid ethyl ester hydrochloride (Compound 360) (VIRADEM-010889): Yield 72%. M.p.=202-203° C. C₂₀H₂₇ClN₆O₄. MS m/z: 415.2 [(M+H)+]. ¹H NMR (400 MHz, DMSO-d₆) δ: 1.31 (t, 3H, COOCH₂CH₃), 3.64 (m, 8H, 2-CH₂NCH₂—), 3.73 (m, 8H, 2-CH₂OCH₂—), 4.30 (q, 2H, COOCH₂CH₃), 7.81 and 7.92 (d-d, 4H, C₆H₄), 10.1 (s, 1H, NH).

4-(4,6-Dimorpholin-4-yl-[1,3,5]triazin-2-ylamino-benzoic acid methyl ester hydrochloride (Compound 361) (VIRADEM-010890): Yield 75%. M.p.=206-207° C. C₁₉H₂₅ClN₆O₄. MS m/z: 401.2 [(M+H)+]. ¹H NMR (400 MHz, DMSO-d₆) δ: 3.65 (m, 8H, 2-CH₂NCH₂—), 3.74 (m, 8H, 2-CH₂OCH₂—), 3.83 (s, 3H, COOCH₃), 7.80 and 7.92 (d-d, 4H, C₆H₄), 10.1 (s, 1H, NH).

(4,6-Dimorpholin-4-yl-[1,3,5]triazin-2-yl)-(4-trifluoromethoxyphenyl)-amine hydrochloride (Compound 362) (VIRADEM-010891): Yield 80%. M.p.=220-221° C. C₁₈H₂₂ClF₃N₆O₃. MS m/z: 427.2 [(M+H)+]. ¹H NMR (400 MHz, DMSO-d₆) δ: 3.62 (m, 8H, 2-CH₂NCH₂—), 3.73 (m, 8H, 2-CH₂OCH₂—), 7.32 and 7.76 (d-d, 4H, C₆H₄), 9.91 (s, 1H, NH).

(4,6-Dimorpholin-4-yl-[1,3,5]triazin-2-yl)-(4-phenoxyphenyl)-amine hydrochloride (Compound 363) (VIRADEM-010892): Yield 85%. M.p.=182-183° C. C₂₃H₂₇ClN₆O₃. MS m/z: 435.3 [(M+H)+]. ¹H NMR (400 MHz, DMSO-d₆) δ: 3.63 (m, 8H, 2-CH₂NCH₂—), 3.74 (m, 8H, 2-CH₂OCH₂—), 6.98-7.56 (m, 9H, C₆H₅+C₆H₄), 10.1 (s, 1H, NH).

(3-Chloro-4-fluorophenyl)-(4,6-dimorpholin-4-yl-[1,3,5]triazin-2-yl)-amine hydrochloride (Compound 364) (VIRADEM-010893): Yield 81%. M.p.=223-224° C. C₁₇H₂₁Cl₂FN₆O₂. MS m/z: 395.6 [(M+H)+]. ¹H NMR (400 MHz, DMSO-d₆) δ: 3.62 (m, 8H, 2-CH₂NCH₂—), 3.73 (m, 8H, 2-CH₂OCH₂—), 7.34-7.93 (m, 3H, C₆H₃), 9.83 (m, 1H, NH).

(4-Chlorophenyl)-(4,6-dimorpholin-4-yl-[1,3,5]triazin-2-yl)-amine hydrochloride (Compound 365) (VIRADEM-010894): Yield 84%. M.p.=218-219° C. C₁₇H₂₂Cl₂N₆O₂. MS m/z: 377.6 [(M+H)+]. ¹H NMR (400 MHz, DMSO-d₆) δ: 3.63 (m, 8H, 2-CH₂NCH₂—), 3.74 (m, 8H, 2-CH₂OCH₂—), 7.37 and 7.66 (d-d, 4H, C₆H₄), 10.0 (m, 1H, NH).

(3-Chlorophenyl)-(4,6-dimorpholin-4-yl-[1,3,5]triazin-2-yl)-amine hydrochloride (Compound 366) (VIRADEM-010902): Yield 82%. M.p.=190-191° C. C₁₇H₂₂Cl₂N₆O₂. MS m/z: 377.6 [(M+H)+]. 1H NMR (400 MHz, DMSO-d₆) δ: 3.64 (m, 8H, 2-CH₂NCH₂—), 3.73 (m, 8H, 2-CH₂OCH₂—), 7.05 (d, 1H, C₆H₄), 7.13 (t, 1H, C₆H₄), 7.52 (d, 1H, C₆H₄), 7.57 (s, 1H, C₆H₄), 9.94 (m, 1H, NH).

2,4-Dimorpholin-4-yl-6-piperidin-1-yl-[1,3,5]triazin hydrochloride (Compound 367) (VIRADEM-010903): Yield 73%. M.p.=231-232° C. C₁₆H₂₇ClN₆O₂. MS m/z: 335.2 [(M+H)+]. ¹H NMR (400 MHz, DMSO-d₆) δ: 1.22-1.31 (m, 6H, CH₂CH₂CH₂), 3.55-3.69 (m, 20H, 3-CH₂NCH₂—+2-CH₂OCH₂—).

(2,4-Dimorpholin-4-yl-[1,3,5]triazin-2-yl)-(3-trifluoromethylphenyl)-amine hydrochloride (Compound 368) (VIRADEM-010993): Yield 79%. M.p.=231-232° C. C₁₈H₂₂ClF₃N₆O₂. MS m/z: 411.2 [(M+H)+]. ¹H NMR (400 MHz, DMSO-d₆) δ: 3.63 (m, 8H, 2-CH₂NCH₂—), 3.74 (m, 8H, 2-CH₂OCH₂—), 7.29 (d, 1H, C₆H₄), 7.51 (t, 1H, C₆H₄), 7.79 (d, 1H, C₆H₄), 8.29 (s, 1H, C₆H₄), 9.81 (m, 1H, NH).

(5-Chloro-2-methylphenyl)-(4,6-dimorpholin-4-yl-[1,3,5]triazin-2-yl)-amine (Compound 369) (VIRADEM-011005): Yield 73%. M.p.=176-177° C. C₁₈H₂₃ClN₆O₂. MS m/z: 391.6 [(M+H)+]. ¹H NMR (400 MHz, DMSO-d₆) δ: 2.08 (s, 3H, CH₃), 3.58 (m, 8H, 2-CH₂NCH₂—), 3.67 (m, 8H, 2-CH₂OCH₂—), 7.03 (d, 1H, C₆H₃), 7.18 (d, 1H, C₆H₃), 7.65 (s, 1H, C₆H₃), 8.38 (m, 1H, NH).

(4,6-Dimorpholin-4-yl-[1,3,5]triazin-2-yl)-o-tolyl-amine (Compound 370) (VIRADEM-012434): Yield 70%. M.p.=201-202° C. C₁₈H₂₅ClN₆O₂. MS m/z: 357.4 [(M+H)+]. ¹H NMR (400 MHz, DMSO-d₆) δ: 2.09 (s, 3H, CH₃), 3.64 (m, 8H, 2-CH₂NCH₂—), 3.75 (m, 8H, 2-CH₂OCH₂—), 7.04-7.39 (m, 4H, C₆H₄), 9.93 (s, 1H, NH).

(4,6-Dimorpholin-4-yl-[1,3,5]triazin-2-yl)-(4-isopropylphenyl)-amine (Compound 371) (VIRADEM-012435): Yield 77%. M.p.=214-215° C. C₂₀H₂₉ClN₆O₂. MS m/z: 385.2 [(M+H)+]. ¹H NMR (400 MHz, DMSO-d₆) δ: 1.19 (d, 6H, CH(CH₃)₂), 2.85 (m, 1H, CH(CH₃)₂), 3.65 (m, 8H, 2-CH₂NCH₂—), 3.75 (m, 8H, 2-CH₂OCH₂—), 7.14-7.59 (d-d, 4H, C₆H₄), 9.89 (s, 1H, NH).

(2,5-Dichlorophenyl)-(4,6-dimorpholin-4-yl-[1,3,5]triazin-2-yl)-amine (Compound 372) (VIRADEM-014270): Yield 78%. M.p.=154-155° C. C₁₇H₂₀Cl₂N₆O₂. MS m/z: 412.1 [(M+H)+]. ¹H NMR (400 MHz, DMSO-d₆) δ: 3.59 (m, 8H, 2-CH₂NCH₂—), 3.68 (m, 8H, 2-CH₂OCH₂—), 7.15 (d, 1H, C₆H₃), 7.38 (d, 1H, C₆H₃), 7.69 (s, 1H, C₆H₃), 8.89 (m, 1H, NH).

3-(4,6-Dimorpholin-4-yl-[1,3,5]triazin-2-ylamino)propan-1-ol (Compound 373) (VIRADEM-014271): Yield 66%. C₁₄H₂₄N₆O₃. MS m/z: 325.2 [(M+H)+].

(2,5-Dimethylphenyl)-(4,6-dimorpholin-4-yl-[1,3,5]triazin-2-yl)-amine (Compound 374) (VIRADEM-014272): Yield 78%. M.p.=182-183° C. C₁₉H₂₇ClN₆O₂. MS m/z: 371.2 [(M+H)+]. ¹H NMR (400 MHz, DMSO-d₆) δ: 2.06 (s, 3H, CH₃), 2.21 (s, 3H, CH₃), 3.57 (m, 8H, 2-CH₂NCH₂—), 3.66 (m, 8H, 2-CH₂OCH₂—), 6.81 (d, 1H, C₆H₃), 7.08 (d, 1H, C₆H₃), 7.29 (s, 1H, C₆H₃), 8.20 (s, 1H, NH).

N-[4-(4,6-Dimorpholin-4-yl-[1,3,5]triazin-2-ylamino)-phenyl]-acetamide hydrochloride (Compound 375) (VIRADEM-014278): Yield 81%. M.p.>260° C. C₁₉H₂₆ClN₇O₂. MS m/z: 400.2 [(M+H)+]. ¹H NMR (400 MHz, DMSO-d₆) δ: 2.04 (s, 3H, NHCOCH₃), 3.66 (m, 4H, CH₂NCH₂), 3.75 (m, 4H, CH₂OCH₂), 7.29 and 7.60 (d-d, 4H, C₆H₄), 9.83 (m, 1H, NH).

(4,6-Dimorpholin-4-yl-[1,3,5]triazin-2-yl)-(4-nitrophenyl)-amine (Compound 376) (VIRADEM-014285): Yield 70%. M.p.>260° C. C₁₇H₂₁N₇O₄. MS m/z: 388.2 [(M+H)+].

Cyclohexyl-(4,6-dimorpholin-4-yl-[1,3,5]triazin-2-yl)-amine (Compound 377) (VIRADEM-014286): Yield 65%. M.p.=184-185° C. C₁₇H₂₈N₆O₂. MS m/z: 349.2 [(M+H)+].

1-[4-[(4,6-Dimorpholin-4-yl-[1,3,5]triazin-2-ylamino)-phenyl]-ethanone hydrochloride (Compound 378) (VIRADEM-014287): Yield 83%. M.p.=241-242° C. C₁₉H₂₅ClN₆O₃. MS m/z: 385.2 [(M+H)+]. ¹H NMR (400 MHz, DMSO-d₆) δ: 2.57 (s, 3H, COCH₃), 3.65 (m, 8H, 2-CH₂NCH₂—), 3.76 (m, 8H, 2-CH₂OCH₂—), 7.46 and 7.79 (d-d, 4H, C₆H₄), 9.91 (s, 1H, NH).

(3-Chloro-2-methylphenyl)-(4,6-dimorpholin-4-yl-[1,3,5]triazin-2-yl)-amine hydrochloride (Compound 379) (VIRADEM-014295): Yield 78%. M.p.=213-214° C. C₁₈H₂₄Cl₂N₆O₂. MS m/z: 391.5 [(M+H)+]. ¹H NMR (400 MHz, DMSO-d₆) δ: 2.09 (s, 3H, CH₃), 3.66 (m, 8H, 2-CH₂NCH₂—), 3.77 (m, 8H, 2-CH₂OCH₂—), 7.08-7.56 (m, 3H, C₆H₃), 9.89 (s, 1H, NH).

(2,3-Dimethylphenyl)-(4,6-dimorpholin-4-yl-[1,3,5]triazin-2-yl)-amine (Compound 380) (VIRADEM-014300): Yield 72%. M.p.=172-173° C. C₁₉H₂₇ClN₆O₂. MS m/z: 371.2 [(M+H)+]. ¹H NMR (400 MHz, DMSO-d₆) δ: 2.08 (s, 3H, CH₃), 2.17 (s, 3H, CH₃), 3.64 (m, 8H, 2-CH₂NCH₂—), 3.75 (m, 8H, 2-CH₂OCH₂—), 7.03-7.42 (m, 3H, C₆H₃), 9.91 (s, 1H, NH).

Benzo[1,3]dioxol-5-yl-(4,6-dimorpholin-4-yl-[1,3,5]triazin-2-yl)-amine hydrochloride (Compound 381) (VIRADEM-014301): Yield 82%. M.p.=211-212° C. C₁₈H₂₃ClN₆O₄. MS m/z: 387.2 [(M+H)+]. ¹H NMR (400 MHz, DMSO-d₆) δ: 3.66 (m, 8H, 2-CH₂NCH₂—), 3.77 (m, 8H, 2-CH₂OCH₂—), 5.95 (s, 2H, OCH₂O), 6.81-7.44 (m, 3H, C₆H₃), 9.39 (s, 1H, NH).

(4-Methoxyphenyl)-acetic acid N₁-(4,6-dimorpholin-4-yl-[1,3,5]triazin-2-yl)-hydrazide (Compound 382) (VIRADEM-014302): Yield 75%. M.p.=207-208° C. C₂₀H₂₈ClN₇O₄. MS m/z: 430.2 [(M+H)+].

Cyclopentyl-(4,6-dimorpholin-4-yl-[1,3,5]triazin-2-yl)-amine (Compound 383) (VIRADEM-014309): Yield 63%. M.p.>260° C. C₁₆H₂₆N₆O₂. MS m/z: 335.2 [(M+H)+].

(2,4-Dimethylphenyl)-(4,6-dimorpholin-4-yl-[1,3,5]triazin-2-yl)-amine hydrochloride (Compound 384) (VIRADEM-014892): Yield 76%. M.p.=176-177° C. C₁₉H₂₇ClN₆O₂. MS m/z: 371.2 [(M+H)+]. ¹H NMR (400 MHz, DMSO-d₆) δ: 2.08 (s, 3H, CH₃), 2.21 (s, 3H, CH₃), 3.64 (m, 8H, 2-CH₂NCH₂—), 3.75 (m, 8H, 2-CH₂OCH₂—), 7.08-7.47 (m, 3H, C₆H₃), 9.79 (s, 1H, NH).

(2,4-Dimethoxylphenyl)-(4,6-dimorpholin-4-yl-[1,3,5]triazin-2-yl)-amine hydrochloride (Compound 385) (VIRADEM-014894): Yield 79%. M.p.=181-182° C. C₁₉H₂₇ClN₆O₄. MS m/z: 403.2 [(M+H)+].

(2-Chlorophenyl)-(4,6-dimorpholin-4-yl-[1,3,5]triazin-2-yl)-amine hydrochloride (Compound 386) (VIRADEM-014895): Yield 77%. M.p.=172-173° C. C₁₇H₂₂Cl₂N₆O₂. MS m/z: 377.6 [(M+H)+]. ¹H NMR (400 MHz, DMSO-d₆) δ: 3.61 (m, 8H, 2-CH₂NCH₂—), 3.69 (m, 8H, 2-CH₂OCH₂—), 7.10 (t, 1H, C₆H₄), 7.31 (t, 1H, C₆H₄), 7.46 (d, 1H, C₆H₄), 7.88 (d, 1H, C₆H₄), 8.13 (s, 1H, NH).

Benzyl-(4,6-dimorpholin-4-yl-[1,3,5]triazin-2-yl)-amine hydrochloride (Compound 387) (VIRADEM-014896): Yield 70%. M.p.=165-166° C. C₁₈H₂₅ClN₆O₂. MS m/z: 357.2 [(M+H)+]. ¹H NMR (400 MHz, DMSO-d₆) δ: 3.61 (m, 8H, 2-CH₂NCH₂—), 3.69 (m, 8H, 2-CH₂OCH₂—), 4.52 (m, 2H, NHCH₂Ph), 6.53 (br. s, 1H, NHCH₂Ph), 7.19-7.30 (m, 5H, C₆H₅), 8.43 (s, 1H, NH).

(4,6-Dimorpholin-4-yl-[1,3,5]triazin-2-yl)-(2-methoxy-5-methylphenyl)-amine hydrochloride (Compound 388) (VIRADEM-014897): Yield 73%. M.p.=187-188° C. C₁₉H₂₇ClN₆O₃. MS m/z: 387.2 [(M+H)+]. ¹H NMR (400 MHz, DMSO-d₆) δ: 2.16 (s, 3H, CH₃), 3.64 (m, 4H, —CH₂NCH₂—), 3.73 (m, 4H, —CH₂OCH₂—), 3.76 (s, 3H, OCH₃), 6.74-7.53 (m, 3H, C₆H₃), 10.0 (s, 1H, NH).

Allyl-(4,6-dimorpholin-4-yl-[1,3,5]triazin-2-yl)-amine hydrochloride (Compound 389) (VIRADEM-014899): Yield 61%. M.p.=170-171° C. C₁₄H₂₃ClN₆O₂. MS m/z: 307.1 [(M+H)+].

(4-Chloro-3-trifluoromethylphenyl)-(4,6-dimorpholin-4-yl-[1,3,5]triazin-2-yl)-amine hydrochloride (Compound 390) (VIRADEM-014901): Yield 80%. M.p.=212-213° C. C₁₈H₂₀ClF₃N₆O₂. MS m/z: 445.6 [(M+H)+].

(2,6-Dimethylphenyl)-(4,6-dimorpholin-4-yl-[1,3,5]triazin-2-yl)-amine hydrochloride (Compound 391) (VIRADEM-014905): Yield 71%. M.p.=173-174° C. C₁₉H₂₇ClN₆O₂. MS m/z: 371.2 [(M+H)+]. ¹H NMR (400 MHz, CDCl₃) δ: 2.24 (s, 6H, 2CH₃), 3.68 (m, 17H, 2-CH₂NCH₂—+2-CH₂OCH₂-+NH), 6.03-7.06 (m, 3H, C₆H₃).

Tert-Butyl-(4,6-dimorpholin-4-yl-[1,3,5]triazin-2-yl)-amine hydrochloride (Compound 392) (VIRADEM-014906): Yield 53%. M.p.=182-183° C. C₁₅H₂₇ClN₆O₂. MS m/z: 323.2 [(M+H)+].

(4,6-Dimorpholin-4-yl-[1,3,5]triazin-2-yl)-methylphenyl-amine hydrochloride (Compound 393) (VIRADEM-014908): Yield 69%. M.p.=181-182° C. C₁₈H₂₅ClN₆O₂. MS m/z: 357.2 [(M+H)+].

4-(4,6-Dimorpholin-4-yl-[1,3,5]triazin-2-yl)-piperazine-1-carboxylic acid (4-chlorophenyl)-amide (Compound 394) (VIRADEM-015104): Yield 78%. M.p.>260° C. C₂₂H₂₉ClN₈O₃. MS m/z: 489.7 [(M+H)+].

2-(4-Chlorophenoxy)-4,6-dimorpholin-4-yl-[1,3,5]triazine (Compound 395) (VIRADEM-015106): Yield 77%. M.p.=218-219° C. C₁₇H₂₀ClN₅O₃. MS m/z: 478.6 [(M+H)+]. ¹H NMR (300 MHz, DMSO-d₆) δ: 3.57 (m, 8H, 2-CH₂NCH₂—), 3.67 (m, 8H, 2-CH₂OCH₂—), 7.20 and 7.45 (d-d, 4H, C₆H₄).

(4,6-Dimorpholin-4-yl-[1,3,5]triazin-2-yl)-pyridin-3-yl-amine (Compound 396) (VIRADEM-015163): Yield 69%. M.p.>260° C. C₁₆H₂₁N₇O₂. MS m/z: 344.1 [(M+H)+].

(4,6-Dimorpholin-4-yl-[1,3,5]triazin-2-yl)-(4-phenylthiazol-2-yl)-amine (Compound 397) (VIRADEM-015186): Yield 63%. M.p.=186-187° C. C₂₀H₂₃N₇O₂S. MS m/z: 426.3 [(M+H)+].

2-(4-Allyl-5-thiophen-2-yl-4H-[1,2,4]triazol-3-ylsulfanyl)-(4,6-dimorpholin-4-yl-[1,3,5]triazin (Compound 398) (VIRADEM-015187): Yield 56%. C₂₀H₂₄N₈O₂S₂. MS m/z: 473.3 [(M+H)+].

2-(4-Benzylpiperazin-1-yl)-4,6-dimorpholin-4-yl-[1,3,5]triazine (Compound 399) (VIRADEM-015199): Yield 81%. M.p.=156-157° C. C₂₂H₃₁N₇O₂. MS m/z: 426.3 [(M+H)+].

(4,6-Dimorpholin-4-yl-[1,3,5]triazin-2-yl)-(1,1-dioxotetrahydro-1lambda*6*thiophen-3-yl)-amine (Compound 400) (VIRADEM-015200): Yield 73%. M.p.=185-185° C. C₁₅H₂₄N₆O₄S. MS m/z: 385.2 [(M+H)+].

Benzo[1,3]dioxol-5-ylmethyl-(4,6-dimorpholin-4-yl-[1,3,5]triazin-2-yl)-amine (Compound 401) (VIRADEM-015201): Yield 78%. M.p.=164-165° C. C₁₉H₂₄NO₄. MS m/z: 401.2 [(M+H)+].

Benzyl-(4,6-dimorpholin-4-yl-[1,3,5]triazin-2-yl)-amine (Compound 402) (VIRADEM-015209): Yield 65%. M.p.=166-167° C. C₁₈H₂₄NO₂. MS m/z: 357.2 [(M+H)+].

(4,6-Dimorpholin-4-yl-[1,3,5]triazin-2-yl)-quinolin-6-yl-amine (Compound 403) (VIRADEM-015219): Yield 75%. M.p.=182-183° C. C₂₀H₂₃N₇O₂. MS m/z: 394.2 [(M+H)+].

(4,6-Dimorpholin-4-yl-[1,3,5]triazin-2-yl)-phenetylamine (Compound 404) (VIRADEM-015230): Yield 77%. M.p.=113-114° C. C₁₉H₂₆NO₂. MS m/z: 371.2 [(M+H)+].

(4,6-Dimorpholin-4-yl-[1,3,5]triazin-2-yl)-furan-2-ylmethylamine (Compound 405) (VIRADEM-015234): Yield 64%. M.p.=148-149° C. C₁H₂₂NO₃. MS m/z: 347.2 [(M+H)+].

2-(3,5-Dimethylpiperidin-1-yl)-4,6-dimorpholin-4-yl-[1,3,5]triazine (Compound 406) (VIRADEM-015239): Yield 69%. M.p.=247-248° C. C₁₈H₃₀N₆O₂. MS m/z: 363.2 [(M+H)+].

[2-(3,4-Dimethoxyphenyl)-ethyl]-(4,6-dimorpholin-4-yl-[1,3,5]triazin-2-yl)-amine (Compound 407) (VIRADEM-015243): Yield 72%. M.p.=124-125° C. C₂₁H₃₀N₆O₄. MS m/z: 431.3 [(M+H)+].

(4,6-Dimorpholin-4-yl-[1,3,5]triazin-2-yl)-(3-ethylphenyl)-amine (Compound 408) (VIRADEM-015248): Yield 70%. M.p.=108-109° C. C₁₉H₂NO₂. MS m/z: 371.2 [(M+H)+].

4,6-Dimorpholin-4-yl-[1,3,5]triazine-2-thiol (Compound 409) (VIRADEM-015293): Yield 75%. C₁₁H₁₇N₅O₂S. MS m/z: 284.2 [(M+H)+].

N-(4,6-Dimorpholin-4-yl-[1,3,5]triazin-2-yl)-N¹-tozylhydrazin (Compound 410) (VIRADEM-015295): Yield 79%. M.p.=232-233° C. C₁₈H₂₅N₇O₄S. MS m/z: 437.3 [(M+H)+].

(4,6-Dimorpholin-4-yl-[1,3,5]triazin-2-yl)-(2,2,6,6-tetramethylpiperidin-4-yl)-amine (Compound 411) (VIRADEM-015296): Yield 66%. M.p.=190-191° C. C₂₀H₃₅N₇O₂. MS m/z: 406.3 [(M+H)+].

1-(4,6-Dimorpholin-4-yl-[1,3,5]triazin-2-yl)-2,3-dihydro-1H-indole (Compound 412) (VIRADEM-015355): Yield 69%. M.p.=219-220° C. C₁₉H₂₄NO₂. MS m/z: 369.2 [(M+H)+].

1-(4,6-Dimorpholin-4-yl-[1,3,5]triazin-2-yl)-piperidine-4-carboxylic acid methyl ester (Compound 413) (VIRADEM-015358): Yield 74%. M.p.=164-165° C. C₁₈H₂₈N₆O₄. MS m/z: 393.2 [(M+H)+].

1-(4,6-Dimorpholin-4-yl-[1,3,5]triazin-2-yl)-piperidine-4-carboxylic acid amide (Compound 414) (VIRADEM-015361): Yield 80%. M.p.>260° C. C₁₇H₂₇N₇O₃. MS m/z: 378.2 [(M+H)+].

2,4-Dimorpholin-4-yl-6-piperazin-1-yl-[1,3,5]triazine (Compound 415) (VIRADEM-015464): Yield 68%. M.p.>260° C. C₁₅H₂₅N₇O₂. MS m/z: 336.2 [(M+H)+].

2,4-Dimorpholin-4-yl-6-(4-phenylpiperazin-1-yl)-[1,3,5]triazine (Compound 416) (VIRADEM-016441): Yield 79%. M.p.=201-202° C. C₂₁H₂₉N₇O₂. MS m/z: 412.3 [(M+H)+].

2-(2,6-Dimethylmorpholin-4-yl)-4,6-dimorpholin-4-yl-[1,3,5]triazine (Compound 417) (VIRADEM-016469): Yield 67%. M.p.=237-238° C. C₁₇H₂₈N₆O₃. MS m/z: 365.3 [(M+H)+].

[4-(2,6-Dimethylmorpholin-4-yl)-6-morpholin-4-yl-[1,3,5]triazin-2-yl]-(4-methoxyphenyl)-amine (Compound 418) (VIRADEM-016512): Yield 73%. M.p.=159-160° C. C₂₀H₂₈N₆O₃. MS m/z: 401.3 [(M+H)+].

[4-(2,6-Dimethylmorpholin-4-yl)-6-morpholin-4-yl-[1,3,5]triazin-2-yl]-p-tolyl-amine (Compound 419) (VIRADEM-016513): Yield 75%. M.p.=183-184° C. C₂₀H₂₈N₆O₂. MS m/z: 384.2 [(M+H)+].

1-(4,6-Dimorpholin-4-yl-[1,3,5]triazin-2-yl)-azepane (Compound 420) (VIRADEM-016523): Yield 69%. M.p.=191-192° C. C₁₇H₂₈N₆O₂. MS m/z: 349.3 [(M+H)+].

3-(4,6-Dimorpholin-4-yl-[1,3,5]triazin-2-ylamino)-1-phenyl-1,5-dihydropyrazol-4-one (Compound 421) (VIRADEM-019970): Yield 51%. M.p.=161-162° C. C₂₀H₂₄N₈O₃. MS m/z: 425.2 [(M+H)+].

(4,6-Dimorpholin-4-yl-[1,3,5]triazin-2-yl)-(2-ethylphenyl)-amine hydrochloride (Compound 422) (VIRADEM-021051): Yield 62%. M.p.=137-138° C. C₁₉H₂₇ClN₆O₂. MS m/z: 371.3 [(M+H)+].

2-(4,6-Dimorpholin-4-yl-[1,3,5]triazin-2-ylamino)-benzoic acid ethyl ester hydrochloride (Compound 423) (VIRADEM-021055): Yield 59%. M.p.=173-174° C. C₂₀H₂₇ClN₆O₄. MS m/z: 451.7 [(M+H)+].

(2,4-Dichlorophenyl)-(4,6-dimorpholin-4-yl-[1,3,5]triazin-2-yl)-amine hydrochloride (Compound 424) (VIRADEM-021057): Yield 69%. M.p.=203-204° C. C₁₇H₂₁Cl₃N₆O₂. MS m/z: 412.1 [(M+H)+].

(4,6-Dimorpholin-4-yl-[1,3,5]triazin-2-yl)-(2-ethoxyphenyl)-amine hydrochloride (Compound 425) (VIRADEM-021058): Yield 65%. M.p.=174-175° C. C₁₉H₂₇ClN₆O₃. MS m/z: 387.2 [(M+H)+].

(2,5-Dimethoxyphenyl)-(4,6-dimorpholin-4-yl-[1,3,5]triazin-2-yl)-amine hydrochloride (Compound 426) (VIRADEM-021059): Yield 62%. M.p.=212-213° C. C₁₉H₂₇ClN₆O₄. MS m/z: 403.3 [(M+H)+].

(2-Chloro-5-trifluoromethylphenyl)-(4,6-dimorpholin-4-yl-[1,3,5]triazin-2-yl)-amine hydrochloride (Compound 427) (VIRADEM-021061): Yield 70%. M.p.=187-188° C. C₁₈H₂₁Cl₂F₃N₆O₂. MS m/z: 445.5 [(M+H)+].

(5-Chloro-2-methoxyphenyl)-(4,6-dimorpholin-4-yl-[1,3,5]triazin-2-yl)-amine hydrochloride (Compound 428) (VIRADEM-021064): Yield 73%. M.p.=236-237° C. C₁₈H₂₄Cl₂N₆O₃. MS m/z: 407.6 [(M+H)+].

(2,3-Dichlorophenyl)-(4,6-dimorpholin-4-yl-[1,3,5]triazin-2-yl)-amine hydrochloride (Compound 429) (VIRADEM-021065): Yield 71%. M.p.=193-194° C. C₁₇H₂₁Cl₃N₆O₂. MS m/z: 412.0 [(M+H)+].

(4,6-Dimorpholin-4-yl-[1,3,5]triazin-2-yl)-(2,4,6-trimethylphenyl)-amine hydrochloride (Compound 430) (VIRADEM-021069): Yield 57%. M.p.=144-145° C. C₂₀H₂₉ClN₆O₂. MS m/z: 385.2 [(M+H)+].

(4,6-Dimorpholin-4-yl-[1,3,5]triazin-2-yl)-naphthalen-1-yl-amine hydrochloride (Compound 431) (VIRADEM-021070): Yield 77%. M.p.=157-158° C. C₂₁H₂₅ClN₆O₂. MS m/z: 393.2 [(M+H)+].

(4,6-Dipyrrolidin-4-yl-[1,3,5]triazin-2-yl)-p-tolyl-amine hydrochloride (Compound 432) (VIRADEM-011139): Yield 79%. M.p.=223-224° C. C₁₈H₂₅ClN₆. MS m/z: 325.2 [(M+H)+]. ¹H NMR (400 MHz, DMSO-d₆) δ: 1.94 (m, 8H, 2-CH₂CH₂—), 3.28 (s, 3H, CH₃), 3.52 (m, 8H, 2-CH₂NCH₂—), 7.19 and 7.55 (d-d, 4H, C₆H₄), 10.0 (s, 1H, NH).

6-Pyrrolidin-1-yl-N,N¹-di-p-tolyl-[1,3,5]triazine-2,4-diamine hydrochloride (Compound 433) (VIRADEM-011244): Yield 77%. M.p.=237-238° C. C₂₁H₂₅ClN₆. MS m/z: 361.2 [(M+H)+]. ¹H NMR (400 MHz, DMSO-d₆) δ: 1.96 (m, 4H, 2-CH₂CH₂—), 3.29 (s, 6H, 2CH₃), 3.59 (m, 4H, 2-CH₂NCH₂—), 7.16 and 7.56 (d-d, 8H, 2C₆H₄), 10.1 (s, 1H, NH).

N,N¹-Bis-(4-fluorophenyl)-6-pyrrolidin-1-yl-[1,3,5]triazine-2,4-diamine (Compound 434) (VIRADEM-011502): Yield 75%. M.p.=231-232° C. C₁₉H₁₈F₂N₆. MS m/z: 369.0 [(M+H)+].

(4,6-Dipyrrolidin-4-yl-[1,3,5]triazin-2-yl)-(4-phenoxyphenyl)-amine hydrochloride (Compound 435) (VIRADEM-011503): Yield 87%. M.p.=214-215° C. C₂₃H₂₇ClN₆O. MS m/z: 403.2 [(M+H)+].

4-(4,6-Dipyrrolidin-4-yl-[1,3,5]triazin-2-ylamino)-benzoic acid ethyl ester hydrochloride (Compound 436) (VIRADEM-011504): Yield 69%. M.p.=241-242° C. C₂₀H₂₇ClN₆O₂. MS m/z: 383.2 [(M+H)+].

3,5-Dichlorophenyl)-(4,6-dipyrrolidin-1-yl-[1,3,5]triazin-2-yl)-amine hydrochloride (Compound 437) (VIRADEM-011505): Yield 79%. M.p.=196-197° C. C₁₇H₂₁Cl₃N₆O. MS m/z: 380.0 [(M+H)+].

(3,4-Dichlorophenyl)-(4,6-dipyrrolidin-1-yl-[1,3,5]triazin-2-yl)-amine hydrochloride (Compound 438) (VIRADEM-011506): Yield 81%. M.p.=228-229° C. C₁₇H₂₁Cl₃N₆O. MS m/z: 380.0 [(M+H)+].

(3,4-Dimethoxyphenyl)-(4,6-dipyrrolidin-1-yl-[1,3,5]triazin-2-yl)-amine hydrochloride (Compound 439) (VIRADEM-011507): Yield 72%. M.p.=242-243° C. C₁₉H₂₇ClN₆O₂. MS m/z: 371.2 [(M+H)+].

N,N¹-Diphenyl-6-pyrrolidin-1-yl-[1,3,5]triazine-2,4-diamine hydrochloride (Compound 440) (VIRADEM-011508): Yield 78%. M.p.=223-224° C. C₁₉H₂₁ClN₆. MS m/z: 333.2 [(M+H)+]. ¹H NMR (400 MHz, DMSO-d₆) δ: 1.91 (m, 4H, —CH₂CH₂—), 3.59 (m, 4H, —CH₂NCH₂—), 7.09-7.71 (m, 10H, 2C₆H₅), 10.5 (s, 2H, 2NH).

4-(4,6-Dipyrrolidin-4-yl-[1,3,5]triazin-2-ylamino)-benzoic acid methyl ester hydrochloride (Compound 441) (VIRADEM-011509): Yield 73%. M.p.=243-244° C. C₁₉H₂₅ClN₆O₂. MS m/z: 369.2 [(M+H)+].

N-[4-(4,6-Dipyrrolidin-1-yl-[1,3,5]triazin-2-ylamino)-phenyl]-acetamide hydrochloride (Compound 442) (VIRADEM-011510): Yield 81%. M.p.=257-258° C. C₁₉H₂₆ClN₇O. MS m/z: 368.2 [(M+H)+].

(4-Butylphenyl)-(4,6-dipyrrolidin-1-yl-[1,3,5]triazin-2-yl)-amine hydrochloride (Compound 443) (VIRADEM-011511): Yield 78%. M.p.=213-214° C. C₂₁H₃₁ClN₆. MS m/z: 367.3 [(M+H)+].

(3-Chloro-4-fluorophenyl)-(4,6-dipyrrolidin-1-yl-[1,3,5]triazin-2-yl)-amine hydrochloride (Compound 444) (VIRADEM-011512): Yield 75%. M.p.=231-232° C. C₁₇H₂₁Cl₂FN₆. MS m/z: 363.5 [(M+H)+].

(3-Chloro-4-methylphenyl)-(4,6-dipyrrolidin-1-yl-[1,3,5]triazin-2-yl)-amine hydrochloride (Compound 445) (VIRADEM-011513): Yield 73%. M.p.=223-224° C. C₁₈H₂₄Cl₂N₆. MS m/z: 359.6 [(M+H)+].

(4,6-Dipyrrolidin-1-yl-[1,3,5]triazin-2-yl)-(3-trifluoromethylphenyl)-amine hydrochloride (Compound 446) (VIRADEM-011514): Yield 73%. M.p.=218-219° C. C₁₈H₂₂ClF₃N₆. MS m/z: 379.2 [(M+H)+].

(4,6-Dipyrrolidin-1-yl-[1,3,5]triazin-2-yl)-(4-ethoxyphenyl)-amine hydrochloride (Compound 447) (VIRADEM-011515): Yield 70%. M.p.=211-212° C. C₁₉H₂₇ClN₆O. MS m/z: 355.2 [(M+H)+].

1-[3-(4,6-Dipyrrolidin-1-yl-[1,3,5]triazin-2-ylamino)-phenyl]-ethanone hydrochloride (Compound 448) (VIRADEM-011516): Yield 77%. M.p.=215-216° C. C₁₉H₂₅ClN₆O. MS m/z: 353.2 [(M+H)+].

(4-Chlorophenyl)-(4,6-dipyrrolidin-1-yl-[1,3,5]triazin-2-yl)-amine hydrochloride (Compound 449) (VIRADEM-011517): Yield 82%. M.p.=240-241° C. C₁₇H₂₂Cl₂N₆. MS m/z: 345.6 [(M+H)+].

(4,6-Dipyrrolidin-1-yl-[1,3,5]triazin-2-yl)-(4-methoxyphenyl)-amine hydrochloride (Compound 450) (VIRADEM-011518): Yield 74%. M.p.=241-242° C. C₁₈H₂₅ClN₆O. MS m/z: 341.2 [(M+H)+].

(4,6-Dipyrrolidin-1-yl-[1,3,5]triazin-2-yl)-(4-ethylphenyl)-amine hydrochloride (Compound 451) (VIRADEM-011519): Yield 71%. M.p.=230-231° C. C₁₉H₂₇ClN₆. MS m/z: 339.2 [(M+H)+].

(3,4-Dimethylphenyl)-(4,6-dipyrrolidin-1-yl-[1,3,5]triazin-2-yl)-amine hydrochloride (Compound 452) (VIRADEM-011520): Yield 77%. M.p.=231-232° C. C₁₉H₂₇ClN₆. MS m/z: 339.3 [(M+H)+]. ¹H NMR (400 MHz, DMSO-d₆) δ: 1.95 (m, 8H, 2-CH₂CH₂—), 2.27 (s, 6H, 2CH₃), 3.57 (m, 8H, 2-CH₂NCH₂—), 7.08-7.45 (m, 3H, C₆H₃), 10.6 (s, 1H, 1NH).

(3,5-Dimethylphenyl)-(4,6-dipyrrolidin-1-yl-[1,3,5]triazin-2-yl)-amine hydrochloride (Compound 453) (VIRADEM-011521): Yield 77%. M.p.=234-235° C. C₁₉H₂₇ClN₆. MS m/z: 339.3 [(M+H)+]. ¹H NMR (400 MHz, DMSO-d₆) δ: 1.96 (m, 8H, 2-CH₂CH₂—), 2.22 (s, 6H, 2CH₃), 3.58 (m, 8H, 2-CH₂NCH₂—), 6.68 (s, 1H, C₆H₃), 7.27 (s, 2H, C₆H₃), 10.5 (s, 1H, 1NH).

Benzo[1,3]dioxol-5-yl-(4,6-dipyrrolidin-1-yl-[1,3,5]triazin-2-yl)-amine hydrochloride (Compound 454) (VIRADEM-011522): Yield 80%. M.p.=246-247° C. C₁₈H₂₃ClN₆O₂. MS m/z: 355.2 [(M+H)+]. ¹H NMR (400 MHz, DMSO-d₆) δ: 1.94 (m, 8H, 2-CH₂CH₂—), 3.55 (m, 8H, 2-CH₂NCH₂—), 6.01 (s, 2H, OCH₂O), 6.90-7.48 (m, 3H, C₆H₃), 10.3 (s, 1H, 1NH).

(4,6-Dipyrrolidin-1-yl-[1,3,5]triazin-2-yl)-(4-fluorophenyl)-amine hydrochloride (Compound 455) (VIRADEM-011523): Yield 74%. M.p.=231-232° C. C₁₇H₂₂ClFN₆. MS m/z: 329.1 [(M+H)+].

(3-Chlorophenyl)-(4,6-dipyrrolidin-1-yl-[1,3,5]triazin-2-yl)-amine hydrochloride (Compound 456) (VIRADEM-011524): Yield 79%. M.p.=222-223° C. C₁₇H₂₂Cl₂N₆. MS m/z: 345.6 [(M+H)+].

(4,6-Dipyrrolidin-1-yl-[1,3,5]triazin-2-yl)-m-tolyl-amine hydrochloride (Compound 457) (VIRADEM-011525): Yield 69%. M.p.=223-224° C. C₁₈H₂₅ClN₆. MS m/z: 325.2 [(M+H)+].

(4,6-Dipyrrolidin-1-yl-[1,3,5]triazin-2-yl)-(3-fluorophenyl)-amine hydrochloride (Compound 458) (VIRADEM-011526): Yield 71%. M.p.=219-220° C. C₁₇H₂₂ClFN₆. MS m/z: 329.1 [(M+H)+].

(4,6-Dipyrrolidin-1-yl-[1,3,5]triazin-2-yl)-(2-fluorophenyl)-amine hydrochloride (Compound 459) (VIRADEM-011527): Yield 67%. M.p.=215-216° C. C₁₇H₂₂ClFN₆. MS m/z: 329.1 [(M+H)+].

(4,6-Dipyrrolidin-1-yl-[1,3,5]triazin-2-yl)-phenylamine hydrochloride (Compound 460) (VIRADEM-011528): Yield 77%. M.p.=217-218° C. C₁₇H₂₃ClN₆. MS m/z: 311.2 [(M+H)+]. ¹H NMR (400 MHz, DMSO-d₆) δ: 1.93 (m, 8H, 2-CH₂CH₂—), 3.58 (m, 8H, 2-CH₂NCH₂—), 7.08-7.69 (m, 5H, C₆H₅), 10.3 (s, 1H, NH).

3-Chloro-4-methoxyphenyl)-(4,6-dipyrrolidin-1-yl-[1,3,5]triazin-2-yl)-amine hydrochloride (Compound 461) (VIRADEM-011529): Yield 70%. M.p.=228-229° C. C₁₈H₂₄Cl₂N₆O. MS m/z: 375.6 [(M+H)+].

N,N¹-Bis-(3-chloro-4-fluorophenyl)-6-pyrrolidin-1-yl-[1,3,5]triazine-2,4-diamine (Compound 462) (VIRADEM-011572): Yield 75%. M.p.=174-175° C. C₁₉H₁₇Cl₂F₂N₆. MS m/z: 438.0 [(M+H)+]. 1H NMR (400 MHz, DMSO-d₆) δ: 1.94 (m, 4H, —CH₂CH₂—), 3.53 (m, 4H, —CH₂NCH₂—), 7.29-8.13 (m, 3H, C₆H₃), 9.36 (s, 2H, 2NH).

N,N¹-Bis-benzo[1,3]dioxol-5-yl-6-pyrrolidin-1-yl-[1,3,5]triazine-2,4-diamine (Compound 463) (VIRADEM-011573): Yield 78%. M.p.=226-227° C. C₂₁H₂₀N₆O₄. MS m/z: 421.2 [(M+H)+]. ¹H NMR (400 MHz, DMSO-d₆) δ: 1.91 (m, 4H, —CH₂CH₂—), 3.51 (m, 4H, —CH₂NCH₂—), 5.96 (s, 4H, 2OCH₂O), 6.79-7.60 (m, 6H, 2C₆H₃), 8.96 (s, 2H, 2NH).

N,N¹-Bis-(3-chlorophenyl)-6-pyrrolidin-1-yl-[1,3,5]triazine-2,4-diamine (Compound 464) (VIRADEM-011585): Yield 77%. C₁₉H₁₈Cl₂N₆. MS m/z: 402.1 [(M+H)+]. ¹H NMR (400 MHz, DMSO-d₆) δ: 1.94 (m, 4H, —CH₂CH₂—), 3.55 (m, 4H, —CH₂NCH₂—), 6.98-8.02 (m, 8H, 2C₆H₄), 9.36 (s, 2H, 2NH).

N,N¹-Bis-(3-fluorophenyl)-6-pyrrolidin-1-yl-[1,3,5]triazine-2,4-diamine (Compound 465) (VIRADEM-011586): Yield 75%. M.p.=178-179° C. C₁₉H₁₈F₂N₆. MS m/z: 369.1 [(M+H)+]. ¹H NMR (400 MHz, DMSO-d₆) δ: 1.94 (m, 4H, —CH₂CH₂—), 3.55 (m, 4H, —CH₂NCH₂—), 6.73-7.96 (m, 8H, 2C₆H₄), 9.40 (s, 2H, 2NH).

N,N¹-Bis-(2-fluorophenyl)-6-pyrrolidin-1-yl-[1,3,5]triazine-2,4-diamine (Compound 466) (VIRADEM-011587): Yield 69%. M.p.=154-146° C. C₁₉H₁₈F₂N₆. MS m/z: 369.1 [(M+H)+]. ¹H NMR (400 MHz, DMSO-d₆) δ: 1.86 (m, 4H, —CH₂CH₂—), 3.44 (m, 4H, —CH₂NCH₂—), 7.03-8.11 (m, 8H, 2C₆H₄).

N,N¹-Bis-(2,3-dihydrobenzo[1,4]dioxin-6-yl)-6-pyrrolidin-1-yl-[1,3,5]triazine-2,4-diamine (Compound 467) (VIRADEM-011588): Yield 79%. M.p.=246-247° C. C₂₃H₂₄N₆O₄. MS m/z: 449.2 [(M+H)+]. ¹H NMR (400 MHz, DMSO-d₆) δ: 1.91 (m, 4H, —CH₂CH₂—), 3.50 (m, 4H, —CH₂NCH₂—), 4.20 (m, 8H, 2-OCH₂CH₂O—), 6.71-7.47 (m, 6H, 2C₆H₃), 8.84 (s, 2H, 2NH).

N,N¹-Bis-(2-methoxyphenyl)-6-pyrrolidin-1-yl-[1,3,5]triazine-2,4-diamine (Compound 468) (VIRADEM-011589): Yield 67%. M.p.=209-210° C. C₂₁H₂₄NO₂. MS m/z: 393.2 [(M+H)+]. ¹H NMR (400 MHz, DMSO-d₆) δ: 1.91 (m, 4H, —CH₂CH₂—), 3.52 (m, 4H, —CH₂NCH₂—), 3.86 (s, 6H, 2OCH₃), 6.90-7.63 (m, 8H, 2C₆H₄), 8.25 (s, 2H, 2NH).

N,N¹-Bis-(4-methoxyphenyl)-6-pyrrolidin-1-yl-[1,3,5]triazine-2,4-diamine (Compound 469) (VIRADEM-011590): Yield 77%. M.p.=231-232° C. C₂₁H₂₄NO₂. MS m/z: 393.2 [(M+H)+]. ¹H NMR (400 MHz, DMSO-d₆) δ: 1.91 (m, 4H, —CH₂CH₂—), 3.51 (m, 4H, —CH₂NCH₂—), 3.72 (s, 6H, 2OCH₃), 6.84 and 7.69 (d-d, 8H, 2C₆H₄), 8.85 (s, 2H, 2NH).

N,N¹-Bis-(4-chlorophenyl)-6-pyrrolidin-1-yl-[1,3,5]triazine-2,4-diamine (Compound 470) (VIRADEM-011591): Yield 85%. M.p.=238-239° C. C₁₉H₁₈Cl₂N₆. MS m/z: 402.1 [(M+H)+]. ¹H NMR (400 MHz, DMSO-d₆) δ: 1.92 (m, 4H, —CH₂CH₂—), 3.53 (m, 4H, —CH₂NCH₂—), 7.31 and 7.85 (d-d, 8H, 2C₆H₄), 9.28 (s, 2H, 2NH).

N,N¹-Bis-(4-ethylphenyl)-6-pyrrolidin-1-yl-[1,3,5]triazine-2,4-diamine (Compound 471) (VIRADEM-011592): Yield 73%. M.p.=197-198° C. C₂₃H₂₈N₆. MS m/z: 389.3 [(M+H)+]. ¹H NMR (400 MHz, DMSO-d₆) δ: 1.17 (t, 6H, 2CH₂CH₃), 1.92 (m, 4H, —CH₂CH₂—), 2.55 (q, 4H, 2CH₂CH₃), 3.53 (m, 4H, —CH₂NCH₂—), 7.09 and 7.70 (d-d, 8H, 2C₆H₄), 8.94 (s, 2H, 2NH).

N,N¹-Bis-(3,4-dimethylphenyl)-6-pyrrolidin-1-yl-[1,3,5]triazine-2,4-diamine (Compound 472) (VIRADEM-011593): Yield 75%. M.p.=165-166° C. C₂₃H₂₈N₆. MS m/z: 389.2 [(M+H)+]. ¹H NMR (400 MHz, DMSO-d₆) δ: 1.92 (m, 4H, —CH₂CH₂—), 2.16 (c, 6H, 2CH₃), 2.18 (c, 6H, 2CH₃), 3.52 (m, 4H, —CH₂NCH₂—), 6.99 and 7.59 (m, 6H, 2C₆H₃), 8.84 (s, 2H, 2NH).

N,N¹-Bis-(3,5-dimethylphenyl)-6-pyrrolidin-1-yl-[1,3,5]triazine-2,4-diamine (Compound 473) (VIRADEM-011594): Yield 77%. M.p.=185-186° C. C₂₃H₂₈N₆. MS m/z: 389.2 [(M+H)+]. ¹H NMR (400 MHz, DMSO-d₆) δ: 1.93 (m, 4H, —CH₂CH₂—), 2.22 (c, 12H, 4CH₃), 3.54 (m, 4H, —CH₂NCH₂—), 6.59 (s, 2H, 2C₆H₃), 7.44 (s, 4H, 2C₆H₃), 8.86 (s, 2H, 2NH).

6-Pyrrolidin-1-yl-N,N¹-di-m-tolyl-[1,3,5]triazine-2,4-diamine (Compound 474) (VIRADEM-011595): Yield 73%. M.p.=218-219° C. C₂₁H₂₅ClN₆. MS m/z: 361.2 [(M+H)+]. ¹H NMR (400 MHz, DMSO-d₆) δ: 1.92 (m, 4H, —CH₂CH₂—), 2.21 (c, 6H, 2CH₃), 3.53 (m, 4H, —CH₂NCH₂—), 6.79-7.58 (m, 8H, 2C₆H₄), 9.95 (s, 2H, 2NH).

-(2,3-dihydrobenzo[1,4]dioxin-6-yl)-(4,6-dipyrrolidin-1-yl-[1,3,5]triazin-2-yl)-amine hydrochloride (Compound 475) (VIRADEM-011596): Yield 77%. M.p.=260-261° C. C₁₉H₂₅ClN₆O₂. MS m/z: 369.2 [(M+H)+]. ¹H NMR (400 MHz, DMSO-d₆) δ: 1.92 (m, 8H, 2-CH₂CH₂—), 3.52 (m, 8H, 2-CH₂NCH₂—), 4.19 (m, 4H, —OCH₂CH₂O—), 6.69-7.45 (m, 3H, C₆H₃), 8.79 (s, 1H, 1NH).

(4,6-Dipyrrolidin-1-yl-[1,3,5]triazin-2-yl)-(2-methoxyphenyl)-amine hydrochloride (Compound 476) (VIRADEM-011597): Yield 65%. M.p.=212-213° C. C₁₈H₂₅ClN₆O. MS m/z: 341.2 [(M+H)+]. ¹H NMR (400 MHz, DMSO-d₆) δ: 1.93 (m, 8H, 2-CH₂CH₂—), 3.54 (m, 8H, 2-CH₂NCH₂—), 3.85 (s, 3H, OCH₃), 6.91-7.62 (m, 4H, C₆H₄), 8.36 (s, 1H, 1NH).

N-(3-Chloro-4-fluorophenyl)-6-pyrrolidin-1-yl-N¹-p-tolyl-[1,3,5]triazine-2,4-diamine hydrochloride (Compound 477) (VIRADEM-011790): Yield 71%. M.p.=231-232° C. C₂₀H₂₁Cl₂FN₆. MS m/z: 399.6 [(M+H)+]. ¹H NMR (400 MHz, DMSO-d₆) δ: 1.94 (m, 4H, —CH₂CH₂—), 2.28 (s, 3H, CH₃), 3.55 (m, 4H, —CH₂NCH₂—), 7.13-8.05 (m, 7H, C₄H₃+C₆H₄), 9.85 (s, 1H, 1NH), 9.94 (s, 1H, 1NH).

N-(3-Chloro-4-methoxyphenyl)-6-pyrrolidin-1-yl-N¹-p-tolyl-[1,3,5]triazine-2,4-diamine hydrochloride (Compound 478) (VIRADEM-011791): Yield 73%. M.p.=250-251° C. C₂₁H₂₄Cl₂N₆O. MS m/z: 411.6 [(M+H)+]. ¹H NMR (400 MHz, DMSO-d₆) δ: 1.93 (m, 4H, —CH₂CH₂—), 2.27 (s, 3H, CH₃), 3.54 (m, 4H, —CH₂NCH₂—), 3.73 (s, 3H, OCH₃), 6.96-8.02 (m, 7H, C₄H₃+C₆H₄), 9.79 (s, 1H, 1NH), 9.85 (s, 1H, 1NH).

N-(4-Methoxyphenyl)-6-pyrrolidin-1-yl-N¹-p-tolyl-[1,3,5]triazine-2,4-diamine hydrochloride (Compound 479) (VIRADEM-011792): Yield 77%. M.p.=243-244° C. C₂₁H₂₅ClN₆O. MS m/z: 377.2 [(M+H)+]. ¹H NMR (400 MHz, DMSO-d₆) δ: 1.92 (m, 4H, —CH₂CH₂—), 2.28 (s, 3H, CH₃), 3.55 (m, 4H, —CH₂NCH₂—), 3.74 (s, 3H, OCH₃), 6.99-7.52 (m, 8H, C₄H₄+C₆H₄), 9.89 (s, 1H, 1NH), 9.97 (s, 1H, 1NH).

6-Pyrrolidin-1-yl-N-m-tolyl-N¹-p-tolyl-[1,3,5]triazine-2,4-diamine hydrochloride (Compound 480) (VIRADEM-011800): Yield 73%. M.p.=241-242° C. C₂₁H₂₅ClN₆. MS m/z: 361.2 [(M+H)+]. ¹H NMR (400 MHz, DMSO-d₆) δ: 1.91 (m, 4H, —CH₂CH₂—), 2.18 (s, 3H, CH₃), 2.27 (s, 3H, CH₃), 3.54 (m, 4H, —CH₂NCH₂—), 7.09-7.67 (m, 8H, C₄H₄+C₆H₄), 9.91 (s, 1H, 1NH), 10.0 (s, 1H, 1NH).

N-(4-Fluorophenyl)-6-pyrrolidin-1-yl-N¹-p-tolyl-[1,3,5]triazine-2,4-diamine hydrochloride (Compound 481) (VIRADEM-011801): Yield 79%. C₂₀H₂₂ClFN₆. MS m/z: 365.2 [(M+H)+]. ¹H NMR (400 MHz, DMSO-d₆) δ: 1.91 (m, 4H, —CH₂CH₂—), 2.26 (s, 3H, CH₃), 3.54 (m, 4H, —CH₂NCH₂—), 7.07-7.69 (m, 8H, C₄H₄+C₆H₄), 9.91 (s, 1H, 1NH), 9.99 (s, 1H, 1NH).

N-Benzo[1,3]dioxol-5-yl-6-pyrrolidin-1-yl-N¹-p-tolyl-[1,3,5]triazine-2,4-diamine hydrochloride (Compound 482) (VIRADEM-011802): Yield 70%. C₂₁H₂₃ClN₆O₂. MS m/z: 391.2 [(M+H)+]. ¹H NMR (400 MHz, DMSO-d₆) δ: 1.97 (m, 4H, —CH₂CH₂—), 2.29 (s, 3H, CH₃), 3.51 (m, 4H, —CH₂NCH₂—), 6.03 (s, 2H, OCH₂O), 6.89-7.66 (m, 6H, 2C₆H₃), 10.2 (s, 2H, 2NH).

N-(3-Chloro-4-methylphenyl)-6-pyrrolidin-1-yl-N¹-p-tolyl-[1,3,5]triazine-2,4-diamine hydrochloride (Compound 483) (VIRADEM-011803): Yield 79%. C₂₁H₂₄Cl₂N₆. MS m/z: 395.6 [(M+H)+]. ¹H NMR (400 MHz, DMSO-d₆) δ: 1.94 (m, 4H, —CH₂CH₂—), 2.27 (s, 6H, 2CH₃), 3.54 (m, 4H, —CH₂NCH₂—), 7.13-7.93 (m, 7H, C₄H₃+C₆H₄), 10.1 (s, 2H, 2NH).

N-(4-Methoxyphenyl)-6-pyrrolidin-1-yl-N¹-m-tolyl-[1,3,5]triazine-2,4-diamine hydrochloride (Compound 484) (VIRADEM-011804): Yield 75%. C₂₁H₂₅ClN₆O. MS m/z: 377.2 [(M+H)+]. ¹H NMR (400 MHz, DMSO-d₆) δ: 1.93 (m, 4H, —CH₂CH₂—), 2.18 (s, 3H, CH₃), 3.54 (m, 4H, —CH₂NCH₂—), 3.76 (s, 3H, OCH₃), 7.02-7.62 (m, 8H, C₄H₄+C₆H₄), 9.89 (s, 1H, 1NH), 10.0 (s, 1H, 1NH).

N-(3,4-Dimethylphenyl)-N¹-(4-methoxyphenyl)-6-pyrrolidin-1-yl-[1,3,5]triazine-2,4-diamine hydrochloride (Compound 485) (VIRADEM-011805): Yield 71%. C₂₂H₂₇ClN₆O. MS m/z: 391.2 [(M+H)+]. ¹H NMR (400 MHz, DMSO-d₆) δ: 1.91 (m, 4H, —CH₂CH₂—), 2.16 (c, 6H, 2CH₃), 2.18 (c, 6H, 2CH₃), 3.54 (m, 4H, —CH₂NCH₂—), 3.76 (s, 3H, OCH₃), 7.00 and 7.67 (m, 7H, C₆H₄+C₆H₃), 10.2 (s, 2H, 2NH).

N-(4-Fluorophenyl)-N¹-(4-methoxyphenyl)-6-pyrrolidin-1-yl-[1,3,5]triazine-2,4-diamine hydrochloride (Compound 486) (VIRADEM-011806): Yield 79%. C₂₀H₂₂ClFN₆O. MS m/z: 381.2 [(M+H)+]. ¹H NMR (400 MHz, DMSO-d₆) δ: 1.92 (m, 4H, —CH₂CH₂—), 3.53 (m, 4H, —CH₂NCH₂—), 3.75 (s, 3H, OCH₃), 6.97 and 7.73 (m, 8H, C₆H₄+C₆H₄), 10.0 (s, 2H, 2NH).

N-(4-Chlorophenyl)-N¹-(4-fluorophenyl)-6-pyrrolidin-1-yl-[1,3,5]triazine-2,4-diamine hydrochloride (Compound 487) (VIRADEM-011807): Yield 83%. C₁₉H₁₉Cl₂FN₆. MS m/z: 385.6 [(M+H)+]. ¹H NMR (400 MHz, DMSO-d₆) δ: 1.92 (m, 4H, —CH₂CH₂—), 3.53 (m, 4H, —CH₂NCH₂—), 7.28-7.87 (m, 8H, 2C₆H₄), 10.1 (s, 2H, 2NH).

N-(4-Fluorophenyl)-6-pyrrolidin-1-yl-N¹-m-tolyl-[1,3,5]triazine-2,4-diamine hydrochloride (Compound 488) (VIRADEM-011808): Yield 76%. C₂₀H₂₂ClFN₆. MS m/z: 365.2 [(M+H)+]. ¹H NMR (400 MHz, DMSO-d₆) δ: 1.91 (m, 4H, —CH₂CH₂—), 2.18 (s, 3H, CH₃), 3.53 (m, 4H, —CH₂NCH₂—), 7.07-7.69 (m, 8H, C₆H₄+C₆H₄), 10.0 (s, 2H, 2NH).

N-(3,4-Dimethylphenyl)-N¹-(4-fluorophenyl)-6-pyrrolidin-1-yl-[1,3,5]triazine-2,4-diamine hydrochloride (Compound 489) (VIRADEM-011809): Yield 71%. C₂₁H₂₄ClFN₆. MS m/z: 379.2 [(M+H)+]. ¹H NMR (400 MHz, DMSO-d₆) δ: 1.92 (m, 4H, —CH₂CH₂—), 2.17 (c, 6H, 2CH₃), 3.55 (m, 4H, —CH₂NCH₂—), 7.08 and 7.58 (m, 7H, C₆H₄+C₆H₃), 10.0 (s, 2H, 2NH).

N-Benzo[1,3]dioxol-5-yl-N¹-(4-fluorophenyl)-6-pyrrolidin-1-yl-[1,3,5]triazine-2,4-diamine hydrochloride (Compound 490) (VIRADEM-011810): Yield 77%. C₂₀H₂₀ClFN₆O₂. MS m/z: 391.2 [(M+H)+]. ¹H NMR (400 MHz, DMSO-d₆) δ: 1.93 (m, 4H, —CH₂CH₂—), 3.55 (m, 4H, —CH₂NCH₂—), 6.05 (s, 2H, OCH₂O), 6.88-7.38 (m, 7H, C₆H₄+C₆H₃), 10.3 (s, 2H, 2NH).

N-(3-Chloro-4-methylphenyl)-N¹-(4-fluorophenyl)-6-pyrrolidin-1-yl-[1,3,5]triazine-2,4-diamine hydrochloride (Compound 491) (VIRADEM-011811): Yield 75%. C₂₀H₂₁Cl₂FN₆. MS m/z: 399.6 [(M+H)+]. ¹H NMR (400 MHz, DMSO-d₆) δ: 1.96 (m, 4H, —CH₂CH₂—), 2.29 (s, 3H, CH₃), 3.54 (m, 4H, —CH₂NCH₂—), 7.16-7.89 (m, 7H, C₄H₃+C₆H₄), 10.3 (s, 2H, 2NH).

N-(3,5-dimethylphenyl)-N¹-(4-fluorophenyl)-6-pyrrolidin-1-yl-[1,3,5]triazine-2,4-diamine hydrochloride (Compound 492) (VIRADEM-011812): Yield 80%. C₂₁H₂₄ClFN₆. MS m/z: 379.5 [(M+H)+]. ¹H NMR (400 MHz, DMSO-d₆) δ: 1.93 (m, 4H, —CH₂CH₂—), 2.18 (s, 6H, 2CH₃), 3.54 (m, 4H, —CH₂NCH₂—), 7.10-7.83 (m, 7H, C₄H₃+C₆H₄), 10.0 (s, 2H, 2NH).

N-(3-Chloro-4-fluorophenyl)-N¹-(4-fluorophenyl)-6-pyrrolidin-1-yl-[1,3,5]triazine-2,4-diamine hydrochloride (Compound 493) (VIRADEM-011813): Yield 81%. C₁₉H₁₈Cl₂F₂N₆. MS m/z: 403.6 [(M+H)+]. ¹H NMR (400 MHz, DMSO-d₆) δ: 1.94 (m, 4H, —CH₂CH₂—), 3.53 (m, 4H, —CH₂NCH₂—), 7.16-8.06 (m, 7H, C₄H₃+C₆H₄), 10.2 (s, 2H, 2NH).

N-(3,4-dimethoxyphenyl)-N¹-(4-fluorophenyl)-6-pyrrolidin-1-yl-[1,3,5]triazine-2,4-diamine hydrochloride (Compound 494) (VIRADEM-011988): Yield 78%. M.p.=224-225° C. C₂₁H₂₄ClFN₆O₂. MS m/z: 411.2 [(M+H)+]. ¹H NMR (400 MHz, DMSO-d₆) δ: 1.95 (m, 4H, —CH₂CH₂—), 3.53 (m, 4H, —CH₂NCH₂—), 3.76 (s, 6H, 2OCH₃), 6.92-7.73 (m, 7H, C₄H₃+C₆H₄), 10.3 (s, 2H, 2NH).

N-(3,5-dimethylphenyl)-N¹-(4-methoxyphenyl)-6-pyrrolidin-1-yl-[1,3,5]triazine-2,4-diamine hydrochloride (Compound 495) (VIRADEM-011989): Yield 77%. M.p.=226-227° C. C₂₂H₂₇ClN₆O. MS m/z: 391.2 [(M+H)+]. ¹H NMR (400 MHz, DMSO-d₆) δ: 1.91 (m, 4H, —CH₂CH₂—), 2.18 (s, 6H, 2CH₃), 3.53 (m, 4H, —CH₂NCH₂—), 3.75 (s, 3H, OCH₃), 6.97-7.63 (m, 7H, C₄H₃+C₆H₄), 10.2 (s, 2H, 2NH).

N-(4-Ethylphenyl)-N¹-(4-fluorophenyl)-6-pyrrolidin-1-yl-[1,3,5]triazine-2,4-diamine hydrochloride (Compound 496) (VIRADEM-012032): Yield 74%. M.p.=226-227° C. C₂₁H₂₄ClFN₆. MS m/z: 379.2 [(M+H)+]. ¹H NMR (400 MHz, DMSO-d₆) δ: 1.18 (t, 6H, 2CH₂CH₃), 1.92 (m, 4H, —CH₂CH₂—), 2.54 (q, 4H, 2CH₂CH₃), 3.53 (m, 4H, —CH₂NCH₂—), 7.08 and 7.69 (d-d, 4H, C₆H₄), 7.19-7.89 (m, 4H, C₆H₄), 10.0 (s, 2H, 2NH).

N-(4-Ethylphenyl)-N¹-(4-methoxyphenyl)-6-pyrrolidin-1-yl-[1,3,5]triazine-2,4-diamine hydrochloride (Compound 497) (VIRADEM-012036): Yield 70%. M.p.=228-9° C. C₂₂H₂₇ClN₆O. MS m/z: 391.2 [(M+H)+]. ¹H NMR (400 MHz, DMSO-d₆) δ: 1.17 (t, 3H, CH₂CH₃), 1.91 (m, 4H, —CH₂CH₂—), 2.55 (q, 2H, CH₂CH₃), 3.54 (m, 4H, —CH₂NCH₂—), 3.75 (s, 3H, OCH₃), 6.95 and 7.52 (d-d, 4H, C₆H₄), 7.08 and 7.69 (d-d, 4H, C₆H₄), 10.1 (s, 2H, 2NH).

N-(4-Chlorophenyl)-N¹-(3,4-dimethylphenyl)-6-pyrrolidin-1-yl-[1,3,5]triazine-2,4-diamine hydrochloride (Compound 498) (VIRADEM-012174): Yield 79%. M.p.=243-244° C. C₂₁H₂₄Cl₂N₆. MS m/z: 395.6 [(M+H)+]. ¹H NMR (400 MHz, DMSO-d₆) δ: 1.96 (m, 4H, —CH₂CH₂—), 2.21 (s, 3H, CH₃), 2.23 (s, 3H, CH₃), 3.53 (m, 4H, —CH₂NCH₂—), 7.05-7.81 (m, 7H, C₆H₃+C₆H₄), 9.81 (s, 1H, NH), 10.3 (s, 1H, 1NH).

N-(4-Chlorophenyl)-N¹-(4-ethoxyphenyl)-6-pyrrolidin-1-yl-[1,3,5]triazine-2,4-diamine hydrochloride (Compound 499) (VIRADEM-012175): Yield 79%. M.p.=235-236° C. C₂₁H₂₄Cl₂N₆O. MS m/z: 411.6 [(M+H)+]. ¹H NMR (400 MHz, DMSO-d₆) δ: 1.33 (t, 6H, OCH₂CH₃), 1.96 (m, 4H, —CH₂CH₂—), 3.52 (m, 4H, —CH₂NCH₂—), 3.99 (q, 2H, OCH₂CH₃), 6.92-7.78 (m, 8H, C₆H₄+C₆H₄), 10.2 (s, 1H, NH), 10.4 (s, 1H, 1NH).

N-(3-Fluorophenyl)-N¹-(4-methoxyphenyl)-6-pyrrolidin-1-yl-[1,3,5]triazine-2,4-diamine hydrochloride (Compound 500) (VIRADEM-012176): Yield 75%. M.p.=217-218° C. C₂₀H₂₂ClFN₆O. MS m/z: 381.2 [(M+H)+]. ¹H NMR (400 MHz, DMSO-d₆) δ: 1.93 (m, 4H, —CH₂CH₂—), 3.52 (m, 4H, —CH₂NCH₂—), 3.76 (s, 3H, OCH₃), 6.89-7.78 (m, 8H, C₆H₄+C₆H₄), 10.3 (s, 1H, 1NH), 10.4 (s, 1H, 1NH).

N-(4-Fluorophenyl)-N¹-(3-Fluorophenyl)-6-pyrrolidin-1-yl-[1,3,5]triazine-2,4-diamine hydrochloride (Compound 501) (VIRADEM-012177): Yield 78%. M.p.=221-222° C. C₁₉H₁₉ClF₂N₆. MS m/z: 369.1 [(M+H)+]. ¹H NMR (400 MHz, DMSO-d₆) δ: 1.94 (m, 4H, —CH₂CH₂—), 3.55 (m, 4H, —CH₂NCH₂—), 6.86-7.79 (m, 8H, C₆H₄+C₆H₄), 10.3 (s, 1H, 1NH), 10.4 (s, 1H, 1NH).

1-{3-[4-(4-Fluorophenylamino)-6-pyrrolidin-1-yl-[1,3,5]triazin-2-ylamino]-phenyl}-ethanone hydrochloride (Compound 502) (VIRADEM-012178): Yield 81%. M.p.=224-225° C. C₂₁H₂₂ClFN₆O. MS m/z: 393.2 [(M+H)+]. ¹H NMR (400 MHz, DMSO-d₆) δ: 1.94 (m, 4H, —CH₂CH₂—), 2.55 (s, 3H, COCH₃), 3.56 (m, 4H, —CH₂NCH₂—), 7.16-7.89 (m, 8H, C₆H₄+C₆H₄), 10.4 (s, 2H, 2NH).

N-(4-Chlorophenyl)-N¹-(3,5-dimethylphenyl)-6-pyrrolidin-1-yl-[1,3,5]triazine-2,4-diamine hydrochloride (Compound 503) (VIRADEM-012179): Yield 83%. M.p.=212-213° C. C₂₁H₂₄Cl₂N₆. MS m/z: 395.6 [(M+H)+]. ¹H NMR (400 MHz, DMSO-d₆) δ: 1.96 (m, 4H, —CH₂CH₂—), 2.24 (s, 6H, 2CH₃), 3.53 (m, 4H, —CH₂NCH₂—), 6.76-7.78 (m, 7H, C₆H₃+C₆H₄), 10.2 (s, 1H, NH), 10.5 (s, 1H, 1NH).

N-(3-Fluorophenyl)-6-pyrrolidin-1-yl-1,3,5]triazine-2,4-diamine hydrochloride (Compound 504) (VIRADEM-012180): Yield 73%. C₂₀H₂₂ClFN₆. M.p.=228-229° C. MS m/z: 365.2 [(M+H)+]. ¹H NMR (400 MHz, DMSO-d₆) δ: 1.93 (m, 4H, —CH₂CH₂—), 2.18 (s, 3H, CH₃), 3.52 (m, 4H, —CH₂NCH₂—), 7.08-7.79 (m, 8H, C₆H₃+C₆H₄), 10.3 (s, 2H, 2NH).

1-{3-[4-(4-Methoxyphenylamino)-6-pyrrolidin-1-yl-[1,3,5]triazin-2-ylamino]-phenyl}-ethanone hydrochloride (Compound 505) (VIRADEM-012184): Yield 80%. M.p.=222-223° C. C₂₂H₂₅ClN₆O₂. MS m/z: 405.2 [(M+H)+]. ¹H NMR (400 MHz, DMSO-d₆) δ: 1.95 (m, 4H, —CH₂CH₂—), 2.55 (s, 3H, COCH₃), 3.56 (m, 4H, —CH₂NCH₂—), 3.74 (s, 3H, OCH₃), 6.90-7.91 (m, 8H, C₆H₄+C₆H₄), 10.3 (s, 2H, 2NH).

N-(3,4-Dimethylphenyl)-N¹-(3-ethoxyphenyl)-6-pyrrolidin-1-yl-[1,3,5]triazine-2,4-diamine hydrochloride (Compound 506) (VIRADEM-012185): Yield 78%. M.p.=233-234° C. C₂₃H₂₉ClN₆O. MS m/z: 405.3 [(M+H)+]. ¹H NMR (400 MHz, DMSO-d₆) δ: 1.32 (t, 6H, OCH₂CH₃), 1.97 (m, 4H, —CH₂CH₂—), 2.20 (s, 6H, 2CH₃), 3.54 (m, 4H, —CH₂NCH₂—), 4.03 (q, 2H, OCH₂CH₃), 6.91-7.55 (m, 7H, C₆H₃+C₆H₄), 10.3 (s, 2H, 2NH).

N-(3,5-Dimethylphenyl)-N¹-(3-fluorophenyl)-6-pyrrolidin-1-yl-[1,3,5]triazine-2,4-diamine hydrochloride (Compound 507) (VIRADEM-012186): Yield 75%. M.p.=214-215° C. C₂₁H₂₄ClFN₆. MS m/z: 379.2 [(M+H)+]. ¹H NMR (400 MHz, DMSO-d₆) δ: 1.97 (m, 4H, —CH₂CH₂—), 2.27 (s, 6H, 2CH₃), 3.55 (m, 4H, —CH₂NCH₂—), 6.76-7.78 (m, 7H, C₆H₃+C₆H₄), 10.3 (s, 2H, 2NH).

N-(3,4-Dimethylphenyl)-N¹-(3-fluorophenyl)-6-pyrrolidin-1-yl-[1,3,5]triazine-2,4-diamine hydrochloride (Compound 508) (VIRADEM-012192): Yield 77%. M.p.=226-227° C. C₂₁H₂₄ClFN₆. MS m/z: 379.2 [(M+H)+]. ¹H NMR (400 MHz, DMSO-d₆) δ: 1.95 (m, 4H, —CH₂CH₂—), 2.18 (s, 3H, CH₃), 2.20 (s, 3H, CH₃), 3.55 (m, 4H, —CH₂NCH₂—), 6.87-7.82 (m, 7H, C₆H₃+C₆H₄), 10.2 (s, 2H, 2NH).

N-(3,4-Dimethylphenyl)-N¹-(3,5-dimethylphenyl)-6-pyrrolidin-1-yl-[1,3,5]triazine-2,4-diamine hydrochloride (Compound 509) (VIRADEM-012193): Yield 71%. M.p.=224-225° C. C₂₃H₂₉ClN₆. MS m/z: 389.3 [(M+H)+]. ¹H NMR (400 MHz, DMSO-d₆) δ: 1.97 (m, 4H, —CH₂CH₂—), 2.18 (s, 6H, 2CH₃), 2.24 (s, 6H, 2CH₃), 3.57 (m, 4H, —CH₂NCH₂—), 6.73-7.58 (m, 6H, C₆H₃+C₆H₃), 9.83 (s, 2H, 2NH).

N-(4-Ethoxyphenyl)-N¹-(4-fluorophenyl)-6-pyrrolidin-1-yl-[1,3,5]triazine-2,4-diamine hydrochloride (Compound 510) (VIRADEM-012194): Yield 75%. M.p.=235-236° C. C₂₁H₂₄ClFN₆O. MS m/z: 395.2 [(M+H)+]. ¹H NMR (400 MHz, DMSO-d₆) δ: 1.31 (t, 6H, OCH₂CH₃), 1.94 (m, 4H, —CH₂CH₂—), 3.53 (m, 4H, —CH₂NCH₂—), 4.01 (q, 2H, OCH₂CH₃), 6.90-7.74 (m, 8H, C₆H₄+C₆H₄), 10.3 (s, 2H, 2NH).

N-(4-Methoxyphenyl)-N¹-phenyl-6-pyrrolidin-1-yl-[1,3,5]triazine-2,4-diamine hydrochloride (Compound 511) (VIRADEM-012198): Yield 73%. M.p.=217-218° C. C₂₀H₂₃ClN₆O. MS m/z: 363.2 [(M+H)+]. ¹H NMR (400 MHz, DMSO-d₆) δ: 1.97 (m, 4H, —CH₂CH₂—), 3.60 (m, 4H, —CH₂NCH₂—), 3.77 (c, 3H, OCH₃), 6.93-7.69 (m, 9H, C₆H₄+C₆H₅), 10.3 (s, 1H, 1NH), 10.4 (s, 1H, 1NH).

N-(4-Chlorophenyl)-N¹-phenyl-6-pyrrolidin-1-yl-[1,3,5]triazine-2,4-diamine hydrochloride (Compound 512) (VIRADEM-012199): Yield 85%. M.p.=221-222° C. C₁₉H₂₀Cl₂N₆. MS m/z: 367.6 [(M+H)+]. ¹H NMR (400 MHz, DMSO-d₆) δ: 1.96 (m, 4H, —CH₂CH₂—), 3.57 (m, 4H, —CH₂NCH₂—), 7.09-7.77 (m, 9H, C₆H₄+C₆H₅), 10.2 (s, 1H, 1NH), 10.4 (s, 1H, 1NH).

N-(4-Chlorophenyl)-6-pyrrolidin-1-yl-N¹-m-tolyl-[1,3,5]triazine-2,4-diamine hydrochloride (Compound 513) (VIRADEM-012200): Yield 81%. M.p.=214-215° C. C₂₀H₂₂Cl₂N₆. MS m/z: 381.5 [(M+H)+]. ¹H NMR (400 MHz, DMSO-d₆) δ: 1.97 (m, 4H, —CH₂CH₂—), 2.30 (s, 3H, CH₃), 3.55 (m, 4H, —CH₂NCH₂—), 6.91-7.79 (m, 8H, C₆H₄+C₆H₄), 10.0 (s, 1H, 1NH), 10.3 (s, 1H, 1NH).

N-(3,4-Dimethylphenyl)-N¹-(4-ethylphenyl)-6-pyrrolidin-1-yl-[1,3,5]triazine-2,4-diamine hydrochloride (Compound 514) (VIRADEM-012201): Yield 69%. M.p.=221-222° C. C₂₃H₂₉ClN₆. MS m/z: 389.3 [(M+H)+]. ¹H NMR (400 MHz, DMSO-d₆) δ: 1.17 (t, 3H, CH₂CH₃), 1.95 (m, 4H, —CH₂CH₂—), 2.19 (s, 3H, CH₃), 2.22 (s, 3H, CH₃), 2.55 (q, 2H, CH₂CH₃), 3.57 (m, 4H, —CH₂NCH₂—), 6.81-7.57 (m, 6H, C₆H₃+C₆H₃), 9.95 (s, 1H, 1NH), 10.1 (s, 2H, 2NH).

1-{3-[4-(3,4-Dimethylphenylamino)-6-pyrrolidin-1-yl-[1,3,5]triazin-2-ylamino]-phenyl}-ethanone hydrochloride (Compound 515) (VIRADEM-012209): Yield 76%. M.p.=216-217° C. C₂₃H₂₇ClN₆O. MS m/z: 403.3 [(M+H)+]. ¹H NMR (400 MHz, DMSO-d₆) δ: 1.95 (m, 4H, —CH₂CH₂—), 2.18 (s, 3H, CH₃), 2.20 (s, 3H, CH₃), 2.55 (s, 3H, COCH₃), 3.56 (m, 4H, —CH₂NCH₂—), 6.93-7.89 (m, 7H, C₆H₃+C₆H₄), 10.2 (s, 2H, 2NH).

N-(4-Ethoxyphenyl)-6-pyrrolidin-1-yl-N¹-m-tolyl-[1,3,5]triazine-2,4-diamine hydrochloride (Compound 516) (VIRADEM-012210): Yield 71%. M.p.=204-205° C. C₂₂H₂₇ClN₆O. MS m/z: 391.2 [(M+H)+]. ¹H NMR (400 MHz, DMSO-d₆) δ: 1.30 (t, 6H, OCH₂CH₃), 1.95 (m, 4H, —CH₂CH₂—), 2.19 (s, 3H, CH₃), 3.54 (m, 4H, —CH₂NCH₂—), 4.03 (q, 2H, OCH₂CH₃), 6.93-7.62 (m, 8H, C₆H₄+C₆H₄), 10.0 (s, 1H, 1NH), 10.3 (s, 1H, 1NH).

N-(3-Chloro-4-methylphenyl)-6-pyrrolidin-1-yl-N¹-m-tolyl-[1,3,5]triazine-2,4-diamine hydrochloride (Compound 517) (VIRADEM-012212): Yield 75%. M.p.=233-234° C. C₂₁H₂₄Cl₂N₆. MS m/z: 395.6 [(M+H)+]. ¹H NMR (400 MHz, DMSO-d₆) δ: 1.96 (m, 4H, —CH₂CH₂—), 2.29 (s, 6H, 2CH₃), 3.57 (m, 4H, —CH₂NCH₂—), 6.89-7.92 (m, 7H, C₆H₃+C₆H₄), 9.95 (s, 2H, 2NH).

N-(4-Chlorophenyl)-N¹-(3-fluorophenyl)-6-pyrrolidin-1-yl-[1,3,5]triazine-2,4-diamine hydrochloride (Compound 518) (VIRADEM-012213): Yield 79%. M.p.=203-204° C. C₂₁H₁₉Cl₂FN₆. MS m/z: 385.6 [(M+H)+]. ¹H NMR (400 MHz, DMSO-d₆) δ: 1.92 (m, 4H, —CH₂CH₂—), 3.54 (m, 4H, —CH₂NCH₂—), 6.84-7.80 (m, 8H, C₆H₄+C₆H₄), 10.2 (s, 2H, 2NH).

N-(3-Chlorophenyl)-N¹-(3,4-dimethylphenyl)-6-pyrrolidin-1-yl-[1,3,5]triazine-2,4-diamine hydrochloride (Compound 519) (VIRADEM-012214): Yield 77%. M.p.=219-220° C. C₂₁H₂₄Cl₂N₆. MS m/z: 395.6 [(M+H)+]. ¹H NMR (400 MHz, DMSO-d₆) δ: 1.95 (m, 4H, —CH₂CH₂—), 2.28 (s, 6H, 2CH₃), 3.56 (m, 4H, —CH₂NCH₂—), 6.93-7.95 (m, 7H, C₆H₃+C₆H₄), 10.1 (s, 2H, 2NH).

N-(4-Ethoxyphenyl)-N¹-(3-fluorophenyl)-6-pyrrolidin-1-yl-[1,3,5]triazine-2,4-diamine hydrochloride (Compound 520) (VIRADEM-012215): Yield 67%. M.p.=202-203° C. C₂₁H₂₄ClFN₆O. MS m/z: 395.3 [(M+H)+]. ¹H NMR (400 MHz, DMSO-d₆) δ: 1.32 (t, 6H, OCH₂CH₃), 1.94 (m, 4H, —CH₂CH₂—), 2.19 (s, 3H, CH₃), 3.53 (m, 4H, —CH₂NCH₂—), 4.02 (q, 2H, OCH₂CH₃), 6.86-7.79 (m, 8H, C₆H₄+C₆H₄), 10.4 (s, 2H, 2NH).

N-(4-Chlorophenyl)-N¹-(4-methoxyphenyl)-6-pyrrolidin-1-yl-[1,3,5]triazine-2,4-diamine hydrochloride (Compound 521) (VIRADEM-012216): Yield 79%. M.p.=221-222° C. C₂₀H₂₂Cl₂N₆O. MS m/z: 397.5 [(M+H)+]. ¹H NMR (400 MHz, DMSO-d₆) δ: 1.95 (m, 4H, —CH₂CH₂—), 3.57 (m, 4H, —CH₂NCH₂—), 3.76 (s, 3H, OCH₃), 6.94-7.78 (m, 8H, C₆H₄+C₆H₄), 10.0 (s, 1H, 1NH), 10.4 (s, 1H, 1NH).

N-(3,4-Dimethylphenyl)-N¹-phenyl-6-pyrrolidin-1-yl-[1,3,5]triazine-2,4-diamine hydrochloride (Compound 522) (VIRADEM-012217): Yield 73%. M.p.=215-216° C. C₂₁H₂₅ClN₆. MS m/z: 361.2 [(M+H)+]. ¹H NMR (400 MHz, DMSO-d₆) δ: 1.98 (m, 4H, —CH₂CH₂—), 2.21 (s, 3H, CH₃), 2.23 (s, 3H, CH₃), 3.59 (m, 4H, —CH₂NCH₂—), 7.09-7.70 (m, 8H, C₆H₃+C₆H₅), 10.4 (s, 2H, 2NH).

N-(4-Fluorophenyl)-N¹-phenyl-6-pyrrolidin-1-yl-[1,3,5]triazine-2,4-diamine hydrochloride (Compound 523) (VIRADEM-012218): Yield 77%. M.p.=219-220° C. C₁₉H₂₀ClFN₆. MS m/z: 351.2 [(M+H)+]. ¹H NMR (400 MHz, DMSO-d₆) δ: 1.95 (m, 4H, —CH₂CH₂—), 3.56 (m, 4H, —CH₂NCH₂—), 7.12-7.92 (m, 9H, C₆H₄+C₆H₅), 10.3 (s, 2H, 2NH).

N-(3-Chlorophenyl)-N¹-(3-fluorophenyl)-6-pyrrolidin-1-yl-[1,3,5]triazine-2,4-diamine hydrochloride (Compound 524) (VIRADEM-012219): Yield 79%. M.p.=213-214° C. C₁₉H₁₉Cl₂FN₆. MS m/z: 385.5 [(M+H)+]. ¹H NMR (400 MHz, DMSO-d₆) δ: 1.96 (m, 4H, —CH₂CH₂—), 3.56 (m, 4H, —CH₂NCH₂—), 6.85-7.97 (m, 8H, C₆H₄+C₆H₄), 10.1 (s, 2H, 2NH).

N-(3,4-Dimethylphenyl)-6-pyrrolidin-1-yl-N¹-m-tolyl-[1,3,5]triazine-2,4-diamine hydrochloride (Compound 525) (VIRADEM-012220): Yield 70%. M.p.=216-217° C. C₂₂H₂₇ClN₆. MS m/z: 375.2 [(M+H)+]. ¹H NMR (400 MHz, DMSO-d₆) δ: 1.94 (m, 4H, —CH₂CH₂—), 2.18 (s, 6H, 2CH₃), 2.20 (s, 3H, CH₃), 3.57 (m, 4H, —CH₂NCH₂—), 7.02-7.67 (m, 7H, C₆H₃+C₆H₄), 10.1 (s, 2H, 2NH).

N-(3-Fluorophenyl)-6-pyrrolidin-1-yl-N¹-p-tolyl-[1,3,5]triazine-2,4-diamine hydrochloride (Compound 526) (VIRADEM-012228): Yield 73%. M.p.=214-215° C. C₂₀H₂₂ClFN₆. MS m/z: 365.2 [(M+H)+]. ¹H NMR (400 MHz, DMSO-d₆) δ: 1.92 (m, 4H, —CH₂CH₂—), 2.18 (s, 3H, CH₃), 3.56 (m, 4H, —CH₂NCH₂—), 6.92-7.77 (m, 8H, C₆H₄+C₆H₄), 10.1 (s, 1H, 1NH), 10.4 (s, 1H, 1NH).

N-(3-Chloro-4-fluorophenyl)-N¹-(3-fluorophenyl)-6-pyrrolidin-1-yl-[1,3,5]triazine-2,4-diamine hydrochloride (Compound 527) (VIRADEM-012229): Yield 77%. M.p.=232-233° C. C₁₉H₁₈Cl₂F₂N₆. MS m/z: 403.6 [(M+H)+]. ¹H NMR (400 MHz, DMSO-d₆) δ: 1.94 (m, 4H, —CH₂CH₂—), 3.56 (m, 4H, —CH₂NCH₂—), 6.84-8.08 (m, 7H, C₆H₃+C₆H₄), 9.95 (s, 1H, 1NH), 10.1 (s, 1H, 1NH).

N-(3-Chloro-4-fluorophenyl)-6-pyrrolidin-1-yl-N¹-m-tolyl-[1,3,5]triazine-2,4-diamine hydrochloride (Compound 528) (VIRADEM-012230): Yield 75%. M.p.=218-219° C. C₂₀H₂₁Cl₂FN₆. MS m/z: 399.5 [(M+H)+]. ¹H NMR (400 MHz, DMSO-d₆) δ: 1.96 (m, 4H, —CH₂CH₂—), 2.29 (s, 3H, CH₃), 3.55 (m, 4H, —CH₂NCH₂—), 6.89-8.02 (m, 7H, C₆H₃+C₆H₄), 10.1 (s, 2H, 2NH).

N-(3-Chloro-4-methoxyphenyl)-6-pyrrolidin-1-yl-N¹-m-tolyl-[1,3,5]triazine-2,4-diamine hydrochloride (Compound 529) (VIRADEM-012231): Yield 69%. M.p.=209-210° C. C₂₁H₂₄Cl₂N₆O. MS m/z: 411.6 [(M+H)+]. ¹H NMR (400 MHz, DMSO-d₆) δ: 1.94 (m, 4H, —CH₂CH₂—), 2.28 (s, 3H, CH₃), 3.54 (m, 4H, —CH₂NCH₂—), 3.76 (s, 3H, OCH₃), 6.78-7.82 (m, 7H, C₆H₃+C₆H₄), 10.0 (s, 2H, 2NH).

N-(4-Ethylphenyl)-N¹-(3-fluorophenyl)-6-pyrrolidin-1-yl-[1,3,5]triazine-2,4-diamine hydrochloride (Compound 530) (VIRADEM-012232): Yield 73%. M.p.=210-211° C. C₂₁H₂₄ClFN₆. MS m/z: 379.2 [(M+H)+]. ¹H NMR (400 MHz, DMSO-d₆) δ: 1.18 (t, 3H, CH₂CH₃), 1.94 (m, 4H, —CH₂CH₂—), 2.54 (q, 2H, CH₂CH₃), 3.56 (m, 4H, —CH₂NCH₂—), 6.89-7.68 (m, 8H, C₆H₄+C₆H₄), 9.89 (s, 1H, 1NH), 10.0 (s, 1H, 1NH).

N-(3-Chlorophenyl)-N¹-(4-chlorophenyl)-6-pyrrolidin-1-yl-[1,3,5]triazine-2,4-diamine hydrochloride (Compound 531) (VIRADEM-012233): Yield 82%. M.p.=216-217° C. C₁₉H₁₉Cl₃N₆. MS m/z: 402.1 [(M+H)+]. ¹H NMR (400 MHz, DMSO-d₆) δ: 1.96 (m, 4H, —CH₂CH₂—), 3.57 (m, 4H, —CH₂NCH₂—), 7.09-7.96 (m, 8H, C₆H₄+C₆H₄), 10.2 (s, 1H, 1NH), 10.3 (s, 1H, 1NH).

N-(3,5-Dimethylphenyl)-N¹-(4-ethylphenyl)-6-pyrrolidin-1-yl-[1,3,5]triazine-2,4-diamine hydrochloride (Compound 532) (VIRADEM-012237): Yield 70%. M.p.=233-234° C. C₂₃H₂₉ClN₆. MS m/z: 389.3 [(M+H)+]. ¹H NMR (400 MHz, DMSO-d₆) δ: 1.17 (t, 3H, CH₂CH₃), 1.95 (m, 4H, —CH₂CH₂—), 2.18 (s, 6H, 2CH₃), 2.55 (q, 2H, CH₂CH₃), 3.55 (m, 4H, —CH₂NCH₂—), 6.91-7.56 (m, 7H, C₆H₃+C₆H₄), 10.0 (s, 1H, 1NH), 10.2 (s, 1H, 1NH).

N-(4-Ethoxyphenyl)-N¹-(4-ethylphenyl)-6-pyrrolidin-1-yl-[1,3,5]triazine-2,4-diamine hydrochloride (Compound 533) (VIRADEM-012238): Yield 79%. M.p.=221-222° C. C₂₃H₂₉ClN₆O. MS m/z: 405.3 [(M+H)+]. ¹H NMR (400 MHz, DMSO-d₆) δ: 1.17 (t, 3H, CH₂CH₃), 1.29 (t, 3H, OCH₂CH₃), 1.95 (m, 4H, —CH₂CH₂—), 2.55 (q, 2H, CH₂CH₃), 3.55 (m, 4H, —CH₂NCH₂—), 4.01 (q, 2H, OCH₂CH₃), 6.92-7.65 (m, 8H, C₆H₄+C₆H₄), 10.0 (s, 1H, 1NH), 10.2 (s, 1H, 1NH).

N-(3-Chlorophenyl)-N¹-(4-ethoxyphenyl)-6-pyrrolidin-1-yl-[1,3,5]triazine-2,4-diamine hydrochloride (Compound 534) (VIRADEM-012239): Yield 77%. M.p.=207-208° C. C₂₁H₂₄Cl₂N₆O. MS m/z: 411.6 [(M+H)+]. ¹H NMR (400 MHz, DMSO-d₆) δ: 1.28 (t, 3H, OCH₂CH₃), 1.94 (m, 4H, —CH₂CH₂—), 3.56 (m, 4H, —CH₂NCH₂—), 4.03 (q, 2H, OCH₂CH₃), 6.85-7.95 (m, 8H, C₆H₄+C₆H₄), 10.3 (s, 2H, 2NH).

N-(3-Chlorophenyl)-N¹-(4-fluorophenyl)-6-pyrrolidin-1-yl-[1,3,5]triazine-2,4-diamine hydrochloride (Compound 535) (VIRADEM-012240): Yield 81%. M.p.=230-231° C. C₁₉H₁₉Cl₂FN₆. MS m/z: 385.6 [(M+H)+]. ¹H NMR (400 MHz, DMSO-d₆) δ: 1.93 (m, 4H, —CH₂CH₂—), 3.55 (m, 4H, —CH₂NCH₂—), 7.15-7.97 (m, 8H, C₆H₄+C₆H₄), 10.4 (s, 2H, 2NH).

N-(4-Ethoxyphenyl)-N¹-phenyl-6-pyrrolidin-1-yl-[1,3,5]triazine-2,4-diamine hydrochloride (Compound 536) (VIRADEM-012241): Yield 75%. M.p.=215-216° C. C₂₁H₂₅ClN₆O. MS m/z: 377.2 [(M+H)+]. ¹H NMR (400 MHz, DMSO-d₆) δ: 1.30 (t, 3H, OCH₂CH₃), 1.93 (m, 4H, —CH₂CH₂—), 3.54 (m, 4H, —CH₂NCH₂—), 4.02 (q, 2H, OCH₂CH₃), 6.90-7.72 (m, 9H, C₆H₄+C₆H₅), 10.4 (s, 2H, 2NH).

N-(4-Ethylphenyl)-N¹-(3-methoxyphenyl)-6-pyrrolidin-1-yl-[1,3,5]triazine-2,4-diamine hydrochloride (Compound 537) (VIRADEM-012242): Yield 70%. M.p.=212-213° C. C₂₂H₂₇ClN₆O. MS m/z: 391.2 [(M+H)+]. ¹H NMR (400 MHz, DMSO-d₆) δ: 1.18 (t, 3H, CH₂CH₃), 1.94 (m, 4H, —CH₂CH₂—), 2.54 (q, 2H, CH₂CH₃), 3.56 (m, 4H, —CH₂NCH₂—), 3.75 (s, 3H, OCH₃), 6.89-7.56 (m, 8H, C₆H₄+C₆H₄), 10.1 (s, 1H, 1NH), 10.3 (s, 1H, 1NH).

N-(4-Ethylphenyl)-N¹-phenyl-6-pyrrolidin-1-yl-[1,3,5]triazine-2,4-diamine hydrochloride (Compound 538) (VIRADEM-012243): Yield 75%. M.p.=213-214° C. C₂₁H₂₅ClN₆. MS m/z: 360.3 [(M+H)+]. ¹H NMR (400 MHz, DMSO-d₆) δ: 1.17 (t, 3H, CH₂CH₃), 1.93 (m, 4H, —CH₂CH₂—), 2.53 (q, 2H, CH₂CH₃), 3.55 (m, 4H, —CH₂NCH₂—), 6.95-7.59 (m, 9H, C₆H₄+C₆H₅), 10.3 (s, 2H, 2NH).

N-(3-Chloro-4-methylphenyl)-N¹-(4-ethylphenyl)-6-pyrrolidin-1-yl-[1,3,5]triazine-2,4-diamine hydrochloride (Compound 539) (VIRADEM-012244): Yield 78%. M.p.=236-237° C. C₂₂H₂₆Cl₂N₆. MS m/z: 409.8 [(M+H)+]. ¹H NMR (400 MHz, DMSO-d₆) δ: 1.17 (t, 3H, CH₂CH₃), 1.93 (m, 4H, —CH₂CH₂—), 2.27 (s, 3H, CH₃), 2.54 (q, 2H, CH₂CH₃), 3.53 (m, 4H, —CH₂NCH₂—), 7.15-7.97 (m, 7H, C₆H₃+C₆H₄), 9.95 (s, 2H, 2NH).

N-(3,5-Dimethylphenyl)-6-pyrrolidin-1-yl-N¹-m-tolyl-[1,3,5]triazine-2,4-diamine hydrochloride (Compound 540) (VIRADEM-012257): Yield 73%. M.p.=222-223° C. C₂₂H₂₇ClN₆. MS m/z: 375.2 [(M+H)+]. ¹H NMR (400 MHz, DMSO-d₆) δ: 1.95 (m, 4H, —CH₂CH₂—), 2.19 (s, 9H, 3CH₃), 3.54 (m, 4H, —CH₂NCH₂—), 6.88-7.65 (m, 7H, C₆H₃+C₆H₄), 10.0 (s, 1H, 1NH), 10.2 (s, 1H, 1NH).

N-Phenyl-6-pyrrolidin-1-yl-N¹-p-tolyl-[1,3,5]triazine-2,4-diamine hydrochloride (Compound 541) (VIRADEM-012258): Yield 77%. M.p.=225-226° C. C₂₀H₂₃ClN₆. MS m/z: 347.2 [(M+H)+]. ¹H NMR (400 MHz, DMSO-d₆) δ: 1.93 (m, 4H, —CH₂CH₂—), 2.28 (s, 3H, CH₃), 3.55 (m, 4H, —CH₂NCH₂—), 6.98-7.67 (m, 9H, C₆H₄+C₆H₅), 10.3 (s, 2H, 2NH).

N-(3-Chloro-4-methoxyphenyl)-N¹-phenyl-6-pyrrolidin-1-yl-[1,3,5]triazine-2,4-diamine hydrochloride (Compound 542) (VIRADEM-012259): Yield 73%. M.p.=226-227° C. C₂₀H₂₂Cl₂N₆O. MS m/z: 397.6 [(M+H)+]. ¹H NMR (400 MHz, DMSO-d₆) δ: 1.95 (m, 4H, —CH₂CH₂—), 3.53 (m, 4H, —CH₂NCH₂—), 3.76 (s, 3H, OCH₃), 7.12-8.02 (m, 8H, C₆H₃+C₆H₅), 10.1 (s, 2H, 2NH).

N-(4-Ethylphenyl)-6-pyrrolidin-1-yl-N¹-m-tolyl-[1,3,5]triazine-2,4-diamine hydrochloride (Compound 543) (VIRADEM-012260): Yield 71%. M.p.=216-217° C. C₂₂H₂₇ClN₆. MS m/z: 375.2 [(M+H)+]. ¹H NMR (400 MHz, DMSO-d₆) δ: 1.18 (t, 3H, CH₂CH₃), 1.94 (m, 4H, —CH₂CH₂—), 2.18 (s, 3H, CH₃), 2.54 (q, 2H, CH₂CH₃), 3.55 (m, 4H, —CH₂NCH₂—), 6.98-7.63 (m, 8H, C₆H₄+C₆H₄), 10.2 (s, 2H, 2NH).

N-Phenyl-6-pyrrolidin-1-yl-N¹-m-tolyl-[1,3,5]triazine-2,4-diamine hydrochloride (Compound 544) (VIRADEM-012261): Yield 75%. M.p.=205-206° C. C₂₀H₂₃ClN₆. MS m/z: 347.2 [(M+H)+]. ¹H NMR (400 MHz, DMSO-d₆) δ: 1.92 (m, 4H, —CH₂CH₂—), 2.18 (s, 3H, CH₃), 3.54 (m, 4H, —CH₂NCH₂—), 6.89-7.56 (m, 9H, C₆H₄+C₆H₅), 10.2 (s, 1H, 1NH), 10.3 (s, 1H, 1NH).

N-(3-Chloro-4-fluorophenyl)-N¹-(4-ethylphenyl)-6-pyrrolidin-1-yl-[1,3,5]triazine-2,4-diamine hydrochloride (Compound 545) (VIRADEM-012274): Yield 77%. M.p.=206-207° C. C₂₁H₂₃Cl₂FN₆. MS m/z: 413.7 [(M+H)+]. ¹H NMR (400 MHz, DMSO-d₆) δ: 1.17 (t, 3H, CH₂CH₃), 1.96 (m, 4H, —CH₂CH₂—), 2.54 (q, 2H, CH₂CH₃), 3.53 (m, 4H, —CH₂NCH₂—), 7.17-8.06 (m, 7H, C₆H₃+C₆H₄), 10.3 (s, 2H, 2NH).

N-(3-Methoxyphenyl)-N¹-phenyl-6-pyrrolidin-1-yl-[1,3,5]triazine-2,4-diamine hydrochloride (Compound 546) (VIRADEM-012275): Yield 73%. M.p.=201-202° C. C₂₀H₂₃ClN₆O. MS m/z: 363.3 [(M+H)+]. ¹H NMR (400 MHz, DMSO-d₆) δ: 1.94 (m, 4H, —CH₂CH₂—), 3.55 (m, 4H, —CH₂NCH₂—), 3.75 (s, 3H, OCH₃), 6.87-7.56 (m, 9H, C₆H₄+C₆H₅), 10.1 (s, 2H, 2NH).

N-(3-Chlorophenyl)-N¹-(4-methoxyphenyl)-6-pyrrolidin-1-yl-[1,3,5]triazine-2,4-diamine hydrochloride (Compound 547) (VIRADEM-012276): Yield 83%. M.p.=221-222° C. C₂₀H₂₂Cl₂N₆O. MS m/z: 397.6 [(M+H)+]. ¹H NMR (400 MHz, DMSO-d₆) δ: 1.93 (m, 4H, —CH₂CH₂—), 3.55 (m, 4H, —CH₂NCH₂—), 3.76 (s, 3H, OCH₃), 6.82-8.01 (m, 8H, C₆H₄+C₆H₄), 10.4 (s, 2H, 2NH).

N-(3-Chlorophenyl)-6-pyrrolidin-1-yl-N¹-p-tolyl-[1,3,5]triazine-2,4-diamine hydrochloride (Compound 548) (VIRADEM-012277): Yield 78%. M.p.=212-213° C. C₂₀H₂₂Cl₂N₆O. MS m/z: 381.6 [(M+H)+]. ¹H NMR (400 MHz, DMSO-d₆) δ: 1.95 (m, 4H, —CH₂CH₂—), 2.28 (s, 3H, CH₃), 3.56 (m, 4H, —CH₂NCH₂—), 7.12-7.98 (m, 8H, C₆H₄+C₆H₄), 10.1 (s, 1H, 1NH), 10.4 (s, 1H, 1NH).

N-(3-Chlorophenyl)-N¹-(3,5-dimethylphenyl)-6-pyrrolidin-1-yl-[1,3,5]triazine-2,4-diamine hydrochloride (Compound 549) (VIRADEM-012278): Yield 80%. M.p.=217-218° C. C₂₁H₂₄Cl₂N₆. MS m/z: 395.7 [(M+H)+]. ¹H NMR (400 MHz, DMSO-d₆) δ: 1.95 (m, 4H, —CH₂CH₂—), 2.18 (s, 6H, 2CH₃), 3.56 (m, 4H, —CH₂NCH₂—), 6.98-7.68 (m, 7H, C₆H₃+C₆H₄), 10.2 (s, 2H, 2NH).

N-(3-Chlorophenyl)-6-pyrrolidin-1-yl-N¹-m-tolyl-[1,3,5]triazine-2,4-diamine hydrochloride (Compound 550) (VIRADEM-012282): Yield 77%. M.p.=214-215° C. C₂₀H₂₂Cl₂N₆. MS m/z: 381.6 [(M+H)+]. ¹H NMR (400 MHz, DMSO-d₆) δ: 1.93 (m, 4H, —CH₂CH₂—), 2.16 (s, 3H, CH₃), 3.55 (m, 4H, —CH₂NCH₂—), 7.08-7.89 (m, 8H, C₆H₄+C₆H₄), 10.4 (s, 2H, 2NH).

N-(4-Ethylphenyl)-N¹-phenyl-6-pyrrolidin-1-yl-[1,3,5]triazine-2,4-diamine hydrochloride (Compound 551) (VIRADEM-012283): Yield 78%. M.p.=204-205° C. C₂₁H₂₅ClN₆. MS m/z: 361.3 [(M+H)+]. ¹H NMR (400 MHz, DMSO-d₆) δ: 1.18 (t, 3H, CH₂CH₃), 1.95 (m, 4H, —CH₂CH₂—), 2.55 (q, 2H, CH₂CH₃), 3.54 (m, 4H, —CH₂NCH₂—), 7.09-7.96 (m, 8H, C₆H₃+C₆H₅), 10.4 (s, 2H, 2NH).

N-(4-Ethylphenyl)-6-pyrrolidin-1-yl-N¹-p-tolyl-[1,3,5]triazine-2,4-diamine hydrochloride (Compound 552) (VIRADEM-012284): Yield 74%. M.p.=222-223° C. C₂₂H₂₇ClN₆. MS m/z: 375.2 [(M+H)+]. ¹H NMR (400 MHz, DMSO-d₆) δ: 1.17 (t, 3H, CH₂CH₃), 1.94 (m, 4H, —CH₂CH₂—), 2.28 (s, 3H, CH₃), 2.54 (q, 2H, CH₂CH₃), 3.55 (m, 4H, —CH₂NCH₂—), 7.03-7.83 (m, 8H, C₆H₄+C₆H₄), 10.3 (s, 2H, 2NH).

N-(3,5-Dimethylphenyl)-N¹-phenyl-6-pyrrolidin-1-yl-[1,3,5]triazine-2,4-diamine hydrochloride (Compound 553) (VIRADEM-012285): Yield 77%. M.p.=216-217° C. C₂₁H₂₅ClN₆. MS m/z: 361.2 [(M+H)+]. ¹H NMR (400 MHz, DMSO-d₆) δ: 1.97 (m, 4H, —CH₂CH₂—), 2.26 (s, 6H, 2CH₃), 3.57 (m, 4H, —CH₂NCH₂—), 6.76-7.70 (m, 8H, C₆H₄+C₆H₄), 10.3 (s, 2H, 2NH).

N-(3-Chloro-4-methylphenyl)-N¹-(-3-chlorophenyl)-6-pyrrolidin-1-yl-[1,3,5]triazine-2,4-diamine (Compound 554) (VIRADEM-012287): Yield 83%. M.p.=171-172° C. C₂₀H₂₀Cl₂N₆. MS m/z: 416.0 [(M+H)+]. ¹H NMR (400 MHz, DMSO-d₆) δ: 1.92 (m, 4H, —CH₂CH₂—), 2.25 (s, 3H, CH₃), 3.55 (m, 4H, —CH₂NCH₂—), 6.99-8.16 (m, 7H, C₆H₃+C₆H₄), 9.27 (s, 1H, 1NH), 9.35 (s, 1H, 1NH).

N-(3-Chloro-4-methoxyphenyl)-N¹-(-3-chlorophenyl)-6-pyrrolidin-1-yl-[1,3,5]triazine-2,4-diamine (Compound 555) (VIRADEM-012291): Yield 79%. M.p.=106-107° C. C₂₀H₂₀Cl₂N₆O. MS m/z: 432.0 [(M+H)+]. ¹H NMR (400 MHz, DMSO-d₆) δ: 1.93 (m, 4H, —CH₂CH₂—), 3.53 (m, 4H, —CH₂NCH₂—), 3.80 (s, 3H, OCH₃), 6.95-7.99 (m, 7H, C₆H₃+C₆H₄), 8.95 (s, 1H, 1NH), 9.11 (s, 1H, 1NH).

N-Phenyl-6-pyrrolidin-1-yl-N¹-(3-trifluoromethylphenyl)-[1,3,5]triazine-2,4-diamine hydrochloride (Compound 556) (VIRADEM-012306): Yield 80%. M.p.=204-205° C. C₂₀H₂₀ClF₃N₆. MS m/z: 401.1 [(M+H)+]. ¹H NMR (400 MHz, DMSO-d₆) δ: 1.96 (m, 4H, —CH₂CH₂—), 3.59 (m, 4H, —CH₂NCH₂—), 7.06-8.28 (m, 9H, C₆H₄+C₆H₅), 10.3 (s, 2H, 2NH).

(4,6-Dipyrrolidin-1-yl-[1,3,5]triazin-2-yl)-(4-isopropylphenyl)-amine hydrochloride (Compound 557) (VIRADEM-012307): Yield 78%. M.p.=208-209° C. C₂₀H₂₉ClN₆. MS m/z: 353.2 [(M+H)+]. ¹H NMR (400 MHz, DMSO-d₆) δ: 1.18 (d, 6H, CH(CH₃)₂), 1.95 (m, 8H, 2-CH₂CH₂—), 2.86 (m, 1H, CH(CH₃)₂), 3.57 (m, 8H, 2-CH₂NCH₂—), 7.14-7.62 (m, 4H, C₆H₄), 10.1 (s, 2H, 2NH).

N-(3-Chlorophenyl)-N¹-phenyl)-6-pyrrolidin-1-yl-[1,3,5]triazine-2,4-diamine hydrochloride (Compound 558) (VIRADEM-012312): Yield 79%. M.p.=237-238. C₁₉H₂₀Cl₂N₆. MS m/z: 367.6 [(M+H)+]. ¹H NMR (400 MHz, DMSO-d₆) δ: 1.94 (m, 4H, —CH₂CH₂—), 3.55 (m, 4H, —CH₂NCH₂—), 7.05-8.06 (m, 9H, C₆H₄+C₆H₅), 9.95 (s, 1H, 1NH), 10.1 (s, 1H, 1NH).

N-(4-Chlorophenyl)-N¹-(4-ethylphenyl)-6-pyrrolidin-1-yl-[1,3,5]triazine-2,4-diamine hydrochloride (Compound 559) (VIRADEM-012313): Yield 82%. M.p.=232-233° C. C₂₁H₂₄Cl₂N₆. MS m/z: 395.7 [(M+H)+]. ¹H NMR (400 MHz, DMSO-d₆) δ: 1.17 (t, 3H, CH₂CH₃), 1.94 (m, 4H, —CH₂CH₂—), 2.54 (q, 2H, CH₂CH₃), 3.55 (m, 4H, —CH₂NCH₂—), 7.08-7.98 (m, 8H, C₆H₄+C₆H₄), 10.3 (s, 2H, 2NH).

N-(4-Isopropylphenyl)-N¹-phenyl-6-pyrrolidin-1-yl-[1,3,5]triazine-2,4-diamine hydrochloride (Compound 560) (VIRADEM-012314): Yield 83%. M.p.=226-227. C₂₂H₂₇ClN₆. MS m/z: 375.2 [(M+H)+]. ¹H NMR (400 MHz, DMSO-d₆) δ: 1.17 (d, 6H, CH(CH₃)₂), 1.95 (m, 4H, —CH₂CH₂—), 2.85 (m, 1H, CH(CH₃)₂), 3.56 (m, 4H, —CH₂NCH₂—), 7.08-7.69 (m, 9H, C₆H₄+C₆H₅), 10.2 (s, 2H, 2NH).

N-(3-Chloro-4-fluorophenyl)-N¹-phenyl-6-pyrrolidin-1-yl-[1,3,5]triazine-2,4-diamine hydrochloride (Compound 561) (VIRADEM-012315): Yield 78%. M.p.=196-197° C. C₁₉H₁₉Cl₂FN₆. MS m/z: 385.5 [(M+H)+]. ¹H NMR (400 MHz, DMSO-d₆) δ: 1.93 (m, 4H, —CH₂CH₂—), 3.55 (m, 4H, —CH₂NCH₂—), 6.95-8.19 (m, 8H, C₆H₃+C₆H₅), 9.31 (s, 1H, NH), 9.40 (s, 1H, 1NH).

N-(3-Chloro-4-methylphenyl)-N¹-phenyl-6-pyrrolidin-1-yl-[1,3,5]triazine-2,4-diamine hydrochloride (Compound 562) (VIRADEM-012316): Yield 75%. M.p.=213-214° C. C₂₀H₂₂Cl₂N₆. MS m/z: 381.6 [(M+H)+]. ¹H NMR (400 MHz, DMSO-d₆) δ: 1.95 (m, 4H, —CH₂CH₂—), 2.28 (s, 3H, CH₃), 3.56 (m, 4H, —CH₂NCH₂—), 7.05-7.98 (m, 8H, C₆H₃+C₆H₅), 10.0 (s, 2H, 2NH).

N-(3-Fluorophenyl)-N¹-phenyl-6-pyrrolidin-1-yl-[1,3,5]triazine-2,4-diamine hydrochloride (Compound 563) (VIRADEM-012317): Yield 77%. M.p.=218-219° C. C₁₉H₂₀ClFN₆. MS m/z: 351.2 [(M+H)+]. ¹H NMR (400 MHz, DMSO-d₆) δ: 1.96 (m, 4H, —CH₂CH₂—), 3.54 (m, 4H, —CH₂NCH₂—), 6.83-7.89 (m, 9H, C₆H₄+C₆H₅), 10.1 (s, 2H, 2NH).

N-(3,4-Dichlorophenyl)-N¹-phenyl-6-pyrrolidin-1-yl-[1,3,5]triazine-2,4-diamine hydrochloride (Compound 564) (VIRADEM-012318): Yield 86%. M.p.=200-201° C. C₁₉H₁₉Cl₃N₆. MS m/z: 402.0 [(M+H)+]. ¹H NMR (400 MHz, DMSO-d₆) δ: 1.92 (m, 4H, —CH₂CH₂—), 3.53 (m, 4H, —CH₂NCH₂—), 7.03-8.23 (m, 8H, C₆H₃+C₆H₅), 9.79 (s, 1H, NH), 9.97 (s, 1H, 1NH).

N-(3,4-Dimethylphenyl)-6-pyrrolidin-1-yl-N¹-p-tolyl-[1,3,5]triazine-2,4-diamine hydrochloride (Compound 565) (VIRADEM-012338): Yield 69%. M.p.=231-232° C. C₂₂H₂₇ClN₆. MS m/z: 375.2 [(M+H)+]. ¹H NMR (400 MHz, DMSO-d₆) δ: 1.96 (m, 4H, —CH₂CH₂—), 2.27 (s, 9H, 3CH₃), 3.57 (m, 4H, —CH₂NCH₂—), 6.86-7.73 (m, 7H, C₆H₃+C₆H₄), 10.3 (s, 2H, 2NH).

N-(3,5-Dimethylphenyl)-N¹-(3-methoxyphenyl)-6-pyrrolidin-1-yl-[1,3,5]triazine-2,4-diamine hydrochloride (Compound 566) (VIRADEM-012339): Yield 73%. M.p.=226-227° C. C₂₂H₂₇ClN₆O. MS m/z: 391.2 [(M+H)+]. ¹H NMR (400 MHz, DMSO-d₆) δ: 1.94 (m, 4H, —CH₂CH₂—), 2.18 (s, 6H, 2CH₃), 3.55 (m, 4H, —CH₂NCH₂—), 3.76 (s, 3H, OCH₃), 6.63-7.79 (m, 7H, C₆H₃+C₆H₄), 9.81 (s, 1H, NH), 10.0 (s, 1H, 1NH).

N-(3,5-Dimethylphenyl)-6-pyrrolidin-1-yl-N¹-p-tolyl-[1,3,5]triazine-2,4-diamine hydrochloride (Compound 567) (VIRADEM-012340): Yield 77%. M.p.=216-217° C. C₂₂H₂₇ClN₆. MS m/z: 375.2 [(M+H)+]. ¹H NMR (400 MHz, DMSO-d₆) δ: 1.93 (m, 4H, —CH₂CH₂—), 2.19 (s, 6H, 2CH₃), 2.29 (s, 3H, CH₃), 3.55 (m, 4H, —CH₂NCH₂—), 7.03-7.68 (m, 7H, C₆H₃+C₆H₄), 10.1 (s, 2H, 2NH).

N-(4-Fluorophenyl)-N¹-(3-methoxyphenyl)-6-pyrrolidin-1-yl-[1,3,5]triazine-2,4-diamine hydrochloride (Compound 568) (VIRADEM-012341): Yield 78%. M.p.=214-215° C. C₂₀H₂₂ClFN₆O. MS m/z: 381.2 [(M+H)+]. ¹H NMR (400 MHz, DMSO-d₆) δ: 1.95 (m, 4H, —CH₂CH₂—), 3.56 (m, 4H, —CH₂NCH₂—), 3.75 (s, 3H, OCH₃), 6.68-7.71 (m, 8H, C₆H₄+C₆H₄), 9.89 (s, 1H, NH), 10.1 (s, 1H, 1NH).

N,N¹-Bis-(3-methoxyphenyl)-6-pyrrolidin-1-yl-[1,3,5]triazine-2,4-diamine (Compound 569) (VIRADEM-012342): Yield 80%. M.p.=134-135° C. C₂₁H₂₄NO₂. MS m/z: 393.2 [(M+H)+]. ¹H NMR (400 MHz, DMSO-d₆) δ: 1.93 (m, 4H, —CH₂CH₂—), 3.55 (m, 4H, —CH₂NCH₂—), 3.73 (s, 6H, 2OCH₃), 6.51-7.59 (m, 8H, C₆H₄+C₆H₄), 9.03 (s, 2H, 2NH).

N-(3,4-Dimethylphenyl)-N¹-(3-methoxyphenyl)-6-pyrrolidin-1-yl-[1,3,5]triazine-2,4-diamine hydrochloride (Compound 570) (VIRADEM-012343): Yield 75%. M.p.=229-230° C. C₂₂H₂₇ClN₆O. MS m/z: 391.2 [(M+H)+]. ¹H NMR (400 MHz, DMSO-d₆) δ: 1.93 (m, 4H, —CH₂CH₂—), 2.19 (s, 6H, 2CH₃), 3.55 (m, 4H, —CH₂NCH₂—), 3.74 (s, 3H, OCH₃), 6.56-7.68 (m, 7H, C₆H₃+C₆H₄), 9.89 (s, 1H, NH), 10.1 (s, 1H, 1NH).

N-(4-Ethoxyphenyl)-N¹-(3-methoxyphenyl)-6-pyrrolidin-1-yl-[1,3,5]triazine-2,4-diamine hydrochloride (Compound 571) (VIRADEM-012344): Yield 67%. M.p.=202-203° C. C₂₂H₂₇ClN₆O₂. MS m/z: 407.2 [(M+H)+]. ¹H NMR (400 MHz, DMSO-d₆) δ: 1.32 (t, 3H, OCH₂CH₃), 1.94 (m, 4H, —CH₂CH₂—), 3.55 (m, 4H, —CH₂NCH₂—), 3.73 (s, 3H, OCH₃), 4.00 (q, 2H, OCH₂CH₃), 6.58-7.61 (m, 8H, C₆H₄+C₆H₄), 9.69 (s, 2H, 2NH).

N-(3-Fluorophenyl)-N¹-(3-methoxyphenyl)-6-pyrrolidin-1-yl-[1,3,5]triazine-2,4-diamine hydrochloride (Compound 572) (VIRADEM-012348): Yield 73%. M.p.=206-207° C. C₂₀H₂₂ClFN₆O. MS m/z: 381.2 [(M+H)+]. ¹H NMR (400 MHz, DMSO-d₆) δ: 1.95 (m, 4H, —CH₂CH₂—), 3.54 (m, 4H, —CH₂NCH₂—), 3.75 (s, 3H, OCH₃), 6.59-7.89 (m, 8H, C₆H₄+C₆H₄), 9.68 (s, 1H, 1NH), 9.84 (s, 1H, 1NH).

N-(3-Methoxyphenyl)-6-pyrrolidin-1-yl-p-tolyl-[1,3,5]triazine-2,4-diamine hydrochloride (Compound 573) (VIRADEM-012350): Yield 75%. M.p.=202-233° C. C₂₁H₂₅ClN₆O. MS m/z: 377.2 [(M+H)+]. ¹H NMR (400 MHz, DMSO-d₆) δ: 1.94 (m, 4H, —CH₂CH₂—), 2.28 (s, 3H, CH₃), 3.55 (m, 4H, —CH₂NCH₂—), 3.76 (s, 3H, OCH₃), 6.57-7.68 (m, 8H, C₆H₄+C₆H₄), 9.73 (s, 1H, 1NH), 9.94 (s, 1H, 1NH).

N-(4-Chlorophenyl)-N¹-(3-methoxyphenyl)-6-pyrrolidin-1-yl-[1,3,5]triazine-2,4-diamine hydrochloride (Compound 574) (VIRADEM-012352): Yield 79%. M.p.=191-192° C. C₂₀H₂₂Cl₂N₆O. MS m/z: 397.6 [(M+H)+]. ¹H NMR (400 MHz, DMSO-d₆) δ: 1.94 (m, 4H, —CH₂CH₂—), 3.55 (m, 4H, —CH₂NCH₂—), 3.76 (s, 3H, OCH₃), 6.63-7.91 (m, 8H, C₆H₄+C₆H₄), 10.2 (s, 2H, 2NH).

N-(3-Chloro-4-methylphenyl)-N¹-(3-methoxyphenyl)-6-pyrrolidin-1-yl-[1,3,5]triazine-2,4-diamine hydrochloride (Compound 575) (VIRADEM-012353): Yield 77%. M.p.=222-223° C. C₂₁H₂₄Cl₂N₆O. MS m/z: 411.6 [(M+H)+]. ¹H NMR (400 MHz, DMSO-d₆) δ: 1.94 (m, 4H, —CH₂CH₂—), 2.29 (s, 3H, CH₃), 3.54 (m, 4H, —CH₂NCH₂—), 3.74 (s, 3H, OCH₃), 6.63-7.92 (m, 7H, C₆H₃+C₆H₄), 10.0 (s, 2H, 2NH).

1-{3-[4-(3-Methoxyphenylamino)-6-pyrrolidin-1-yl-[1,3,5]triazin-2-ylamino]-phenyl}-ethanone hydrochloride (Compound 576) (VIRADEM-012354): Yield 80%. M.p.=206-207° C. C₂₂H₂₅ClN₆O₂. MS m/z: 405.1 [(M+H)+]. ¹H NMR (400 MHz, DMSO-d₆) δ: 1.95 (m, 4H, —CH₂CH₂—), 2.54 (s, 3H, COCH₃), 3.55 (m, 4H, —CH₂NCH₂—), 3.76 (s, 3H, OCH₃), 6.64-8.12 (m, 8H, C₆H₄+C₆H₄), 10.3 (s, 2H, 2NH).

N-(3-Methoxyphenyl)-6-pyrrolidin-1-yl-m-tolyl-[1,3,5]triazine-2,4-diamine hydrochloride (Compound 577) (VIRADEM-012355): Yield 70%. M.p.=205-206° C. C₂₁H₂₅ClN₆O. MS m/z: 377.2 [(M+H)+]. ¹H NMR (400 MHz, DMSO-d₆) δ: 1.93 (m, 4H, —CH₂CH₂—), 2.19 (s, 3H, CH₃), 3.54 (m, 4H, —CH₂NCH₂—), 3.74 (s, 3H, OCH₃), 6.59-7.71 (m, 8H, C₆H₄+C₆H₄), 9.87 (s, 1H, 1NH), 9.99 (s, 1H, 1NH).

2-Morpholin-4-yl-4,6-dipyrrolidin-1-yl-[1,3,5]triazine hydrochloride (Compound 578) (VIRADEM-014273): Yield 62%. C₁₅H₂₅ClN₆O. MS m/z: 305.1 [(M+H)+]. ¹H NMR (400 MHz, DMSO-d₆) δ: 1.93 (m, 8H, 2-CH₂CH₂—), 3.54 (m, 8H, 2-CH₂NCH₂—), 3.65 (m, 4H, CH₂NCH₂), 3.74 (m, 4H, CH₂OCH₂).

(4,6-Dipiperidine-1-yl-[1,3,5]triazin-2-yl)-(4-methoxyphenyl)-amine hydrochloride (Compound 579) (VIRADEM-011041): Yield 76%. M.p.=193-194° C. C₂₀H₂₉ClN₆O. MS m/z: 369.2 [(M+H)+]. 1H NMR (400 MHz, DMSO-d₆) δ: 1.54-1.64 (m, 12H, 2-CH₂CH₂CH₂—), 3.73 (m, 8H, 2-CH₂NCH₂—), 3.74 (s, 3H, OCH₃), 6.89 and 7.57 (d-d, 4H, C₆H₄), 9.54 (s, 1H, NH).

2-Morpholin-4-yl-4,6-dipiperidin-1-yl-[1,3,5]triazine hydrochloride (Compound 580) (VIRADEM-014273): Yield 84%. M.p.=113-114° C. C₁₇H₂₉ClN₆O. MS m/z: 333.3 [(M+H)+]. ¹H NMR (400 MHz, DMSO-d₆) δ: 1.44-1.60 (m, 12H, 2-CH₂CH₂CH₂—), 3.57-3.68 (m, 16H, 3-CH₂NCH₂—+—CH₂OCH₂—).

(4,6-Dipiperidin-1-yl-[1,3,5]triazin-2-yl)-m-tolyl-amine hydrochloride (Compound 581) (VIRADEM-011492): Yield 79%. M.p.=206-207° C. C₂₀H₂₉ClN₆. MS m/z: 353.2 [(M+H)+]. ¹H NMR (400 MHz, DMSO-d₆) δ: 1.51-1.64 (m, 12H, 2-CH₂CH₂CH₂—), 3.68-3.77 (m, 8H, 2-CH₂NCH₂—), 7.12 and 7.84 (m, 4H, C₆H₄), 9.63 (s, 1H, NH).

(3,4-Dichlorophenyl)-(4,6-dipiperidine-1-yl-[1,3,5]triazin-2-yl)-amine hydrochloride (Compound 582) (VIRADEM-011493): Yield 88%. M.p.=215-216° C. C₁₉H₂₅Cl₃N₆. MS m/z: 408.0 [(M+H)+]. ¹H NMR (400 MHz, DMSO-d₆) δ: 1.54-1.66 (m, 12H, 2-CH₂CH₂CH₂—), 3.73 (m, 8H, 2-CH₂NCH₂—), 7.51-8.09 (m, 3H, C₆H₃), 10.1 (s, 1H, 1NH).

(3-Chloro-4-methylphenyl)-(4,6-dipiperidine-1-yl-[1,3,5]triazin-2-yl)-amine hydrochloride (Compound 583) (VIRADEM-011494): Yield 75%. C₂₀H₂₈Cl₂N₆. MS m/z: 387.6 [(M+H)+]. ¹H NMR (400 MHz, DMSO-d₆) δ: 1.57-1.65 (m, 12H, 2-CH₂CH₂CH₂—), 2.29 (s, 3H, CH₃), 3.76 (m, 8H, 2-CH₂NCH₂—), 7.26-7.82 (m, 3H, C₆H₃), 9.82 (s, 1H, 1NH).

(4-Chlorophenyl)-(4,6-dipiperidine-1-yl-[1,3,5]triazin-2-yl)-amine hydrochloride (Compound 584) (VIRADEM-011495): Yield 83%. C₁₉H₂₆Cl₂N₆. MS m/z: 373.6 [(M+H)+]. ¹H NMR (400 MHz, DMSO-d₆) δ: 1.56-1.65 (m, 12H, 2-CH₂CH₂CH₂—), 3.76 (m, 8H, 2-CH₂NCH₂—), 7.38 and 7.62 (d-d, 4H, C₆H₄), 9.69 (s, 1H, 1NH).

(3,5-Dichlorophenyl)-(4,6-dipiperidine-1-yl-[1,3,5]triazin-2-yl)-amine hydrochloride (Compound 585) (VIRADEM-011496): Yield 86%. M.p.=196-197° C. C₁₉H₂₅Cl₃N₆. MS m/z: 408.1 [(M+H)+]. ¹H NMR (400 MHz, DMSO-d₆) δ: 1.53-1.64 (m, 12H, 2-CH₂CH₂CH₂—), 3.74 (m, 8H, 2-CH₂NCH₂—), 7.15 (s, 1H, C₆H₃), 7.77 (s, 2H, C₆H₃), 9.99 (s, 1H, 1NH).

(4-Chloro-6-piperidine-1-yl-[1,3,5]triazin-2-yl)-(3,5-dimethylphenyl)-amine (Compound 586) (VIRADEM-011497): Yield 80%. C₁₆H₂₁Cl₂N₅. MS m/z: 355.0 [(M+H)+]. ¹H NMR (400 MHz, DMSO-d₆) δ: 1.60-1.67 (m, 6H, —CH₂CH₂CH₂—), 2.25 (s, 6H, 2CH₃), 3.80 (m, 4H, —CH₂NCH₂—), 6.77 (s, 1H, C₆H₃), 7.24 (s, 2H, C₆H₃), 10.2 (s, 1H, 1NH).

(4,6-Dipiperidine-1-yl-[1,3,5]triazin-2-yl)-(4-fluorophenyl)-amine hydrochloride (Compound 587) (VIRADEM-011498): Yield 78%. C₁₉H₂₆ClFN₆. MS m/z: 357.2 [(M+H)+]. ¹H NMR (400 MHz, DMSO-d₆) δ: 1.55-1.64 (m, 12H, 2-CH₂CH₂CH₂—), 3.76 (m, 8H, 2-CH₂NCH₂—), 7.18-7.60 (m, 4H, C₆H₄), 9.76 (s, 1H, 1NH).

(3-Chlorophenyl)-(4,6-dipiperidine-1-yl-[1,3,5]triazin-2-yl)-amine hydrochloride (Compound 588) (VIRADEM-011499): Yield 83%. M.p.=205-206° C. C₁₉H₂₆Cl₂N₆. MS m/z: 373.6 [(M+H)+]. ¹H NMR (400 MHz, DMSO-d₆) δ: 1.57-1.66 (m, 12H, 2-CH₂CH₂CH₂—), 3.77 (m, 8H, 2-CH₂NCH₂—), 7.29-8.02 (m, 4H, C₆H₄), 10.1 (s, 1H, 1NH).

(4-Chloro-6-piperidine-1-yl-[1,3,5]triazin-2-yl)-p-tolyl-amine (Compound 589) (VIRADEM-011500): Yield 77%. C₁₅H₁₈ClN₅. MS m/z: 304.5 [(M+H)+]. ¹H NMR (400 MHz, DMSO-d₆) δ: 1.60-1.67 (m, 6H, —CH₂CH₂CH₂—), 2.30 (s, 3H, CH₃), 3.78 (m, 4H, —CH₂NCH₂—), 7.18 and 7.50 (d-d, 4H, C₆H₄), 10.1 (s, 1H, 1NH).

N,N¹-Diisopropyl-6-piperidine-1-yl-[1,3,5]triazine-2,4-diamine (Compound 590) (VIRADEM-016575): Yield 67%. C₁₄H₂₆N₆. MS m/z: 279.2 [(M+H)+].

N-Ethyl-N¹-isopropyl-6-piperidine-1-yl-[1,3,5]triazine-2,4-diamine (Compound 591) (VIRADEM-023843): Yield 65%. C₁₃H₂₄N₆. MS m/z: 265.1 [(M+H)+].

(6-tert-Butyl-3-morpholin-4-yl-[1,2,4]triazin-5-yl)-phenyl-amine (Compound 592) (VIRADEM-013044): Yield 73%. C₁₇H₂₃N₅O. MS m/z: 314.3 [(M+H)+]. ¹H NMR (400 MHz, DMSO-d₆) δ: 1.42 (s, 9H, C(CH₃)₃), 3.51 (m, 4H, —CH₂NCH₂—), 3.60 (m, 4H, —CH₂OCH₂—), 7.15 (t, 1H, C₆H₅), 7.36 (t, 2H, C₆H₅), 7.50 (d, 2H, C₆H₅), 8.01 (s, 1H, 1NH).

(6-tert-Butyl-3-morpholin-4-yl-[1,2,4]triazin-5-yl)-(4-methoxyphenyl)-amine (Compound 593) (VIRADEM-023899): Yield 67%. C₁₈H₂₅N₅O₂. MS m/z: 344.3 [(M+H)+]. ¹H NMR (400 MHz, DMSO-d₆) δ: 1.41 (s, 9H, C(CH₃)₃), 3.52 (m, 4H, —CH₂NCH₂—), 3.61 (m, 4H, —CH₂OCH₂—), 6.89 and 7.55 (d-d, 4H, C₆H₄), 8.27 (s, 1H, 1NH).

(6-tert-Butyl-3-morpholin-4-yl-[1,2,4]triazin-5-yl)-(4-chlorophenyl)-amine (Compound 594) (VIRADEM-023900): Yield 79%. C₁₇H₂₂ClN₅O. MS m/z: 348.6 [(M+H)+]. ¹H NMR (400 MHz, DMSO-d₆) δ: 1.44 (s, 9H, C(CH₃)₃), 3.51 (m, 4H, —CH₂NCH₂—), 3.60 (m, 4H, —CH₂OCH₂—), 7.16 and 7.75 (d-d, 4H, C₆H₄), 8.12 (s, 1H, 1NH).

(6-tert-Butyl-3-morpholin-4-yl-[1,2,4]triazin-5-yl)-p-tolyl-amine (Compound 595) (VIRADEM-023901): Yield 77%. C₁₈H₂₅N₅O. MS m/z: 328.3 [(M+H)+]. ¹H NMR (400 MHz, DMSO-d₆) δ: 1.41 (s, 9H, C(CH₃)₃), 2.28 (s, 3H, CH₃), 3.52 (m, 4H, —CH₂NCH₂—), 3.61 (m, 4H, —CH₂OCH₂—), 7.14 and 7.51 (d-d, 4H, C₆H₄), 8.07 (s, 1H, 1NH).

(6-tert-Butyl-3-morpholin-4-yl-[1,2,4]triazin-5-yl)-(3,4-dimethylphenyl)-amine (Compound 596) (VIRADEM-023902): Yield 75%. C₁₉H₂₇N₅O. MS m/z: 342.3 [(M+H)+]. ¹H NMR (400 MHz, DMSO-d₆) δ: 1.42 (s, 9H, C(CH₃)₃), 2.16 (s, 3H, CH₃), 2.27 (s, 3H, CH₃), 3.50 (m, 4H, —CH₂NCH₂—), 3.59 (m, 4H, —CH₂OCH₂—), 7.09-7.42 (m, 3H, C₆H₃), 8.15 (s, 1H, 1NH).

(6-tert-Butyl-3-morpholin-4-yl-[1,2,4]triazin-5-yl)-(3,5-dimethylphenyl)-amine (Compound 597) (VIRADEM-023903): Yield 79%. C₁₉H₂₇N₅O. MS m/z: 342.3 [(M+H)+]. ¹H NMR (400 MHz, DMSO-d₆) δ: 1.41 (s, 9H, C(CH₃)₃), 2.25 (s, 6H, 2CH₃), 3.49 (m, 4H, —CH₂NCH₂—), 3.58 (m, 4H, —CH₂OCH₂—), 6.76 (s, 1H, C₆H₃), 7.36 (s, 1H, C₆H₃), 8.13 (s, 1H, 1NH).

(6-tert-Butyl-3-morpholin-4-yl-[1,2,4]triazin-5-yl)-(3,4-dimethoxyphenyl)-amine (Compound 598) (VIRADEM-023904): Yield 70%. C₁₉H₂₇N₅O₃. MS m/z: 374.3 [(M+H)+]. ¹H NMR (400 MHz, DMSO-d₆) δ: 1.41 (s, 9H, C(CH₃)₃), 3.51 (m, 4H, —CH₂NCH₂—), 3.60 (m, 4H, —CH₂OCH₂—), 3.76 (s, 2H, 2OCH₃), 6.85-7.41 (m, 3H, C₆H₃), 8.05 (s, 1H, 1NH).

(6-tert-Butyl-3-morpholin-4-yl-[1,2,4]triazin-5-yl)-m-tolyl-amine (Compound 599) (VIRADEM-023905): Yield 73%. C₁₈H₂₅N₅O. MS m/z: 328.3 [(M+H)+]. ¹H NMR (400 MHz, DMSO-d₆) δ: 1.42 (s, 9H, C(CH₃)₃), 2.25 (s, 3H, CH₃), 3.53 (m, 4H, —CH₂NCH₂—), 3.62 (m, 4H, —CH₂OCH₂—), 6.91 and 7.59 (d-d, 4H, C₆H₄), 8.09 (s, 1H, 1NH).

(2-Morpholin-4-yl-pyrimidin-4-yl)-phenylamine was obtained by the method [Tetrahedron.-2015.-v. 71.-1515-1522](Compound 600) (VIRADEM-023906): Yield 69%. M.p.=142-143° C. C₁₄H₁N₄O. MS m/z: 257.1 [(M+H)+].

(4-Morpholin-4-yl-pyrimidin-2-yl)-phenylamine was obtained by the method [Tetrahedron.-2015.-v. 71.-1515-1522](Compound 601) (VIRADEM-023907): Yield 65%. M.p.=131-132° C. C₁₄H₁N₄O. MS m/z: 257.2 [(M+H)+].

N²,N⁴-Diphenylpyrimidin-2,4-diamine was obtained by the method [Tetrahedron.-2015.-v. 71.-1515-1522](Compound 602) (VIRADEM-023908): C₁H₁₄N₄. MS m/z: 263.1 [(M+H)+].

N⁴-(3-Chlorophenyl)-N²-phenylpyrimidine-2,4-diamine was obtained by the method [Tetrahedron.-2015.-v. 71.-1515-1522](Compound 603) (VIRADEM-023909): C₁H₁₃ClN₄. MS m/z: 297.7 [(M+H)+].

(3-Chlorophenyl)-(2-morpholin-4-yl-pyrimidin-4-yl)-amine was obtained by the method [Tetrahedron.-2015.-v. 71.-1515-1522](Compound 604) (VIRADEM-023910): C₁₄H₁₅ClN₄O. MS m/z: 291.1 [(M+H)+].

N⁴-(3-Methoxyphenyl)-N²-phenylpyrimidine-2,4-diamine was obtained by the method [Tetrahedron.-2015.-v. 71.-1515-1522](Compound 605) (VIRADEM-023911): C₁₇H₁N₄O. MS m/z: 293.3 [(M+H)+].

(3-Methoxyphenyl)-(2-morpholin-4-yl-pyrimidine-4-yl)-amine was obtained by the method [Tetrahedron.-2015.-v. 71.-1515-1522](Compound 606) (VIRADEM-023912): C₁₅H₁₈N₄O₂. MS m/z: 287.2 [(M+H)+].

3-(2-Phenylaminopyrimidin-4-ylamino)-benzoic acid methyl ester was obtained by the method [Tetrahedron.-2015.-v. 71.-1515-1522](Compound 607) (VIRADEM-023913): C₁₈H₁N₄O₂. MS m/z: 321.1 [(M+H)+].

3-(2-Morpholin-4-yl-pyrimidin-4-ylamino)-benzoic acid methyl ester was obtained by the method [Tetrahedron.-2015.-v. 71.-1515-1522](Compound 608) (VIRADEM-023914): C₁H₁₈N₄O₃. MS m/z: 315.2 [(M+H)+].

N⁴-(2-Chlorophenyl)-N²-phenylpyrimidine-2,4-diamine was obtained by the method [Tetrahedron.-2015.-v. 71.-1515-1522](Compound 609) (VIRADEM-023915): C₁H₁₃ClN₄. MS m/z: 297.1 [(M+H)+].

(2-Chlorophenyl)-(2-morpholin-4-yl-pyrimidin-4-yl)-amine was obtained by the method [Tetrahedron.-2015.-v. 71.-1515-1522](Compound 610) (VIRADEM-023916): C₁₄H₁₅ClN₄O. MS m/z: 291.1 [(M+H)+].

Methyl-(2-morpholin-4-yl-pyrimidin-4-yl)-phenylamine was obtained by the method [Tetrahedron.-2015.-v. 71.-1515-1522](Compound 611) (VIRADEM-023917): C₁₅H₁₈N₄O. MS m/z: 271.2 [(M+H)+].

N²-Phenyl-N⁴-pyridin-3-yl-pyrimidine-2,4-diamine was obtained by the method [Tetrahedron.-2015.-v. 71.-1515-1522](Compound 612) (VIRADEM-023918): C₁₅H₁₃N₅. MS m/z: 264.1 [(M+H)+].

3-(2-Morpholin-4-yl-pyrimidin-4-yl)-pyridin-3-yl-amine was obtained by the method [Tetrahedron.-2015.-v. 71.-1515-1522](Compound 613) (VIRADEM-023919): C₁₃H₁₅N₅O. MS m/z: 258.2 [(M+H)+].

[4-(4-Methylpiperazine-1-yl)-pyrimidine-2-yl]-phenylamine was obtained by the method [Tetrahedron.-2015.-v. 71.-1515-1522](Compound 614) (VIRADEM-023920): C₁₅H₁₉1N. MS m/z: 270.2 [(M+H)+].

4-[4-(4-Methylpiperazin-1-yl)-pyrimidin-2-yl]-morpholine was obtained by the method [Tetrahedron.-2015.-v. 71.-1515-1522](Compound 615) (VIRADEM-023921): C₁₃H₂₁N₅O. MS m/z: 264.2 [(M+H)+].

4,41-(pyrimidine-2,4-diyl)dimorpholine was obtained by the method [Tetrahedron.-2015.-v. 71.-1515-1522](Compound 616) (VIRADEM-023922): C₁₂H₁₈N₄O₂. MS m/z: 251.1 [(M+H)+].

N⁴-Cyclopentyl-N²-phenylpyrimidine-2,4-diamine was obtained by the method [Tetrahedron.-2015.-v. 71.-1515-1522](Compound 617) (VIRADEM-023923): C₁₅H₁₈N₄. MS m/z: 255.2 [(M+H)+].

Cyclopentyl-(2-morpholin-4-yl-pyrimidine-4-yl)-amine was obtained by the method [Tetrahedron.-2015.-v. 71.-1515-1522](Compound 618) (VIRADEM-023924): C₁₃H₂₀N₄O. MS m/z: 249.2 [(M+H)+].

(3,4-Dimethylphenyl)-(2-morpholin-4-yl-pyrimidin-4-yl)-amine (Compound 619) (VIRADEM-023925): C₁H₂₀N₄O. MS m/z: 285.2 [(M+H)+].

(3,4-Dimethylphenyl)-(4-morpholin-4-yl-pyrimidin-2-yl)-amine (Compound 620) (VIRADEM-023926): C₁H₂₀N₄O. MS m/z: 285.2 [(M+H)+].

(3,4-Dimethylphenyl)-(5-methyl-2-morpholin-4-yl-pyrimidin-4-yl)-amine (Compound 621) (VIRADEM-023927): C₇H₂₂N₄O. MS m/z: 299.2 [(M+H)+].

1. A method of treating a disease caused by SARS-CoV-2 and other coronaviruses comprising administering to a subject in need thereof a therapeutically effective amount of a compound of Formula I: 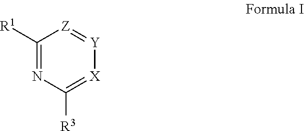
or a pharmaceutically acceptable salt thereof;
wherein:
each X, Y, and Z is independently selected from N or C—R²;
each R¹, R², and R³ is independently from the group consisting of hydrogen, halogen, nitro, cyano, hydroxyl, amino, alkyl, cycloalkyl, optionally substituted cycloalkyl, (cycloalkyl)alkyl, bicycloalkyl, spiroalkyl, optionally substituted alkyl, haloalkyl, hydroxyalkyl, alkoxy, haloalkoxy, alkylthio, alkoxyalkyl, (cyano)alkyl, alkenyl, optionally substituted alkenyl, alkynyl, cycloalkylenyl, optionally substituted alkynyl, sulfonamide, alkylcarbonyl, arylcarbonyl, alkylsulfonyl, arylsulfonyl, mercaptoalkyl, carboxy, carboxamido, ureido, (heterocyclo)alkyl, (heteroaryl)alkyl, (amino)(heteroaryl)alkyl, (amino)(aryl)alkyl, heteroalkyl, heterocyclo, optionally substituted heterocyclo, hetero-substituted cycloalkyl, hetero-substituted bicycloalkyl, heteroaryl, optionally substituted heteroaryl, hetero-substituted spiroalkyl, aryl, optionally substituted aryl, N-substituted optionally substituted aryl, N-substituted heteroaryl, N-substituted optionally substituted heteroaryl, aryloxy, heteroaryloxy, aralkyloxy, alkylamino, dialkylamino, hydroxyalkylamino, cycloalkylamino, aralkylamino, (cycloalkyl)alkylamino, aralkyl, (aryl)alkyl, (heterocyclo)alkylamino, (amino)alkyl, heteroalkylenyl, heterocyclenyl, N-aminoacid, 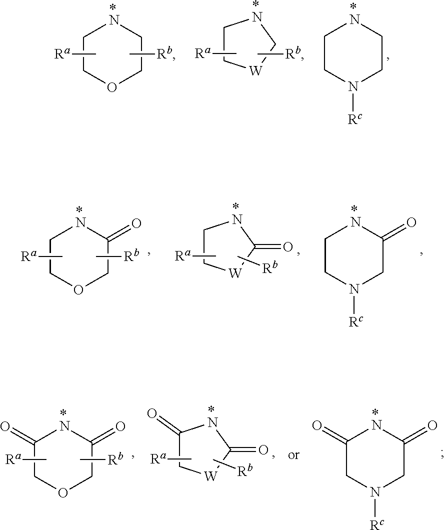
and
R¹ and R² could be the same, but, in this case, always different from R³,
wherein:
each R^{a}, R^{b}, and R^{c} independently selected from the group consisting of hydrogen, C₁-C₁₆ alkyl, or an optionally substituted alkyl, aryl, optionally substituted aryl, and heteroalkyl.

2. The method of claim 1, wherein the compound of Formula I is represented by Formula II: 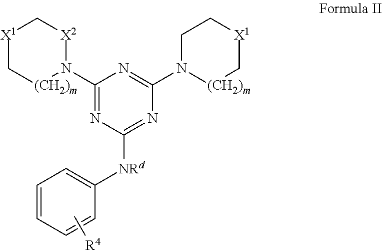
or a pharmaceutically acceptable salt thereof;
wherein:
each m is independently 0, 1, or 2;
each R^{d} is selected from the group consisting of hydrogen, C₁-C₁₆ alkyl, optionally substituted alkyl, aryl, optionally substituted aryl, and heteroalkyl;
each X¹ is independently selected from the group consisting of —O—, —CH₂—, and —N(R⁶)—;
wherein:
R⁶ is selected from hydrogen, C₁-C₁₆ alkyl, an optionally substituted alkyl, aryl, optionally substituted aryl, and heteroalkyl;
each X² is selected from —CH₂— and —C(═O)—; and
each R⁴ is selected from the group consisting of hydrogen, halogen, nitro, cyano, hydroxyl, amino, alkyl, cycloalkyl, optionally substituted cycloalkyl, (cycloalkyl)alkyl, bicycloalkyl, spiroalkyl, —NC(O)R⁶, and —OR⁶;
wherein:
R⁶ is selected from alkyl, optionally substituted alkyl, aryl, optionally substituted aryl, heteroalkyl, and optionally substituted heteroaryl.

3. The method of claim 1, wherein the compound of Formula I is represented by Formula III: 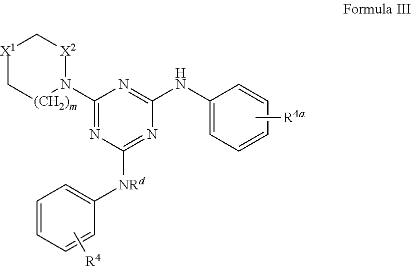
or a pharmaceutically acceptable salt thereof;
wherein:
each m is independently 0, 1, or 2;
each R^{d} is selected from the group consisting of hydrogen, C₁-C₁₆ alkyl, optionally substituted alkyl, aryl, optionally substituted aryl, and heteroalkyl;
each X¹ is independently selected from the group consisting of —O—, —CH₂—, and —N(R⁶)—;
wherein:
R⁶ is selected from hydrogen, C₁-C₁₆ alkyl, an optionally substituted alkyl, aryl, optionally substituted aryl, and heteroalkyl;
each X² is selected from —CH₂— and —C(═O)—; and
each R⁴ and R^{4a} is independently selected from the group consisting of hydrogen, halogen, nitro, cyano, hydroxyl, amino, alkyl, cycloalkyl, optionally substituted cycloalkyl, (cycloalkyl)alkyl, bicycloalkyl, spiroalkyl, —NC(O)R⁶, and —OR⁶;
wherein:
R⁶ is selected from alkyl, optionally substituted alkyl, aryl, optionally substituted aryl, heteroalkyl, and optionally substituted heteroaryl.

4. A method of treating a disease caused by a SARS-CoV-2 virus, said method comprising administering a therapeutically effective amount of a compound of claim 1.

5. A method of treating a disease caused by a SARS virus, said method comprising administering a therapeutically effective amount of a compound of claim 1.

6. A method of treating a disease caused by a MERS virus, said method comprising administering a therapeutically effective amount of a compound of claim 1.
